SEQUENCE LISTING (10 sequences)

SEQ ID NO: 1 - DNA, 735 bp, Artificial Sequence
  SARS-CoV-2 RBD (codon-optimized)
atgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccattgataa

SEQ ID NO: 2 - protein, 243 aa, Artificial Sequence
  SARS-CoV-2 RBD
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 3 - DNA, 3774 bp, Artificial Sequence
  SARS-CoV-2 Spike with CS and PP (codon-optimized)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatga

SEQ ID NO: 4 - protein, 1256 aa, Artificial Sequence
  SARS-CoV-2 Spike with CS and PP
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 5 - DNA, 3774 bp, Artificial Sequence
  SARS-CoV-2 Spike with CS (codon-optimized)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatga

SEQ ID NO: 6 - protein, 1256 aa, Artificial Sequence
  SARS-CoV-2 Spike with CS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 7 - DNA, 8529 bp, Artificial Sequence
  Plasmid Sequence
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaaggccaccatgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatgaaattcgagctcgcggccgcatcgatcttaagtcgcgactcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 8 - DNA, 5490 bp, Artificial Sequence
  Plasmid Sequence
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaaggccaccatgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccattgataaaattcgagctcgcggccgcatcgatcttaagtcgcgactcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 9 - DNA, 8529 bp, Artificial Sequence
  Plasmid Sequence
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaaggccaccatgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatgaaattcgagctcgcggccgcatcgatcttaagtcgcgactcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 10 - protein, 229 aa, Artificial Sequence
  SARS-CoV-2 Spike protein receptor binding domain and hexahistidine tag
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

(19) United States
(12) Patent Application Publication
(10) Pub. No.: US 2024/0210415 A1
(43) Pub. Date: Jun. 27, 2024

(54) RECOMBINANT SARS-COV-2 SPIKE PROTEIN AND USES THEREOF

(71) Applicant: ICAHN SCHOOL OF MEDICINE AT MOUNT SINAI, New York, NY (US)

(72) Inventors: Florian KRAMMER, New York, NY (US); Adolfo FIRPO-BETANCOURT, New York, NY (US); Carlos CORDON-CARDO, New York, NY (US); Damodara Rao MENDU, Ellicott City, MD (US); Viviana SIMON, New York, NY (US); Fatima AMANAT, Jamaica, NY (US); Daniel STADLBAUER, New York, NY (US)

(73) Assignee: ICAHN SCHOOL OF MEDICINE AT MOUNT SINAI, New York, NY (US)

(21) Appl. No.: 17/913,783

(22) PCT Filed: Mar. 17, 2021
(86) PCT No.: PCT/US2021/022848

§ 371 Date: Sep. 22, 2022

Related U.S. Application Data
(60) Provisional application No. 63/059,924, filed on Jul. 31, 2020.
(60) Provisional application No. 63/051,858, filed on Jul. 14, 2020.
(60) Provisional application No. 63/024,436, filed on May 13, 2020.
(60) Provisional application No. 63/020,503, filed on May 5, 2020.
(60) Provisional application No. 63/018,457, filed on Apr. 30, 2020.
(60) Provisional application No. 62/994,252, filed on Mar. 24, 2020.

(51) Int. Cl. G01N 33/68; G01N 1/34; G01N 1/44; G01N 21/59; G01N 33/543; G01N 33/569

(52) U.S. Cl. CPC G01N 33/6854; G01N 1/34; G01N 1/44; G01N 21/59; G01N 33/54366; G01N 33/56983; G01N 2333/165

(57) ABSTRACT

Provided herein are recombinant SARS-CoV-2 spike proteins, cells producing such proteins, and kits comprising such proteins. Also provided herein are compositions comprising recombinant SARS-CoV-2 spike proteins and methods of detecting antibodies using such SARS-CoV-2 spike proteins.

### CROSS-REFERENCE TO RELATED APPLICATIONS

This application claims the benefit of U.S. Provisional Application No. 63/059,924, filed Jul. 31, 2020, U.S. Provisional Application No. 63/051,858, filed Jul. 14, 2020, U.S. Provisional Application No. 63/024,436, filed May 13, 2020, U.S. Provisional Application No. 63/020,503, filed May 5, 2020, U.S. Provisional Application No. 63/018,457, filed Apr. 30, 2020, and U.S. Provisional Application No. 62/994,252, filed Mar. 24, 2020, the disclosure of each of which is incorporated by reference herein in its entirety.

### STATEMENT REGARDING FEDERALLY SPONSORED RESEARCH

This invention was made with government support under HHSN272201400008C and 75N93019C00051 awarded by the NIAID Centers of Excellence for Influenza Research and Surveillance (CEIRS) and Collaborative Influenza Vaccine Innovation Centers (CIVIC). The government has certain rights in the invention.

### SEQUENCE LISTING

This application incorporates by reference a Sequence Listing submitted with this application as a text file entitled “06923-342-228_SEQ_LISTING.txt,” was created on Mar. 14, 2021, and is 67,689 bytes in size.

### 1. INTRODUCTION

Provided herein are recombinant SARS-CoV-2 spike proteins, cells producing such proteins, and kits comprising such proteins. Also provided herein are compositions comprising recombinant SARS-CoV-2 spike proteins and methods of detecting antibodies using such SARS-CoV-2 spike proteins.

### 2. BACKGROUND

On Dec. 31, 2019, China reported first cases of atypical pneumonia in Wuhan, the capital of Hubei province. The causative virus was found to be a betacoronavirus, closely related to the severe acute respiratory syndrome coronavirus (SARS-CoV-1) from 2003 and similar to Sarbecoviruses isolated from bats (Wu et al, A new coronavirus associated with human respiratory disease in China. Nature 2020; Zhou et al., A pneumonia outbreak associated with a new coronavirus of probable bat origin. Nature 2020). It was therefore termed SARS-CoV-2 and the disease it causes was named COVID19 (Corona Virus Disease 2019; Gorbalenya et al. The species Severe acute respiratory syndrome-related coronavirus: classifying 2019-nCoV and naming it SARS-CoV-2. Nature Microbiology 2020). The outbreak in Wuhan expanded quickly and led to the lockdown of Wuhan, the Hubei province and other parts of China. While the lockdown, at least temporarily, brought the situation under control in China, SARS-CoV-2 spread globally causing a pandemic with 150,000 infections and 5,500 fatalities as of Mar. 16, 2020. As of Apr. 13, 2020, Severe acute respiratory syndrome coronavirus 2 (SARS-CoV-2), a Sarbecoviruses, has spread globally causing a pandemic with 2.5 million infections and 185,000 fatalities. As of May 13, 2020, more than 4,310,000 people have tested positive for SARS-CoV-2. In addition, as of May 13, 2020, more than 82,246 Americans have died from COVID-19 and globally more than 294,000 people have died from COVID-19. Currently, there is no vaccine or therapeutic to prevent or treat COVID-19. As of Jul. 31, 2020, more than 17.3 million people worldwide have tested positive for SARS-CoV-2 and approximately 674,000 people worldwide have died from COVID-19. There is an urgent need to develop therapeutics to treat COVID-19 and diagnostics to detect severe acute respiratory syndrome coronavirus-2 (SARS-CoV-2).

Nucleic acid tests that detect the SARS-CoV-2 RNA genome were quickly developed and are now widely employed to diagnose COVID19 disease (Chu et al., Molecular Diagnosis of a Novel Coronavirus (2019-nCoV) Causing an Outbreak of Pneumonia. ClIn Chem 2020; Corman et al., Detection of 2019 novel coronavirus (2019-nCoV) by real-time RT-PCR. Euro Surveill 2020; 25). However, there remains a great need for assays that measure antibody responses and determine seroconversion. While such serological assays are not well suited to detect acute infections, they support a number of highly relevant applications. First, serological assays allow us to study the immune response(s) to SARS-CoV-2 in a qualitative and quantitative manner. Second, serosurveys are needed to determine the precise rate of infection in an affected area, which is an essential variable to accurately determine the infection fatality rate. Third, serological assays will allow for the identification of individuals who mounted strong antibody responses and who could serve as donors for the generation of convalescent serum therapeutics. Lastly, serological assays can help inform studies that aim to identify antibody responses that correlate with disease protection. Serological assays will permit to determine who is immune and who is not. This would be very useful for deploying immune health care workers in a strategic manner as to limit the risk of exposure and spread of the virus inadvertently.

Sarbecoviruses express a large (approximately 140 kDa) glycoprotein termed spike protein (S, a homotrimer), which mediates binding to host cells via interactions with the human receptor angiotensin converting enzyme 2 (ACE2) (Letko et al., Functional assessment of cell entry and receptor usage for SARS-CoV-2 and other lineage B betacoronaviruses. Nat Microbiol 2020.6-8; Wrapp et al., Cryo-E M structure of the 2019-nCoV spike In the prefusion conformation. Science 2020; Walls et al., Structure, Function, and Antigenicity of the SARS-CoV-2 Spike Glycoprotein. Cell 20 20). The S protein is highly immunogenic with the receptor-binding domain (RBD) being the target of many neutralizing antibodies (Berry et al., Neutralizing epitopes of the SARS-CoV S-protein cluster independent of repertoire, antigen structure or mAb technology. MAbs 2010; 2:53-66). Individuals infected with coronaviruses typically mount neutralizing antibodies (Huang et al., A systemic review of antibody mediate immunity to coronaviruses: antibody kinetics, correlates of protection, and association of antibody responses with severity of disease. medRxiv, 2020.2004.2014.20065771 (2020)) and a neutralizing response has been demonstrated for SARS-CoV-2 in an individual case from day 9 onwards. (Haveri et al., Serological and molecular findings during SARS-CoV-2 infection: the first study in Finland, January to February 2020 (Eurosurveillance, 2020); Liu et al., Two-year prospective study of the humoral immune response of patients with severe acute respiratory syndrome. J Infect Dis 2006; 193:792-5; Callow et al., The time course of the immune response to experimental coronavirus infection of man. Epidemiol Infect 1990; 105:435-46; Choe et al., MERS-COV Antibody Responses 1 Year after Symptom Onset, South Korea, 2015. Emerg Infect Dis 2017; 23:1079-84). For human coronaviruses these responses have been linked to protection for a period of time and future studies will show if there is a correlation between neutralizing antibodies and protection from SARS-CoV-2 infection as well (Huang et al., A systemic review of antibody mediate immunity to coronaviruses: antibody kinetics, correlates of protection, and association of antibody responses with severity of disease. medRxiv, 2020.2004.2014.20065771 (2020)). Serum neutralization can be measured using replication competent virus but the process requires several days and must be conducted in a biosafety level 3 laboratory for containment of SARS-CoV-2. Potentially, pseudotyped viral particle based entry assays using lentiviruses or vesicular stomatitis virus could be used but these reagents are not trivial to produce.

Thus, there is a need for qualitative and quantitative serological assays for SARS-CoV-2.

### 3. SUMMARY

In one aspect, provided herein are recombinant SARS-CoV-2 spike proteins. In one embodiment, provided herein is a recombinant soluble SARS-CoV-2 spike protein, wherein the recombinant soluble SARS-CoV-2 spike protein comprises the receptor binding domain of a SARS-CoV-2 spike protein and a tag (e.g., a hexahistidine tag). In another embodiment, provided herein is a recombinant soluble SARS-CoV-2 spike protein, wherein the recombinant soluble SARS-CoV-2 spike protein comprises amino acid residues corresponding to amino acid residues 319-541 of the amino acid sequence found at GenBank Accession No. MN908947.3 and a tag (e.g., hexahistidine tag). In another embodiment, provided herein is a recombinant soluble SARS-CoV-2 spike protein, wherein the recombinant soluble SARS-CoV-2 spike protein comprises the ectodomain of a SARS-CoV-2 spike protein, a C-terminal cleavage site (e.g., C-terminal thrombin cleavage site), trimerization domain (e.g., T4 foldon trimerization domain), and a tag (e.g., hexahistidine tag), wherein the recombinant soluble SARS-CoV-2 spike protein does not contain a polybasic cleavage site. In some embodiments, the recombinant soluble SARS-CoV-2 spike protein does not include the signal sequence (e.g., amino acid residues 1-14 of GenBank No. MN908947.3). In another embodiment, provided herein is a recombinant soluble SARS-CoV-2 spike protein comprising amino acid residues corresponding to amino acid residues 1-1213 of the amino acid sequence of the SARS-CoV-2 spike protein found at GenBank Accession No. MN908947.3, a C-terminal thrombin cleavage site, T4 foldon trimerization domain and hexahistidine tag, wherein the recombinant soluble SARS-CoV-2 spike protein does not contain the polybasic cleavage site (RRAR). In another embodiment, provided herein is a recombinant soluble SARS-CoV-2 spike protein comprising amino acid residues corresponding to amino acid residues 15-1213 of the amino acid sequence of the SARS-CoV-2 spike protein found at GenBank Accession No. MN908947.3, a C-terminal thrombin cleavage site, T4 foldon trimerization domain and hexahistidine tag, wherein the recombinant soluble SARS-CoV-2 spike protein does not contain the polybasic cleavage site (RRAR). In a specific embodiment, the polybasic cleavage site (RRAR) is replaced by a single A. In certain embodiments, the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3. In some embodiments, the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3.

In another embodiment, provided herein is a recombinant soluble SARS-CoV-2 spike protein comprising amino acids 1-1213 of the spike protein found at GenBank Accession No. MN908947.3, a C-terminal thrombin cleavage site, T4 foldon trimerization domain and hexahistidine tag, wherein the protein does not contain the polybasic cleavage site (RRAR to A). In another embodiment, provided herein is a recombinant soluble SARS-CoV-2 spike protein comprising amino acids 15-1213 of the spike protein found at GenBank Accession No. MN908947.3, a C-terminal thrombin cleavage site, T4 foldon trimerization domain and hexahistidine tag, wherein the protein does not contain the polybasic cleavage site (RRAR to A). In another embodiment, provided herein is a recombinant soluble SARS-CoV-2 spike protein comprising amino acids 1-1213 of the spike protein found at GenBank Accession No. MN908947.3, a C-terminal thrombin cleavage site, T4 foldon trimerization domain and hexahistidine tag, wherein the protein does not contain the polybasic cleavage site (RRAR to A) and the protein contains two stabilizing mutations (K986P and V987P, wild type numbering). In another embodiment, provided herein is a recombinant soluble SARS-CoV-2 spike protein comprising amino acid residues 319-541 of the spike protein found at GenBank Accession No. MN908947.3 and a tag (e.g., hexahistidine tag). In a specific embodiment, provided herein is a recombinant SARS-CoV-2 spike protein comprising the amino acid sequence of SEQ ID NO: 2, 4, or 6. In a specific embodiment, provided herein is a recombinant SARS-CoV2 spike protein consisting of the amino acid sequence of SEQ ID NO: 2, 4, or 6. In a specific embodiment, provided herein is a recombinant SARS-CoV-2 spike protein comprising the amino acid sequence of SEQ ID NO: 10. In a specific embodiment, provided herein is a recombinant SARS-CoV2 spike protein consisting of the amino acid sequence of SEQ ID NO: 10. In a specific embodiment, provided herein is a recombinant SARS-CoV2 spike protein comprising (or consisting of) the amino acid sequence of SEQ ID NO: 2, 4, or 6 without the first 14 amino acid residues.

In another specific embodiment, provided herein is a recombinant SARS-CoV-2 spike protein comprising (or consisting of) the amino acid sequence set forth in GenBank Accession No. MT380725.1. In another specific embodiment, provided herein is a recombinant SARS-CoV-2 spike protein comprising (or consisting of) the amino acid sequence set forth in GenBank Accession No. MT380724.1.

In another aspect, provided herein are nucleic acid sequences encoding a recombinant SARS-CoV-2 spike protein described herein (e.g., the nucleotide sequences found in Section 8, infra). In one embodiment, provided herein is an isolated nucleic acid sequence comprising a nucleotide sequence encoding a recombinant soluble SARS-CoV-2 spike protein described herein. In a specific embodiment, provided herein is an isolated nucleic acid sequence comprising the nucleotide sequence of SEQ ID NO: 1, 3 or 5. In another specific embodiment, provided herein is a recombinant SARS-CoV-2 spike protein encoded by a nucleic acid sequence comprising (or consisting of) the nucleic acid sequence set forth in GenBank Accession No. MT380725.1. In another specific embodiment, provided herein is a recombinant SARS-CoV-2 spike protein encoded by a nucleic acid sequence comprising (or consisting of) the nucleic acid sequence set forth in GenBank Accession No. MT380724.1. In another embodiment, provided herein is a vector comprising a nucleotide sequence encoding a recombinant SARS-CoV-2 spike protein described herein (e.g., the sequences in Section 8, infra).

In another aspect, provided herein are cells transfected with a nucleic acid sequence comprising a nucleotide sequence encoding a recombinant SARS-CoV-2 spike protein described herein. In one embodiment, provided herein are isolated cells stably transfected with a nucleic acid sequence comprising a nucleotide sequence encoding a recombinant SARS-CoV-2 spike protein described herein. In another embodiment, provided herein are isolated cells engineered to express a recombinant SARS-CoV-2 spike protein described herein. The cells may be Vero, MDCK, 293 T, HeLa, CHO, Cos, 293, HEK293, or Expi293F cells. The cells may also be S2, High-Five or Sf9 insect cells. In a specific embodiment, the cells are cells described in Example 5, infra.

In another aspect, provided herein are compositions comprising a recombinant SARS-CoV-2 spike protein described herein. In a specific embodiment, provided herein is a composition comprising a recombinant soluble SARS-CoV-2 spike protein, wherein the recombinant soluble SARS-CoV-2 spike protein comprises the amino acid sequence of SEQ ID NO: 2, 4 or 6. In a specific embodiment, provided herein is a composition comprising a recombinant soluble SARS-CoV-2 spike protein, wherein the recombinant soluble SARS-CoV-2 spike protein comprises the amino acid sequence of SEQ ID NO: 10.

In another aspect, provided herein is a composition comprising a nucleic acid sequence comprising a nucleotide sequence encoding a recombinant soluble SARS-CoV-2 spike protein. In one embodiment, provided herein is a composition comprising a nucleic acid sequence comprising a nucleotide sequence encoding a recombinant soluble SARS-CoV-2 spike protein, wherein the recombinant soluble SARS-CoV-2 spike protein comprises the amino acid sequence of SEQ ID NO: 2, 4 or 6. In another embodiment, provided herein is a composition comprising a nucleic acid sequence comprising a nucleotide sequence encoding a recombinant soluble SARS-CoV-2 spike protein, wherein the recombinant soluble SARS-CoV-2 spike protein comprises the amino acid sequence of SEQ ID NO: 10. In another embodiment, provided herein is a composition comprising a nucleic acid sequence comprising the nucleotide sequence of SEQ ID NO: 1, 3 or 5. In another embodiment, provided herein is a composition comprising a vector comprising a nucleotide sequence encoding a recombinant soluble SARS-CoV-2 spike protein (e.g., a vector described in Section 8, infra). In another embodiment, provided herein is a composition comprising a vector comprising a nucleotide sequence encoding a recombinant soluble SARS-CoV-2 spike protein, wherein the recombinant soluble SARS-CoV-2 spike protein comprises the amino acid sequence of SEQ ID NO: 2, 4 or 6. In another embodiment, provided herein is a composition comprising a vector comprising a nucleotide sequence encoding a recombinant soluble SARS-CoV-2 spike protein, wherein the recombinant soluble SARS-CoV-2 spike protein comprises the amino acid sequence of SEQ ID NO: 10.

In another aspect, provided herein are methods for immunizing against SARS-CoV-2 using a recombinant SARS-CoV-2 spike protein. In a specific embodiment, provided herein is a method for immunizing against SARS-CoV-2, comprising administering to a subject (e.g., a human) a recombinant soluble SARS-CoV-2 spike protein described herein, or a nucleic acid sequence comprising a nucleotide sequence encoding a recombinant soluble SARS-CoV-2 spike protein described herein.

In another aspect, provided herein are methods for inducing an immune response against SARS-CoV-2 using a recombinant SARS-CoV-2 spike protein. In a specific embodiment, provided herein is a method for inducing an immune response against SARS-CoV-2, comprising administering to a subject (e.g., a human) a recombinant soluble SARS-CoV-2 spike protein described herein, or a nucleic acid sequence comprising a nucleotide sequence encoding a recombinant soluble SARS-CoV-2 spike protein described herein.

In another aspect, provided herein are methods for preventing COVID-19 using a recombinant SARS-CoV-2 spike protein. In a specific embodiment, provided herein is a method preventing COVID-19, comprising administering to a subject (e.g., a human) a recombinant soluble SARS-CoV-2 spike protein described herein, or a nucleic acid sequence comprising a nucleotide sequence encoding a recombinant soluble SARS-CoV-2 spike protein described herein.

In another aspect, provided herein are methods for detecting antibody that binds to SARS-CoV-2 spike protein. In one embodiment, provided herein is a method for detecting an antibody that specifically binds to SARS-CoV-2 spike protein, comprising contacting a recombinant soluble SARS-CoV-2 spike protein with a biological sample obtained from a subject (e.g. a human) and detecting the binding of antibody(ies) present in the biological sample to the recombinant soluble SARS-CoV-2 spike protein. In a specific embodiment, the recombinant soluble SARS-CoV-2 spike protein comprises the amino acid sequence of SEQ ID NO: 2, 4, or 6. In another specific embodiment, the recombinant soluble SARS-CoV-2 spike protein comprises the amino acid sequence of SEQ ID NO: 10. In one embodiment, the biological sample is plasma or sera. In some embodiments, the method comprises quantitating the amount of antibody.

In one embodiment, provided herein is a method for detecting an antibody that specifically binds to SARS-CoV-2 spike protein, comprising: (1) incubating a specimen in a well coated with a recombinant SARS-CoV-2 spike protein for a first period time; (2) washing the well; (3) incubating a labeled antibody that binds to an isotype or subtype of immunoglobulin in the well for a second period of time; (4) washing the well; and (5) detecting the binding of the labeled antibody to the recombinant SARS-CoV-2 spike protein in the well. In certain embodiments, the specimen is a biological sample or antibody sample. The biological sample may be blood, sera or plasma from a subject (e.g., a human). The biological sample may be inactivated. In a specific embodiment, the recombinant SARS-CoV-2 spike protein comprises the amino acid sequence of SEQ ID NO: 2, 4, or 6. In another specific embodiment, the recombinant SARS-CoV-2 spike protein consists of the amino acid sequence of SEQ ID NO: 2, 4, or 6. In another specific embodiment, the recombinant SARS-CoV-2 spike protein comprises (or consists of) the amino acid sequence of SEQ ID NO: 2, 4, or 6 without the first 14 amino acid residues. In a specific embodiment, the recombinant SARS-CoV-2 spike protein comprises the amino acid sequence of SEQ ID NO: 10. In another specific embodiment, the recombinant SARS-CoV-2 spike protein consists of the amino acid sequence of SEQ ID NO: 10.

In another embodiment, provided herein is a method for the detection of antibody that specifically binds to human SARS-CoV-2 spike protein, comprising: (a) incubating a specimen in a well coated with a first recombinant soluble SARS-CoV-2 spike protein for a first period of time, wherein the first recombinant soluble SARS-CoV-2 spike protein comprises amino acid residues corresponding to amino acid residues 319-541 of GenBank Accession No. MN908947.3 and a tag (e.g., hexahistidine tag); (b) washing the well; (c) incubating a first labeled antibody that binds to an isotype or subtype of an immunoglobulin for a second period of time in the well; (d) washing the well; (e) detecting the binding of the first labeled antibody to the first recombinant soluble SARS-CoV-2 spike protein in the well. In a specific embodiment, the method comprises the steps for the ELISA described in Section 6, infra. In certain embodiments, if binding of the labeled antibody to the recombinant soluble SARS-CoV-2 spike protein is detected, then the method may further comprising: (f) incubating the specimen in a well coated with a second recombinant soluble SARS-CoV-2 spike protein for a third period of time, wherein the second recombinant soluble SARS-CoV-2 spike protein comprises amino acid residues corresponding to amino acid residues 15-1213 or amino acid residues 1-1213 of GenBank Accession No. MN908947.3, a C-terminal cleavage site (e.g., C-terminal thrombin cleavage site), trimerization domain (e.g., T4 foldon trimerization domain), and a tag (e.g., hexahistidine tag), and wherein the recombinant soluble SARS-CoV-2 spike protein does not contain a polybasic cleavage site; (g) washing the well; (h) incubating a second labeled antibody that binds to an isotype or subtype of an immunoglobulin for a fourth period of time in the well; (i) washing the well; and (j) detecting the binding of the labeled antibody to the second recombinant soluble SARS-CoV-2 spike protein in the well. In certain embodiments, the second recombinant soluble SARS-CoV-2 spike protein does not comprise a signal sequence (e.g., amino acid residues corresponding to amino acid residues 1-14 of GenBank Accession No. MN908947.3. In some embodiments, the first recombinant soluble SARS-CoV-2 spike protein, the second recombinant soluble SARS-CoV-2 spike protein, or both do not comprise a tag. In some embodiments, the specimen is considered positive if the binding of the first and second labeled antibodies to the first and second recombinant soluble SARS-CoV-2 spike protein is detected. In certain embodiments, the polybasic cleavage site (RRAR) is replaced by a single A. In certain embodiments, the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3. In some embodiments, the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3. In a specific embodiment, the first recombinant soluble SARS-CoV-2 spike protein comprises the amino acid sequence of SEQ ID NO:2. In another specific embodiment, the first recombinant soluble SARS-CoV-2 spike protein comprises the amino acid sequence of SEQ ID NO:10. In another specific embodiment, the second recombinant soluble SARS-CoV-2 spike protein comprises the amino acid sequence of SEQ ID NO: 4 or 6. In another specific embodiment, the second recombinant soluble SARS-CoV-2 spike protein comprises the amino acid sequence of SEQ ID NO: 4 or 6 without the first 14 amino acid residues. The first and second labeled antibodies may be the same or different. The first time period and second time period may be 30 minutes, 45 minutes, 1 hour, 2 hours, 2.5 hours or 3 hours. In a specific embodiment, the specimen is a biological sample. The biological sample may be blood, sera, or plasma. In certain embodiments, the biological sample is inactivated before being incubated with the well coated with recombinant soluble SARS-CoV-2 spike protein. In some embodiments, the specimen is serially diluted. The third and fourth time periods may be 30 minutes, 45 minutes, 1 hour, 2 hours, 2.5 hours or 3 hours.

In another embodiment, provided herein is a method for the detection of antibody that specifically binds to human SARS-CoV-2 spike protein, comprising: (a) incubating a specimen in a well coated with a first recombinant soluble SARS-CoV-2 spike protein for a first period of time, wherein the first recombinant soluble SARS-CoV-2 spike protein comprises amino acid residues corresponding to amino acid residues 319-541 of GenBank Accession No. MN908947.3 and a tag (e.g., hexahistidine tag); (b) washing the well; (c) incubating a first labeled antibody that binds to an isotype or subtype of an immunoglobulin for a second period of time in the well; (d) washing the well; (e) detecting the binding of the first labeled antibody to the first recombinant soluble SARS-CoV-2 spike protein; (f) incubating the specimen in a well coated with a second recombinant soluble SARS-CoV-2 spike protein for a third period of time, wherein the second recombinant soluble SARS-CoV-2 spike protein comprises amino acid residues corresponding to amino acid residues 15-1213 or amino acid residues 1-1213 of GenBank Accession No. MN908947.3, a C-terminal cleavage site (e.g., C-terminal thrombin cleavage site), trimerization domain (e.g., T4 foldon trimerization domain), and a tag (e.g., hexahistidine tag), and wherein the recombinant soluble SARS-CoV-2 spike protein does not contain a polybasic cleavage site; (g) washing the well; (h) incubating a second labeled antibody that binds to an isotype or subtype of an immunoglobulin for a fourth period of time in the well; (i) washing the well; and (j) detecting the binding of the second labeled antibody to the second recombinant soluble SARS-CoV-2 spike protein. In certain embodiments, the second recombinant soluble SARS-CoV-2 spike protein does not comprise a signal sequence (e.g., amino acid residues corresponding to amino acid residues 1-14 of GenBank Accession No. MN908947.3. In some embodiments, the first recombinant soluble SARS-CoV-2 spike protein, the second recombinant soluble SARS-CoV-2 spike protein, or both do not comprise a tag. In certain embodiments, the polybasic cleavage site (RRAR) is replaced by a single A. In certain embodiments, the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3. In some embodiments, the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3. In a specific embodiment, the first recombinant soluble SARS-CoV-2 spike protein comprises the amino acid sequence of SEQ ID NO:2. In another specific embodiment, the first recombinant soluble SARS-CoV-2spike protein comprises the amino acid sequence of SEQ ID NO:10. In another specific embodiment, the second recombinant soluble SARS-CoV-2 spike protein comprises the amino acid sequence of SEQ ID NO: 4 or 6. In another specific embodiment, the second recombinant soluble SARS-CoV-2 spike protein comprises the amino acid sequence of SEQ ID NO: 4 or 6 without the first 14 amino acid residues. The first and second labeled antibodies may be the same or different. The first time period and second time period may be 30 minutes, 45 minutes, 1 hour, 2 hours, 2.5 hours or 3 hours. In a specific embodiment, the specimen is a biological sample. The biological sample may be blood, sera, or plasma. In certain embodiments, the biological sample is inactivated before being incubated with the well coated with recombinant soluble SARS-CoV-2 spike protein. In some embodiments, the specimen is serially diluted. The third and fourth time periods may be 30 minutes, 45 minutes, 1 hour, 2 hours, 2.5 hours or 3 hours. The third or fourth periods may be 30 minutes, 45 minutes, 1 hour, 2 hours, 2.5 hours or 3 hours.

In another embodiment, provided herein is a method for the detection of antibody that specifically binds to human SARS-CoV-2 spike protein, comprising: (a) incubating a specimen in a well coated with a first recombinant soluble SARS-CoV-2 spike protein for a first period of time, wherein the first recombinant soluble SARS-CoV-2 spike protein comprises amino acid residues 319-541 of GenBank Accession No. MN908947.3 and a tag (e.g., hexahistidine tag); (b) washing the well; (c) incubating a first labeled antibody that binds to an isotype or subtype of an immunoglobulin for a second period of time in the well; (d) washing the well; (e) detecting the binding of the first labeled antibody to the first recombinant SARS-CoV-2 spike protein; (f) incubating the specimen in a well coated with a second recombinant soluble SARS-CoV-2 spike protein for a third period of time, wherein the second recombinant soluble SARS-CoV-2 spike protein comprises amino acid residues 15-1213 or amino acid residues 1-1213 of GenBank Accession No. MN908947.3, a C-terminal cleavage site (e.g., C-terminal thrombin cleavage site), trimerization domain (e.g., T4 foldon trimerization domain), and a tag (e.g., hexahistidine tag), and wherein the second recombinant soluble SARS-CoV-2 spike protein does not contain a polybasic cleavage site; (g) washing the well; (h) incubating a second labeled antibody that binds to an isotype or subtype of an immunoglobulin for a fourth period of time in the well; (i) washing the well; and (j) detecting the binding of the second labeled antibody to the second recombinant soluble SARS-CoV-2 spike protein. In certain embodiments, the second recombinant soluble SARS-CoV-2 spike protein does not comprise a signal sequence (e.g., amino acid residues 1-14 of GenBank Accession No. MN908947.3). In some embodiments, the first recombinant soluble SARS-CoV-2 spike protein, the second recombinant soluble SARS-CoV-2 spike protein, or both do not comprise a tag. In certain embodiments, the polybasic cleavage site (RRAR) is replaced by a single A. In certain embodiments, the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3. In some embodiments, the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3. In a specific embodiment, the first recombinant soluble SARS-CoV-2 spike protein comprises the amino acid sequence of SEQ ID NO:2. In a specific embodiment, the first recombinant soluble SARS-CoV-2 spike protein comprises the amino acid sequence of SEQ ID NO:10. In another specific embodiment, the second recombinant soluble SARS-CoV-2 spike protein comprises the amino acid sequence of SEQ ID NO: 4 or 6. In another specific embodiment, the second recombinant soluble SARS-CoV-2 spike protein comprises the amino acid sequence of SEQ ID NO: 4 or 6 without the first 14 amino acid residues. The first and second labeled antibodies may be the same or different. The first time period and second time period may be 30 minutes, 45 minutes, 1 hour, 2 hours, 2.5 hours or 3 hours. In a specific embodiment, the specimen is a biological sample. The biological sample may be blood, sera, or plasma. In certain embodiments, the biological sample is inactivated before being incubated with the well coated with the recombinant soluble SARS-CoV-2 spike protein. In some embodiments, the specimen is serially diluted. The third and fourth time periods may be 30 minutes, 45 minutes, 1 hour, 2 hours, 2.5 hours or 3 hours. The third or fourth time period may be 30 minutes, 45 minutes, 1 hour, 2 hours, 2.5 hours or 3 hours

In another aspect, provided herein is a kit comprises in one or more containers: (a) a multi-well (e.g., a 96 well) microtiter ELISA plate coated with a first recombinant soluble SARS-CoV-2 spike protein, wherein the first recombinant soluble SARS-CoV-2 spike protein comprises amino acid residues 319-541 of GenBank Accession No. MN908947.3 and a tag (e.g., hexahistidine tag); and (b) a multi-well (e.g., a 96 well) ELISA microtiter plate coated with a second recombinant soluble SARS-CoV-2 spike protein, wherein the second recombinant soluble SARS-CoV-2 spike protein comprises amino acid residues 15-1213 or amino acid residues 1-1213 of GenBank Accession No. MN908947.3, a C-terminal cleavage site (e.g., C-terminal thrombin cleavage site), trimerization domain (e.g., T4 foldon trimerization domain), and a tag (e.g., hexahistidine tag), and wherein the second recombinant soluble SARS-CoV-2 spike protein does not contain a polybasic cleavage site. In certain embodiments, the second recombinant soluble SARS-CoV-2 spike protein does not comprise a signal sequence (e.g., amino acid residues 1-14 of GenBank Accession No. MN908947.3. In some embodiments, the first recombinant soluble SARS-CoV-2 spike protein, the second recombinant soluble SARS-CoV-2 spike protein, or both do not comprise a tag. In a specific embodiment, the polybasic cleavage site (RRAR) is replaced by a single A. In some embodiments, the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3. In certain embodiments, the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3. In a specific embodiment, the first recombinant soluble SARS-CoV-2 spike protein comprises the amino acid sequence of SEQ ID NO:2. In a specific embodiment, the first recombinant soluble SARS-CoV-2 spike protein comprises the amino acid sequence of SEQ ID NO:10. In another specific embodiment, the second recombinant soluble SARS-CoV-2 spike protein comprises the amino acid sequence of SEQ ID NO:4 or 6. In another specific embodiment, the second recombinant soluble SARS-CoV-2 spike protein comprises the amino acid sequence of SEQ ID NO:4 or 6 without the first 14 amino acid residues. In some embodiments, the kit further comprises a labeled secondary antibody. In certain embodiments, the labeled secondary antibody is anti-human IgG horseradish perioxidase or alkaline phosphatase. In some embodiments, the kit further comprises o-pheylenediamine dihydrochloride. In certain embodiments, the kit further comprises a positive control antibody that binds to the recombinant soluble SARS-CoV-2 spike protein. The positive control antibody may be monoclonal antibody CR3022 or antibodies from COVID-19 patients. In some embodiments, the kit further comprises a negative control antibody. In certain embodiments, the kit comprises calibrators, such as described in Section 6, infra.

In a specific embodiment, a kit provided herein is one described in Section 6, infra (e.g., Example 11, 12 or 13).

### 4. BRIEF DESCRIPTION OF THE DRAWINGS

FIG. 1. Graphical protocol overview.

FIG. 2. RBD screening ELISA reference plate layout. The layout in which samples should be prepared in a 96-well, cell culture plate (dilution plate) is shown. Wells designated for positive (+) and negative (−) controls are indicated.

FIG. 3. Confirmatory Spike ELISA reference plate layout. The sample layout on the ELISA plate is shown, including the serial dilution steps that need to be performed. Wells designated for positive (+) and negative (−) controls are indicated.

FIGS. 4A-4B. Vector map showing the pCAGGS expression vectors. FIG. 4A shows the plasmid map of pCAGGS containing the sequence of the stabilized, soluble Spike. The schematic below indicates the signal peptide, receptor binding domain, ectodomain with stabilizing mutations, thrombin cleavage site, T4 trimerization domain and His-tag. FIG. 4B illustrates the pCAGGS vector encoding for the soluble receptor binding domain. The signal peptide, receptor binding domain and His-tag are indicated.

FIGS. 5A-5F. Constructs for recombinant protein expression. FIG. 5A. Visualization of the trimeric spike protein of SARS-CoV-2 based on PBD #6VXX using Pymol (Walls et al., Structure, Function, and Antigenicity of the SARS-CoV-2 Spike Glycoprotein. Cell (2020)). One monomer is colored in dark grey while the remaining two monomers are held in light grey. The receptor binding domain (RBD) of the dark grey trimer noted. FIG. 5B. Schematic of the wild type full length spike protein with signal peptide, ectodomain, receptor binding domain, furin cleavage site, S1, 52, and transmembrane and endodomain domain indicated. FIG. 5C. Schematic of the soluble trimeric spike. The polybasic/furin cleavage site (RRAR) was replaced by a single A. The transmembrane and endodomain were replaced by a furin cleavage site, a T4 foldon tetramerization domain and a hexahistidine tag. Introduction of K986P and V987P has been shown to stabilize the trimer in the pre-fusion conformation. FIG. 5D. Schematic of the soluble receptor binding domain construct. All constructs are to scale. FIG. 5E. Reducing SDS PAGE of insect cell and mammalian cell derived soluble trimerized spike protein (iSpike and rS pike). FIG. 5F. Reducing SDS PAGE of insect cell derived and mammalian cell derived recombinant receptor binding domain (iRBD and mRBD). Experiments were performed six times with the same result.

FIGS. 6A-6H. Reactivity of control and SARS-CoV-2 convalescent sera to different spike antigens. FIGS. 6A-6D. Reactivity to insect cell derived RBD (iRBD), mammalian cell derived RBD (mRBD), insect cell derived soluble spike protein {iSpike) and mammalian cell derived soluble spike protein (sSpike). Sera from three SARS-CoV-2 infected individuals were used and are shown in shades of grey. Two samples are from the same patient but from different time points (SARS-CoV-2 #3A and #3B). One sample, shown in green, is a convalescent serum sample post NL63 infection. FIGS. 6E-6H shows data from the same experiment but graphed as area under the curve (AUC) to get a better quantitative impression. The n for the control samples is 50 except for the iRBD were it is 59. Statistics were performed using a student's t-test in Graphpad Prism.

FIGS. 7A-7B. Isotypes and subtypes of antibodies from SARS-CoV-2 convalescent sera to the soluble spike protein. Insect cell derived (FIG. 7A) and mammalian cell derived (FIG. 7B) spike protein was used to study isotype/subclass distribution of antibodies. The different samples are indicated by different symbols. Sera from three SARS-CoV-2 infected individuals were used and are shown in shades of black. Two samples are from the same patient but from different time points (SARS-CoV-2 #3A and #3B).

FIGS. 8A-8K. Reactivity of control and SARS-CoV-2 convalescent sera to different spike antigens. FIGS. 8A-8D. Reactivity to insect cell derived RBD (iRBD), mammalian cell derived RBD (mRBD), insect cell derived soluble spike protein (iSpike) and mammalian cell derived soluble spike protein (mSpike). Sera from SARS-CoV-2 infected individuals are shown in black or dark grey. One sample, shown inlight grey, is a convalescent serum sample post NL63 infection. FIGS. 8E-8H shows data from the same experiment but graphed as area under the curve (AUC) to get a better quantitative impression. Then for the control samples is 50 except for the iRBD where it is 59. For iRBD and iSpike the n of convalescent samples was 4, for mRDB and mSpike the n was 16. Statistics were performed using an unpaired two-tailed student's t-test in Graphpad Prism, lines represent the mean. FIGS. 8I-8J shows reactivity of the 50 negative control samples from A-F against spike protein from human coronaviruses 229E and NL63. FIG. 8K. Correlation between ELISA titers and microneutralization titers (n=12, the three samples from negative control sera overlap and are displayed as single point). Statistics were performed using Pearson's rank test in Graphpad Prism. Experiments were performed once.

FIGS. 9A-9D. Effect of heat treatment and serum versus plasma on assay performance. FIGS. 9A-9B. Reactivity of paired non-treated serum and heat treated serum samples to mRBD and mSpike of SARS-CoV-2 (n=5). FIGS. 9C-9D. Reactivity of paired serum and plasma samples to mRBD and mSpike of SARS-CoV-2 (n=7). Statistics were performed using a paired two-tailed student's t-test in Graphpad Prism. Experiments were performed once.

FIGS. 10A-10D. Human normal immunoglobulin preparations and historic sera from HIV+ patients do not react with the SAR-CoV-2 spike. FIGS. 10A-10B. Reactivity of 21 different pools of human normal immunoglobulin (HNIG) preparations (27 different vials) to mRBO and mSpike of SARS-CoV-2. MAb CR3022 was used as positive control, three different irrelevant human mAbs were used as negative control. FIGS. 10C-10D shows reactivity of historic samples from 50 HIV+ individuals to mRBO and mSpike of SARS-CoV-2. Both HNIG and serum samples from HIV+ donors were collected before the SARS-CoV-2 pandemic. MAb CR3022 was used as positive control at a starting concentration of 100 μg/ml. Of note, the experiments in A and C as well as B and D were done at the same time and their positive contras are shared and displayed in both panels. Experiments were performed once.

FIGS. 11A-11B. Isotypes and subtypes of antibodies from COVID19 patients to the soluble spike protein and microneutralization titers. FIG. 11A. Mammalian cell derived spike protein was used to study isotype/subclass distribution of antibodies (n=13 positive samples). Lines represent the geometric mean. FIG. 11B. Microneutralization assay (n=12) performed with authentic SARS-CoV-2. Lines represent curves fitted using an inhibitor (log) versus response variable slope with four parameters function in Graphpad Prism. Experiments were performed once.

FIG. 12. Results of OD490 readouts from serial dilutions of reference standard was plotted on 4PL curves to determine their IC50.

FIG. 13. Representative standard curve for reference standard.

FIG. 14. Sixty previously tested specimens were analyzed as per the currently in-use qualitative EUA procedure. The raw data was analyzed by GraphPad Prism (version 8) and Bioteck Gen 5 software. Both statistical packages used 4-PL curves and data was compared.

FIG. 15. Assay precision within day and between day precision evaluated using low controls for quantitative ELISA.

FIG. 16. Assay precision within day and between day precision evaluated using medium controls for quantitative ELISA.

FIG. 17. Assay precision within day and between day precision evaluated using high controls for quantitative ELISA.

FIGS. 18A-18B. The linearity of the analytical measurement range (AMR) was evaluated for two patients specimens serum with various SARS-CoV-2 IgG antibody concentrations diluted with phosphate buffered saline serially (1:2, 1:4, 1:8, 1:16, and 1:32) to obtain values that cover the AMR and clinically reportable rante (CRR).

FIG. 19. COVAB Detection Performance. Results for a study comparing paired plasma and serum specimens from five SARS-CoV-2 antibody titer positive patients are shown in the table depicted for the RBD screen and titers to 1:2880.

FIGS. 20A-20C. Comparison of neutralization activity of patient serum with ELISA endpoints.

FIGS. 21A-21B. SARS-CoV-2 spike antibody titers in 19,860 individuals. FIG. 21A shows the percentage of individuals with antibody titers of 1:80 (low), 1:160 (low), 1:320 (moderate), 1:960 (high) and ≥1:2880 (high). FIG. 21B Absolute numbers and percent of individuals with titers of 1:320 over time. Over time, the screening program shifted from plasma donors (mild to moderate cases) to employee screening (with likely a higher number of asymptomatic infections) which likely caused the decrease and fluctuation in % above 1:320 at later time points.

FIGS. 22A-22B. Neutralizing activity of serum samples in relation to ELISA titers. FIG. 22A shows a correlation analysis between ELISA titers on the x-axis and neutralization titers in a microneutralization assay on the y-axis. The Spearman r was determined. FIG. 22B shows the proportion of sera that exert any neutralizing activity in each of the ELISA titer categories.

FIGS. 23A-23F Antibody titer stability over time. FIG. 23A shows titers of 121 volunteers who were initially bled approximately 30 days post COVID-19 symptom onset and were then recalled and bled again approximately 82 days post symptom onset. FIG. 23B, FIG. 23C, FIG. 23D, FIG. 23E and FIG. 23F shows the same data but stratified by initial/day 30 titer. Titers are graphed as geometric mean titers (GMT).

FIG. 24. Exemplary 7-point standard curve generated by reducing exemplary data using computer software capable of generating a four-parameter logistic (4-PL) curve fit.

FIG. 25. Detection of Antibody Class Specificity. This figure shows the class specificity of the monocal antibody in an antigen-down ELISA study.

FIGS. 26A-26B. Percentage of Samples with Neutralizing Activity by Concentration. This figure shows the correlation of the quantitative levels of anti-Spike protein IgG antibodies to viral neutralization in a microneutralization assay.

FIG. 27. Representative standard curve using reference standard.

### 5. DETAILED DESCRIPTION

### 5.1 Recombinant SARS-CoV-2 Spike Proteins

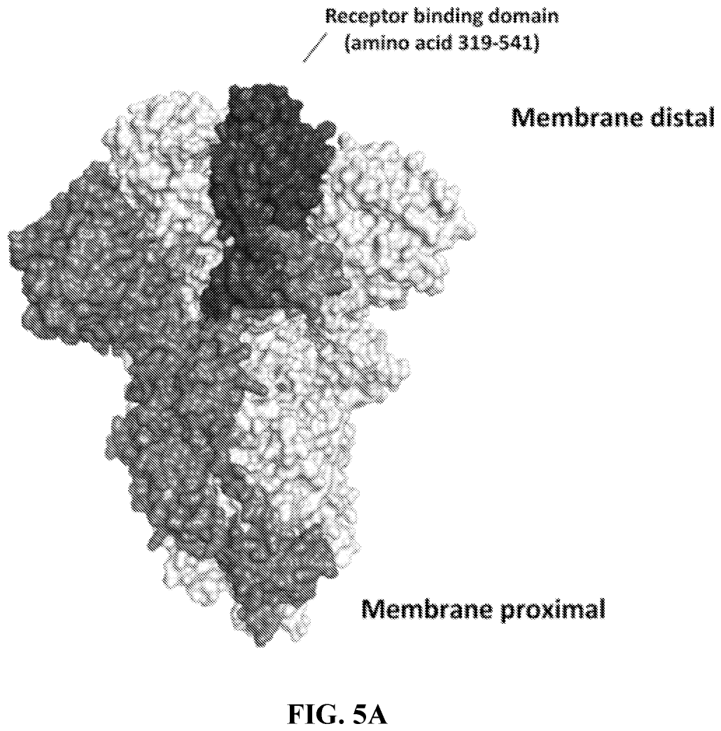

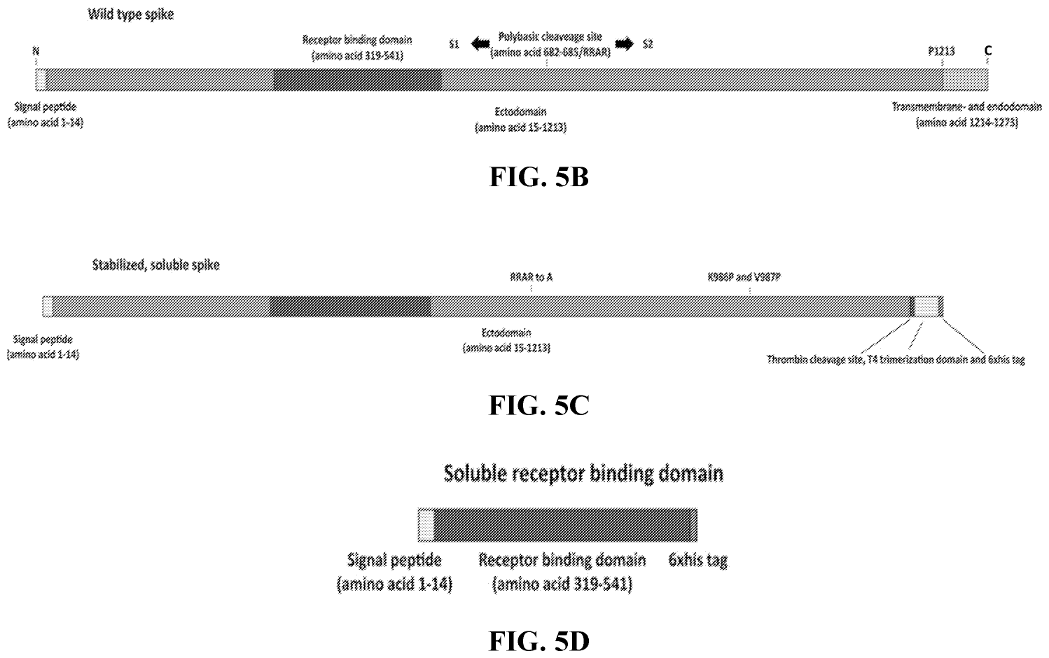

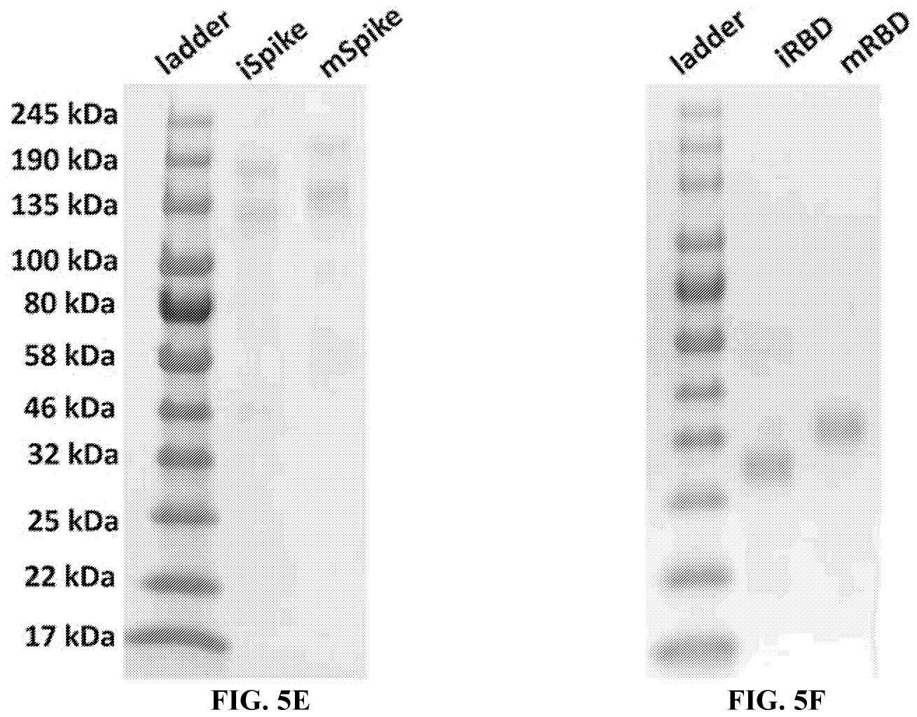

In one aspect, provided herein are recombinant severe acute respiratory syndrome coronavirus-2 (SARS-CoV-2) spike proteins. See Example 1, Example 2, Example 3, Example 4, Example 5, Example 7, and Example 8 infra, for examples of recombinant SARS-CoV-2 spike proteins encompassed herein. In one embodiment, such a recombinant SARS-CoV-2 spike protein is a soluble protein. In a specific embodiment, a recombinant SARS-CoV-2 spike protein is one described in Example 1, Example 2, Example 3, Example 4, Example 5, Example 7, and Example 8, infra. In another specific embodiment, a recombinant SARS-CoV-2 spike protein is one described in FIG. 5C or 5D. In another specific embodiment, a recombinant SARS-CoV-2 spike protein comprises an amino acid sequence described herein. In another specific embodiment, a recombinant SARS-CoV-2 spike protein is encoded by a nucleotide sequence comprising a nucleotide sequence described herein.

In one embodiment, provided herein is a recombinant SARS-CoV-2 spike protein described herein maintains the structure of a SARS-CoV-2 spike protein found in nature. In certain embodiments, a SARS-CoV-2 spike protein is not a full length SARS-CoV-2 spike protein found in nature. In specific embodiments, a SARS-CoV-2 spike protein described herein has been altered by man by, e.g., genetic engineering other techniques. In certain embodiments, a SARS-CoV-2 spike protein described herein is monomeric. In certain embodiments, a SARS-CoV-2 spike protein described herein is multimeric. In a specific embodiment, a SARS-CoV-2 spike protein described herein is trimeric. See, e.g., Examples 1 to 5 and Examples 7 and 8, infra, for examples of SARS-CoV-2 spike protein described herein. In some embodiments, a SARS-CoV-2 spike protein described herein retains the ability to bind to the host cell receptor for SARS-CoV-2 (e.g., ACE-2).

Examples of amino acid and nucleotide sequences of SARS-CoV-2 spike proteins known to those of skill in the art include found at GenBank Accession Nos. MN908947.3, MT049951, MT093631, MT121215, MT447160, MT44636, MT446360, MT444593, MT444529, MT370887, and MT334558. A typical spike protein comprises domains known to those of skill in the art including an S1 domain, a receptor binding domain, an S2 domain, a transmembrane domain and a cytoplasmic domain. See, e.g., Wrapp et al., 2020, Science 367: 1260-1263 for a description of SARS-CoV-2 spike protein (in particular, the structure of such protein) and Walls et al., 2020, Cell 181: 282-292. The spike protein may be characterized has having a signal peptide (e.g., a signal peptide of 1-14 amino acid residues of the amino acid sequence of GenBank Accession No. MN908947.3), a receptor binding domain (e.g., a receptor binding domain of 319-541 amino acid residues of GenBank Accession No. MN908947.3), an ectodomain (e.g., an ectodomain of 15-1213 amino acid residues of GenBank Accession No. MN908947.3), and a transmembrane and endodomain (e.g.,a transmembrane and endodomain of 1214-1273 amino acid residues of GenBank Accession No. MN908947.3).

In a specific embodiment, a recombinant SARS-CoV-2 spike protein described herein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215. In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site. In a specific embodiment, the polybasic/furin cleavage site (RRAR) is replaced by a single A (RRAR to A) at the amino acid residue corresponding to amino acid residues 682-685 of the amino acid sequence of the SARS-CoV-2 spike protein found at GenBank Accession No. MN908947.3. In another specific embodiment, the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3. In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (RRAR to A) and the recombinant SARS-CoV-2 spike protein contains a stabilizing mutation (e.g., lysine to proline at the amino acid residue corresponding to amino acids residue 986 of the amino acid sequence of the SARS-CoV-2 spike protein found at GenBank Accession No. MN908947.3, or valine to proline at the amino acid residue corresponding to amino acids residue 987 of the amino acid sequence of the SARS-CoV-2 spike protein found at GenBank Accession No. MN908947.3). In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (e.g., the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3) and the recombinant SARS-CoV-2 spike protein contains a stabilizing mutation (e.g., lysine to proline at the amino acid residue corresponding to amino acids residue 986 of the amino acid sequence of the SARS-CoV-2 spike protein found at GenBank Accession No. MN908947.3, or valine to proline at the amino acid residue corresponding to amino acids residue 987 of the amino acid sequence of the SARS-CoV-2 spike protein found at GenBank Accession No. MN908947.3). In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (RRAR to A) and the recombinant SARS-CoV-2 spike protein contains two stabilizing mutations (lysine to proline and valine to proline at the amino acid residues corresponding to amino acids residues 986 and 987 of the amino acid sequence of the SARS-CoV-2 spike protein found at GenBank Accession No. MN908947.3). In some embodiments, a recombinant SARS-CoV-2 spike protein described herein further comprises a transmembrane domain of a SARS-CoV-2 spike protein known to one of skill in the art, such as disclosed at GenBank Accession No. MN908947.3, MT049951, MT093631, or MT121215. In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (e.g., the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3) and the recombinant SARS-CoV-2 spike protein contains two stabilizing mutations (lysine to proline and valine to proline at the amino acid residues corresponding to amino acid residues 986 and 987 of the amino acid sequence of the SARS-CoV-2 spike protein found at GenBank Accession No. MN908947.3). In some embodiments, a recombinant SARS-CoV-2 spike protein described herein further comprises a transmembrane domain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein does not comprise a transmembrane domain of a SARS-CoV-2 spike protein known to one of skill in the art, such as disclosed at GenBank Accession No. MN908947.3, MT049951, MT093631, or MT121215. In specific embodiments, a recombinant SARS-CoV-2 spike protein described herein does not comprise a transmembrane domain and endodomain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, such a recombinant spike protein comprises a signal peptide, such as the signal peptide of a SARS-CoV-2 spike protein known to one of skill in the art, such as disclosed at GenBank Accession No. MN908947.3, MT049951, MT093631, or MT121215. In a specific embodiment, the recombinant SARS-CoV-2 spike protein is soluble.

In another embodiment, a recombinant SARS-CoV-2 spike protein comprises (or consists of) the ectodomain of a SARS-CoV-2 spike protein (otherwise known as the S or structural protein) known to one of skill in the art (e.g., the SARS-CoV-2 spike protein found at GenBank Accession No. MN908947.3, MT049951, MT093631, or MT121215), a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)), wherein the recombinant SARS-CoV-2 spike protein does not contain the polybasic cleavage site. In a specific embodiment, the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3. In some embodiments, the recombinant SARS-CoV-2 spike protein comprises one or two stabilizing mutations (e.g., lysine to proline at the amino acid residue corresponding to amino acids residue 986 of the SARS-CoV-2 spike protein found at GenBank Accession No. MN908947.3, or valine to proline at the amino acid residue corresponding to amino acid residue 987 of the amino acid sequence of the SARS-CoV-2 spike protein found at GenBank Accession No. MN908947.3, or both amino acid substitutions). In certain embodiments, a recombinant spike protein comprises a signal peptide, such as the signal peptide of a SARS-CoV-2 spike protein known to one of skill in the art, such as disclosed at GenBank Accession No. MN908947.3, MT049951, MT093631, or MT121215.

In another embodiment, a recombinant SARS-CoV-2 spike protein comprises (or consists of) amino acid residues corresponding to amino acid residues 1-1213 of the amino acid sequence of the SARS-CoV-2 spike protein found at GenBank Accession No. MN908947.3, wherein the recombinant SARS-CoV-2 spike protein does not contain the polybasic cleavage site. In another embodiment, a recombinant SARS-CoV-2 spike protein comprises (or consists of) amino acid residues corresponding to amino acid residues 15-1213 of the amino acid sequence of the SARS-CoV-2 spike protein found at GenBank Accession No. MN908947.3, wherein the recombinant SARS-CoV-2 spike protein does not contain the polybasic cleavage site. In a specific embodiment, the polybasic/furin cleavage site (RRAR) is replaced by a single A (RRAR to A) at the amino acid residue corresponding to amino acid residue 685 of the amino acid sequence of the SARS-CoV-2 spike protein found at GenBank Accession No. MN908947.3. In another specific embodiment, the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3. In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (RRAR to A) and the recombinant SARS-CoV-2 spike protein contains a stabilizing mutation (e.g., lysine to proline at the amino acid residue corresponding to amino acids residue 986 of the amino acid sequence of the SARS-CoV-2 spike protein found at GenBank Accession No. MN908947.3, or valine to proline at the amino acid residue corresponding to amino acids residue 987 of the amino acid sequence of the SARS-CoV-2 spike protein found at GenBank Accession No. MN908947.3). In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (e.g., the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3) and the recombinant SARS-CoV-2 spike protein contains a stabilizing mutation (e.g., lysine to proline at the amino acid residue corresponding to amino acids residue 986 of the amino acid sequence of the SARS-CoV-2 spike protein found at GenBank Accession No. MN908947.3, or valine to proline at the amino acid residue corresponding to amino acids residue 987 of the amino acid sequence of the SARS-CoV-2 spike protein found at GenBank Accession No. MN908947.3). In another embodiment, a recombinant SARS-CoV-2 spike protein comprises (or consists of) the ectodomain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215, wherein the recombinant SARS-CoV-2 spike protein does not contain the polybasic cleavage site (e.g., the polybasic cleavage site (RRAR) is replaced by a single A (RRAR to A) at the amino acid residues corresponding to amino acid residues 682 to 685 of the amino acid sequence of the SARS-CoV-2 spike protein found at GenBank Accession No. MN908947.3) and the recombinant SARS-CoV-2 spike protein contains two stabilizing mutations (e.g., lysine to proline at the amino acid residue corresponding to amino acids residue 986 of the amino acid sequence of the SARS-CoV-2 spike protein found at GenBank Accession No. MN908947.3, and valine to proline at the amino acid residue corresponding to amino acids residue 987 of the amino acid sequence of the SARS-CoV-2 spike protein found at GenBank Accession No. MN908947.3).

In another embodiment, a recombinant SARS-CoV-2 spike protein comprises (or consists of) amino acid residues corresponding to amino acid residues 1-1213 of the amino acid sequence of the SARS-CoV-2 spike protein found at GenBank Accession No. MN908947.3, wherein the protein does not contain the polybasic cleavage site (RRAR to A) and the protein contains two stabilizing mutations (lysine to proline and valine to proline at the amino acid residues corresponding to amino acids residues 986 and 987 of the amino acid sequence of the SARS-CoV-2 spike protein found at GenBank Accession No. MN908947.3). In some embodiments, a recombinant SARS-CoV-2 spike protein described herein further comprises a transmembrane domain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein does not comprise a transmembrane domain of a SARS-CoV-2 spike protein known to one of skill in the art, such as disclosed at GenBank Accession No. MN908947.3, MT049951, MT093631, or MT121215. In specific embodiments, a recombinant SARS-CoV-2 spike protein described herein does not comprise a transmembrane domain and endodomain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In a specific embodiment, the recombinant SARS-CoV-2 spike protein is soluble.

In another embodiment, a recombinant soluble SARS-CoV-2 spike protein comprises (or consists of) amino acid residues corresponding to amino acid residues 15-1213 of the amino acid sequence of the SARS-CoV-2 spike protein found at amino acid residues found at GenBank Accession No. MN908947.3, wherein the protein does not contain the polybasic cleavage site (RRAR to A) and the protein contains two stabilizing mutations (lysine to proline and valine to proline at the amino acid residues corresponding to amino acids residues 986 and 987 of the amino acid sequence of the SARS-CoV-2 spike protein found at GenBank Accession No. MN908947.3). In some embodiments, a recombinant SARS-CoV-2 spike protein described herein further comprises a transmembrane domain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein does not comprise a transmembrane domain of a SARS-CoV-2 spike protein known to one of skill in the art, such as disclosed at GenBank Accession No. MN908947.3, MT049951, MT093631, or MT121215. In specific embodiments, a recombinant SARS-CoV-2 spike protein described herein does not comprise a transmembrane domain and endodomain of a SARS-CoV-2 spike protein known to one of skill in the art, such as disclosed at GenBank Accession No. MN908947.3, MT049951, MT093631, or MT121215. In certain embodiments, a recombinant spike protein comprises a signal peptide, such as the signal peptide of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In specific embodiments, the recombinant SARS-CoV-2 spike protein is soluble.

In another embodiment, a recombinant soluble SARS-CoV-2 spike protein comprises (or consists of), in the following order: (i) amino acid residues corresponding to amino acid residues 15-1213 of the amino acid sequence of the SARS-CoV-2 spike protein found at amino acid residues found at GenBank Accession No. MN908947.3, (ii) a C-terminal cleavage site (e.g., a C-terminal thrombin cleavage site or other known cleavage site), (iii) trimerization domain (e.g., a T4 foldon trimerization domain), and (iv) a tag (e.g., a flag tag or histidine tag (such as, e.g., hexahistidine tag)), wherein the protein does not contain the polybasic cleavage site (RRAR to A) and the protein contains two stabilizing mutations (lysine to proline and valine to proline at the amino acid residues corresponding to amino acids residues 986 and 987 of the amino acid sequence of the SARS-CoV-2 spike protein found at GenBank Accession No. MN908947.3. In certain embodiments, the recombinant spike protein comprises a signal peptide, such as the signal peptide of a SARS-CoV-2 spike protein known to one of skill in the art, such as disclosed at GenBank Accession No. MN908947.3, MT049951, MT093631, or MT121215. In specific embodiments, the recombinant SARS-CoV-2 spike protein is soluble.

In another embodiment, a recombinant soluble SARS-CoV-2 spike protein comprises (or consists of) amino acid residues corresponding to amino acid residues 15-1213 of the amino acid sequence of the SARS-CoV-2 spike protein found at GenBank Accession No. MN908947.3±1, 2, 3, 4, 5, 6, 7, 8, 9 or 10 amino acid residues at the N-terminus, wherein the recombinant soluble SARS-CoV-2 spike protein does not contain the polybasic cleavage site (RRAR to A) and the recombinant soluble SARS-CoV-2 spike protein contains one or two stabilizing mutations (e.g., lysine to proline at the amino acid residue corresponding to amino acid residues 986 of the amino acid sequence of the SARS-CoV-2 spike protein found at GenBank Accession No. MN908947.3, or valine to proline at the amino acid residue corresponding to amino acid residue 987 of the amino acid sequence of the SARS-CoV-2 spike protein found at GenBank Accession No. MN908947.3, or both amino acid substitutions). In another embodiment, a recombinant soluble SARS-CoV-2 spike protein comprises (or consists of) amino acid residues corresponding to amino acid residues 15-1213 of the amino acid sequence of the SARS-CoV-2 spike protein found at GenBank Accession No. MN908947.3±1, 2, 3, 4, 5, 6, 7, 8, 9 or 10 amino acid residues at the C-terminus, wherein the recombinant soluble SARS-CoV-2 spike protein does not contain the polybasic cleavage site (RRAR to A) and the recombinant soluble SARS-CoV-2 spike protein contains one or two stabilizing mutations (e.g., lysine to proline at the amino acid residue corresponding to amino acid residues 986 of the amino acid sequence of the SARS-CoV-2 spike protein found at GenBank Accession No. MN908947.3, or valine to proline at the amino acid residue corresponding to amino acid residue 987 of the amino acid sequence of the SARS-CoV-2 spike protein found at GenBank Accession No. MN908947.3, or both amino acid substitutions). In another embodiment, a recombinant soluble SARS-CoV-2 spike protein comprises (or consists of) amino acid residues corresponding to amino acid residues 15-1213 of the amino acid sequence of the SARS-CoV-2 spike protein found at GenBank Accession No. MN908947.3±1, 2, 3, 4, 5, 6, 7, 8, 9 or 10 amino acid residues at the N-terminus and ±1, 2, 3, 4, 5, 6, 7, 8, 9 or 10 amino acid residues at the C-terminus, wherein the recombinant soluble SARS-CoV-2 spike protein does not contain the polybasic cleavage site (RRAR to A) and the recombinant soluble SARS-CoV-2 spike protein contains one or two stabilizing mutations (e.g., lysine to proline at the amino acid residue corresponding to amino acid residues 986 of the amino acid sequence of the SARS-CoV-2 spike protein found at GenBank Accession No. MN908947.3, or valine to proline at the amino acid residue corresponding to amino acid residue 987 of the amino acid sequence of the SARS-CoV-2 spike protein found at GenBank Accession No. MN908947.3, or both amino acid substitutions). In some embodiments, a recombinant SARS-CoV-2 spike protein described herein further comprises a transmembrane domain of a SARS-CoV-2 spike protein known to one of skill in the art, such as disclosed at GenBank Accession No. MN908947.3, MT049951, MT093631, or MT121215. In certain embodiments, a recombinant SARS-CoV-2 spike protein described herein does not comprise a transmembrane domain of a SARS-CoV-2 spike protein known to one of skill in the art, such as disclosed at GenBank Accession No. MN908947.3, MT049951, MT093631, or MT121215. In specific embodiments, a recombinant SARS-CoV-2 spike protein described herein does not comprise a transmembrane domain and endodomain of a SARS-CoV-2 spike protein known to one of skill in the art, such as disclosed at GenBank Accession No. MN908947.3, MT049951, MT093631, or MT121215. In certain embodiments, a recombinant soluble SARS-CoV-2 spike protein comprises a signal peptide, such as the signal peptide of a SARS-CoV-2 spike protein known to one of skill in the art, such as disclosed at GenBank Accession No. MN908947.3, MT049951, MT093631, or MT121215. In certain embodiments, a recombinant soluble SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In specific embodiments, the cleavage site, trimerization domain and tag are at the C-terminus of the sequence. In certain embodiments, a recombinant soluble SARS-CoV-2 spike protein described herein further comprises all of the following in the recited order: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, a recombinant soluble SARS-CoV-2 spike protein described herein further comprises the following: a C-terminal cleavage site (e.g., a C-terminal thrombin cleavage site or other known cleavage site) and trimerization domain (e.g., a T4 foldon trimerization domain). In certain embodiments, the C-terminal cleavage site and trimerization domain are at the C-terminus of the sequence.

In certain embodiments, a recombinant SARS-CoV-2 spike protein comprises (or consists of) the amino acid sequence of the ectodomain of the SARS-CoV-2 spike protein UK variant known as 201/501Y.VI, VOC 202012/01, or B.1.1.7. In some embodiments, a recombinant SARS-CoV-2 spike protein comprises the amino acid sequence of the ectodomain of the SARS-CoV-2 spike protein South African variant known as 20H/501Y.V2 or B.351. In certain embodiments, a recombinant SARS-CoV-2 spike protein comprises the amino acid sequence of the ectodomain of the SARS-CoV-2 spike protein Brazil variant known as P.1. In some embodiments, a recombinant SARS-CoV-2 spike protein comprises the amino acid sequence of the ectodomain of the SARS-CoV-2 spike protein California variant known as CAL20.C. In some embodiments, a recombinant SARS-CoV-2 spike protein comprises the amino acid sequence of the ectodomain of the SARS-CoV-2 spike protein New York variant known as B1.526 New York. In certain embodiments, a recombinant soluble SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In specific embodiments, the cleavage site, trimerization domain and tag are at the C-terminus of the sequence. In certain embodiments, a recombinant soluble SARS-CoV-2 spike protein described herein further comprises all of the following in the recited order: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, a recombinant soluble SARS-CoV-2 spike protein described herein further comprises the following: a C-terminal cleavage site (e.g., a C-terminal thrombin cleavage site or other known cleavage site) and trimerization domain (e.g., a T4 foldon trimerization domain). In certain embodiments, the C-terminal cleavage site and trimerization domain are at the C-terminus of the sequence. In certain embodiments, the recombinant soluble SARS-CoV-2 spike protein comprises a signal sequence (e.g., a signal sequence of a SARS-CoV-2 spike protein or a heterologous signal sequence). In other embodiments, the recombinant soluble SARS-CoV-2 spike protein does not comprise a signal sequence.

In certain embodiments, a recombinant SARS-CoV-2 spike protein comprises (or consists of) the amino acid sequence of the ectodomain of the SARS-CoV-2 spike protein with one or more deletions and/or one, two, three, four, five or more of the amino acid substitutions found in the ectodomain of the SARS-CoV-2 spike protein UK variant known as 201/501Y.VI, VOC 202012/01, or B.1.1.7; the SARS-CoV-2 spike protein South African variant known as 20H/501Y.V2 or B.351; the SARS-CoV-2 spike protein Brazil variant known as P.1; the SARS-CoV-2 spike protein California variant known as CAL20.C; or the SARS-CoV-2 spike protein New York variant known as B1.526 New York. In some embodiments, a recombinant SARS-CoV-2 spike protein comprises (or consists of) the amino acid sequence of the ectodomain of the SARS-CoV-2 spike protein with one, two, three, four, five or more of the amino acid substitutions found in the ectodomain of the SARS-CoV-2 spike protein UK variant known as 201/501Y.VI, VOC 202012/01, or B.1.1.7; the SARS-CoV-2 spike protein South African variant known as 20H/501Y.V2 or B.351; the SARS-CoV-2 spike protein Brazil variant known as P.1; the SARS-CoV-2 spike protein California variant known as CAL20.C; or the SARS-CoV-2 spike protein New York variant known as B1.526 New York. In certain embodiments, a recombinant SARS-CoV-2 spike protein comprises (or consists of) the amino acid sequence of the ectodomain of the SARS-CoV-2 spike protein with 1, 2, 3, 4, 5, 6, 7, 8, 9 or more of the following amino acid substitutions: N501Y, A570D, D614G, P681H, S982A, D1118H, L18F, D80A, D215G, K417N, E484K, D614G, A701V, T20N, P26S, D138Y, R190S, K417T, E484K, H655Y, T1027I, S13I, W152C, L452R, L5F, T95I, D253G, or S477N. In certain embodiments, a recombinant soluble SARS-CoV-2 spike protein described herein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In specific embodiments, the cleavage site, trimerization domain and tag are at the C-terminus of the sequence. In certain embodiments, a recombinant soluble SARS-CoV-2 spike protein described herein further comprises all of the following in the recited order: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, a recombinant soluble SARS-CoV-2 spike protein described herein further comprises the following: a C-terminal cleavage site (e.g., a C-terminal thrombin cleavage site or other known cleavage site) and trimerization domain (e.g., a T4 foldon trimerization domain). In certain embodiments, the C-terminal cleavage site and trimerization domain are at the C-terminus of the sequence. In certain embodiments, the recombinant soluble SARS-CoV-2 spike protein comprises a signal sequence (e.g., a signal sequence of a SARS-CoV-2 spike protein or a heterologous signal sequence). In other embodiments, the recombinant soluble SARS-CoV-2 spike protein does not comprise a signal sequence.

In certain embodiments, the C-terminal cleavage site of a recombinant SARS-CoV-2 spike protein described herein is one known to one of skill in the art. In some embodiments, the C-terminal cleavage site of a recombinant SARS-CoV-2 spike protein described herein is a thrombin, 2A, 2B, 2C, 3A, 3B, 3C, or thermolysin cleavage site. In certain embodiments, the trimerization domain of a recombinant SARS-CoV-2 spike protein described herein is one known to one of skill in the art. For example, the trimerization domain of a recombinant SARS-CoV-2 spike protein described herein may be a leucine zipper. In some embodiments, the trimerization domain of a recombinant SARS-CoV-2 spike protein described herein is the trimerization domain (foldon) of T4 phage fibritin and a leucine zipper trimerization motif derived from the yeast transcription activator GCN. In certain embodiments, the trimerization domain of a recombinant SARS-CoV-2 spike protein described herein is one known to one of skill in the art or described herein (e.g, in the Examples, infra). In some embodiments, the tag of a recombinant SARS-CoV-2 spike protein described herein is a flag tag, histidine tag (e.g., hexahistidine tag), HA tag, Myc tag, V5, glutathione-S-transferase (GST) tag, Maltose Binding Protein (MBP) tag, or Vesicular Stomatitis Virus Glycoprotein (VSV-G) tag.

In a specific embodiment, a recombinant SARS-CoV-2 spike protein comprises amino acids 1-1213 (MFVF . . . IKWP) of the spike protein found at GenBank Accession No. MN908947.3, a C-terminal thrombin cleavage site, T4 foldon trimerization domain and hexahistidine tag, wherein the protein does not contain the polybasic cleavage site (RRAR to A). In another specific embodiment, a recombinant SARS-CoV-2 spike protein comprises amino acids 1-1213 (MFVF . . . IKWP) of the spike protein found at GenBank Accession No. MN908947.3, a C-terminal thrombin cleavage site, T4 foldon trimerization domain and hexahistidine tag, wherein the protein does not contain the polybasic cleavage site (RRAR to A) and the protein contains two stabilizing mutations (K986P and V987P, wild type numbering). In some embodiments, such a recombinant soluble spike protein comprises a signal peptide, such as the signal peptide of the spike protein disclosed at GenBank Accession No. MN908947.3.

In a specific embodiment, a recombinant soluble SARS-CoV-2 spike protein comprises amino acids 1-1213 (MFVF . . . IKWP) of the spike protein (otherwise known as the S or structural protein) found at GenBank Accession No. MN908947.3. In another embodiment, a recombinant soluble SARS-CoV-2 spike protein comprises amino acids 1-1213 (MFVF . . . IKWP) of the spike protein found at GenBank Accession No. MN908947.3, wherein the protein does not contain the polybasic cleavage site. In a specific embodiment, the polybasic/furin cleavage site (RRAR) 682-685 is replaced by a single A (RRAR to A). In another specific embodiment, the polybasic/furin cleavage site (RRAR) found at amino acid residues 682-685 of SARS-CoV-2 spike protein found at GenBank Accession No. MN908947.3 is replaced by a single A (RRAR to A). In another embodiment, a recombinant soluble SARS-CoV-2 spike protein comprises amino acids 1-1213 (MFVF . . . IKWP) of the spike protein found at GenBank Accession No. MN908947.3, wherein the protein does not contain the polybasic cleavage site (RRAR to A) and the protein contains two stabilizing mutations (K986P and V987P, wild-type numbering). In certain embodiments, such a recombinant soluble spike protein comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In certain embodiments, such a recombinant soluble spike protein comprises a C-terminal cleavage site (e.g., a C-terminal thrombin cleavage site or other known cleavage site) and trimerization domain (e.g., a T4 foldon trimerization domain). In specific embodiments, such additional amino acid residues are at the C-terminus of the sequence. In some embodiments, such a recombinant soluble spike protein comprises a signal peptide, such as the signal peptide of the spike protein disclosed at GenBank Accession No. MN908947.3.

In a specific embodiment, a recombinant soluble SARS-CoV-2 spike protein comprises amino acids 15-1213 (CVNL . . . IKWP) of the spike protein (otherwise known as the S or structural protein) found at GenBank Accession No. MN908947.3. In another embodiment, a recombinant soluble SARS-CoV-2 spike protein comprises amino acids 15-1213 (CVNL . . . IKWP) of the spike protein found at GenBank Accession No. MN908947.3, wherein the recombinant soluble SARS-CoV-2 spike protein protein does not contain the polybasic cleavage site. In a specific embodiment, the polybasic/furin cleavage site (RRAR) at amino acid residues 682-685 of the SARS-CoV-2 spike protein found at GenBank Accession No. MN908947.3 is replaced by a single A (RRAR to A). In another embodiment, a recombinant soluble SARS-CoV-2 spike protein comprises amino acids 1-1213 (MFVF . . . IKWP) of the spike protein found at GenBank Accession No. MN908947.3, wherein the recombinant soluble SARS-CoV-2 spike protein does not contain the polybasic cleavage site (RRAR to A) and the recombinant soluble SARS-CoV-2 spike protein contains two stabilizing mutations (K986P and V987P, wild-type numbering). In certain embodiments, such a recombinant soluble spike protein comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, such a recombinant soluble spike protein comprises a signal peptide, such as the signal peptide of the spike protein disclosed at GenBank Accession No. MN908947.3.

In a specific embodiment, a recombinant soluble SARS-CoV-2 spike protein comprises, in the following order: (i) amino acids 15-1213 (CVNL . . . IKWP) of the spike protein (otherwise known as the S or structural protein) found at GenBank Accession No. MN908947.3, (ii) a C-terminal cleavage site (e.g., a C-terminal thrombin cleavage site or other known cleavage site), (iii) a trimerization domain (e.g., a T4 foldon trimerization domain), and (iv) a tag (e.g., a flag tag or histidine tag (such as, e.g., hexahistidine tag)), wherein the recombinant soluble SARS-CoV-2 spike protein does not contain the polybasic cleavage site and the recombinant soluble SARS-CoV-2 spike protein contains one or two stabilizing mutations (K986P and/or V987P, wild-type numbering). In some embodiments, such a recombinant soluble spike protein comprises a signal peptide, such as the signal peptide of the spike protein disclosed at GenBank Accession No. MN908947.3.

In a specific embodiment, a recombinant soluble SARS-CoV-2 spike protein comprises amino acids 1-1213 (MFVF . . . IKWP) of the spike protein found at GenBank Accession No. MN908947.3, a C-terminal thrombin cleavage site, T4 foldon trimerization domain and hexahistidine tag, wherein the protein does not contain the polybasic cleavage site (RRAR to A). In another specific embodiment, a recombinant soluble SARS-CoV-2 spike protein comprises amino acids 1-1213 (MFIVF . . . IKWP) of the spike protein found at GenBank Accession No. MN908947.3, a C-terminal thrombin cleavage site, T4 foldon trimerization domain and hexahistidine tag, wherein the protein does not contain the polybasic cleavage site (RRAR to A) and the protein contains two stabilizing mutations (K986P and V987P, wild type numbering). In some embodiments, such a recombinant soluble spike protein comprises a signal peptide, such as the signal peptide of the spike protein disclosed at GenBank Accession No. MN908947.3.

In a specific embodiment, a recombinant SARS-CoV-2 spike protein described herein comprises the receptor binding domain of a SARS-CoV-2 spike protein (otherwise known as the S or structural protein) known to one of skill in the art, such as the SARS-CoV-2 spike protein found at GenBank Accession No. MN908947.3, MT049951, MT093631, or MT121215. In another embodiment, a recombinant soluble SARS-CoV-2 spike protein comprises (or consists of) the receptor binding domain of a SARS-CoV-2 protein known to one of skill in art (e.g., the SARS-CoV-2 spike protein found at GenBank Accession No. MT049951, MT093631, or MT121215) corresponding to amino acid residues corresponding to amino acid residues 319-541 of the SARS-CoV-2 spike protein found at GenBank Accession No. MN908947.3. In certain embodiments, such a recombinant spike protein further comprises one, two or all of the following: a C-terminal cleavage site (e.g., a C-terminal thrombin cleavage site or other known cleavage site), trimerization domain (e.g., a T4 foldon trimerization domain) and a tag (e.g., a flag tag or histidine tag (such as, e.g., hexahistidine tag)). In some embodiments, such a recombinant SARS-CoV-2 spike protein comprises a signal peptide, such as the signal peptide of the spike protein disclosed at GenBank Accession No. MN908947.3, MT049951, MT093631, or MT121215.

In another embodiment, a recombinant soluble SARS-CoV-2 spike protein comprises amino acid residues corresponding to amino acid residues 319-541 of the SARS-CoV-2 spike protein found at GenBank Accession No. MN908947.3. In certain embodiments, such a recombinant soluble spike protein comprises a tag, such as a histidine tag (e.g., hexahistidine tag) or flag tag. In a specific embodiment, the tag is at the C-terminus of the sequence. In some embodiments, such a recombinant soluble SARS-CoV-2 spike protein comprises a signal peptide, such as the signal peptide of the spike protein disclosed at GenBank Accession No. MN908947.3.

In some embodiments, a recombinant soluble SARS-CoV-2 spike protein that comprises a receptor binding domain of a SARS-CoV-2 spike protein comprises less than the S1 domain of the SARS-CoV-2 spike protein. In other words, a recombinant soluble SARS-CoV-2 spike protein includes the receptor binding domain of a SARS-CoV-2 spike protein but not the entire S1 domain. In other embodiments, a recombinant soluble SARS-CoV-2 spike protein that comprises a receptor binding domain of a SARS-CoV-2 spike protein includes the S1 domain of the SARS-CoV-2 spike protein. In other words, a recombinant soluble SARS-CoV-2 spike protein includes the receptor binding domain of a SARS-CoV-2 spike protein comprises (or consists of) the S1 domain of the SARS-CoV-2 spike protein. In some embodiments, a recombinant soluble SARS-CoV-2 spike protein that comprises a receptor binding domain of a SARS-CoV-2 spike protein includes the S1 domain and the S2 domain of the SARS-CoV-2 spike protein or a fragment thereof. In other words, a recombinant soluble SARS-CoV-2 spike protein includes the receptor binding domain of a SARS-CoV-2 spike protein comprises (or consists of) the S1 domain and the S2 domain of the SARS-CoV-2 spike protein or a fragment thereof. The fragment of the S2 domain may be 5, 10, 20, 30 or more amino acid residues in length. In certain embodiments, a recombinant soluble SARS-CoV-2 spike protein that comprises a receptor binding domain of a SARS-CoV-2 spike protein includes less than the entire ectodomain of the SARS-CoV-2 spike protein. In other words, a recombinant soluble SARS-CoV-2 spike protein includes the receptor binding domain of a SARS-CoV-2 spike protein comprises (or consists of) less than the ectodomain of the SARS-CoV-2 spike protein. In some embodiments, a recombinant soluble SARS-CoV-2 spike protein that comprises a receptor binding domain of a SARS-CoV-2 spike protein does not include the S2 domain of the SARS-CoV-2 spike protein or a fragment thereof.

In another embodiment, a recombinant soluble SARS-CoV-2 spike protein comprises amino acid residues 319-541 of the spike protein found at GenBank Accession No. MN908947.3±1, 2, 3, 4, 5, 6, 7, 8, 9 or 10 amino acid residues at the N-terminus, and a tag (e.g., a hexahistidine tag). In another embodiment, a recombinant soluble SARS-CoV-2 spike protein comprises amino acid residues 319-541 of the spike protein found at GenBank Accession No. MN908947.3±1, 2, 3, 4, 5, 6, 7, 8, 9 or 10 amino acid residues at the C-terminus and a tag (e.g., a hexahistidine tag). In another specific embodiment, a recombinant soluble SARS-CoV-2 spike protein comprises amino acid residues 319-541 of the spike protein found at GenBank Accession No. MN908947.3±1, 2, 3, 4, 5, 6, 7, 8, 9 or 10 amino acid residues at the N-terminus and ±1, 2, 3, 4, 5, 6, 7, 8, 9 or 10 amino acid residues at the C-terminus, and a tag (e.g., a hexahistidine tag). In a specific embodiment, the tag is at the C-terminus of the sequence.

In another embodiment, a recombinant soluble SARS-CoV-2 spike protein comprises amino acid residues 319-541 of the spike protein found at GenBank Accession No. MN908947.3. In certain embodiments, such a recombinant soluble spike protein comprises a tag, such as a histidine tag (e.g., hexahistidine tag) or flag tag. In some embodiments, such a recombinant soluble SARS-CoV-2 spike protein comprises a signal peptide, such as the signal peptide of the spike protein disclosed at GenBank Accession No. MN908947.3. In a specific embodiment, a recombinant soluble SARS-CoV-2 spike protein comprises the signal peptide of the spike protein disclosed at GenBank Accession No. MN908947.3, amino acid residues 319-541 of the spike protein found at GenBank Accession No. MN908947.3, and a hexahistidine tag.

In certain embodiments, a recombinant SARS-CoV-2 spike protein comprises (or consists of) the amino acid sequence of the receptor binding domain of the SARS-CoV-2 spike protein UK variant known as 201/501Y.VI, VOC 202012/01, or B.1.1.7. In some embodiments, a recombinant SARS-CoV-2 spike protein comprises (or consists of) the amino acid sequence of the receptor binding domain of the SARS-CoV-2 spike protein South African variant known as 20H/501Y.V2 or B.351. In certain embodiments, a recombinant SARS-CoV-2 spike protein comprises (or consists of) the amino acid sequence of the receptor binding domain of the SARS-CoV-2 spike protein Brazil variant known as P.1. In some embodiments, a recombinant SARS-CoV-2 spike protein comprises (or consists of) the amino acid sequence of the receptor binding domain of the SARS-CoV-2 spike protein California variant known as CAL20.C. In some embodiments, a recombinant SARS-CoV-2 spike protein comprises (or consists of) the amino acid sequence of the receptor binding domain of the SARS-CoV-2 spike protein New York variant known as B1.526 New York. In certain embodiments, such recombinant SARS-CoV-2 spike proteins comprise±1, 2, 3, 4, 5, 6, 7, 8, 9 or 10 amino acid residues at the N-terminus, ±1, 2, 3, 4, 5, 6, 7, 8, 9 or 10 amino acid residues at the C-terminus, or both. In some embodiments, such recombinant SARS-CoV-2 spike protein comprise a tag (e.g., a hexahistidine tag or other tag described herein or known to one of skill in the art). The tag may be at the C-terminus of the sequence.

In certain embodiments, a recombinant SARS-CoV-2 spike protein comprises (or consists of) the amino acid sequence of the receptor binding domain of the SARS-CoV-2 spike protein with one, two, three or more of the amino acid substitutions found in the receptor binding domain of the SARS-CoV-2 spike protein UK variant known as 201/501Y.VI, VOC 202012/01, or B.1.1.7; the SARS-CoV-2 spike protein South African variant known as 20H/501Y.V2 or B.351; the SARS-CoV-2 spike protein Brazil variant known as P.1; the SARS-CoV-2 spike protein California variant known as CAL20.C; or the SARS-CoV-2 spike protein New York variant known as B1.526 New York. In certain embodiments, such recombinant SARS-CoV-2 spike proteins comprise±1, 2, 3, 4, 5, 6, 7, 8, 9 or 10 amino acid residues at the N-terminus, ±1, 2, 3, 4, 5, 6, 7, 8, 9 or 10 amino acid residues at the C-terminus, or both. In some embodiments, such recombinant SARS-CoV-2 spike protein comprise a tag (e.g., a hexahistidine tag or other tag described herein or known to one of skill in the art). The tag may be at the C-terminus of the sequence.

In a specific embodiment, a recombinant SARS-CoV-2 spike protein comprises the amino acid sequence of SEQ ID NO:2. In a specific embodiment, a recombinant SARS-CoV-2 spike protein comprises the amino acid sequence of SEQ ID NO:4. In a specific embodiment, a recombinant SARS-CoV-2 spike protein comprises the amino acid sequence of SEQ ID NO:6. In a specific embodiment, a recombinant SARS-CoV-2 spike protein comprises the amino acid sequence of SEQ ID NO:2, 4 or 6 without the first 14 amino acid residues. In a specific embodiment, a recombinant SARS-CoV-2 spike protein comprises the amino acid sequence of SEQ ID NO: 10.

In another specific embodiment, provided herein is a recombinant SARS-CoV-2 spike protein comprising (or consisting of) the amino acid sequence set forth in GenBank Accession No. MT380725.1. In another specific embodiment, provided herein is a recombinant SARS-CoV-2 spike protein comprising (or consisting of) the amino acid sequence set forth in GenBank Accession No. MT380724.1.

In another specific embodiment, provided herein is a recombinant SARS-CoV-2 spike protein comprising (or consisting of) a derivative of a SARS-CoV-2 spike protein receptor binding domain. In specific embodiments, the recombinant SARS-CoV-2 spike protein only comprises the derivative of the SARS-CoV-2 spike protein receptor binding domain and no other portion of the SARS-CoV-2 spike protein. In certain embodiments, the recombinant SARS-CoV-2 spike protein further comprises a heterologous amino acid sequence, such as a tag (e.g., a flag tag, histidine tag or other tag described herein or known to one of skill in the art). In specific embodiments, the heterologous sequence is at the C-terminus of the derivative of the SARS-CoV-2 spike protein receptor binding domain. In certain embodiments, the recombinant SARS-CoV-2 spike protein further comprises a signal peptide. The signal peptide may be heterologous to the SARS-CoV-2 spike protein or may be the signal peptide of a SARS-CoV-2 spike protein. One of skill in the art would be able to determine the receptor binding domain of a SARS-CoV-2 spike protein using techniques known to one of skill in the art. In a specific embodiment, a derivative of a SARS-CoV-2 spike protein receptor binding domain comprises amino acid substitutions in a certain number of amino acid residues. For example, a derivative of a SARS-CoV-2 spike protein receptor binding domain may comprises 1, 2, 3, 4, 5, 6, 7, 8, 9, 10, 11, 12, 13, 14, 15 or more amino acid substitutions. In another example, a derivative of a SARS-CoV-2 spike protein receptor binding domain may comprises 1-5, 5-10, 1-10, 1-15, 5-15 or 10-15 amino acid substitutions. In another embodiment, a derivative a SARS-CoV-2 spike protein receptor binding domain may be a certain number of residues shorter than the full receptor binding domain. For example, a derivative a SARS-CoV-2 spike protein receptor binding domain may be 1, 2, 3, 4, 5, 6, 7, 8, 9, 10, 11, 12, 13, 14, 15 or more amino acid residues shorter at the N-terminus. In another example, a derivative a SARS-CoV-2 spike protein receptor binding domain may be 1, 2, 3, 4, 5, 6, 7, 8, 9, 10, 11, 12, 13, 14, 15, or more amino acid residues shorter at the C-terminus. In another example, a derivative a SARS-CoV-2 spike protein receptor binding domain may be 1, 2, 3, 4, 5, 6, 7, 8, 9, 10, 11, 12, 13, 14, 15, or more amino acid residues shorter at the N-terminus and 1, 2, 3, 4, 5, 6, 7, 8, 9, 10, 11, 12, 13, 14, 15 or more amino acid residues shorter at the C-terminus. In another embodiment, a derivative of a SARS-CoV-2 spike protein receptor binding domain comprises amino acid substitutions in a certain number of amino acid residues (e.g., 1, 2, 3, 4, 5, 6, 7, 8, 9, 10, 11, 12, 13, 14, 15, or more amino acid substitutions) and be shorter than the full receptor binding domain at the N-terminus, C-terminus or both by a certain number of amino acid residues (e.g., 1, 2, 3, 4, 5, 6, 7, 8, 9, 10, 11, 12, 13, 14, 15 or more amino acid residues).

In another embodiment, a derivative of a SARS-CoV-2 spike protein receptor binding domain comprises an amino acid sequence encoded by a nucleotide sequence that hybridizes under stringent conditions (e.g., moderate or high stringency conditions) to a nucleotide sequence encoding a SARS-CoV-2 spike protein receptor binding domain known to one of skill in the art. Hybridization conditions have been described in the art and are known to one of skill in the art (see, e.g., the descriptions of hybridizations conditions provided herein). For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F. M. et al, eds., 1989, Current Protocols in Molecular Biology, Vol. I, Green Publishing Associates, Inc. and John Wiley & Sons, Inc., New York at pages 6.3.1-6.3.6 and 2.10.3. In certain embodiments, the terms “about” and “approximately” in the context of a value refer to a value within 1%, 2%, 3%, 4%, or 5% of the recited value and includes the recited value. In certain embodiments, the terms “about” and “approximately” in the context of a value refer to a value within 6%, 7%, 8%, 9%, or 10% of the recited value and includes the recited value.

In another embodiment, a derivative of a SARS-CoV-2 spike protein receptor binding domain comprises an amino acid sequence that is at least 80% or 85%, 90%, 95%, 96%, 97%, 98% or 99% identical to the amino acid sequence of a SARS-CoV-2 spike protein receptor binding domain known to one of skill in the art. In another embodiment, a derivative of a SARS-CoV-2 spike protein receptor binding domain comprises an amino acid sequence that is at least 80% or 85%, identical to the amino acid sequence of a SARS-CoV-2 spike protein receptor binding domain known to one of skill in the art. In another embodiment, a derivative of a SARS-CoV-2 spike protein receptor binding domain comprises an amino acid sequence that is at least 90% or 95% identical to the amino acid sequence of a SARS-CoV-2 spike protein receptor binding domain known to one of skill in the art. In another embodiment, a derivative of a SARS-CoV-2 spike protein receptor binding domain comprises an amino acid sequence that is at least 98% identical to the amino acid sequence of a SARS-CoV-2 spike protein receptor binding domain known to one of skill in the art. See, e.g., GenBank Accession No. MN908947.3, MT049951, MT093631, or MT121215 for SARS-CoV-2 spike proteins known to one of skill in the art.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence. The determination of percent identity between two sequences (e.g., amino acid sequences or nucleic acid sequences) can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). In another non-limiting example of a mathematical algorithm utilized for the comparison of sequences is the algorithm of Myers and Miller, 1988, CABIOS 4: 11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In another specific embodiment, provided herein is a recombinant SARS-CoV-2 spike protein comprising (or consisting of) a derivative of a SARS-CoV-2 spike protein ectodomain. In specific embodiments, the recombinant SARS-CoV-2 spike protein only comprises the derivative of the SARS-CoV-2 spike protein ectdomain and no other portion of the SARS-CoV-2 spike protein. In certain embodiments, the recombinant SARS-CoV-2 spike protein further comprises one, two or all of the following: a C-terminal cleavage site (e.g., a C-terminal thrombin cleavage site or other cleavage site described herein or known to one of skill in the art), a trimerization domain (e.g., a T4 foldon trimerization domain or other trimerization domain described herein or known to one of skill in the art) and a tag (e.g., a flag tag or histidine tag (such as, e.g., hexahistidine tag), or other tag described herein or known to one of skill in the art). In specific embodiments, a recombinant SARS-CoV-2 spike protein comprises the following in order: a C-terminal cleavage site (e.g., a C-terminal thrombin cleavage site or other cleavage site described herein or known to one of skill in the art), a trimerization domain (e.g., a T4 foldon trimerization domain or other trimerization domain described herein or known to one of skill in the art) and a tag (e.g., a flag tag or histidine tag (such as, e.g., hexahistidine tag), or other tag described herein or known to one of skill in the art). Those amino acid residues may be at the C-terminus of the sequence. In some embodiments, a recombinant SARS-CoV-2 spike protein comprises the following in order: a C-terminal cleavage site (e.g., a C-terminal thrombin cleavage site or other cleavage site described herein or known to one of skill in the art) and a trimerization domain (e.g., a T4 foldon trimerization domain or other trimerization domain described herein or known to one of skill in the art) at the C-terminus. In certain embodiments, the recombinant SARS-CoV-2 spike protein further comprises a signal peptide. The signal peptide may be heterologous to the SARS-CoV-2 spike protein or may be the signal peptide of a SARS-CoV-2 spike protein. One of skill in the art would be able to determine the ectodomain of a SARS-CoV-2 spike protein using techniques known to one of skill in the art. In a specific embodiment, a derivative of a SARS-CoV-2 spike protein ectodomain comprises amino acid substitutions in a certain number of amino acid residues. For example, a derivative of a SARS-CoV-2 spike protein ectodomain may comprises 1, 2, 3, 4, 5, 6, 7, 8, 9, 10, 11, 12, 13, 14, 15, 16, 17, 18, 19, 20 or more amino acid substitutions. In another example, a derivative of a SARS-CoV-2 spike protein ectodomain may comprises 1-5, 5-10, 1-10, 1-15, 5-15, 10-15, 1-25, 10-25, or 15-25 amino acid substitutions. In another embodiment, a derivative a SARS-CoV-2 spike protein ectodomain may be a certain number of residues shorter than the full ectodomain. For example, a derivative a SARS-CoV-2 spike protein ectodomain may be 1, 2, 3, 4, 5, 6, 7, 8, 9, 10, 11, 12, 13, 14, 15, 16, 17, 18, 19, 20 or more amino acid residues shorter at the N-terminus. In another example, a derivative a SARS-CoV-2 spike protein ectodomain may be 1, 2, 3, 4, 5, 6, 7, 8, 9, 10, 11, 12, 13, 14, 15, 16, 17, 18, 19, 20 or more amino acid residues shorter at the C-terminus. In another example, a derivative a SARS-CoV-2 spike protein ectodomain may be 1, 2, 3, 4, 5, 6, 7, 8, 9, 10, 11, 12, 13, 14, 15, 16, 17, 18, 19 or 20 amino acid residues shorter at the N-terminus and 1, 2, 3, 4, 5, 6, 7, 8, 9, 10, 11, 12, 13, 14, 15, 16, 17, 18, 19, or 20 amino acid residues shorter at the C-terminus. In another embodiment, a derivative of a SARS-CoV-2 spike protein ectodomain comprises amino acid substitutions in a certain number of amino acid residues (e.g., 1, 2, 3, 4, 5, 6, 7, 8, 9, 10, 11, 12, 13, 14, or 15) and be shorter than the full ectodomain at the N-terminus, C-terminus or both by a certain number of amino acid residues (e.g., 1, 2, 3, 4, 5, 6, 7, 8, 9, 10, 11, 12, 13, 14, or 15).

In another embodiment, a derivative of a SARS-CoV-2 spike protein ectodomain comprises an amino acid sequence encoded by a nucleotide sequence that hybridizes under stringent conditions (e.g., moderate or high stringency conditions) to a nucleotide sequence encoding a SARS-CoV-2 spike protein ectodomain known to one of skill in the art. Hybridization conditions have been described in the art and are known to one of skill in the art (see, e.g., the descriptions of hybridizations conditions provided herein).

In another embodiment, a derivative of a SARS-CoV-2 spike protein ectodomain comprises an amino acid sequence that is at least 80% or 85%, 90%, 95%, 96%, 97%, 98% or 99% identical to the amino acid sequence of a SARS-CoV-2 spike protein ectodomain known to one of skill in the art. In another embodiment, a derivative of a SARS-CoV-2 spike protein ectodomain comprises an amino acid sequence that is at least 80% or 85%, identical to the amino acid sequence of a SARS-CoV-2 spike protein ectodomain known to one of skill in the art. In another embodiment, a derivative of a SARS-CoV-2 spike protein ectodomain comprises an amino acid sequence that is at least 90% or 95% identical to the amino acid sequence of a SARS-CoV-2 spike protein ectodomain known to one of skill in the art. In another embodiment, a derivative of a SARS-CoV-2 spike protein ectodomain comprises an amino acid sequence that is at least 98% identical to the amino acid sequence of a SARS-CoV-2 spike protein ectodomain known to one of skill in the art. See, e.g., GenBank Accession No. MN908947.3, MT049951, MT093631, or MT121215 for SARS-CoV-2 spike proteins known to one of skill in the art.

In another embodiment, a derivative of a SARS-CoV-2 spike protein ectodomain comprises a fragment of the amino acid sequence of a SARS-CoV-2 spike protein ectodomain known to one of skill in the art. In certain embodiments, the fragment is at least 600, 650, 700, 750, 800, 850, 900, 950, 1000, 1050, 1100, 1150, 1175 or 1200 amino acid residues in length. In some embodiments, the fragment is at least 800, 850, 900, 950, 1000, 1050, 1100, 1150, 1175 or 1200 amino acid residues in length. In certain embodiments, the fragment is at least 1000, 1100, 1150, 1175 or 1200 amino acid residues in length. In a specific embodiment, the fragment comprises the receptor binding domain of the SARS-CoV-2 spike protein. In certain embodiments, the fragment is at least 1150, 1175 or 1200 amino acid residues in length. In a specific embodiment, the fragment comprises the receptor binding domain of the SARS-CoV-2 spike protein. In another specific embodiment, the fragment retains the ability to bind to the host receptor (e.g., ACE-2). In another specific embodiment, the derivative is able to bind to the host receptor (e.g., ACE-2).

In another embodiment, a derivative of a SARS-CoV-2 spike protein ectodomain comprises an amino acid sequence that is at least 80%, 85%, 90%, 95%, or 98% identical to a fragment of the amino acid sequence of a SARS-CoV-2 spike protein ectodomain known to one of skill in the art. In certain embodiments, the fragment is at least 600, 650, 700, 750, 800, 850, 900, 950, 1000, 1050, 1100, 1150, 1175 or 1200 amino acid residues in length. In some embodiments, the fragment is at least 800, 850, 900, 950, 1000, 1050, 1100, 1150, 1175 or 1200 amino acid residues in length. In certain embodiments, the fragment is at least 1000, 1100, 1150, 1175 or 1200 amino acid residues in length. In a specific embodiment, the fragment comprises the receptor binding domain of the SARS-CoV-2 spike protein. In certain embodiments, the fragment is at least 1150, 1175 or 1200 amino acid residues in length. In a specific embodiment, the derivative comprises the receptor binding domain of the SARS-CoV-2 spike protein. In another specific embodiment, the derivative is able to bind to the host receptor (e.g., ACE-2).

In another embodiment, a recombinant SARS-CoV-2 spike protein described herein comprises an amino acid sequence that is at least 85%, 90%, 95%, or 98% identical to the amino acid sequence of SEQ ID NO:2. In another embodiment, a recombinant SARS-CoV-2 spike protein described herein comprises an amino acid sequence that is at least 85%, 90%, 95%, or 98% identical to the amino acid sequence of SEQ ID NO:4. In another embodiment, a recombinant SARS-CoV-2 spike protein described herein comprises an amino acid sequence that is at least 85%, 90%, 95%, or 98% identical to the amino acid sequence of SEQ ID NO:6. In another embodiment, a recombinant SARS-CoV-2 spike protein described herein comprises an amino acid sequence that is at least 85%, 90%, 95%, or 98% identical to the amino acid sequence of SEQ ID NO:2, 4 or 6 without the first 14 amino acid residues. In another embodiment, a recombinant SARS-CoV-2 spike protein described herein comprises an amino acid sequence that is at least 85%, 90%, 95%, or 98% identical to the amino acid sequence of SEQ ID NO:10. In some embodiments, a recombinant SARS-CoV-2 spike protein(s) described herein retains the ability to bind to ACE-2 or another receptor found on a host cell.

In another specific embodiment, provided herein is a recombinant SARS-CoV-2 spike protein comprising (or consisting of) an amino acid sequence that is at least 80%, 85%, 90%, 95% or 98% identical to the amino acid sequence set forth in GenBank Accession No. MT380725.1. In another specific embodiment, provided herein is a recombinant SARS-CoV-2 spike protein comprising (or consisting of) an amino acid sequence that is at least 80% or 85% identical to the amino acid sequence set forth in GenBank Accession No. MT380725.1. In another specific embodiment, provided herein is a recombinant SARS-CoV-2 spike protein comprising (or consisting of) an amino acid sequence that is at least 90% or 95% identical to the amino acid sequence set forth in GenBank Accession No. MT380725.1. In another specific embodiment, provided herein is a recombinant SARS-CoV-2 spike protein comprising (or consisting of) an amino acid sequence that is at least 98% identical to the amino acid sequence set forth in GenBank Accession No. MT380725.1. In another specific embodiment, provided herein is a recombinant SARS-CoV-2 spike protein comprising (or consisting of) the amino acid sequence set forth in GenBank Accession No. MT380725.1.

In another specific embodiment, provided herein is a recombinant SARS-CoV-2 spike protein comprising (or consisting of) an amino acid sequence that is at least 80%, 85%, 90%, 95% or 98% identical to the amino acid sequence set forth in GenBank Accession No. MT380724.1. In another specific embodiment, provided herein is a recombinant SARS-CoV-2 spike protein comprising (or consisting of) an amino acid sequence that is at least 80% or 85% identical to the amino acid sequence set forth in GenBank Accession No. MT380724.1. In another specific embodiment, provided herein is a recombinant SARS-CoV-2 spike protein comprising (or consisting of) an amino acid sequence that is at least 90% or 95% identical to the amino acid sequence set forth in GenBank Accession No. MT380724.1. In another specific embodiment, provided herein is a recombinant SARS-CoV-2 spike protein comprising (or consisting of) an amino acid sequence that is at least 98% identical to the amino acid sequence set forth in GenBank Accession No. MT380724.1. In another specific embodiment, provided herein is a recombinant SARS-CoV-2 spike protein comprising (or consisting of) the amino acid sequence set forth in GenBank Accession No. MT380724.1.

In certain embodiments, a recombinant SARS-CoV-2 spike protein is purchased from a vendor. For example, a recombinant SARS-CoV-2 spike protein comprising a SARS-CoV-2 spike protein receptor binding domain may be purchased from BioVendor Research and Diagnostic Products (Catalog number RI973599100), Cayman Chemical (Item No. 30429), Creative Diagnostics (Item No. DAGC089), Lake Pharma (Item No. 46438); GeneScript, AdipoGen Life Sciences (Item No. CHI-B232004 or CHI-B249001), or Sino Biological. In another example, a recombinant SARS-CoV-2 spike protein comprising a SARS-CoV-2 spike protein ectodomain may be purchased from bei Resources (Item No. 61516; NR-52308) or Lake Pharma (Item No. 46328).

In specific embodiments, a recombinant SARS-CoV-2 spike protein provided herein is capable of forming a three dimensional structure that is similar to the three dimensional structure of a wild-type SARS-CoV-2 spike protein. Structural similarity might be evaluated based on any technique deemed suitable by those of skill in the art. For instance, reaction, e.g. under non-denaturing conditions, of a recombinant SARS-CoV-2 spike protein with an antibody or antiserum that recognizes a native SARS-CoV-2 spike protein might indicate structural similarity. In certain embodiments, the antibody or antiserum is an antibody or antiserum that reacts with a non-contiguous epitope (i.e., not contiguous in primary sequence) that is formed by the tertiary or quaternary structure of a SARS-CoV-2 spike protein.

In certain embodiments, a recombinant SARS-CoV-2 spike protein described herein retains one, two, or more, or all of the functions of a wild-type SARS-CoV-2 spike protein. In a specific embodiment, a recombinant SARS-CoV-2 spike protein described herein binds to a receptor(s) on human cells (e.g., ACE2). Assays known to one skilled in the art can be utilized to assess the ability of a recombinant SARS-CoV-2 spike protein to bind to a receptor(s) (e.g., ACE2).

It will be understood by those of skill in the art that a recombinant SARS-CoV-2 spike protein provided herein can be prepared according to any technique known by and deemed suitable to those of skill in the art, including the techniques described herein. In certain embodiments, a recombinant SARS-CoV-2 spike protein described herein is isolated.

### 5.2 Nucleic Acid Sequences Encoding Recombinant SARS-CoV-2 Spike Proteins

In another aspect, provided herein are nucleic acid sequences comprising a nucleotide sequence encoding a recombinant SARS-CoV-2 spike protein described herein (e.g., a recombinant soluble SARS-CoV-2 spike protein described herein). Due to the degeneracy of the genetic code, any nucleic acid sequence that encodes a recombinant SARS-CoV-2 spike protein described herein (e.g., a recombinant soluble SARS-CoV-2 spike protein described herein) is encompassed herein. In specific embodiments, provided herein is a nucleic acid sequence comprising a nucleotide sequence encoding a recombinant SARS-CoV-2 spike protein described herein (with or without the signal peptide). In a specific embodiment, a nucleic acid sequence comprises a nucleotide sequence described herein.

In one embodiment, provided herein are nucleotide sequences encoding a recombinant SARS-CoV-2 spike protein described herein (e.g., a recombinant soluble SARS-CoV-2 spike protein described herein) that are optimized, e.g., by codon/RNA optimization, replacement with heterologous signal sequences, and elimination of mRNA instability elements. Methods to generate optimized nucleic acids encoding a recombinant SARS-CoV-2 spike protein described herein (e.g., a recombinant soluble SARS-CoV-2 spike protein described herein) for recombinant expression by introducing codon changes and/or eliminating inhibitory regions in the mRNA can be carried out by adapting the optimization methods described in, e.g., U.S. Pat. Nos. 5,965,726; 6, 174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In a specific embodiment, a nucleotide sequence encoding a recombinant SARS-CoV-2 spike protein described herein (e.g., a recombinant soluble SARS-CoV-2 spike protein described herein) is codon optimized. Techniques known to those of skill in the art may be used to codon optimize a nucleotide sequence encoding a recombinant SARS-CoV-2 spike protein described herein (e.g., a recombinant soluble SARS-CoV-2 spike protein described herein). In a specific embodiment, a recombinant SARS-CoV-2 spike protein described herein is encoded by a nucleotide sequence comprising the nucleic acid sequence of SEQ ID NO: 1, 3 or 5. In a specific embodiment, a recombinant SARS-CoV-2 spike protein described herein is encoded by a nucleotide sequence comprising a nucleic acid sequence that is at least 85%, 90%, 95%, or 98% identical to SEQ ID NO: 1, 3 or 5. In a specific embodiment, a recombinant SARS-CoV-2 spike protein described herein is encoded by a nucleotide sequence comprising a nucleic acid sequence that hybridizes to SEQ ID NO: 1, 3 or 5 under high, intermediate or low stringency conditions. In another specific embodiment, a nucleotide sequence encoding a recombinant SARS-CoV-2 spike protein described herein is isolated.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3.

In another specific embodiment, provided herein is a nucleotide sequence encoding a recombinant SARS-CoV-2 spike protein, wherein the nucleotide sequence comprises (or consists of) a nucleotide sequence that is at least 75%, 80%, 85%, 90%, 95% or 98% identical to the nucleotide sequence set forth in GenBank Accession No. MT380725.1. In another specific embodiment, provided herein is a nucleotide sequence encoding a recombinant SARS-CoV-2 spike protein, wherein the nucleotide sequence comprises (or consists of) a nucleotide sequence that is at least 80% or 85% identical to the nucleotide sequence set forth in GenBank Accession No. MT380725.1. In another specific embodiment, provided herein is a nucleotide sequence encoding a recombinant SARS-CoV-2 spike protein, wherein the nucleotide sequence comprises (or consists of) a nucleotide sequence that is at least 90%, 95% or 98% identical to the nucleotide sequence set forth in GenBank Accession No. MT380725.1. In another specific embodiment, provided herein is a nucleotide sequence encoding a recombinant SARS-CoV-2 spike protein, wherein the nucleotide sequence comprises (or consists of) the nucleotide sequence set forth in GenBank Accession No. MT380725.1.

In another specific embodiment, provided herein is a nucleotide sequence encoding a recombinant SARS-CoV-2 spike protein wherein the nucleotide sequence comprises (or consists of) a nucleotide sequence that is at least 75%, 80%, 85%, 90%, 95% or 98% identical to the nucleotide sequence set forth in GenBank Accession No. MT380724.1. In another specific embodiment, provided herein is s a nucleotide sequence encoding a recombinant SARS-CoV-2 spike protein wherein the nucleotide sequence comprises (or consists of) a nucleotide sequence that is at least 80% or 85% identical to the nucleotide sequence set forth in GenBank Accession No. MT380724.1. In another specific embodiment, provided herein is s a nucleotide sequence encoding a recombinant SARS-CoV-2 spike protein wherein the nucleotide sequence comprises (or consists of) a nucleotide sequence that is at least 90%, 95% or 98% identical to the nucleotide sequence set forth in GenBank Accession No. MT380724.1. In another specific embodiment, provided herein is s a nucleotide sequence encoding a recombinant SARS-CoV-2 spike protein wherein the nucleotide sequence comprises (or consists of) the nucleotide sequence set forth in GenBank Accession No. MT380724.1.

In certain embodiments, an “isolated” nucleic acid sequence or nucleotide sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, in certain embodiments, a nucleic acid sequence that is substantially free of cellular material includes preparations of nucleic acid sequence having less than about 30%, 20%, 10%, or 5% (by dry weight) of other nucleic acids. The term “substantially free of culture medium,” in certain embodiments, includes preparations of nucleic acid sequence in which the culture medium represents less than about 50%, 20%, 10%, or 5% of the volume of the preparation.

As used herein, the terms “nucleic acid” and “nucleotide” are intended to include DNA molecules (e.g., cDNA or genomic DNA) and RNA molecules (e.g., mRNA) and analogs of the DNA or RNA generated using nucleotide analogs. A nucleic acid sequence or nucleotide sequence can be single-stranded or double-stranded.

### 5.3 Methods for Recombinant Expression

In another aspect, provided herein are vectors (e.g., plasmids or viral vectors) comprising a nucleic acid sequence comprising a nucleotide sequence encoding a recombinant SARS-CoV-2 spike protein described herein (e.g., a recombinant soluble SARS-CoV-2 spike protein described herein). In a specific embodiment, the vector is an expression vector that is capable of directing the expression of a nucleic acid sequence encoding a recombinant SARS-CoV-2 spike protein described herein (e.g., a recombinant soluble SARS-CoV-2 spike protein described herein). Non-limiting examples of expression vectors include, but are not limited to, plasmids and viral vectors, such as replication defective retroviruses, adenoviruses, vesicular stomatitis virus (VSV), herpes virus, Newcastle disease virus (NDV), vaccinia virus (e.g., Modified Vaccinia Ankara virus), adeno-associated viruses (AAV), plant viruses, and baculoviruses. In a specific embodiment, provided herein is a mammalian vector (e.g., pCAGGS) comprising a nucleotide sequence encoding a recombinant spike protein described herein (e.g., a recombinant soluble SARS-CoV-2 spike protein described herein). In another specific embodiment, provided herein is a baculovirus vector (e.g., a modified pFastBacDual vector) comprising a nucleotide sequence encoding a recombinant SARS-CoV-2 spike protein described herein. In another specific embodiment, provided herein is a vector comprising a nucleotide sequence described herein (e.g., a vector described in Section 8, infra). In another specific embodiment, provided herein is a vector comprising the nucleotide sequence of SEQ ID NO: 7, 8 or 9.

Vectors comprising a nucleotide sequence described herein may be used to express the components in one or more cells and the components may be isolated and conjugated together with a linker using techniques known to one of skill in the art. In a specific embodiment, an expression vector comprises a nucleic acid sequence comprising a nucleotide sequence encoding a recombinant SARS-CoV-2 spike protein described herein (e.g., a recombinant soluble SARS-CoV-2 spike protein described herein) and in a form suitable for expression of the nucleic acid sequence in a cell. In a specific embodiment, an expression vector includes one or more regulatory sequences, selected on the basis of the cells to be used for expression, which is operably linked to the nucleic acid to be expressed. Within an expression vector, “operably linked” is intended to mean that a nucleic acid sequence of interest is linked to the regulatory sequence(s) in a manner which allows for expression of the nucleic acid sequence (e.g., in an in vitro transcription/translation system or in a cell when the vector is introduced into the cell). Regulatory sequences include promoters, enhancers and other expression control elements (e.g., polyadenylation signals). Regulatory sequences include those which direct constitutive expression of a nucleic acid in many types of cells, those which direct expression of the nucleic acid sequence only in certain cells (e.g., tissue-specific regulatory sequences), and those which direct the expression of the nucleic acid sequence upon stimulation with a particular agent (e.g., inducible regulatory sequences). It will be appreciated by those skilled in the art that the design of the expression vector can depend on such factors as the choice of the cell to be transformed, the level of expression of protein desired, etc.

Expression vectors can be designed for expression of a recombinant SARS-CoV-2 spike protein described herein (e.g., a recombinant soluble SARS-CoV-2 spike protein described herein) using prokaryotic (e.g., E. coli) or eukaryotic cells (e.g., insect cells (using baculovirus expression vectors, see, e.g., Treanor et al., 2007, JAMA, 297(14): 1577-1582 incorporated by reference herein in its entirety), yeast cells, plant cells, algae, avian, or mammalian cells). Examples of yeast cells include, but are not limited to S. pombe and S. cerevisiae and examples, infra. An example of avian cells includes, but is not limited to EB66 cells. Examples of mammalian cells include, but are not limited to, A549 cells, Crucell Per.C6 cells, Vero cells, CHO cells, VERO cells, BHK cells, HeLa cells, COS cells, MDCK cells, 293 cells, 3T3 cells or WI38 cells. In certain embodiments, the cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include Sf9, Sf21, Trichoplusia ni, Spodoptera frugiperda and Bombyx mori. In a particular embodiment, a mammalian cell culture system (e.g. Chinese hamster ovary or baby hamster kidney cells) is used for expression of a recombinant SARS-CoV-2 spike protein described herein. In another embodiment, a plant cell culture system is used for expression of a recombinant SARS-CoV-2 spike protein described herein. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. Cells comprising a nucleic acid sequence encoding a recombinant SARS-CoV-2 spike protein described herein (e.g., a recombinant soluble SARS-CoV-2 spike protein described herein) may be isolated, i.e., the cells are outside of the body of a subject. Cells comprising a nucleic acid sequence encoding a recombinant SARS-CoV-2 spike protein described herein (e.g., a recombinant soluble SARS-CoV-2 spike protein described herein) may be isolated from other cells, such as cells not comprising a nucleic acid sequence encoding a recombinant SARS-CoV-2 spike protein described herein (e.g., a recombinant soluble SARS-CoV-2 spike protein described herein). In certain embodiments, cells are engineered to express a recombinant SARS-CoV-2 spike protein described herein (e.g., a recombinant soluble SARS-CoV-2 spike protein described herein).

As an alternative to recombinant expression of a recombinant SARS-CoV-2 spike protein described herein (e.g., a recombinant soluble SARS-CoV-2 spike protein described herein) using a cell, an expression vector containing a nucleic acid sequence encoding a recombinant SARS-CoV-2 spike protein described herein can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a recombinant SARS-CoV-2 spike protein described herein (e.g., a recombinant soluble SARS-CoV-2 spike protein described herein).

Once a recombinant SARS-CoV-2 spike protein described herein (e.g., a recombinant soluble SARS-CoV-2 spike protein described herein) has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

In another aspect, provided herein are cells comprising a nucleic acid sequence comprising a nucleotide sequence encoding a recombinant SARS-CoV-2 spike protein described herein (e.g., a recombinant soluble SARS-CoV-2 spike protein described herein). In a specific embodiment, provided herein are cells comprising a vector comprising a nucleotide sequence encoding a recombinant SARS-CoV-2 spike protein described herein. The cells may be mammalian, insect (e.g., Sf9, Sf21, High Five, Trichoplusia ni, Spodoptera frugiperda and Bombyx mori etc.), or plant cells. In a specific embodiment, the cells are cell lines. In one embodiment, the cell lines are cell lines, such as Vero cells, CHO cells, MDCK cells, 293 T cells, HEK293T cells, Expi293F cells, BHK cells, HEK 293 cells, NS0 cells, PER.C6 cells, CRL7030 cells, HsS78Bst cells, HeLa cells, NIH 3T3 cells or other cells lines. In certain embodiments, a cell is one described herein, such as in Section 5 or 6, infra. In a specific embodiment, cells are those such as described herein below in Example 1, Example 2, Example 3, Example 4, or Example 5. Techniques known to one of skill in the art may be used to transfect or transform cells. Such techniques include, but are not limited to, calcium phosphate or calcium chloride co-precipitation, DEAE-dextran-mediated transfection, lipofection, and electroporation. Suitable methods for transforming or transfecting cells can be found in Sambrook et al., 1989, Molecular Cloning—A Laboratory Manual, 2nd Edition, Cold Spring Harbor Press, New York, and other laboratory manuals. The cells may be stably or transiently transfected with a nucleic acid sequence comprising a nucleotide sequence encoding a recombinant SARS-CoV-2 spike protein described herein (e.g., a recombinant soluble SARS-CoV-2 spike protein described herein). In certain embodiments, a cell is transiently transfected with an expression vector containing a nucleic acid sequence encoding a recombinant SARS-CoV-2 spike protein described herein (e.g., a recombinant soluble SARS-CoV-2 spike protein described herein). In other embodiments, a cell is stably transfected with an expression vector containing a nucleic acid sequence encoding a recombinant SARS-CoV-2 spike protein described herein (e.g., a recombinant soluble SARS-CoV-2 spike protein described herein). In a specific embodiment, the cells transfected or transformed with a nucleic acid sequence comprising a nucleotide sequence encoding a recombinant SARS-CoV-2 spike protein described herein (e.g., a recombinant soluble SARS-CoV-2 spike protein described herein) are isolated. In addition, bacteria or yeast may be transformed or transfected with a nucleotide sequence or vector described herein.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the cells along with the nucleic acid of interest. Examples of selectable markers include those which confer resistance to drugs, such as G418, hygromycin and methotrexate. Cells stably transfected with the introduced nucleic acid sequence can be identified by drug selection (e.g., cells that have incorporated the selectable marker gene will survive, while the other cells die).

In another aspect, provided herein are cells engineered to express a recombinant soluble SARS-CoV-2 spike protein described herein (e.g., a recombinant soluble SARS-CoV-2 spike protein described herein). In a specific embodiment, the cells are engineered to constitutively express a recombinant soluble SARS-CoV-2 spike protein described herein. In another embodiment, the cells may be induced to express a recombinant SARS-CoV-2 spike protein described herein. Techniques known to one of skill in the art may be used to engineer cells to express a recombinant soluble SARS-CoV-2 spike protein described herein. The cells may be mammalian, insect or plant cells. In a specific embodiment, the cells are cell lines. In one embodiment, the cell lines are cell lines, such as Vero, MDCK, 293 T cells, HeLa cells, CHO cells, Cos cells, 293 cells, HEK293F cells, Expi293F cells, HEK293T cells, BHK cells, HEK 293 cells, NS0 cells, PER.C6 cells, CRL7030 cells, HsS78Bst cells, NIH 3T3 cells or other cells lines. In a specific embodiment, cells are those such as described herein below. In a particular embodiment, cells provided herein are produced as described in Example 5, infra. In another embodiment, cells provided herein are those described in Example 5, infra. In a specific embodiment, the cells are isolated.

Cells transfected or transformed with a nucleotide sequence or vector described herein include the progeny or potential progeny of such cells. Progeny of such a cell may not be identical to the parent cell transfected or transformed with the nucleotide sequence or vector due to mutations or environmental influences that may occur in succeeding generations or integration of the polynucleotide into the cell genome.

Cells to be transformed or transfected with a nucleotide sequence or vector described herein can be chosen for those that modulate the expression of the nucleotide sequence or modifies and processes the product in the specific fashion desired. Such modifications (e.g., glycosylation) and processing (e.g., cleavage) of protein products can be important for the function of the protein. Different cells have characteristic and specific mechanisms for the post-translational processing and modification of proteins and gene products. Appropriate cell lines or host systems can be chosen to ensure the correct modification and processing of the foreign protein expressed. To this end, eukaryotic cells which possess the cellular machinery for proper processing of the primary transcript, glycosylation, and phosphorylation of the gene product can be used. Such mammalian cells include but are not limited to CHO, VERO, BHK, Hela, COS, MDCK, HEK 293, NIH 3T3, W138, BT483, Hs578T, HTB2, BT20 and T47D, NS0 (a murine myeloma cell line that does not endogenously produce any immunoglobulin chains), CRL7030 and HsS78Bst cells. In certain embodiments, a SARS-CoV-2 spike protein described herein (e.g., a recombinant soluble SARS-CoV-2 spike protein described herein) is produced in mammalian cells, such as CHO cells (e.g., the CHO cells described in Example 5, infra).

In another aspect, provided herein are methods for producing a recombinant SARS-CoV-2 spike protein described herein (e.g., a recombinant soluble SARS-CoV-2 spike protein described herein). In one embodiment, the method comprises culturing a cell(s) containing a nucleic acid sequence comprising a nucleotide sequence encoding a SARS-CoV-2 spike protein described herein in a suitable medium such that the protein is produced. In some embodiments, the method further comprises isolating the protein from the medium. In certain embodiments, a recombinant SARS-CoV-2 spike protein described herein is produced in a manner described in Section 6, infra. In a specific embodiment, a recombinant SARS-CoV-2 spike protein described herein is produced as described in Example 1, Example 3, or both. In another specific embodiment, a recombinant SARS-CoV-2 spike protein described herein is produced as described in Example 2, Example 4, or Example 5. Once a recombinant SARS-CoV-2 spike protein described herein has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins. In a specific embodiment, a recombinant soluble SARS-CoV-2 spike protein described herein is purified as described in Example 1, Example 3, or both. In a specific embodiment, a recombinant SARS-CoV-2 spike protein described herein is purified as described in Example 1, Example 2, Example 3, Example 4, or Example 5.

In a specific embodiment, the terms “purified” and “isolated” when used in the context of a protein that is obtained from cells refers to a protein which is substantially free of contaminating materials, e.g. cell debris, cell wall materials, membranes, organelles, the bulk of the nucleic acids, carbohydrates, proteins, and/or lipids present in cells. Thus, in certain embodiments, a protein that is isolated includes preparations of a protein having less than about 30%, 20%, 10%, 5%, 2%, or 1% (by dry weight) of cellular materials and/or contaminating materials. In a specific embodiment, a recombinant a recombinant soluble SARS-CoV-2 spike protein described herein is purified or isolated.

As an alternative to recombinant expression of a SARS-CoV-2 protein described herein using a cell, an expression vector containing a polynucleotide encoding a SARS-CoV-2 spike protein can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a SARS-CoV-2 spike protein described herein (e.g., a recombinant soluble SARS-CoV-2 spike protein described herein).

### 5.4 Compositions

In another aspect, provided herein are compositions comprising a recombinant SARS-CoV-2 spike protein described herein (e.g., a recombinant soluble SARS-CoV-2 spike protein described herein). In a specific embodiment, provided herein is a pharmaceutical composition (e.g., immunogenic composition) comprising a recombinant SARS-CoV-2 spike protein described herein (e.g., a recombinant soluble SARS-CoV-2 spike protein described herein) and a pharmaceutically acceptable carrier. The pharmaceutical compositions (e.g., immunogenic compositions) may be formulated to be suitable for the intended route of administration to a subject. For example, the pharmaceutical composition may be formulated to be suitable for parenteral, oral, intradermal, intranasal, transdermal, colorectal, intraperitoneal, and rectal administration. In a specific embodiment, the pharmaceutical composition (e.g., an immunogenic composition) may be formulated for intravenous, oral, intraperitoneal, intranasal, intratracheal, subcutaneous, intramuscular, topical, intradermal, transdermal or pulmonary administration. In a specific embodiment, the pharmaceutical composition (e.g., an immunogenic composition) may be formulated for intramuscular administration. In a specific embodiment, the pharmaceutical composition (e.g., an immunogenic composition) may be formulated for subcutaneous administration. An immunogenic composition described herein may be used to immunize a subject against SARS-CoV-2. An immunogenic composition described herein may also be used to prevent COVID-19 in a subject.

In certain embodiments, an immunogenic composition described herein comprises a polynucleotide (e.g., an RNA, an mRNA or cDNA) encoding a SARS-CoV-2 polypeptide. Such compositions may be formulated as a nanoparticle (e.g., a lipid nanoparticle) encapsulating or containing such a polynucleotide. See, e.g., Richner et al., 2017, Cell 168: 1114 and Richner et al, 2017, Cell 170(2):273 for examples of such formulations for mRNA delivery.

In another specific embodiment, a pharmaceutical composition (e.g., immunogenic composition) comprises a recombinant SARS-CoV-2 polypeptide described herein, and optionally an adjuvant.

In a specific embodiment, a pharmaceutical composition (e.g., immunogenic composition) comprises a recombinant SARS-CoV-2 polypeptide described herein, and optionally an adjuvant. In another specific embodiment, a pharmaceutical composition (e.g., immunogenic composition) comprises a recombinant SARS-CoV-2 polypeptide described herein in an admixture with a pharmaceutically acceptable carrier. In a specific embodiment, a pharmaceutical composition (e.g., immunogenic composition) comprises an adjuvant (e.g., an adjuvant described herein) and a recombinant SARS-CoV-2 polypeptide described herein, in an admixture with a pharmaceutically acceptable carrier. In another embodiment, a pharmaceutical composition (e.g., immunogenic composition) comprises an adjuvant (e.g., an adjuvant described herein) and a nucleic acid sequence comprising a nucleotide sequence encoding a recombinant SARS-CoV-2 polypeptide described herein.

In some embodiments, a pharmaceutical composition (e.g., an immunogenic composition) may comprise one or more other therapies (e.g., acetaminophen, ibuprofen, throat lozenges, cough suppressants, inhalers, antibiotics and oxygen) in addition to a recombinant SARS-CoV-2 polypeptide described herein. In certain embodiments, a pharmaceutical composition (e.g., an immunogenic composition) may comprise one or more other therapies (e.g., acetaminophen, ibuprofen, throat lozenges, cough suppressants, inhalers, antibiotics and oxygen) in addition to a therapy that utilizes a nucleic acid sequence comprising a nucleotide sequence encoding a recombinant SARS-CoV-2 polypeptide described herein.

In a specific embodiment, something is considered “pharmaceutically acceptable” if it is approved by a regulatory agency of the Federal or a state government or listed in the U.S. Pharmacopeia or other generally recognized pharmacopeiae for use in animals, and more particularly in humans. In a specific embodiment, a carrier is a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E.W. Martin. The formulation should suit the mode of administration.

In certain embodiments, the compositions described herein comprise, or are administered in combination with, an adjuvant. The adjuvant for administration in combination with a composition described herein may be administered before, concommitantly with, or after administration of said composition. In some embodiments, the adjuvant enhance or boosts an immune response to SARS-CoV-2 spike protein and does not produce an allergy or other adverse reaction. Adjuvants can enhance an immune response by several mechanisms including, e.g., lymphocyte recruitment, stimulation of B and/or T cells, and stimulation of macrophages.

In certain embodiments, an adjuvant augments the intrinsic response to a recombinant SARS-CoV-2 spike protein without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine, or other immunopotentiating agents.

### 5.5 Methods for Immunizing Against SARS-CoV-2 Spike Protein

In another aspect, provided herein is a method of immunizing against SARS-CoV-2 comprising administering to a subject a recombinant SARS-CoV-2 spike protein described herein or a pharmaceutical composition comprising such a protein. In another aspect, provided herein is a method of inducing an immune response against SARS-CoV-2 comprising administering to a subject a recombinant SARS-CoV-2 spike protein described herein or a pharmaceutical composition comprising such a protein. In another aspect, provided herein is a method of preventing against COVID-19 comprising administering to a subject a recombinant SARS-CoV-2 spike protein described herein or a pharmaceutical composition comprising such a protein. In a specific embodiment, the terms “subject” or “patient” are used interchangeably to refer to an animal (e.g., birds, reptiles, and mammals). In a specific embodiment, a subject is a human. In certain embodiments, the subject is a healthcare worker (e.g., a physician, nurse, physician's assistant, technician, etc.). In other embodiments, a subject is a non-human subject (e.g., rat, mouse, primate, etc.). In certain embodiments, a subject is a non-human mammal, such as a cat, cow, dog, pig, horse, ape, monkey or sheep.

In another aspect, provided herein are methods for inducing an immune response in a subject utilizing a recombinant SARS-CoV-2 spike protein described herein, a polynucleotide encoding such a protein, a vector containing or expressing such a protein or a composition described herein. In a specific embodiment, a method for inducing an immune response to SARS-CoV-2 spike protein in a subject comprises administering to a subject an effective amount of a recombinant SARS-CoV-2 spike protein, or an immunogenic composition thereof. In another embodiment, a method for inducing an immune response to SARS-CoV-2 spike protein in a subject comprises administering to a subject an effective amount of a polynucleotide (e.g., mRNA or DNA) encoding a recombinant SARS-CoV-2 spike protein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168: 1114 for examples of such formulations for mRNA delivery. In certain embodiments, the subject is a non-human subject. In some embodiments, the subject is a non-human subject, which produces human antibodies. In other embodiments, the subject is a human subject.

In another aspect, provided herein are methods for immunizing against SARS-CoV-2 in a subject utilizing a recombinant SARS-CoV-2 spike protein described herein, a polynucleotide encoding such a protein, a vector containing or expressing such a protein or a composition described herein. In a specific embodiment, a method for immunizing against SARS-CoV-2 in a subject comprises administering to a subject an effective amount of a recombinant SARS-CoV-2 spike protein, or an immunogenic composition thereof. In another embodiment, a method for immunizing against SARS-CoV-2 in a subject comprises administering to a subject an effective amount of a polynucleotide (e.g., mRNA or DNA) encoding a recombinant SARS-CoV-2 spike protein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168: 1114 for examples of such formulations for mRNA delivery. In certain embodiments, the subject is a non-human subject. In some embodiments, the subject is a non-human subject, which produces human antibodies. In other embodiments, the subject is a human subject.

In another aspect, provided herein are methods for preventing COVID-19 in a subject utilizing a recombinant SARS-CoV-2 spike protein described herein, a polynucleotide encoding such a protein, a vector containing or expressing such a protein or a composition described herein. In a specific embodiment, a method for preventing COVID-19 in a subject comprises administering to a subject in need thereof an effective amount of a recombinant SARS-CoV-2 spike protein, or an immunogenic composition thereof. In another embodiment, a method for preventing COVID-19 in a subject comprises administering to a subject in need thereof an effective amount of a polynucleotide (e.g., mRNA or DNA) encoding a recombinant SARS-CoV-2 spike protein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168: 1114 for examples of such formulations for mRNA delivery. In certain embodiments, the subject is a non-human subject. In some embodiments, the subject is a non-human subject, which produces human antibodies. In other embodiments, the subject is a human subject.

In certain embodiments, a subject is administered a dose of 0.1-100 mg/kg (e.g., 1-15 mg/kg or 10-15 mg/kg) of a recombinant SARS-CoV-2 spike protein described herein. In some embodiments, a subject is administered dose of 1-100 μg (e.g., 25 μg, 40 μg, 50 μg or 75 μg) of a polynucleotide encoding a recombinant SARS-CoV-2 spike protein described herein or an expression vector comprising such a polynucleotide. In some embodiments, a recombinant SARS-CoV-2 spike protein, a polynucleotide encoding a recombinant SARS-CoV-2 spike protein described herein, or a pharmaceutical composition described herein (e.g., an immunogenic composition described herein) is administered to a subject as a single dose to a subject. In certain embodiments, a recombinant SARS-CoV-2 spike protein, a polynucleotide encoding a recombinant SARS-CoV-2 spike protein described herein, or a pharmaceutical composition described herein (e.g., an immunogenic composition described herein) is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In accordance with these embodiments, booster inoculations may be administered to the subject at 3 to 6 month or 6 to 12 month intervals following the second inoculation.

In some embodiments, the administration of a SARS-CoV-2 spike protein described herein, a nucleotide sequence encoding a SARS-CoV-2 spike protein described herein, or a pharmaceutical composition described herein (e.g., an immunogenic composition described herein) further comprises the administration of an additional therapy (e.g., acetaminophen, ibuprofen, throat lozenges, cough suppressants, inhalers, antibiotics and oxygen).

In a specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject prevents the onset or development of one, two or more symptoms of COVID-19, reduces the severity of one, two or more symptoms of COVID-19, or prevents the onset or development of one, two or more symptoms of COVID-19 and reduces the severity of one, two or more symptoms of COVID-19. Symptoms of COVID-19 include congested or runny nose, cough, fever, sore throat, headache, wheezing, rapid or shallow breathing or difficulty breathing, bluish color the skin due to lack of oxygen, chills, muscle pain, loss of taste and/or smell, nausea, vomiting, and diarrhea.

In a specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject prevents the spread of SARS-CoV-2 infection. In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject prevents hospitalization. In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject prevents COVID-19. In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject prevents recurring SARS-CoV-2 infections.

In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein induces antibodies to SARS-CoV-2 spike protein. In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein induces both mucosal and systemic antibodies to SARS-CoV-2 spike protein (e.g., neutralizing antibodies). In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject induces neutralizing antibody to SARS-CoV-2 spike protein. In another specific embodiment, the administration of a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein to a subject induces robust, long-lived (e.g., 6 months, 1 year, 2 years, 3 years or more), antigen-specific humoral immunity.

In some embodiments a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a subject predisposed or susceptible to COVID-19.

In certain embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a human. In specific embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to an elderly human. In certain embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a human infant. In some embodiments, a SARS-CoV-2 spike protein described herein, a nucleotide sequence encoding a SARS-CoV-2 spike protein described herein, a pharmaceutical composition described herein, or a combination therapy described herein is administered to a human child. In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a human toddler. In certain embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a human adult. As used herein, the term “elderly human” refers to a human 65 years or older. As used herein, the term “human adult” refers to a human that is 18 years or older. As used herein, the term “human child” refers to a human that is 1 year to 18 years old. As used herein, the term “human toddler” refers to a human that is 1 year to 3 years old. As used herein, the term “human infant” refers to a newborn to 1 year old year human.

In a specific embodiment, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered a subject (e.g., a human subject) in close contact with an individual with increased risk of COVID-19 or SARS-CoV-2 infection. In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered a subject (e.g., a human subject) with a condition that increases susceptibility to SARS-CoV-2 complications or for which SARS-CoV-2 increases complications associated with the condition. Examples of conditions that increase susceptibility to SARS-CoV-2 complications or for which SARS-CoV-2 increases complications associated with the condition include conditions that affect the lung, such as cystic fibrosis, chronic obstructive pulmonary disease (COPD), emphysema, asthma, or bacterial infections (e.g., infections caused by Haemophilus influenzae, Streptococcus pneumoniae, Legionella pneumophila, and Chlamydia trachomatus); cardiovascular disease (e.g., congenital heart disease, congestive heart failure, and coronary artery disease); and endocrine disorders (e.g., diabetes).

In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a subject (e.g., a human subject) that resides in a group home, such as a nursing home. In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a subject (e.g., a human subject) that works in, or spends a significant amount of time in, a group home, e.g., a nursing home. In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a subject (e.g., a human subject) that is a health care worker (e.g., a doctor or nurse). In some embodiments, a SARS-CoV-2 spike protein described herein, a nucleotide sequence encoding a SARS-CoV-2 spike protein described herein, a pharmaceutical composition described herein (e.g., an immunogenic composition described herein), or a combination therapy described herein is administered to a subject (e.g., a human subject) that is a smoker.

In some embodiments, a SARS-CoV-2 spike protein described herein, a nucleotide sequence encoding a SARS-CoV-2 spike protein described herein, a pharmaceutical composition described herein, or a combination therapy described herein is administered to (1) a subject (e.g., a human subject) who can transmit SARS-CoV-2 to those at high risk for complications, such as, e.g., members of households with high-risk subjects, including households that will include human infants (e.g., infants younger than 6 months), (2) a subject coming into contact with human infants (e.g., infants less than 6 months of age), (3) a subject who will come into contact with subjects who live in nursing homes or other long-term care facilities, (4) a subject who is or will come into contact with an elderly human, or (5) a subject who will come into contact with subjects with long-term disorders of the lungs, heart, or circulation; individuals with metabolic diseases (e.g., diabetes) or subjects with weakened immune systems (including immunosuppression caused by medications, malignancies such as cancer, organ transplant, or HIV infection).

### 5.6 Methods for Detecting Antibodies Specific for SARS-CoV-2 Spike Protein

In another aspect, a recombinant SARS-CoV-2 spike protein described herein may be used in an immunoassay, such as a Western blot, an ELISA, or flow cytometry, for the detection of antibodies that bind to the spike protein of SARS-CoV-2. Techniques known to those skilled in the art may be used to produce and run such an immunoassay. In a specific embodiment, a recombinant SARS-CoV-2 spike protein described herein is used an ELISA described herein. In one embodiment, an ELISA using a recombinant SARS-CoV-2 spike protein described herein comprises two, three or more, or all of the steps of an ELISA described herein (see, e.g., Examples 1, 2, 3, 4, 6, 7, 8, 10, 11, 12, 13, and 14 infra).

In another aspect, a recombinant SARS-CoV-2 spike protein described herein can be used to assess the antibody response of a subject or a population of subjects to a SARS-CoV-2 spike protein. In specific embodiments, a recombinant SARS-CoV-2 spike protein described herein can be used to assess the presence of SARS-CoV-2 spike protein receptor binding domain-specific antibodies in a subject or population of subjects. In another specific embodiment, an antibody response of a subject or a population of subjects that has/have been infected by SARS-CoV-2 or immunized with a vaccine that includes a SARS-CoV-2 spike protein, may be assessed in an immunoassay (e.g., an ELISA described herein) to identify the types of antibodies (e.g., IgG, IgA, IgM, etc) in the subject or population of subjects specific for the SARS-CoV-2 spike protein. In specific embodiments, a biological sample (e.g., blood, sera or plasma) from a subject or population of subjects may be isolated and tested directly for the presence of antibodies, or may be processed (e.g., to obtain sera) and subsequently tested for the presence of antibodies. In another embodiment, a biological sample is obtained and processed as described in Example 1, Example 3, or both. In another embodiment, a biological sample is obtained and processed as described in Example 2, 4 or 6. In another embodiment, a biological sample is obtained and processed as described in Example 7, 8, 10, 11, 12, 13, 14 or 16. For example, a biological sample may be heat inactivated (e.g., heat inactivated at 56° C. for 30 minutes to 1 hour, such as described in the Examples, infra). Such antibody testing can utilize assays known in the art, e.g., ELISA such as an ELISA described herein (see Example 1, 2, 3, 4, 6, 7, 8, 10, 11, 12, 13, 15 or 16 infra). In a specific embodiment, a recombinant SARS-CoV-2 spike protein described herein is used in an ELISA described herein, such as described in Example 1, Example 3, or both. In another specific embodiment, a recombinant SARS-CoV-2 spike protein described herein is used in an ELISA described herein, such as described in Example 2, 4, 6, 7, 8, 10, 11, 12, 13, 14 or 16.

In one embodiment, an antibody profile of subject or a population of subjects may allow for the identification surrogate markers/endpoints important in determining the clinical response following administration of a vaccine for SARS-CoV-2 or immunoprotection against SARS-CoV-2. In a specific embodiment, an antibody profile of a subject or a population of subjects is assessed to determine whether said subject or population of subjects possesses antibodies against a SARS-CoV-2 spike protein. Such an assessment may allow identification of a subject or population of subjects that may be protected from COVID-19. Such an assessment may also identify sera that may be useful to passively immunize subjects. In another embodiment, an antibody profile of subject or a population of subjects may be useful to assess responses to vaccination, correlates of protection, and/or standardization of therapeutic approaches, such as monoclonal antibody and plasma transfer.

In another embodiment, provided herein is a method of assessing/detecting the presence of antibodies in a subject that are specific for SARS-CoV-2 spike protein comprising contacting in vitro a biological sample (e.g., blood, plasma or sera) from said subject with a recombinant soluble SARS-CoV-2 spike protein. In another specific embodiment, provided herein is a method of assessing/detecting the presence of antibodies in a subject that are specific for the receptor binding domain of SARS-CoV-2 spike protein comprising contacting in vitro a biological sample (e.g., blood, plasma or sera) from said subject with a recombinant soluble SARS-CoV-2 spike protein. The biological sample (e.g., plasma or sera) may be inactivated (e.g., heat inactivated, such as described in Example 2, infra) and/or diluted, such as described in Examples 1-4, 6, 7 and 8, infra, prior to use. The biological sample may be inactivated and/or diluted as described in Example 11, 12, 13, or 14 infra, prior to use. The biological sample may be processed and diluted as described in Example 16.

In a specific embodiment, provided herein is a method of assessing/detecting the presence of antibodies in a subject that are specific for SARS-CoV-2 spike protein comprising contacting in vitro a biological sample (e.g., blood, plasma or sera) from said subject with a recombinant soluble SARS-CoV-2 spike protein described herein. In another specific embodiment, provided herein is a method of assessing/detecting the presence of antibodies in a subject that are specific for the receptor binding domain of SARS-CoV-2 spike protein comprising contacting in vitro a biological sample (e.g., blood, plasma or sera) from said subject with a recombinant soluble SARS-CoV-2 spike protein described herein. The biological sample (e.g., plasma or sera) may be inactivated (e.g., heat inactivated, such as described in Example 2, infra) and/or diluted, such as described in any one of Examples 1-4, 6, 7 and 8, infra, prior to use. See also Examples 11-14 and 16 for methods for inactivating and diluting a biological sample.

In a specific embodiment, provided herein is a method for detecting an antibody(ies) that specifically binds to SARS-CoV-2 spike protein, comprising contacting a recombinant soluble SARS-CoV-2 spike protein (e.g., a recombinant SARS-CoV-2 spike protein described herein) with a biological sample from a subject and detecting the binding of antibody(ies) to the recombinant soluble SARS-CoV-2 spike protein. The binding of the antibody(ies) to the recombinant soluble SARS-CoV-2 spike protein may be detected by using an antibody that binds to the constant region of the antibody(ies). The antibody(ies) that binds to the constant region may be labeled with a chemiluminescent agent, radioactive label, or other label known to one of skill in the art or described herein. In a specific embodiment, the method involves an ELISA or other immunoassay described herein. An immunoassay described herein (e.g., an ELISA) may be run in a high-throughput format, such as described below in, e.g., Example 1, Example 2, Example 3, Example 4, Example 6, Example 7, or Example 8, infra. See also Examples 11-14 and 16 for methods for detecting antibody that specifically binds to SARS-CoV-2 spike protein.

In certain embodiments, a biological sample is obtained from a subject 5, 6, 7, 8, 9, 10 or more days after vaccination of the subject with a SARS-CoV-2 vaccine (e.g., an mRNA-based vaccine or viral-based vaccine), after a suspected SARS-CoV-2 infection in the subject, after the subject has tested positive for a SARS-CoV-2 infection, or after the subject has been exposed to another subject suspected of having or to have had a SARS-CoV-2 infection or tested positive for a SARS-CoV-2 infection. In some embodiments, a biological sample is obtained from a subject 11, 12, 13, 14 or 15 or more days after vaccination of the subject with a SARS-CoV-2 vaccine (e.g., an mRNA-based vaccine or viral-based vaccine), after a suspected SARS-CoV-2 infection in the subject, after the subject has tested positive for a SARS-CoV-2 infection, or after the subject has been exposed to another subject suspected of having or to have had a SARS-CoV-2 infection or tested positive for a SARS-CoV-2 infection. In certain embodiments, a biological sample is obtained from a subject 16, 17, 18, 19, 20, 21, 22, 23, 24 or more days after vaccination of the subject with a SARS-CoV-2 vaccine (e.g., an mRNA-based vaccine or viral-based vaccine), after a suspected SARS-CoV-2 infection in the subject, after the subject has tested positive for a SARS-CoV-2 infection, or after the subject has been exposed to another subject suspected of having or to have had a SARS-CoV-2 infection or tested positive for a SARS-CoV-2 infection. The subject from which the biological sample is obtained may be asymptotic or symptomic for a SARS-CoV-2 infection. In some embodiments, the subject from which the biological sample is obtained has been diagnosed with a SARS-CoV-2 infection, or COVID-19. The biological sample may be a blood sample, such as sera or plasma, or saliva. A specimen may be a biological sample or a therapeutic antibody or polyclonal antibody sample. The biological sample may be heat inactivated (e.g, as described in Section 6, infra) and/or diluted (e.g, as described in Section 6, infra). In a particular embodiment, the biological sample is heat inactivated (e.g, as described in Section 6, infra) and diluted (e.g, as described in Section 6, infra). In some embodiments, a monoclonal antibody or polyclonal antibody sample (e.g., a therapeutic monoclonal antibody) is tested for binding to a recombinant soluble SARS-CoV-2 spike protein described herein. The samples (e.g., biological sample or antibody sample) may be diluted 5 to 500 fold, 250 to 500 fold, or 500 to 1000 fold in buffer. The samples (e.g., biological sample or antibody sample) may be diluted 5-, 10-, 15-, 20-, 25-30-, 40-, or 50-fold in a buffer. The samples (e.g., biological sample or antibody sample) may be diluted 75-, 100-, 125-, 150-, 200-, 250-, 275-, 300-, 350-, 400-, 450- or 500-fold in a buffer. The samples may be diluted 550-, 600-650-, 700-, 750-, 800-, 850-, 900-, 950- or 1000-fold in a buffer. The samples (e.g., biological sample or antibody sample) may be diluted 1000 to 5,000 fold, 5,000 to 10,000 fold, or 1,000 to 10,000 fold in a buffer. The buffer may be one described herein or known to one of skill in the art. In some embodiments, a protein based solution with preservatives, such as described in Section 6, infra.

In certain embodiments, a biological sample is a saliva sample. The saliva sample may be processed and diluted. For example, a saliva sample (e.g., 1 ml of saliva) may be centrifuged for a certain period of time (e.g., 5-15 minutes, 5 minutes, 10 minutes or 15 minutes), diluted in buffer (e.g., 100 μl is diluted 1:5 by adding buffer, such as saline buffer) and the vortexed. In a specific embodiment, a saliva sample is obtained and analyzed for SARS-CoV-2 antibody as described in Example 16.

In a specific embodiment, provided herein is a method for detecting an antibody(ies) that specifically binds to SARS-CoV-2 spike protein, comprising contacting a recombinant soluble SARS-CoV-2 spike protein (e.g., a recombinant SARS-CoV-2 spike protein described herein) with a specimen (e.g., an antibody sample, such as a monoclonal antibody or polyclonal antibodies) and detecting the binding of antibody to the recombinant soluble SARS-CoV-2 spike protein. The binding of the antibody to the recombinant soluble SARS-CoV-2 spike protein may be detected by using an antibody that binds to the constant region of the antibody(ies). The antibody that binds to the constant region may be labeled with a chemiluminescent agent, radioactive label, or other label known to one of skill in the art or described herein.

In certain embodiments, a recombinant soluble SARS-CoV-2 spike protein described herein is immobilized (e.g., coated) on a solid support and the binding of antibody in a sample (e.g., a biological sample) to the recombinant soluble SARS-CoV-2 spike protein is detected. Solid supports include silica gels, resins, derivatized plastic films, glass surfaces, cellulose, polyacrylamide, nylon, polystyrene, polyvinyl chloride, polypropylene, beads (e.g., glass beads, plastic beads, magnetic beads, or polystyrene beads), or alumina gels. In some embodiments, a recombinant soluble SARS-CoV-2 spike protein described herein is immobilized (e.g., coated) on a bead (e.g., a glass bead, plastic bead, magnetic bead, or polystyrene bead), a test strip, a microtiter plate, a membrane, a glass surface, a slide (e.g., a microscopy slide), a microarray, a column (e.g., a chromatography column), or a biochip.

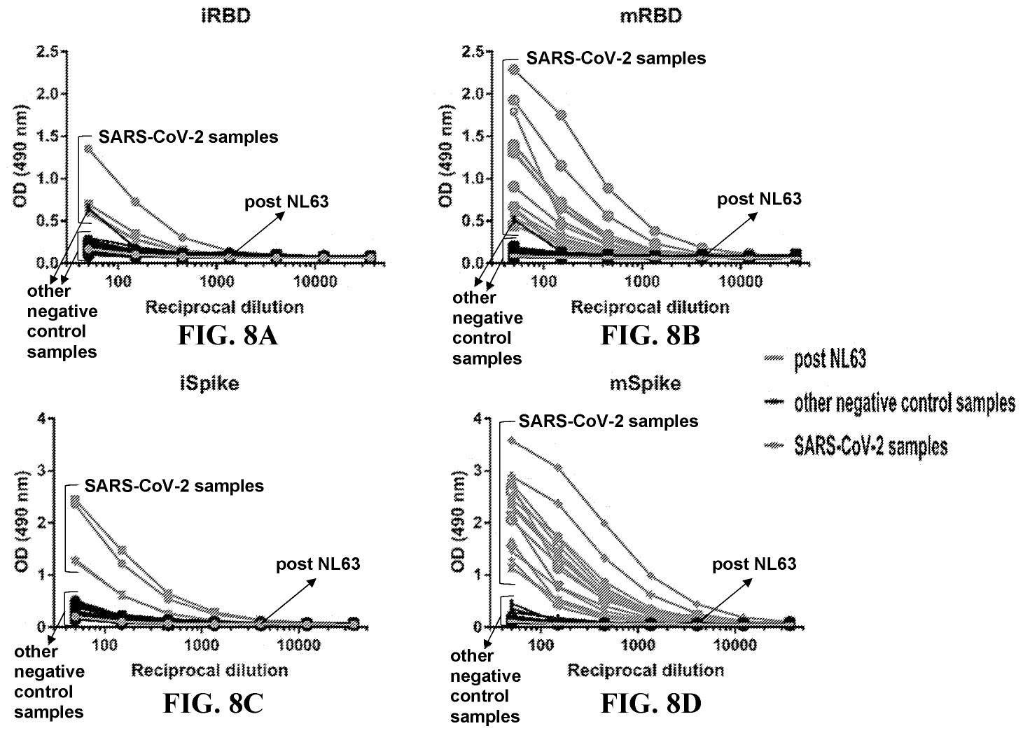

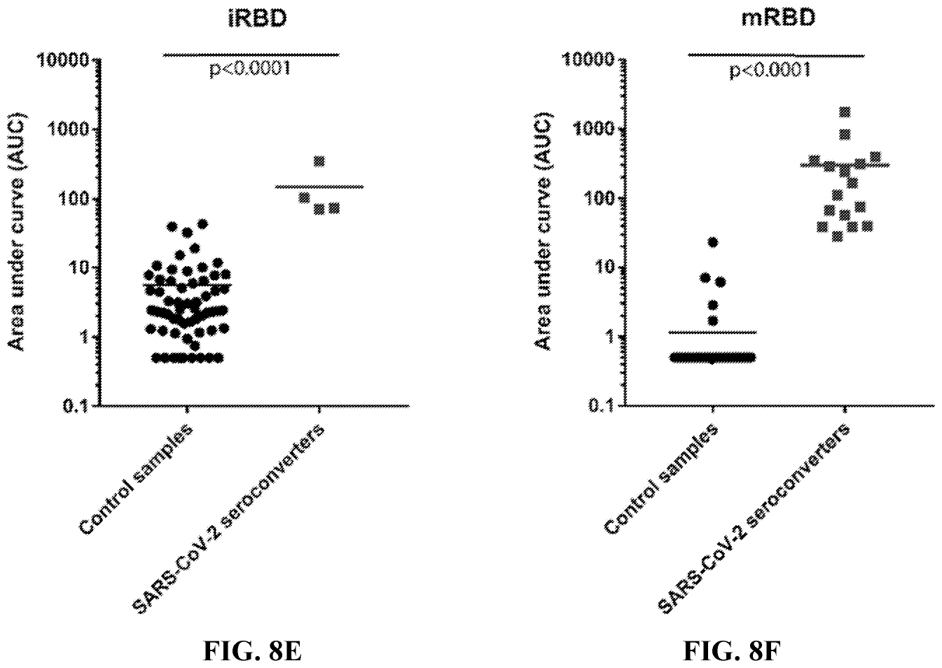

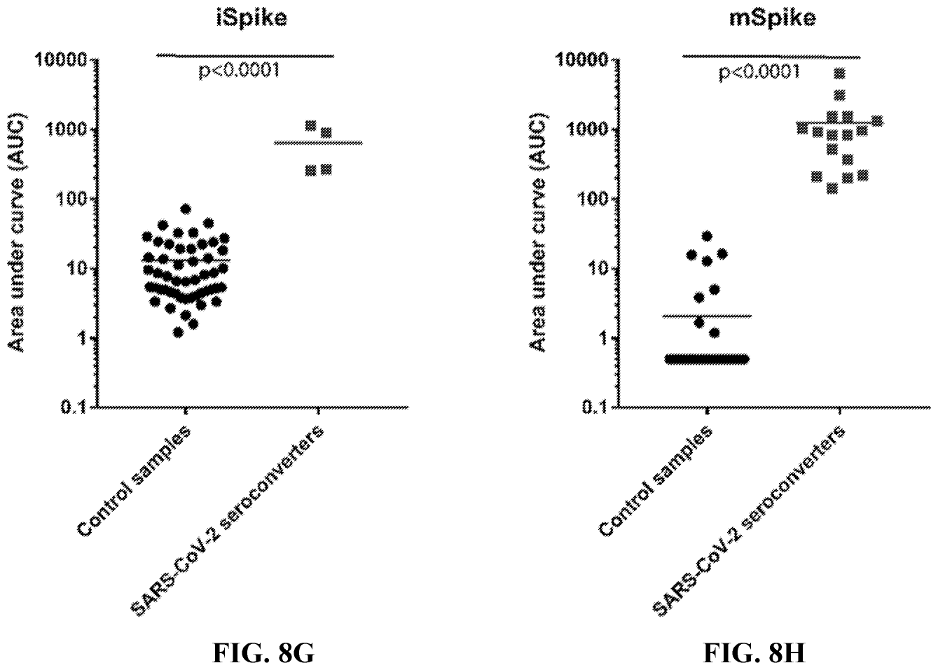

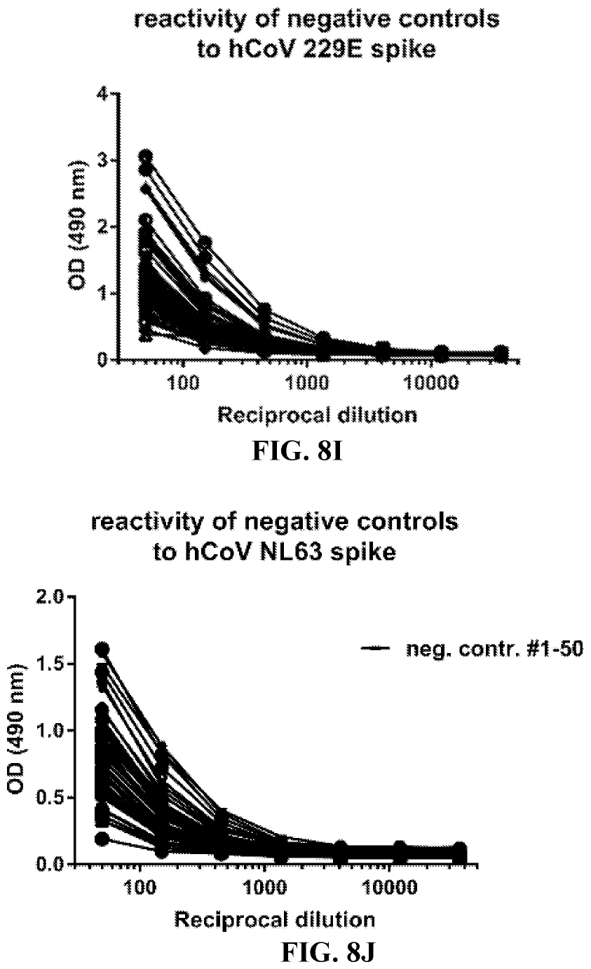

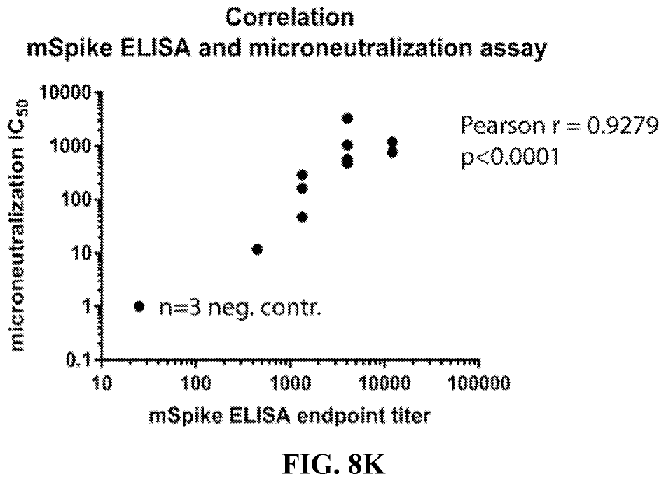

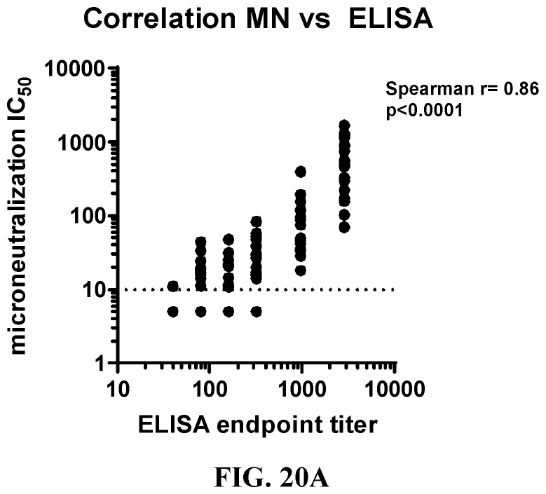

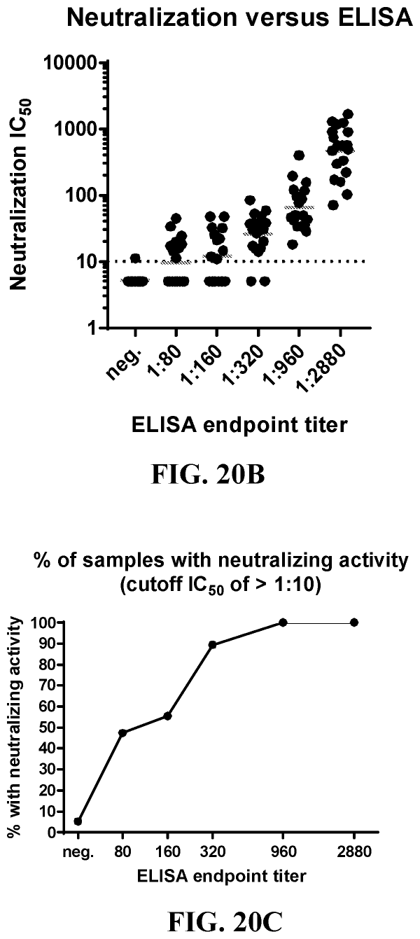

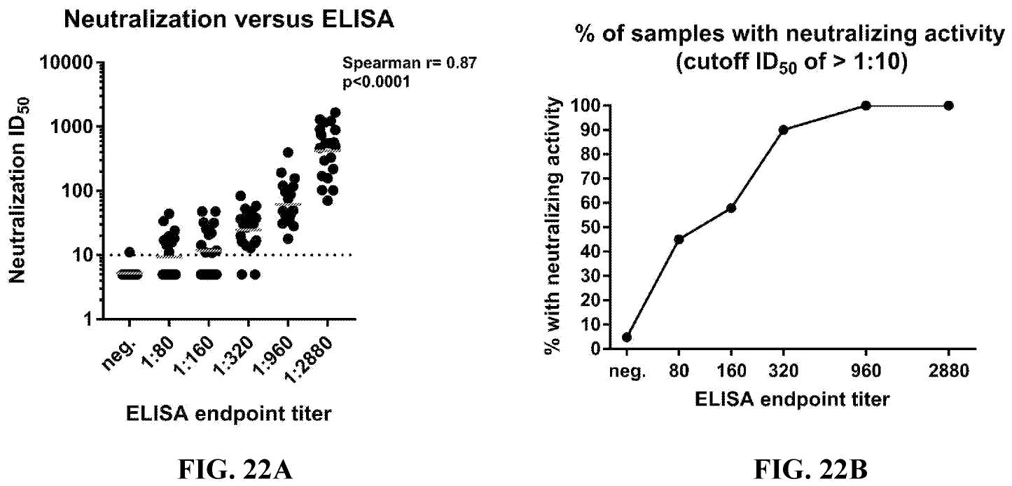

In a specific embodiment, provided herein are methods for detecting an antibody(ies) that specifically binds to SARS-CoV-2 spike protein, comprising: (1) incubating a specimen (e.g., a biological sample or antibody sample) in a well coated with recombinant SARS-CoV-2 spike protein (e.g., a recombinant SARS-CoV-2 spike protein described herein) for a first period time; (2) washing (e.g., using a washing solution described herein, such as in Example 2, 3, 7 or 8, infra) the well; (3) incubating a labeled antibody that binds to an isotype or subtype of immunoglobulin in the well for a second period of time; (4) washing (e.g., using a washing solution described herein, such as in Example 2, 3, 7, or 8, infra) the well; and (5) detecting the binding of the labeled antibody to the recombinant SARS-CoV-2 spike protein in the well. In some embodiments, the well is coated with a certain concentration (e.g., 2 micrograms/mL) of the recombinant SARS-CoV-2 spike protein. In certain embodiments, the first period of time and/or second period of time is 30 minutes, 45 minutes, 1 hour, 1.5 hours, 2 hours, 2.5 hours, 3 hours, or more. In some embodiments, the specimen is a biological sample is blood, sera, or plasma. In certain embodiments, the biological sample (e.g., sera or plasma) is inactivated before being incubated with a well coated with recombinant SARs-CoV-2 spike protein. In some embodiments, the specimen is serially diluted, such as described in Example 1, 2, 3, 4, 6, 7 or 8, infra. In specific embodiments, the method comprises use of a negative control, such as an antibody(ies) that specifically binds to a spike protein of an alphacoronavirus, such as, e.g., an antibody(ies) that specifically binds to a spike protein of NL63, 229E or both, or a betacoronavirus, such as, e.g., an antibody(ies) that specifically binds to a spike protein of OC43, HKU1 or both. In some embodiments, the method comprises use of a positive control, such as an antibody(ies) that specifically binds to the spike protein of SARS-CoV-2 (e.g., antibodies from COVID-19 patients or monoclonal antibodies (mAbs) like CR3022). In certain embodiments, the method comprises use of a negative control and a positive control. In specific embodiments, when a negative and/or positive control is used the method involves the same steps as with the specimen in different wells. In certain embodiments, the method is run in a high-throughput format so that the detection of antibody(ies) in multiple specimens may be conducted concurrently. For example, in certain embodiments, a 96 well microtiter plate is used with different specimens or controls in different wells, or different dilutions of a specimen in different wells, wherein the wells are coated with a recombinant SARS-CoV-2 spike protein. In some embodiments, the labeled antibody is labeled with a radioactive moiety, a chemiluminescent moiety, a fluorescent moiety or other detectable label. In a specific embodiment, the labeled antibody is labeled with horse radish peroxidase (HRP) conjugated to antibody that binds to the a particular immunoglobulin isotype or subtype (e.g., anti-human IgG antibody) and detection of the binding of the labeled antibody to the recombinant SARS-CoV-2 spike protein comprises washing the well, incubating substrate (e.g., o-phenylenediamine dihydrocloride) in the well, stopping the reaction (e.g., with 3 M HCl stop solution), and reading the optical density of the well at, e.g., 490 nm. In some embodiments, the method described above is run once with a recombinant SARS-CoV-2 spike protein comprising (or consisting of) the receptor binding domain of SARS-CoV-2 spike protein (as such as, e.g., described herein) and run a second time with a recombinant SARS-CoV-2 spike protein comprising the full length SAR-CoV-2 spike protein (as such as, e.g., described herein). In certain embodiments, the optical density for a series of specimens with different antibody titers may be used to arbitrarily assign optical density measurements to the different specimens, such as done in Section 6, infra. In specific embodiments, an IC50 optical density is assigned and used to quantify antibody(ies). See, e.g., Section 6 or 8, infra, for methods of quantifying and calibrating to arrive at the IC50. In a particular embodiment, an IC50 optical density of 331.4 is assigned. Such an IC50 may be used to provide more reliable values for antibody levels that correlate with neutralization titers and efficacy of vaccines (e.g., quantitative ELISA that measures IgG levels is used in the evaluation of vaccines as a surrogate assay for efficacy, which may be used a gold standard standard). For example, an optical density lower than 331.4 would mean that less antibody(ies) is present in a specimen whereas an IC50 higher than 331.4 would mean that there is more antibody(ies) specimen. In some embodiments, the quantity of antibody(ies) in an immunoassay described herein correlates with the microneutralization titer. For example, the higher the IC50 optical density, the higher the microneutralization titer. See, e.g, FIG. 8K, or FIGS. 20A-20C, for correlation between an ELISA IgG titer and microneutralization. See also, e.g, FIGS. 22A and 22B for correlation between an ELISA IgG titer and microneutralization.

In certain embodiments, the optical density for a series of specimens with different antibody titers may be used to arbitrarily assign optical density measurements to the different specimens, such as done in Section 6, infra. In specific embodiments, an ID50 optical density is assigned and used to quantify antibody(ies). In a particular embodiment, an ID50 optical density of 331.4 is assigned. Such an ID50 may be used to provide more reliable values for antibody levels that correlate with neutralization titers and efficacy of vaccines (e.g., quantitative ELISA that measures IgG levels is used in the evaluation of vaccines as a surrogate assay for efficacy, which may be used a gold standard standard). For example, an optical density lower than 331.4 would mean that less antibody(ies) is present in a specimen whereas an ID50 higher than 331.4 would mean that there is more antibody(ies) specimen. In some embodiments, the quantity of antibody(ies) in an immunoassay described herein correlates with the microneutralization titer. For example, the higher the ID50 optical density, the higher the microneutralization titer.

In a specific embodiment, provided herein is a two-step test for IgG against SARS-CoV-2 in a direct Enzyme-Linked Immunosorbent Assay (ELISA) for the qualitative detection of IgG antibodies against recombinant receptor binding domain of SARS-CoV-2 (such as described herein) in serum followed by confirmatory ELISA of positive specimen against full-length SARS-CoV-2 spike protein in serum. In a specific embodiment, the two-step test for IgG against SARS-CoV-2 is one described in Section 6, infra. In another specific embodiment, provided herein is a quantitative ELISA of positive specimen against full length SARS-CoV-2 spike protein in serum and plasma. In one embodiment, a first step of such quantitative ELISA comprises the qualitative detection of IgG antibodies against a recombinant receptor binding domain of SARS-CoV-2 (such as described herein) in serum or plasma (see, e.g., Section 6.8.1 of Example 8 for a description of such an assay). In another embodiment, a second step of such quantitative ELISA comprises in vitro quantification of IgG antibody to SARS-CoV-2 in serum an plasma from individuals positive from the qualitative detection of IgG antibodies against recombinant a receptor binding domain of SARS-CoV-2 spike protein (such as described herein), for the assessment of seroconversion from an antibody negative status to an antibody positive status in infected patients. In a specific embodiment, the in vitro qualitative identification of IgG antibody in serum and plasma from individuals suspected of seroconversion from an antibody negative status to an antibody positive status in acutely infected patients, and identification of patients with SARS-CoV-2 IgG titers of up to 1:2880. See, e.g., Example 8, infra, for a description of such an assay how it may be performed.

In some embodiments, a microneutralization assay, such as described in Example 1, 2, 7, or 9, infra, is conducted with a specimen for which an immunoassay described herein is conducted. In certain embodiments, the combination of microneutralization titer and the results from an immunoassay described herein provide a surrogate for immunoprotection.

In certain embodiments, a result of ≤5 AU/ml in an ELISA, such as described in Example 7 or 8, infra, indicates that a negative result for antibodies against SARS-CoV-2. In some embodiments, a result of ≥5 AU/ml in an ELISA, such as described in Example 7 or 8, infra, indicates that levels of anti-SARS-CoV-2 IgG has been detected at levels consistent with protective immunity against SARS-CoV-2 infection.

In certain embodiments, a result of <5 AU/ml in an ELISA, such as described in Example 7, infra, indicates that a negative result for antibodies against SARS-CoV-2. In some embodiments, a result of <5 AU/ml in an ELISA, such as described in Example 7, infra, indicates that the result is below the test's limit of quantitation (LoQ) for antibodies against SARS-CoV-2. In some embodiments, a result of >5 AU/ml in an ELISA, such as described in Example 7 or 8, infra, indicates that levels of anti-SARS-CoV-2 IgG has been detected at levels consistent with protective immunity against SARS-CoV-2 infection. In certain embodiments, the methods described in Examples 11, 12, 13, 14 or 16 for interpreting ELISA assay results are used.

In some embodiments, provided herein is a method for the detection of antibody that specifically binds to human SARS-CoV-2 spike protein, comprising: (a) incubating a specimen in a well coated with a first recombinant soluble SARS-CoV-2 spike protein for a first period of time; (b) washing the well; (c) incubating a labeled antibody that binds to an isotype or subtype of an immunoglobulin (e.g., IgG) for a second period of time in the well; (d) washing the well; and (e) detecting the binding of the labeled antibody to the first recombinant SARS-CoV-2 spike protein. In certain embodiment, the method further comprises (f) incubating the specimen in a well coated with a second recombinant soluble SARS-CoV-2 spike protein for a third period of time, wherein the second recombinant soluble SARS-CoV-2 spike protein is different from the first recombinant soluble SARS-CoV-2 spike protein (e.g., the first recombinant soluble SARS-CoV-2 spike protein may comprise the receptor binding domain of a SARS-CoV-2 spike protein but not the entire ectodomain and the second recombinant soluble SARS-CoV-2 spike protein may comprise the ectodomain of a SARS-CoV-2 spike protein); (g) washing the well; (h) incubating a second labeled antibody that binds to an isotype or subtype of an immunoglobulin (e.g., IgG) for a fourth period of time in well; (i) washing the well; and (j) detecting the binding of the second labeled antibody to the second recombinant SARS-CoV-2 spike protein. In certain embodiments, the first time period, second time period, or both are 30 minutes, 45 minutes, 1 hour, 2 hours, 2.5 hours or 3 hours. In some embodiments, the third and fourth time periods are 30 minutes, 45 minutes, 1 hour, 2 hours, 2.5 hours or 3 hours. In certain embodiments, the second labeled antibody is the same as the first labeled antibody. The specimen may be serially diluted, such as described in Section 6, infra. In a specific embodiment, the specimen is a biological sample, such as, e.g., blood, sera, or plasma. The biological sample may be from an asymptomic subject, a symptomic subject or those suspected of having a SARS-CoV-2 infection or COVID-19. The biological sample may be inactivated before being incubated with the well coated with recombinant SARS-CoV-2 spike protein. In another embodiment, the specimen is an antibody or antibodies against SARS-CoV-2 spike protein.

In some embodiments, provided herein is a method for the detection of antibody that specifically binds to human SARS-CoV-2 spike protein, comprising: (a) incubating a specimen in a well coated with a first recombinant soluble SARS-CoV-2 spike protein for a first period of time, wherein the first recombinant soluble SARS-CoV-2 spike protein comprises the receptor binding domain of a SARS-CoV-2 spike protein; (b) washing the well; (c) incubating a first labeled antibody that binds to an isotype or subtype of an immunoglobulin (e.g., IgG) for a second period of time in the well; (d) washing the well; and (e) detecting the binding of the first labeled antibody to the first recombinant SARS-CoV-2 spike protein. In certain embodiment, the method further comprises (f) incubating the specimen in a well coated with a second recombinant soluble SARS-CoV-2 spike protein for a third period of time, wherein the second recombinant soluble SARS-CoV-2 spike protein comprises the ectodomain of the SARS-CoV-2 spike protein, and wherein the first recombinant soluble SARS-CoV-2 spike protein is different than the first recombinant soluble SARS-CoV-2 spike protein; (g) washing the well; (h) incubating a second labeled antibody that binds to an isotype or subtype of an immunoglobulin (e.g., IgG) for a fourth period of time in well; (i) washing the well; and (j) detecting the binding of the second labeled antibody to the second recombinant SARS-CoV-2 spike protein. In certain embodiments, the first time period, second time period, or both are 30 minutes, 45 minutes, 1 hour, 2 hours, 2.5 hours or 3 hours. In some embodiments, the third and fourth time periods are 30 minutes, 45 minutes, 1 hour, 2 hours, 2.5 hours or 3 hours. In certain embodiments, the second labeled antibody is the same as the first labeled antibody. In other embodiments, the second labeled antibody is different than the first labeled antibody. For example, the first labeled antibody binds to human IgG and second labeled antibodies bind to a different human immunoglobulin isotype (e.g., IgA or IgM) or subtype. In a specific embodiment, the first recombinant soluble SARS-CoV-2 spike protein comprises a receptor binding domain of a SARS-CoV-2 spike protein known in the art but not the entire ectodomain, and the second recombinant soluble SARS-CoV-2 spike protein comprises the ectodomain of a SARS-CoV-2 spike protein known in the art. The specimen may be serially diluted, such as described in Section 6, infra. In a specific embodiment, the specimen is a biological sample, such as, e.g., blood, sera, or plasma. The biological sample may be from an asymptomic subject, a symptomic subject or those suspected of having a SARS-CoV-2 infection or COVID-19. The biological sample may inactivated before being incubated with the well coated with recombinant SARS-CoV-2 spike protein. In another embodiment, the specimen is an antibody or antibodies against SARS-CoV-2 spike protein.

In some embodiments, provided herein is a method for the detection of antibody that specifically binds to human SARS-CoV-2 spike protein, comprising: (a) incubating a specimen in a well coated with a first recombinant soluble SARS-CoV-2 spike protein for a first period of time, wherein the recombinant soluble SARS-CoV-2 spike protein comprises amino acid residues 319-541 of GenBank Accession No. MN908947.3 and a tag; (b) washing the well; (c) incubating a first labeled antibody that binds to an isotype or subtype of an immunoglobulin (e.g., IgG) for a second period of time in the well; (d) washing the well; and (e) detecting the binding of the first labeled antibody to the first recombinant SARS-CoV-2 spike protein. In certain embodiment, the method further comprises (f) incubating the specimen in a well coated with a second recombinant soluble SARS-CoV-2 spike protein for a third period of time, wherein the second recombinant soluble SARS-CoV-2 spike protein comprises amino acid residues 1-1213 of GenBank Accession No. MN908947.3, a C-terminal cleavage site, trimerization domain, and a tag, and wherein the recombinant second soluble SARS-CoV-2 spike protein does not contain a polybasic cleavage site; (g) washing the well; (h) incubating a second labeled antibody that binds to an isotype or subtype of an immunoglobulin (e.g., IgG) for a fourth period of time in the well; (i) washing the well; and (j) detecting the binding of the second labeled antibody to the second recombinant SARS-CoV-2 spike protein. See, e.g, Sections 5.1 and 6 for examples of tag, trimerization domains and cleavage sites. In certain embodiments, the tag is a hexahistidine tag. In some embodiments, the C-terminal cleavage site is a C-terminal thrombin cleavage site. In certain embodiments, the trimerization domain is a T4 foldon trimerization domain. In some embodiments, the tag is a hexahistidine tag, the C-terminal cleavage site is a C-terminal thrombin cleavage site, and the trimerization domain is a T4 foldon trimerization domain. In a specific embodiment, the first recombinant soluble spike protein comprises the amino acid sequence of SEQ ID NO:2. In another specific embodiment, the second recombinant soluble SARS-CoV-2 spike protein comprises the amino acid sequence of SEQ ID NO: 4 or 6. In another specific embodiment, the second recombinant soluble SARS-CoV-2 spike protein comprises the amino acid sequence of SEQ ID NO: 4 or 6 without the first 14 amino acid residues. In certain embodiments, the first time period, second time period, or both are 30 minutes, 45 minutes, 1 hour, 2 hours, 2.5 hours or 3 hours. In some embodiments, the third and fourth time periods are 30 minutes, 45 minutes, 1 hour, 2 hours, 2.5 hours or 3 hours. The specimen may be serially diluted, such as described in Section 6, infra. In a specific embodiment, the specimen is a biological sample, such as, e.g., blood, sera, or plasma. Th specimen may also be saliva. The biological sample may be from an asymptomic subject, a symptomic subject or those suspected of having a SARS-CoV-2 infection or COVID-19. The biological sample may inactivated before being incubated with the well coated with recombinant SARS-CoV-2 spike protein. In another embodiment, the specimen is an antibody or antibodies against SARS-CoV-2 spike protein.

In another embodiment, provided herein is a method for detecting antibody that specifically binds to human SARS-CoV-2 spike protein, comprising: (a) incubating a specimen in a well of, e.g., a microtiter plate (e.g., a 96-well microtiter plate) coated with a first recombinant SARS-CoV-2 spike protein described herein for a first period of time (e.g., about 30 minutes, 45 minutes, 1 hour, 1.25 hours, 1.5 hours, 2 hours, 2.25 hours, 2.5 hours or 3 hours at room temperature); (b) washing the well (e.g., washing the well 1, 2, 3 or more times with a buffer, such as, e.g., PBS or Tween 20 PBS (PBS-T)); and (c) detecting the binding of antibody present in the specimen to the first recombinant SARS-CoV-2 spike protein in the well. In certain embodiments, the binding of antibody present in the specimen to the first recombinant SARS-CoV-2 spike protein is detected using a labeled antibody that binds to an isotype(s) (e.g., IgG) or subtype(s) of antibody, or is pan-specific for human isotypes. In some embodiments, the labeled antibody is labeled with a chemiluminescent moiety, a fluorescent moiety, radioactive moiety or other detectable moiety. In a specific embodiment, the labeled antibody is labeled (e.g., conjugated to) horseradish perioxidase or alkaline phosphatase. In certain embodiments, the well coated with the first recombinant SARS-CoV-2 spike protein is blocked with a blocking solution (e.g., PBS-T+3% non-fat milk powder) for a period of time (e.g., about 30 minutes, 45 minutes, 1 hour, 1.25 hours, 1.5 hours or 2 hours at room temperature) prior to step (a). In certain embodiments, the specimen is an antibody sample. In certain embodiments, the specimen is a biological sample. The biological sample may be heat inactivated by a technique known to one of skill in the art or described herein (e.g., 56° C. for about 15 minutes, 30 minutes, 45 hours, 60 minutes 1.25 hours) prior to use. The biological sample may be also diluted (e.g., diluted in buffers, such as PBS) prior to use in the method. In certain embodiments (e.g., if antibody is detected in the first immunoassay), the method further comprises the following: (d) incubating the specimen in a well of, e.g., a microtiter plate (e.g., a 96-well microtiter plate) coated with a second recombinant SARS-CoV-2 spike protein described herein for a first period of time (e.g., about 30 minutes, 45 minutes, 1 hour, 1.25 hours, 1.5 hours, 2 hours, 2.25 hours, 2.5 hours or 3 hours at room temperature), wherein the second recombinant SARS-CoV-2 spike protein is different than the first recombinant SARS-CoV-2 spike protein; (e) washing the well (e.g., washing the well 1, 2, 3 or more times with a buffer, such as, e.g., PBS or Tween 20 PBS (PBS-T)); and (f) detecting the binding of antibody present in the specimen to the second recombinant SARS-CoV-2 spike protein in the well. In certain embodiments, the binding of antibody present in the specimen to the second recombinant SARS-CoV-2 spike protein is detected using a labeled antibody that binds to an isotype(s) (e.g., IgG) or subtype(s) of antibody, or is pan-specific for human isotypes. In some embodiments, the labeled antibody is labeled with a chemiluminescent moiety, a fluorescent moiety, radioactive moiety or other detectable moiety. In a specific embodiment, the labeled antibody is labeled (e.g., conjugated to) horseradish perioxidase or alkaline phosphatase. In certain embodiments, the well coated with the second recombinant SARS-CoV-2 spike protein is blocked with a blocking solution (e.g., PBS-T+3% non-fat milk powder) for a period of time (e.g., about 30 minutes, 45 minutes, 1 hour, 1.25 hours, 1.5 hours or 2 hours at room temperature) prior to step (d). In certain embodiments, the specimen is an antibody sample. In certain embodiments, the specimen is a biological sample. The biological sample may be heat inactivated by a technique known to one of skill in the art or described herein (e.g., 56° C. for about 15 minutes, 30 minutes, 45 hours, 60 minutes 1.25 hours) prior to use. The biological sample may be also diluted (e.g., diluted in buffers, such as PBS) prior to use in the method. In a specific embodiment, the first recombinant SARS-CoV-2 spike protein comprises a receptor binding domain of a SARS-CoV-2 spike protein but not the entire ectodomain, and the second recombinant SARS-CoV-2 spike protein comprises the ectodomain of a SARS-CoV-2 spike protein. See Sections 5.1 and 6 for examples of such recombinant SARS-CoV-2 spike proteins. In another specific embodiment, the first recombinant SARS-CoV-2 spike protein comprises the amino acid sequence of SEQ ID NO: 2 and the second recombinant SARS-CoV-2 spike protein comprises the amino acid sequence of SEQ ID NO: 4 or 6. In another specific embodiment, the first recombinant SARS-CoV-2 spike protein comprises the amino acid sequence of SEQ ID NO: 2 without the first 14 amino acid residues and the second recombinant SARS-CoV-2 spike protein comprises the amino acid sequence of SEQ ID NO: 4 or 6 without the first 14 amino acid residues. In certain embodiments, the method is a high-throughput assay. In specific embodiments, the method involves including wells with positive and negative controls, such as described herein. In specific embodiments, the binding of antibody in the specimen to the labeled antibody, which is bound to the recombinant SARS-CoV-2 spike protein, is detected using a spectrophotometer at a certain wavelength (e.g., 490 nm).

In another embodiment, provided herein is a method for detecting antibody that specifically binds to human SARS-CoV-2 spike protein, comprising: (a) incubating a specimen in a well of, e.g., a microtiter plate (e.g., a 96-well microtiter plate) coated with a first recombinant SARS-CoV-2 spike protein described herein for a first period of time (e.g., about 30 minutes, 45 minutes, 1 hour, 1.25 hours, 1.5 hours, 2 hours, 2.25 hours, 2.5 hours or 3 hours at room temperature); (b) washing the well (e.g., washing the well 1, 2, 3 or more times with a buffer, such as, e.g., PBS or Tween 20 PBS (PBS-T)); (c) incubating a first labeled antibody in the well for a second period of time (e.g., about 30 minutes, 45 minutes, 1 hour, 1.25 hours, 1.5 hours, or 2 hours), wherein the first labeled antibody binds to an isotype(s) (e.g., IgG) or subtype(s) of antibody, or is pan-specific for human isotypes; (d) washing the well (e.g., washing the well 1, 2, 3 or more times with a buffer, such as, e.g., PBS or Tween 20 PBS (PBS-T)); and (e) detecting the binding of the first labeled antibody to antibody present in the specimen, which is bound to the first recombinant SARS-CoV-2 spike protein in the well. In some embodiments, the first labeled antibody is labeled with a chemiluminescent moiety, a fluorescent moiety, radioactive moiety or other detectable moiety. In certain embodiments, the first labeled antibody is labeled (e.g., conjugated to) horseradish perioxidase or alkaline phosphatase. In a specific embodiment, the first labeled antibody is labeled (e.g., conjugated to) horseradish perioxidase, o-phenylenediamine solution is used as substrate for the enzyme and the reaction between the horseradish perioxidase and substrate is stopped using 3M HCl. In certain embodiments, the well coated with the first recombinant SARS-CoV-2 spike protein is blocked with a blocking solution (e.g., PBS-T+3% non-fat milk powder) for a period of time (e.g., about 30 minutes, 45 minutes, 1 hour, 1.25 hours, 1.5 hours or 2 hours at room temperature) prior to step (a). In certain embodiments, the specimen is an antibody sample. In certain embodiments, the specimen is a biological sample. The biological sample may be heat inactivated by a technique known to one of skill in the art or described herein (e.g., 56° C. for about 15 minutes, 30 minutes, 45 hours, 60 minutes 1.25 hours) prior to use. The biological sample may be also diluted (e.g., diluted in buffers, such as PBS) prior to use in the method. In certain embodiments (e.g., if antibody is detected in the first immunoassay), the method further comprises the following: (f) incubating the specimen in a well of, e.g., a microtiter plate (e.g., a 96-well microtiter plate) coated with a second recombinant SARS-CoV-2 spike protein described herein for a first period of time (e.g., about 30 minutes, 45 minutes, 1 hour, 1.25 hours, 1.5 hours, 2 hours, 2.25 hours, 2.5 hours or 3 hours at room temperature), wherein the second recombinant SARS-CoV-2 spike protein is different than the first recombinant SARS-CoV-2 spike protein; (g) washing the well (e.g., washing the well 1, 2, 3 or more times with a buffer, such as, e.g., PBS or Tween 20 PBS (PBS-T)); (h) incubating a second labeled antibody in the well for a second period of time (e.g., about 30 minutes, 45 minutes, 1 hour, 1.25 hours, 1.5 hours, or 2 hours), wherein the second labeled antibody binds to an isotype(s) (e.g., IgG) or subtype(s) of antibody, or is pan-specific for human isotypes; (i) washing the well (e.g., washing the well 1, 2, 3 or more times with a buffer, such as, e.g., PBS or Tween 20 PBS (PBS-T)); and (j) detecting the binding of the second labeled antibody to antibody present in the specimen, which is bound to the second recombinant SARS-CoV-2 spike protein in the well. In some embodiments, the second labeled antibody is labeled with a chemiluminescent moiety, a fluorescent moiety, radioactive moiety or other detectable moiety. In certain embodiments, the labeled antibody is labeled (e.g., conjugated to) horseradish perioxidase or alkaline phosphatase. In a specific embodiment, the second labeled antibody is labeled (e.g., conjugated to) horseradish perioxidase, o-phenylenediamine solution is used as substrate for the enzyme and the reaction between the horseradish perioxidase and substrate is stopped using 3M HCl. The first and second labeled antibodies may be the same or different. In certain embodiments, the well coated with the second recombinant SARS-CoV-2 spike protein is blocked with a blocking solution (e.g., PBS-T+3% non-fat milk powder) for a period of time (e.g., about 30 minutes, 45 minutes, 1 hour, 1.25 hours, 1.5 hours or 2 hours at room temperature) prior to step (f). In certain embodiments, the specimen is an antibody sample. In certain embodiments, the specimen is a biological sample. The biological sample may be heat inactivated by a technique known to one of skill in the art or described herein (e.g., 56° C. for about 15 minutes, 30 minutes, 45 hours, 60 minutes 1.25 hours) prior to use. The biological sample may be also diluted (e.g., diluted in buffers, such as PBS) prior to use in the method. In a specific embodiment, the first recombinant SARS-CoV-2 spike protein comprises a receptor binding domain of a SARS-CoV-2 spike protein but not the entire ectodomain, and the second recombinant SARS-CoV-2 spike protein comprises the ectodomain of a SARS-CoV-2 spike protein. See Sections 5.1 and 6 for examples of such recombinant SARS-CoV-2 spike proteins. In another specific embodiment, the first recombinant SARS-CoV-2 spike protein comprises the amino acid sequence of SEQ ID NO: 2 and the second recombinant SARS-CoV-2 spike protein comprises the amino acid sequence of SEQ ID NO: 4 or 6. In another specific embodiment, the first recombinant SARS-CoV-2 spike protein comprises the amino acid sequence of SEQ ID NO: 2 without the first 14 amino acid residues and the second recombinant SARS-CoV-2 spike protein comprises the amino acid sequence of SEQ ID NO: 4 or 6 without the first 14 amino acid residues. In certain embodiments, the method is a high-throughput assay. In specific embodiments, the method involves including wells with positive and negative controls, such as described herein. In specific embodiments, the binding of antibody in the specimen to the labeled antibody, which is bound to the recombinant SARS-CoV-2 spike protein, is detected using a spectrophotometer at a certain wavelength (e.g., 490 nm).

In some embodiments, antibody titers of 1:80 and 1:160 are characterized as low titers, titers of 1:320 are characterized as moderate, and titers of 1:960 and ≥1:2880 are characterized as high titers in an immunoassay described herein (e.g., in Section 6, infra). In certain embodiments, antibody titers are correlated with a microneutralization activity, such as described in Section 6, infra.

In certain embodiment, antibody levels detected in an immunoassay described herein are quantitated. See Section 6, infra, for how antibody levels detected in an immunoassay described herein may be quantitated. In a specific embodiment, antibody levels are reported in an arbitrary unit based upon a four parameter logistic curve generated from a serial dilution of a strong positive (high titer ≥2880 using qualitative assay) patient pool that that is included on each ELISA. In some embodiments, an AU/ml >5 indicates anti-SARS-CoV-2 IgG has been detected at levels consistent with protective immunity against SARS-CoV-2 infection and an AU/ml<5 indicates that results is negative.

In another embodiment, provided herein is a method for detecting antibody specific for SARS-CoV-2 spike protein, comprising: (a) incubating a diluted heat inactivated (e.g., heat inactivated at 56° C. for 30 minutes to 1 hour, or 30, 45, or 60 minutes) biological sample in a well of a multi-well microtiter plate (e.g., a 96-well microtiter plate) coated with a first recombinant soluble SARS-CoV-2 spike protein for 1.5 to 2.5 hours at room temperature, wherein the first recombinant soluble SARS-CoV-2 spike protein comprises a SARS-CoV-2 spike protein receptor binding domain (e.g., amino acid residues 319 to 541 of a SARS-CoV-2 spike protein) or a derivative thereof and optionally a tag (e.g., a histidine tag, at the C-terminus), and wherein the biological sample is serum or plasma from a human subject; (b) washing the well with wash buffer (e.g., wash buffer comprises phosphate buffered saline and 0.5% to 2% of a surfactant, such as polysorbate (e.g., polysorbate-20 or polysorbate-80) or Triton X-100; (c) incubating a monoclonal antibody specific to human IgG or another immunoglobulin isotype or subtype, or a pan-specific human Ig conjugated to horseradish peroxidase, alkaline phosphatase or another detectable moiety in the well for a certain period of time (e.g., 30 minutes to 1.5 hours, or 30 minutes, 45 minutes, 60 minutes, 75 minutes or 90 minutes) at, e.g., room temperature; (d) washing the well with the wash buffer; (e) incubating a substrate solution (e.g., substrate solution comprising 3,3′,5,5′-Tetramethylbenzidine (TMB) substrate) in the well for a certain period of time (e.g., 10 to 40 minutes, or 10, 15, 20, 30 or 40 minutes) e.g., at room temperature; (f) adding stop solution (e.g., stop solution comprises an acid, such an alkyl sulfonic acid (e.g., 1-5% methanesulfonic acid), IN HCl or 2 N H₂SO₄) to the well; (g) determining the optical density of the well within a certain period of time (e.g., 10-30 minutes, or 10, 15, 20 or 30 minutes) using a microtiter plate reader set to a certain wavelength (e.g., 450 nm) to detect antibody specific for SARS-CoV-2 spike protein. In certain embodiments, the wavelength is 450 nm and if wavelength correction is available set at 540 nm or 570 nm, or if wavelength correction is not available, readings at 540 nm or 570 nm are taken and subtracted from readings at 450 nm. In some embodiments, 100 μl of diluted heat inactivated biological sample is incubated in the well of the multi-well microtiter plate in step (a). In certain embodiments, the heat inactivated biological sample is diluted to a final dilution of 1:100 in sample buffer (e.g., sample buffer comprising phosphate buffered saline (PBS) with 0.5% to 5% milk). In specific embodiments, the well is washed in steps (b) and (d) two or three times. In certain embodiments, each time the well is washed 400 μl of the wash buffer is used. In some embodiments, 100 μl of the substrate solution is incubated in the well in step (e). In some embodiments, 100 μl of the stop solution is added to the well. In specific embodiments, the microtiter plate comprises a second well and a third well, and wherein steps (a) through (g) are concurrently performed with respect to the second and third wells except that in step (a): (i) a positive control monoclonal antibody specific for SARS-CoV-2 spike protein is used in the second well instead of the diluted biological sample and (ii) a negative control is used in the third well instead of the diluted biological sample. In certain embodiments, the positive control monoclonal antibody is diluted in the sample buffer (e.g., diluted 1:2, 1:4, 1:5, 1:8 or 1:10 in sample buffer). In specific embodiments, the positive control monoclonal antibody binds to the receptor binding domain of the SARS-CoV-2 spike protein. In certain embodiments, the negative control is a monoclonal antibody that does not bind to the SARS-CoV-2 spike protein. In some embodiments, the negative control antibody is diluted 1:5 in the sample buffer (e.g., diluted 1:2, 1:4, 1:5, 1:8 or 1:10 in sample buffer). In certain embodiments, the negative control is a control buffer. In certain embodiments, the biological samples and controls are run in duplicate. In specific embodiments, corrected optical density values for the biological sample are divided by corrected optical density values for the second well to calculate the confidence interval (CI) for the biological sample. In particular embodiments, a CI value of greater than or equal to a certain value indicates that the biological sample is positive for antibody specific for the SARS-CoV-2 spike protein and a CI value of less than the certain value indicates that the biological sample is negative for antibody specific for the SARS-CoV-2 spike protein. In a specific embodiment, a CI value of greater than or equal to 0.5 indicates that the biological sample is positive for antibody specific for the SARS-CoV-2 spike protein and a CI value of less than 0.5 indicates that the biological sample is negative for antibody specific for the SARS-CoV-2 spike protein. In another specific embodiment, a CI value of greater than or equal to 0.6 indicates that the biological sample is positive for antibody specific for the SARS-CoV-2 spike protein and a CI value of less than 0.6 indicates that the biological sample is negative for antibody specific for the SARS-CoV-2 spike protein. In another specific embodiment, a CI value of greater than or equal to 0.7 indicates that the biological sample is positive for antibody specific for the SARS-CoV-2 spike protein and a CI value of less than 0.7 indicates that the biological sample is negative for antibody specific for the SARS-CoV-2 spike protein. In another specific embodiment, a CI value of 0.8 indicates that the biological sample is positive for antibody specific for the SARS-CoV-2 spike protein and a CI value of less than 0.8 indicates that the biological sample is negative for antibody specific for the SARS-CoV-2 spike protein. In another specific embodiment, a CI value of greater than or equal to 0.9 indicates that the biological sample is positive for antibody specific for the SARS-CoV-2 spike protein and a CI value of less than 0.9 indicates that the biological sample is negative for antibody specific for the SARS-CoV-2 spike protein. In another specific embodiment, a CI value of greater than or equal to 1 indicates that the biological sample is positive for antibody specific for the SARS-CoV-2 spike protein and a CI value of less than 1 indicates that the biological sample is negative for antibody specific for the SARS-CoV-2 spike protein. In specific embodiments, the method further comprises a quantitative assay to assess quantitative levels of immunoglobulin (e.g., IgG) antibodies (e.g., neutralizing antibodies) against SARS-CoV-2 spike protein for those biological samples identified as positive, wherein the quantitative assay comprises: (I) incubating for a certain period of time (e.g., 1.5 to 2.5 hours, or 1.5, 2 or 2.5 hours) at, e.g., room temperature of a multi-well microtiter plate (e.g., a 96-well microtiter plate) coated with a second recombinant soluble SARS-CoV-2 spike protein, wherein different wells of the microtiter plate contain either a diluted heat inactivated biological sample(s) (e.g., heat inactivated at 56° C. for 30 minutes to 1 hour, or 30, 45, or 60 minutes), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the second recombinant soluble SARS-CoV-2 spike protein comprises the amino acid sequence of a SARS-CoV-2 spike protein ectodomain or a derivative thereof, and wherein the biological sample is serum or plasma from a human subject; (II) washing the well with a wash buffer (e.g., wash buffer comprises phosphate buffered saline and 0.5% to 2% of a surfactant, such as polysorbate (e.g., polysorbate-20 or polysorbate-80) or Triton X-100; (III) incubating a monoclonal antibody specific to human IgG or another immunoglobulin isotype or subtype, or a pan-specific human Ig conjugated to horseradish peroxidase, alkaline phosphatase or another detectable moiety in the well for a certain period of time (e.g., 30 minutes to 1.5 hours, or 30 minutes, 45 minutes, 60 minutes, 75 minutes or 90 minutes) at, e.g., room temperature; (IV) washing the well with the wash buffer; (V) incubating a substrate solution (e.g., substrate solution comprising 3,3′,5,5′-Tetramethylbenzidine (TMB) substrate) in the well for a certain period of time (e.g., 10 to 40 minutes, or 10, 15, 20, 30 or 40 minutes) e.g., at room temperature; (VI) adding stop solution (e.g., stop solution comprises an acid, such an alkyl sulfonic acid (e.g., 1-5% methanesulfonic acid), IN HCl or 2 N H₂SO₄) to the well; (VII) determining the optical density of the well within a certain period of time (e.g., 10-30 minutes, or 10, 15, 20 or 30 minutes) using a microtiter plate reader set to a certain wavelength (e.g., 450 nm) to detect antibody specific for SARS-CoV-2 spike protein. In specific embodiments, each well is washed in steps (II) or (IV) two or three times. In certain embodiments, each time the well is washed in steps (II) and/or (IV) 400 μl of the wash buffer is used. In some embodiments, 100 μl of the monoclonal antibody specific to human IgG or another immunoglobulin isotype or subtype, or a pan-specific human Ig conjugated to horseradish peroxidase, alkaline phosphatase or another detectable moiety is incubated in each of the wells in step (III). In certain embodiments, 100 μl of the substrate solution is incubated in each of the wells in step (V). In some embodiments, 100 μl of the stop solution is incubated in each of the wells in step (VI). In certain embodiments, the heat inactivated biological sample is diluted to a final dilution of 1:200 in sample buffer. In some embodiments, the three controls are diluted 1:5 in the sample buffer. In a specific embodiment, the calibrators are those set forth in Example 11. In another specific embodiment, the calibrators are those set forth in Example 12, 13, 14 or 16. In another specific embodiment, the three controls are those set forth in Example 11. In another specific embodiment, the controls are those set forth in Example 12, 13, 14 or 16. The method may use less than seven (e.g., 3, 4, 5 or 6 calibrators) or more than calibrators. In some embodiments, 1 or 2 positive controls are used instead of 3. In certain embodiments, the biological samples, controls and calibrators are run in duplicate. In a specific embodiment, the is 450 nm and if wavelength correction is available set at 540 nm or 570 nm, or if wavelength correction is not available, readings at 540 nm or 570 nm are taken and subtracted from readings at 450 nm. In another specific embodiment, the quantitative assay further comprises: (VIII) generating a calibration curve and comparing the signal from the diluted heat inactivated biological sample(s) to the calibration curve to generate a final result of IgG levels in arbitrary units per milliliter (AU/ml).

In another embodiment, provided herein is a method assessing the quantitative levels of immunoglobulin (e.g., IgG) antibodies (e.g., neutralizing antibodies) against SARS-CoV-2 spike protein, wherein the method comprises a quantitative assay comprising: (I) incubating for a certain period of time (e.g., 1.5 to 2.5 hours, or 1.5, 2 or 2.5 hours) at, e.g., room temperature of a multi-well microtiter plate (e.g., a 96-well microtiter plate) coated with a second recombinant soluble SARS-CoV-2 spike protein, wherein different wells of the microtiter plate contain either a diluted heat inactivated biological sample(s) (e.g., heat inactivated at 56° C. for 30 minutes to 1 hour, or 30, 45, or 60 minutes), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the second recombinant soluble SARS-CoV-2 spike protein comprises the amino acid sequence of a SARS-CoV-2 spike protein ectodomain or a derivative thereof, and wherein the biological sample is serum or plasma from a human subject; (II) washing the well with a wash buffer (e.g., wash buffer comprises phosphate buffered saline and 0.5% to 2% of a surfactant, such as polysorbate (e.g., polysorbate-20 or polysorbate-80) or Triton X-100; (III) incubating a monoclonal antibody specific to human IgG or another immunoglobulin isotype or subtype, or a pan-specific human Ig conjugated to horseradish peroxidase, alkaline phosphatase or another detectable moiety in the well for a certain period of time (e.g., 30 minutes to 1.5 hours, or 30 minutes, 45 minutes, 60 minutes, 75 minutes or 90 minutes) at, e.g., room temperature; (IV) washing the well with the wash buffer; (V) incubating a substrate solution (e.g., substrate solution comprising 3,3′,5,5′-Tetramethylbenzidine (TMB) substrate) in the well for a certain period of time (e.g., 10 to 40 minutes, or 10, 15, 20, 30 or 40 minutes) e.g., at room temperature; (VI) adding stop solution (e.g., stop solution comprises an acid, such an alkyl sulfonic acid (e.g., 1-5% methanesulfonic acid), IN HCl or 2 N H₂SO₄) to the well; (VII) determining the optical density of the well within a certain period of time (e.g., 10-30 minutes, or 10, 15, 20 or 30 minutes) using a microtiter plate reader set to a certain wavelength (e.g., 450 nm) to detect antibody specific for SARS-CoV-2 spike protein. In specific embodiments, each well is washed in steps (II) or (IV) two or three times. In certain embodiments, each time the well is washed in steps (II) and/or (IV) 400 μl of the wash buffer is used. In some embodiments, 100 μl of the monoclonal antibody specific to human IgG or another immunoglobulin isotype or subtype, or a pan-specific human Ig conjugated to horseradish peroxidase, alkaline phosphatase or another detectable moiety is incubated in each of the wells in step (III). In certain embodiments, 100 μl of the substrate solution is incubated in each of the wells in step (V). In some embodiments, 100 μl of the stop solution is incubated in each of the wells in step (VI). In certain embodiments, the heat inactivated biological sample is diluted to a final dilution of 1:200 in sample buffer. In a specific embodiment, the calibrators are those set forth in Example 11. In another specific embodiment, the calibrators are those set forth in Example 12, 13 or 14. In another specific embodiment, the three controls are those set forth in Example 11. In another specific embodiment, the controls are those set forth in Example 12, 13 or 14. The method may use less than seven (e.g., 3, 4, 5 or 6 calibrators) or more than calibrators. In some embodiments, 1 or 2 positive controls are used instead of 3. In certain embodiments, the biological samples, controls and calibrators are run in duplicate. In some embodiments, the three controls are diluted 1:5 in the sample buffer. In a specific embodiment, the is 450 nm and if wavelength correction is available set at 540 nm or 570 nm, or if wavelength correction is not available, readings at 540 nm or 570 nm are taken and subtracted from readings at 450 nm. In another specific embodiment, the quantitative assay further comprises: (VIII) generating a calibration curve and comparing the signal from the diluted heat inactivated biological sample(s) to the calibration curve to generate a final result of IgG levels in arbitrary units per milliliter (AU/ml).

In a specific embodiment, provided herein is method for detecting antibody specific for SARS-CoV-2 spike protein, comprising: (a) incubating a diluted heat inactivated biological sample (e.g., heat inactivated at 56° C. for 30 minutes to 1 hour) in a well of a multi-well microtiter plate coated with a first recombinant soluble SARS-CoV-2 spike protein for 1.5 to 2.5 hours at room temperature, wherein the first recombinant soluble SARS-CoV-2 spike protein comprises the receptor binding domain of a SARS-CoV-2 spike protein (e.g., amino acid residues 319-541 of the spike protein found at GenBank Accession No. NM908947.3) or a derivative thereof and optionally a tag (e.g., a histidine tag) at the C-terminus, and wherein the biological sample is serum or plasma from a human subject; (b) washing the well (e.g., two or three times) with wash buffer, wherein the wash buffer comprises phosphate buffered saline and 0.5% to 2% of a surfactant; (c) incubating a monoclonal antibody specific to human IgG conjugated to horseradish peroxidase in the well for 30 minutes to 1.5 hours at room temperature; (d) washing the well (e.g., two or three times) with the wash buffer; (e) incubating a substrate solution in the well for 10 to 40 minutes (e.g., about 20 minutes) at room temperature, wherein the substrate solution comprises 3,3′,5,5′-Tetramethylbenzidine (TMB) substrate; (f) adding stop solution to the well, wherein the stop solution comprises an acid; and (g) determining the optical density of the well within 30 minutes using a microtiter plate reader set to 450 nm to detect antibody specific for SARS-CoV-2 spike protein. See Section 5.1 and Section 6 for examples of recombinant SARS-CoV-2 spike proteins. In certain embodiments, if wavelength correction is available set at 540 nm or 570 nm, or if wavelength correction is not available, readings at 540 nm or 570 nm are taken and subtracted from readings at 450 nm. In a specific embodiment, the the acid is an alkylsuphonic acid (e.g. 1-5% methanesulfonic acid), 1 N HCl or 2 N H₂SO₄. In some embodiments, 100 μl of diluted heat inactivated biological sample is incubated in the well of the multi-well microtiter plate in step (a). In certain embodiments, the heat inactivated biological sample is diluted to a final dilution of 1:100 in sample buffer, wherein the sample buffer comprises phosphate buffered saline (PBS) with 0.5% to 5% milk. In certain embodiments, each time the well is washed 400 μl of the wash buffer is used. In some embodiments, 100 μl of the substrate solution is incubated in the well in step (e). In some embodiments, 100 μl of the stop solution is added to the well. In specific embodiments, the microtiter plate comprises a second well and a third well, and wherein steps (a) through (g) are concurrently performed with respect to the second and third wells except that in step (a): (i) a positive control monoclonal antibody specific for SARS-CoV-2 spike protein is used in the second well instead of the diluted biological sample and (ii) a negative control is used in the third well instead of the diluted biological sample. In a specific embodiment, the positive control monoclonal antibody (e.g., monoclonal antibody that binds to the receptor binding domain of the SARS-CoV-2 spike protein) is diluted in the sample buffer (e.g., the positive control monoclonal antibody specific is diluted 1:5 in the sample buffer). In another specific embodiment, the negative control is a monoclonal antibody that does not bind to the SARS-CoV-2 spike protein. In certain embodiments, the negative control antibody (e.g. control buffer) is diluted 1:5 in the sample buffer. In specific embodiments, corrected optical density values for the biological sample are divided by corrected optical density values for the second well to calculate the cutoff interval (sometimes referred to as the confidence interval) (CI) for the biological sample. In certain embodiments, a CI value of greater than or equal to 0.5 indicates that the biological sample is positive for antibody specific for the SARS-CoV-2 spike protein and a CI value of less than 0.5 indicates that the biological sample is negative for antibody specific for the SARS-CoV-2 spike protein. In some embodiments, a CI value of greater than or equal to 0.6 indicates that the biological sample is positive for antibody specific for the SARS-CoV-2 spike protein and a CI value of less than 0.6 indicates that the biological sample is negative for antibody specific for the SARS-CoV-2 spike protein. In certain embodiments, a CI value of greater than or equal to 0.7 indicates that the biological sample is positive for antibody specific for the SARS-CoV-2 spike protein and a CI value of less than 0.7 indicates that the biological sample is negative for antibody specific for the SARS-CoV-2 spike protein. In some embodiments, a CI value of greater than or equal to 0.8 indicates that the biological sample is positive for antibody specific for the SARS-CoV-2 spike protein and a CI value of less than 0.8 indicates that the biological sample is negative for antibody specific for the SARS-CoV-2 spike protein. In certain embodiments, a CI value of greater than or equal to 0.9 indicates that the biological sample is positive for antibody specific for the SARS-CoV-2 spike protein and a CI value of less than 0.9 indicates that the biological sample is negative for antibody specific for the SARS-CoV-2 spike protein. In some embodiments, a CI value of greater than or equal to 1 indicates that the biological sample is positive for antibody specific for the SARS-CoV-2 spike protein and a CI value of less than 1 indicates that the biological sample is negative for antibody specific for the SARS-CoV-2 spike protein. See Example 11, 12, 13 or 14, infra, for the interpretation of the results.

In a specific embodiment, the method further comprises a quantitative assay to assess quantitative levels of IgG antibodies against SARS-CoV-2 spike protein for those biological samples identified as positive, wherein the quantitative assay comprises: (I) incubating for 1.5 to 2.5 hours at room temperature of a multi-well microtiter plate coated with a second recombinant soluble SARS-CoV-2 spike protein, wherein different wells of the microtiter plate contain either a diluted heat inactivated biological sample(s) (e.g., heat inactivated at 56° C. for 30 minutes to 1 hour), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the second recombinant soluble SARS-CoV-2 spike protein comprises the amino acid sequence of a SARS-CoV-2 spike protein ectodomain or a derivative thereof, and wherein the biological sample is serum or plasma from a human subject; (II) washing each of the wells (e.g., two or three times) with the wash buffer; (III) incubating monoclonal antibody specific to human IgG conjugated to horseradish peroxidase in each of the wells for 30 minutes to 1.5 hours at room temperature; (IV) washing each of the wells (e.g., two or three times) with the wash buffer; (V) incubating the substrate solution in each of the wells for 10 to 40 minutes at room temperature; (VI) adding the stop solution to each of the wells; and (VII) determining the optical density of each of the wells within 30 minutes using a microtiter plate reader set to 450 nm to detect antibody specific for SARS-CoV-2 spike protein. In a specific embodiment, the second recombinant soluble SARS-CoV-2 spike protein comprises a C-terminal cleavage site and trimerization domain, and optionally a tag. In another specific embodiment, the second recombinant soluble SARS-CoV-2 spike protein comprises the amino acid of SEQ ID NO: 4 or 6 without the first 14 amino acid residues. See Section 5.1 and Section 6 for examples of recombinant SARS-CoV-2 spike proteins. In some embodiments, each time the well is washed 400 μl of the wash buffer is used. In certain embodiments, 100 μl of the monoclonal antibody specific to human IgG conjugated to horseradish peroxidase is incubated in each of the wells in step (III). In some embodiments, 100 μl of the substrate solution is incubated in each of the wells in step (V). In certain embodiments, 100 μl of the stop solution is incubated in each of the wells in step (VI). In some embodiments, the heat inactivated biological sample is diluted to a final dilution of 1:200 in sample buffer. In certain embodiments, the three controls are diluted 1:5 in the sample buffer. In specific embodiments, if wavelength correction is available set at 540 nm or 570 nm, or if wavelength correction is not available, readings at 540 nm or 570 nm are taken and subtracted from readings at 450 nm. In a specific embodiment, the quantitative assay further comprises: (VIII) generating a calibration curve and comparing the signal from the diluted heat inactivated biological sample(s) to the calibration curve to generate a final result of IgG levels in arbitrary units per milliliter (AU/ml). In specific embodiments, less than 3.2 AU/ml indicates that the human subject is negative for SARS-CoV-2 antibody or that there was no detectable immune response, 3.2 AU/ml to 10 AU/MI indicates that the human subject is likely positive, 10 AU/ml to 25 AU/ml indicates that the human subject has moderate antibody levels, and greater than 25 AU/mL indicates that the human subject has high antibody levels. In specific embodiments, 10 AU/ml to 25 AU ml and greater than 25 AU/ml indicates that the IgG levels specific for SARS-CoV-2 exhibit viral neutralizing activity in vitro. See Example 14, infra, for the interpretation of the results.

In another specific embodiment, provided herein is a method assessing the quantitative levels of IgG antibodies against SARS-CoV-2 spike protein, wherein the method comprises a quantitative assay comprising: (I) incubating for 1.5 to 2.5 hours at room temperature of a multi-well microtiter plate coated with a first recombinant soluble SARS-CoV-2 spike protein, wherein different wells of the microtiter plate contain either a diluted heat inactivated biological sample(s) (e.g., heat inactivated at 56° C. for 30 minutes to 1 hour), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the first recombinant soluble SARS-CoV-2 spike protein comprises the amino acid sequence of a SARS-CoV-2 spike protein ectodomain or a derivative thereof, and wherein the biological sample is serum or plasma from a human subject; (II) washing each of the wells (e.g., two or three times) with the wash buffer, wherein the wash buffer comprises phosphate buffered saline and 0.5% to 2% of a surfactant (e.g., polysorbate (e.g., polysorbate 20 or polysorbate 80) or Triton X-100); (III) incubating monoclonal antibody specific to human IgG conjugated to horseradish peroxidase in each of the wells for 30 minutes to 1.5 hours at room temperature; (IV) washing each of the wells (e.g., two or three times) with the wash buffer; (V) incubating a substrate solution in each of the wells for 10 to 40 minutes at room temperature, wherein the substrate solution comprises 3,3,5,5-Tetramethylbenzidine (TMB) substrate; (VI) adding a stop solution to each of the wells, wherein the stop solution comprises an acid (e.g. an alkyl sulfonic acid (e.g., 1-5% methanesulfonic acid), 1 N HCl or 2 N H₂SO₄); and (VII) determining the optical density of each of the wells within 30 minutes using a microtiter plate reader set to 450 nm to detect antibody specific for SARS-CoV-2 spike protein. In a specific embodiment, the first recombinant soluble SARS-CoV-2 spike protein comprises a C-terminal cleavage site and trimerization domain, and optionally a tag. In another specific embodiment, the first recombinant soluble SARS-CoV-2 spike protein comprises the amino acid of SEQ ID NO: 4 or 6 without the first 14 amino acid residues. See Section 5.1 and Section 6 for examples of recombinant SARS-CoV-2 spike proteins. In some embodiments, each time the well is washed 400 μl of the wash buffer is used. In certain embodiments, 100 μl of the monoclonal antibody specific to human IgG conjugated to horseradish peroxidase is incubated in each of the wells in step (III). In some embodiments, 100 μl of the substrate solution is incubated in each of the wells in step (V). In certain embodiments, 100 μl of the stop solution is incubated in each of the wells in step (VI). In some embodiments, the heat inactivated biological sample is diluted to a final dilution of 1:200 in sample buffer. In certain embodiments, the three controls are diluted 1:5 in the sample buffer. In specific embodiments, if wavelength correction is available set at 540 nm or 570 nm, or if wavelength correction is not available, readings at 540 nm or 570 nm are taken and subtracted from readings at 450 nm. In a specific embodiment, the quantitative assay further comprises: (VIII) generating a calibration curve and comparing the signal from the diluted heat inactivated biological sample(s) to the calibration curve to generate a final result of IgG levels in arbitrary units per milliliter (AU/ml). In specific embodiments, less than 3.2 AU/ml indicates that the human subject is negative for SARS-CoV-2 antibody or that there was no detectable immune response, 3.2 AU/ml to 10 AU/Ml indicates that the human subject is likely positive, 10 AU/ml to 25 AU/ml indicates that the human subject has moderate antibody levels, and greater than 25 AU/mL indicates that the human subject has high antibody levels. In specific embodiments, 10 AU/ml to 25 AU ml and greater than 25 AU/ml indicates that the IgG levels specific for SARS-CoV-2 exhibit viral neutralizing activity in vitro. See Example 14, infra, for the interpretation of the results.

In another specific embodiment, provided herein is a method assessing the quantitative levels of IgG antibodies against SARS-CoV-2 spike protein, wherein the method comprises a quantitative assay comprising: (I) incubating for 1.5 to 2.5 hours at room temperature of a multi-well microtiter plate coated with a first recombinant soluble SARS-CoV-2 spike protein, wherein different wells of the microtiter plate contain either a saliva sample (e.g., heat inactivated at 56° C. for 30 minutes to 1 hour), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the first recombinant soluble SARS-CoV-2 spike protein comprises the amino acid sequence of a SARS-CoV-2 spike protein ectodomain or a derivative thereof, and wherein the biological sample is serum or plasma from a human subject; (II) washing each of the wells (e.g., two or three times) with the wash buffer, wherein the wash buffer comprises phosphate buffered saline and 0.5% to 2% of a surfactant (e.g., polysorbate (e.g., polysorbate 20 or polysorbate 80) or Triton X-100); (III) incubating monoclonal antibody specific to human IgG conjugated to horseradish peroxidase in each of the wells for 30 minutes to 1.5 hours at room temperature; (IV) washing each of the wells (e.g., two or three times) with the wash buffer; (V) incubating a substrate solution in each of the wells for 10 to 40 minutes at room temperature, wherein the substrate solution comprises 3,3,5,5-Tetramethylbenzidine (TMB) substrate; (VI) adding a stop solution to each of the wells, wherein the stop solution comprises an acid (e.g. an alkyl sulfonic acid (e.g., 1-5% methanesulfonic acid), 1 N HCl or 2 N H₂SO₄); and (VII) determining the optical density of each of the wells within 30 minutes using a microtiter plate reader set to 450 nm to detect antibody specific for SARS-CoV-2 spike protein. In a specific embodiment, the first recombinant soluble SARS-CoV-2 spike protein comprises a C-terminal cleavage site and trimerization domain, and optionally a tag. In another specific embodiment, the first recombinant soluble SARS-CoV-2 spike protein comprises the amino acid of SEQ ID NO: 4 or 6 without the first 14 amino acid residues. See Section 5.1 and Section 6 for examples of recombinant SARS-CoV-2 spike proteins. In some embodiments, each time the well is washed 400 μl of the wash buffer is used. In certain embodiments, 100 μl of the monoclonal antibody specific to human IgG conjugated to horseradish peroxidase is incubated in each of the wells in step (III). In some embodiments, 100 μl of the substrate solution is incubated in each of the wells in step (V). In certain embodiments, 100 μl of the stop solution is incubated in each of the wells in step (VI). In some embodiments, the heat inactivated biological sample is diluted to a final dilution of 1:200 in sample buffer. In certain embodiments, the three controls are diluted 1:5 in the sample buffer. In specific embodiments, if wavelength correction is available set at 540 nm or 570 nm, or if wavelength correction is not available, readings at 540 nm or 570 nm are taken and subtracted from readings at 450 nm. In a specific embodiment, the quantitative assay further comprises: (VIII) generating a calibration curve and comparing the signal from the diluted heat inactivated biological sample(s) to the calibration curve to generate a final result of IgG levels in arbitrary units per milliliter (AU/ml). See Section 16 for methods and as well as assessment of the results. In certain embodiments, RT-PCR assays are also conducted, such as described in Example 16.

In a specific embodiment, the method set forth in Example 11 is used to assess IgG against SARS-CoV-2 spike protein present in a patient sample. In another specific embodiment, the method set forth in Example 11 used used to quantitate IgG against SARS-CoV-2 spike protein present in a patient sample. In another specific embodiment, the method set forth in Example 11 used used to quantitate neutralizing IgG against SARS-CoV-2 spike protein present in a patient sample.

In a specific embodiment, the method set forth in Example 12, 13, 14 or 16 is used to assess IgG against SARS-CoV-2 spike protein present in a patient sample. In another specific embodiment, the method set forth in Example 12, 13, 14 or 16 used used to quantitate IgG against SARS-CoV-2 spike protein present in a patient sample. In another specific embodiment, the method set forth in Example 12, 13, 14 or 16 used used to quantitate neutralizing IgG against SARS-CoV-2 spike protein present in a patient sample.

In certain embodiments, conducting an immunoassay described herein (e.g., an ELISA) provides a method for monitoring subjects (e.g, human subjects, such as healthcare workers). In some embodiments, conducting an immunoassay described herein provides a method for identifying subject (e.g., human subjects, such as healthcare workers) with antibody(ies) (including, e.g, monoclonal antibody) that may have use in a passive immunization regimen. In certain embodiments, antibody isolated from a subject identified as having antibody that specifically binds to SARS-CoV-2 spike protein may be used to treat a SARS-CoV-2 infection or COVID-19. In a specific embodiment, an antibody that specifically binds to the spike protein of SARS-CoV-2 does not bind to the spike protein of SARS-CoV-1 as assessed by an assay known to one of skill in the art. In another specific embodiment, an antibody that specifically binds to the spike protein of SARS-CoV-2 does not bind to the spike protein of SARS-CoV-1 as assessed by an assay described herein, and the antibody neutralizes SARS-CoV-2. In some embodiments, conducting an immunoassay described herein provides a method for dosing subjects (e.g., human subjects, such as healthcare workers) with antibody(ies) (including, e.g, monoclonal antibody) in a passive immunization regimen. In certain embodiments, conducting an immunoassay described herein provides a method for assessing the efficacy of subjects (e.g., human subjects, such as healthcare workers) immunized with a SARS-CoV-2 vaccine (e.g., a SARS-CoV-2 spike protein-based vaccine). In some embodiments, conducting an immunoassay described herein (e.g., an ELISA) provides a method for determining if a subject has seroconverted from anti-SARs-CoV-2 spike protein antibody negative status to anti-SARs-CoV-2 spike protein antibody positive status in infected patients. In certain embodiments, conducting an immunoassay described herein (e.g., an ELISA) provides an indication if the subject may have some immunoprotection against SARS-CoV-2.

In a specific embodiment, a kit described herein (e.g., in Section 5.8 or Section 6) is used in a method for detecting antibody specific for SARS-CoV-2 spike protein. In some embodiments, the detection of antibody allows for a clinical interpretation, such as described in Example 14, infra. The detection of antibody in an assay such as described in Example 14, provides an indication of concentration of IgG levels specific for SARS-CoV-2 that exhibit viral neutralizing activity in vitro. In certain embodiments, these results indicate a subject's potential risk of a subject for reinfection. In some embodiments, the results are assistance in the clinical diagnosis of SARS-CoV-2 infection, or COVID-19.

In a specific embodiment, an antibody specific for SARS-CoV-2 spike protein or a receptor binding domain thereof binds to SARS-CoV-2 and does not exhibit cross-reactivity with other coronaviruses, such as described herein. In another specific embodiment, an antibody specific for SARS-CoV-2 spike protein or a receptor binding domain thereof binds to SARS-CoV-2 with a higher affinity than the antibody binds to other antigens, such as the spike protein of a coronavirus or another antigen described herein (e.g., in Section 6, infra) as assessed using BIAcore™ surface plasmon resonance technology, Kinexa, or biolayer interferometry.

### 5.7 Other Uses of Recombinant SARS-CoV-2 Spike Protein

As noted below in Example 3, SARS-CoV2 spike protein has been found to mediate binding to host cells via the interaction with the human receptor angiotensin converting enzyme 2 (ACE2). An immunoassay using a recombinant SARS-CoV-2 protein described herein may be used to identify agents that inhibit the interaction between SARS-CoV2 spike protein and ACE2. Thus, in another aspect, provided herein are in vitro methods for identifying agents (e.g., compounds) that inhibit the interaction between ACE2 and SARS-CoV-2 protein. In one embodiment, a recombinant SARS-CoV-2 spike protein described herein is incubated for a period of time (e.g., about 5 minutes, 10 minutes, 15 minutes, 30 minutes, 45 minutes, 1 hour, 1.5 hours, 2 hours, 3 hours, 4 hours, 5 hours, 6 hours, 12 hours, 18 hours, 24 hours, or more, or 5 to 30 minutes, 15 to 60 minutes, 1 hour to 3 hours, 3 to 6 hours, 6 to 12 hours, or 18 to 24 hours) with an agent of interest prior to addition of antibody(ies) or a biological sample (e.g., blood, plasma or sera) containing an antibody(ies) identified to bind to the recombinant SARS-CoV-2 spike protein in an immunoassay described herein. In another embodiment, a recombinant SARS-CoV-2 spike protein described herein is incubated concurrently with an agent of interest and an antibody(ies) or a biological sample (e.g., blood, plasma or sera) containing an antibody(ies) identified to bind to the recombinant SARS-CoV-2 spike protein for a period of time (e.g., about 5 minutes, 10 minutes, 15 minutes, 30 minutes, 45 minutes, 1 hour, 1.5 hours, 2 hours, 3 hours, 4 hours, 5 hours, 6 hours, 12 hours, 18 hours, 24 hours, or more, or 5 to 30 minutes, 15 to 60 minutes, 1 hour to 3 hours, 3 to 6 hours, 6 to 12 hours, or 18 to 24 hours) prior to the detection of the antibody in an immunoassay. In another embodiment, a recombinant SARS-CoV-2 spike protein described herein is incubated with antibody(ies) or a biological sample (e.g., blood, plasma or sera) containing an antibody(ies) identified to bind to the recombinant SARS-CoV-2 spike protein for a period of time (e.g., about 5 minutes, 10 minutes, 15 minutes, 30 minutes, 45 minutes, 1 hour, 1.5 hours, 2 hours, 3 hours, 4 hours, 5 hours, 6 hours, 12 hours, 18 hours, 24 hours, or more, or 5 to 30 minutes, 15 to 60 minutes, 1 hour to 3 hours, 3 to 6 hours, 6 to 12 hours, or 18 to 24 hours) prior to the addition of an agent of interest in an immunoassay. Once the agent is identified that inhibits the interaction between a recombinant SARS-CoV-2 spike protein described herein and an antibody or antibodies in a biological sample known to bind to the recombinant SARS-CoV-2 spike protein,t then the ability of that agent to inhibit the direct interaction between recombinant SARS-CoV-2 spike protein and its receptor(s) (e.g., ACE-2) may be assessed using techniques known to one of skill in the art. The agent of interest can be any agent (e.g., a compound, or an antibody or other biological agent) that may inhibit or reduce the interaction between SARS-CoV-2 spike protein and ACE-2. The immunoassay may be any immunoassay described herein or known to one of skill in the art. An agent identified in the in vitro assay described herein may have utility in preventing a SARS-CoV-2 infection or COVID-19 in a subject (e.g., human subject), or treating COVID-19 in a subject (e.g., human subject). Once an agent is identified in such an in vitro, then animal model assays as well as human studies with the agent may be conducted.

In another aspect, provided herein are methods for generating antibodies comprising administering a recombinant SARS-CoV-2 spike protein or composition described herein administered to a subject (e.g., a non-human subject). In some embodiments, a recombinant SARS-CoV-2 spike protein or composition described herein may be administered to a subject (e.g., a non-human subject) and antibodies may be isolated. The non-human subject may be a mouse, rat, or monkey. The non-human subject may produced human antibodies. The isolated antibodies may be cloned. An antibody(ies) isolated from the subject may be optimized, humanized or both. Alternatively, an antibody(ies) may be optimized, made chimeric or both. In some embodiments, hybridomas are produced which produce a particular antibody of interest. Techniques for isolating, cloning, chimerizing, humanizing, optimizing and for generating hybridomas are known to one of skill in the art. In a specific embodiment, antibodies generated by a method described herein may be utilized in assays (e.g., ELISAs, FACs or other immunoassays) as well as in passive immunization of a subject (e.g., a human subject). Thus, provided herein, in certain embodiments, are methods for treating SARS-CoV-2 infection or COVID-19, or preventing COVD-19, comprising administering an antibody(ies) generated by a method described herein to a subject (e.g., a human subject).

### 5.8 Kits

In another aspect, provided herein is a kit comprising one or more containers filled with one or more of the ingredients of an ELISA described herein, such as one or more recombinant SARS-CoV-2 spike proteins described herein. In a specific embodiment, the spike protein is a soluble protein described herein. Optionally associated with such container(s) can be a notice in the form prescribed by a governmental agency regulating the manufacture, use or sale of such kits, which notice reflects approval by the agency of manufacture, use or sale for human use (e.g., human administration). The kits may include instructions for use.

The kits encompassed herein can be used in accordance with the methods described herein. In one embodiment, a kit comprises a recombinant SARS-CoV-2 spike protein described herein in one or more containers. In a specific embodiment, provided herein are kits comprising a recombinant SARS-CoV-2 spike protein described herein and instructions for using the recombinant SARS-CoV-2 spike protein to assess the antibodies present in a subject. In another specific embodiment, provided herein are kits comprising a recombinant SARS-CoV-2 spike protein described herein for use in methods of assaying for the presence of SARS-CoV-2 spike protein receptor binding domain-specific antibodies in a biological sample (e.g., blood, plasma or sera). The biological sample may be saliva. In certain embodiments, a kit may further comprise a negative control, such as an antibody(ies) that specifically binds to a spike protein of an alphacoronavirus, such as, e.g., an antibody(ies) that specifically binds to a spike protein of NL63, 229E or both, or a betacoronavirus, such as, e.g., an antibody(ies) that specifically binds to a spike protein of OC43, HKU1 or both. In some embodiments, a kit may comprise a positive control, such as an antibody(ies) that specifically binds to the spike protein of SARS-CoV-2 (e.g., antibodies from COVID-19 patients or monoclonal antibodies (mAbs) like CR3022). In certain embodiments, a kit may comprise a negative control (such as an antibody(ies) that specifically binds to a spike protein of an alphacoronavirus, such as, e.g., an antibody(ies) that specifically binds to a spike protein of NL63, 229E or both, or a betacoronavirus, such as, e.g., an antibody(ies) that specifically binds to a spike protein of OC43, HKU1 or both) and a positive control (such as an antibody(ies) that specifically binds to the spike protein of SARS-CoV-2, e.g., antibodies from COVID-19 patients or monoclonal antibodies (mAbs) like CR3022). In some embodiments, a kit may comprise one, two or more, or all of the components of the ELISA described in Example 1 or Example 3, or both. In certain embodiments, a kit may comprise one, two or more, or all of the components of the ELISA described in Example 2, 4, 6, 7 or 8. In some embodiments, a kit may comprise one, two or more, or all of the components of the kit described in Example 11. In certain embodiments, a kit may comprise one, two or more, or all of the components of the kit described in Example 12, 13, 14 or 16.

In certain embodiments, a kit comprises a container, wherein the container comprises a recombinant soluble SARS-CoV-2 spike protein described herein immobilized (e.g., coated) on a solid support. In certain embodiments, a kit comprises two containers, wherein one container comprises a first recombinant soluble SARS-CoV-2 spike protein immobilized (e.g., coated) on a solid support, and the other container comprises a second soluble SARS-CoV-2 spike protein immobilized (e.g., coated) on a solid support, wherein the first recombinant soluble SARS-CoV-2 spike protein comprises a SARS-CoV-2 receptor binding domain or derivative thereof and optionally a tag, and the second recombinant soluble SARS-CoV-2 spike protein comprises a SARs-CoV-2 ectodomain or derivative thereof. The second recombinant soluble SARS-CoV-2 spike protein may further comprises one, two or or all of the following: C-terminal cleavage site, trimerization domain and tag. The second recombinant soluble SARS-CoV-2 spike protein may include stabilizing mutations of lysine to proline, such as described in Section 5.1 or Section 6. See Section 5.1 and Section 6 for examples of recombinant soluble SARS-CoV-2 spike proteins that may be included in such a kit. Solid supports include silica gels, resins, derivatized plastic films, glass surfaces, cellulose, polyacrylamide, nylon, polystyrene, polyvinyl chloride, polypropylene, beads (e.g., glass beads, plastic beads, magnetic beads, or polystyrene beads), or alumina gels. In some embodiments, a recombinant soluble SARS-CoV-2 spike protein described herein is immobilized (e.g., coated) on a bead (e.g., a glass bead, plastic bead, magnetic bead, or polystyrene bead), a test strip, a microtiter plate, a membrane, a glass surface, a slide (e.g., a microscopy slide), a microarray, a column (e.g., a chromatography column), or a biochip. In a specific embodiment, a recombinant soluble SARS-CoV-2 spike protein described herein is immobilized (e.g., coated) on a microtiter plate or well of a microtiter plate.

In a specific embodiment, a kit comprises: (a) a multi-well microtiter ELISA plate coated with a first recombinant soluble SARS-CoV-2 spike protein described herein; and (b) a multi-well ELISA microtiter plate coated with a second recombinant soluble SARS-CoV-2 spike protein described herein, wherein the first recombinant soluble SARS-CoV-2 spike protein and second recombinant soluble SARS-CoV-2 spike protein are different from each (e.g., the first recombinant soluble SARS-CoV-2 spike protein may comprise the receptor binding domain of a SARS-CoV-2 spike protein but not the entire ectodomain and the second recombinant soluble SARS-CoV-2 spike protein may comprise the ectodomain of a SARS-CoV-2 spike protein). In one embodiment, the first recombinant soluble SARS-CoV-2 spike protein comprises the amino acid sequence of SEQ ID NO:2. In one embodiment, the first recombinant soluble SARS-CoV-2 spike protein comprises the amino acid sequence of SEQ ID NO:2 without the first 14 amino acid residues. In another embodiment, the second recombinant soluble SARS-CoV-2 spike protein comprises the amino acid sequence of SEQ ID NO:4 or 6. In another embodiment, the second recombinant soluble SARS-CoV-2 spike protein comprises the amino acid sequence of SEQ ID NO: 4 or 6 without the first 14 amino acid residues. In another specific embodiment, the kit further comprises a labeled secondary antibody that binds to a constant region of an immunoglobulin (e.g., IgG). In certain embodiments, the kit further comprises a positive control antibody that binds to the recombinant soluble SARS-CoV-2 spike protein. In a specific embodiment, the positive control antibody is monoclonal antibody CR3022 or antibodies from COVID-19 patients. In certain embodiments, the kit further comprises a negative control antibody. In some embodiments, the kit further comprises a negative control and a positive control. The antibody may be labeled with a radioactive moiety, a chemiluminescent moiety, a fluorescent moiety or other detectable label, known to one of skill in the art or described herein. In a specific embodiment, the antibody is labeled with horseradish peroxidase. In certain embodiments, the kit further comprises a substrate for detection of the labeled antibody, such as, e.g., o-Phenylenediamine dihydrochloride (Sigma-Aldrich SIGMAFAST™ OPD). In some embodiments, the kit further comprises a stop solution to stop the reaction of the labeled antibody with the subtract, such as, e.g., 3M Hydrochloric acid.

In a specific embodiment, a kit comprises: (a) a 96 well microtiter ELISA plate coated with a first recombinant soluble SARS-CoV-2 spike protein described herein; and (b) a 96 well ELISA microtiter plate coated with a second recombinant soluble SARS-CoV-2 spike protein described herein, wherein the first recombinant soluble SARS-CoV-2 spike protein and second recombinant soluble SARS-CoV-2 spike protein are different from each (e.g., the first recombinant soluble SARS-CoV-2 spike protein may comprise the receptor binding domain of a SARS-CoV-2 spike protein but not the entire ectodomain and the second recombinant soluble SARS-CoV-2 spike protein may comprise the ectodomain of a SARS-CoV-2 spike protein). See also Section 5.1 for examples of recombinant SARS-CoV-2 spike proteins comprising the amino acid sequence of a SARS-CoV-2 spike protein receptor binding domain or a derivative thereof that may be used as the first recombinant soluble SARS-CoV-2 spike protein. See also Section 5.1 for examples of recombinant SARS-CoV-2 spike proteins comprising the amino acid sequence of a SARS-CoV-2 spike protein ectodomain or a derivative thereof that may be used as the second recombinant soluble SARS-CoV-2 spike protein. In one embodiment, the first recombinant soluble SARS-CoV-2 spike protein comprises the amino acid sequence of SEQ ID NO:2. In another embodiment, the first recombinant soluble SARS-CoV-2 spike protein comprises the amino acid sequence of SEQ ID NO:2 without the first 14 amino acid residues. In another embodiment, the second recombinant soluble SARS-CoV-2 spike protein comprises the amino acid sequence of SEQ ID NO:4 or 6. In another embodiment, the second recombinant soluble SARS-CoV-2 spike protein comprises the amino acid sequence of SEQ ID NO:4 or 6 without the first 14 amino acid residues. In another specific embodiment, the kit further comprises a labeled secondary antibody that binds to a constant region of an immunoglobulin (e.g., IgG). In certain embodiments, the kit further comprises a positive control antibody that binds to the recombinant soluble SARS-CoV-2 spike protein. In a specific embodiment, the positive control antibody is monoclonal antibody CR3022 or antibodies from COVID-19 patients. In certain embodiments, the kit further comprises a negative control antibody. In some embodiments, the kit further comprises a negative control and a positive control. The antibody may be labeled with a radioactive moiety, a chemiluminescent moiety, a fluorescent moiety or other detectable label, known to one of skill in the art or described herein. In a specific embodiment, the antibody is labeled with horseradish peroxidase. In certain embodiments, the kit further comprises a substrate for detection of the labeled antibody, such as, e.g., o-Phenylenediamine dihydrochloride (Sigma-Aldrich SIGMAFAST™ OPD). In some embodiments, the kit further comprises a stop solution to stop the reaction of the labeled antibody with the subtract, such as, e.g., 3M Hydrochloric acid.

In a specific embodiment, a kit comprises: (a) a multi-well microtiter ELISA plate coated with a first recombinant soluble SARS-CoV-2 spike protein described herein; and (b) a multi-well ELISA microtiter plate coated with a second recombinant soluble SARS-CoV-2 spike protein described herein, wherein the first recombinant soluble SARS-CoV-2 spike protein and second recombinant soluble SARS-CoV-2 spike protein. In one embodiment, the first recombinant soluble SARS-CoV-2 spike protein comprises the amino acid sequence of SEQ ID NO:2. In another embodiment, the first recombinant soluble SARS-CoV-2 spike protein comprises the amino acid sequence of SEQ ID NO:2 without the first 14 amino acid residues. In another embodiment, the second recombinant soluble SARS-CoV-2 spike protein comprises the amino acid sequence of SEQ ID NO:4 or 6. In another embodiment, the second recombinant soluble SARS-CoV-2 spike protein comprises the amino acid sequence of SEQ ID NO:4 or 6 without the first 14 amino acid residues. In certain embodiments, the kit further comprises one, two, three, four or more, or all of the following: (1) a labeled secondary antibody that binds to an isotype(s) or subtype(s) of an immunoglobulin (e.g., IgG), or is pan-specific for isotypes of an immunoglobulin; (2) a positive control antibody that binds to the recombinant soluble SARS-CoV-2 spike protein; (3) a negative control antibody; (4) a substrate or reagent for detection of the labeled antibody, such as described herein, e.g., o-Phenylenediamine dihydrochloride (Sigma-Aldrich SIGMAFAST™ OPD); (5) a stop solution to stop the reaction of the labeled antibody with the substrate, such as, e.g., 3M Hydrochloric acid; (6) blocking solution, such as described herein, including Section 6, infra; and (7) wash solution, such as described herein, including Section 6, infra (e.g., PBS-T). In certain embodiments, a kit comprises one, two or more of the reagents described below to conduct an ELISA, such as a blocking solution, wash solution, substrate, and/or stop solution. The antibody may be labeled with a radioactive moiety, a chemiluminescent moiety, a fluorescent moiety or other detectable label, known to one of skill in the art or described herein. In a specific embodiment, the antibody is labeled with horseradish peroxidase or alkaline phosphatase.

In a specific embodiment, a kit comprises: (a) a 96 well microtiter ELISA plate coated with a first recombinant soluble SARS-CoV-2 spike protein described herein; and (b) a 96 well ELISA microtiter plate coated with a second recombinant soluble SARS-CoV-2 spike protein described herein, wherein the first recombinant soluble SARS-CoV-2 spike protein and second recombinant soluble SARS-CoV-2 spike protein. In one embodiment, the first recombinant soluble SARS-CoV-2 spike protein comprises the amino acid sequence of SEQ ID NO:2. In another embodiment, the first recombinant soluble SARS-CoV-2 spike protein comprises the amino acid sequence of SEQ ID NO:2 without the first 14 amino acid residues. In another embodiment, the second recombinant soluble SARS-CoV-2 spike protein comprises the amino acid sequence of SEQ ID NO:4 or 6. In another embodiment, the second recombinant soluble SARS-CoV-2 spike protein comprises the amino acid sequence of SEQ ID NO:4 or 6 without the first 14 amino acid residues. In certain embodiments, the kit further comprises one, two, three, four or more, or all of the following: (1) a labeled secondary antibody that binds to constant region of an immunoglobulin (e.g., IgG); (2) a positive control antibody that binds to the recombinant soluble SARS-CoV-2 spike protein; (3) a negative control antibody; (4) a substrate or reagent for detection of the labeled antibody, such as described herein, e.g., o-Phenylenediamine dihydrochloride (Sigma-Aldrich SIGMAFAST™ OPD); (5) a stop solution to stop the reaction of the labeled antibody with the substrate, such as, e.g., 3M Hydrochloric acid; (6) blocking solution, such as described herein, including Section 6, infra; and (7) wash solution, such as described herein, including Section 6, infra (e.g., PBS-T). In certain embodiments, a kit comprises one, two or more of the reagents described below to conduct an ELISA, such as a blocking solution, wash solution, substrate, and/or stop solution. See Section 6, infra, for examples of such reagents. The antibody may be labeled with a radioactive moiety, a chemiluminescent moiety, a fluorescent moiety or other detectable label, known to one of skill in the art or described herein. In a specific embodiment, the antibody is labeled with horseradish peroxidase.

In a specific embodiment, a kit comprises: (a) a multi-well microtiter ELISA plate coated with a first recombinant soluble SARS-CoV-2 spike protein, wherein the first recombinant soluble SARS-CoV-2 spike protein comprises amino acid residues 319-541 of GenBank Accession No. MN908947.3 or amino acid residues corresponding to amino acid residues 319-541 of GenBank Accession No. MN908947.3, and a tag; and (b) a multi-well ELISA microtiter plate coated with a second recombinant soluble SARS-CoV-2 spike protein, wherein the second recombinant soluble SARS-CoV-2 spike protein comprises amino acid residues 1-1213 of GenBank Accession No. MN908947.3 or amino acid residues corresponding to amino acid residues 1-1213 of GenBank Accession No. MN908947.3, a C-terminal cleavage site, trimerization domain, and a tag, and wherein the recombinant soluble SARS-CoV-2 spike protein does not contain a polybasic cleavage site. In certain embodiments, the tag is a hexahistidine tag. In some embodiments, the second recombinant soluble SARS-CoV-2 spike protein lacks the signal sequence at amino acid residues 1-14 of GenBank Accession No. MN908947.3. In some embodiments, the C-terminal cleavage site is a C-terminal thrombin cleavage site. In certain embodiments, the trimerization domain is a T4 foldon trimerization domain. In a specific embodiment, the tag is a hexahistidine tag, the C-terminal cleavage site is a C-terminal thrombin cleavage site, and the trimerization domain is a T4 foldon trimerization domain. In another specific embodiment, the first recombinant soluble SARS-CoV-2 spike protein comprises the amino acid sequence of SEQ ID NO:2. In another specific embodiment, the first recombinant soluble SARS-CoV-2 spike protein comprises the amino acid sequence of SEQ ID NO:2 without the first 14 amino acid residues. In another specific embodiment, the second recombinant soluble SARS-CoV-2 spike protein comprises the amino acid sequence of SEQ ID NO:4 or 6. In another specific embodiment, the second recombinant soluble SARS-CoV-2 spike protein comprises the amino acid sequence of SEQ ID NO:4 or 6 without the first 14 amino acid residues. In another specific embodiment, the kit further comprises a labeled secondary antibody that binds to an isotype(s) or subtype(s) of an immunoglobulin (e.g., IgG), or is pan-specific for isotypes of an immunoglobulin. In certain embodiments, the kit further comprises a positive control antibody that binds to the recombinant soluble SARS-CoV-2 spike protein. In a specific embodiment, the positive control antibody is monoclonal antibody CR3022 or antibodies from COVID-19 patients. In certain embodiments, the kit further comprises a negative control antibody. In some embodiments, the kit further comprises a negative control and a positive control. See Section 6, infra, for examples of such controls. The antibody may be labeled with a radioactive moiety, a chemiluminescent moiety, a fluorescent moiety or other detectable label, known to one of skill in the art or described herein. In a specific embodiment, the antibody is labeled with horseradish peroxidase or alkaline phosphatase. In some embodiments, a kit further comprises one, two or more or all of the following: (1) a substrate or reagent for detection of the labeled antibody, such as described herein, e.g., o-Phenylenediamine dihydrochloride (Sigma-Aldrich SIGMAFAST™ OPD); (2) a stop solution to stop the reaction of the labeled antibody with the substrate, such as, e.g., 3M Hydrochloric acid; (3) blocking solution, such as described herein, including Section 6, infra; and (4) wash solution, such as described herein, including Section 6, infra (e.g., PBS-T). In certain embodiments, a kit further comprises one, two or more of the reagents described below to conduct an ELISA, such as a blocking solution, wash solution, substrate, and/or stop solution. See Section 6, infra, for examples of such reagents.

In a specific embodiment, a kit comprises: (a) a 96 well microtiter ELISA plate coated with a first recombinant soluble SARS-CoV-2 spike protein, wherein the first recombinant soluble SARS-CoV-2 spike protein comprises amino acid residues 319-541 of GenBank Accession No. MN908947.3 and a tag; and (b) a 96 well ELISA microtiter plate coated with a second recombinant soluble SARS-CoV-2 spike protein, wherein the second recombinant soluble SARS-CoV-2 spike protein comprises amino acid residues 1-1213 of GenBank Accession No. MN908947.3, a C-terminal cleavage site, trimerization domain, and a tag, and wherein the recombinant soluble SARS-CoV-2 spike protein does not contain a polybasic cleavage site. In some embodiments, the second recombinant soluble SARS-CoV-2 spike protein lacks the signal sequence at amino acid residues 1-14 of GenBank Accession No. MN908947.3. In certain embodiments, the tag is a hexahistidine tag. In some embodiments, the C-terminal cleavage site is a C-terminal thrombin cleavage site. In certain embodiments, the trimerization domain is a T4 foldon trimerization domain. In a specific embodiment, the tag is a hexahistidine tag, the C-terminal cleavage site is a C-terminal thrombin cleavage site, and the trimerization domain is a T4 foldon trimerization domain. In another specific embodiment, the first recombinant soluble SARS-CoV-2 spike protein comprises the amino acid sequence of SEQ ID NO:2. In another specific embodiment, the first recombinant soluble SARS-CoV-2 spike protein comprises the amino acid sequence of SEQ ID NO:2 without the first 14 amino acid residues. In another specific embodiment, the second recombinant soluble SARS-CoV-2 spike protein comprises the amino acid sequence of SEQ ID NO:4 or 6. In another specific embodiment, the second recombinant soluble SARS-CoV-2 spike protein comprises the amino acid sequence of SEQ ID NO:4 or 6 without the first 14 amino acid residues. In another specific embodiment, the kit further comprises a labeled secondary antibody that binds to a constant region of an immunoglobulin (e.g., IgG). In certain embodiments, the kit further comprises a positive control antibody that binds to the recombinant soluble SARS-CoV-2 spike protein. In a specific embodiment, the positive control antibody is monoclonal antibody CR3022 or antibodies from COVID-19 patients. In certain embodiments, the kit further comprises a negative control antibody. In some embodiments, the kit further comprises a negative control and a positive control. The antibody may be labeled with a radioactive moiety, a chemiluminescent moiety, a fluorescent moiety or other detectable label, known to one of skill in the art or described herein. In a specific embodiment, the antibody is labeled with horseradish peroxidase. In some embodiments, a kit further comprises one, two or more or all of the following: (1) a substrate or reagent for detection of the labeled antibody, such as described herein, e.g., o-Phenylenediamine dihydrochloride (Sigma-Aldrich SIGMAFAST™ OPD); (2) a stop solution to stop the reaction of the labeled antibody with the substrate, such as, e.g., 3M Hydrochloric acid; (3) blocking solution, such as described herein, including Section 6, infra; and (4) wash solution, such as described herein, including Section 6, infra (e.g., PBS-T). In certain embodiments, a kit further comprises one, two or more of the reagents described below to conduct an ELISA, such as a blocking solution, wash solution, substrate, and/or stop solution. See Section 6, infra, for examples of such reagents.

In another embodiment, provided herein is a kit comprising: (a) a first multi-well microtiter plate (e.g., a 96-well microtiter plate) coated with a first recombinant soluble SARS-CoV-2 spike protein, wherein the first recombinant soluble SARS-CoV-2 spike protein comprises a SARS-CoV-2 spike protein receptor binding domain (e.g., amino acid residues 319 to 541 of a SARS-CoV-2 spike protein) or a derivative thereof, and optionally a tag (e.g., histidine tag at the C-terminus); (b) a vial containing concentrated monoclonal antibody specific to human IgG or another immunoglobulin isotype or subtype, or an antibody pan-specific for human immunoglobulin conjugated to horseradish peroxidase, alkaline phosphatase or another detectable moiety; (c) a vial containing substrate solution (e.g., substrate solution comprising 3,3′,5,5′-Tetramethylbenzidine (TMB) substrate); (d) a vial containing a stop solution (e.g., a stop solution comprising an acid); (e) a vial containing a sample buffer (e.g., a sample buffer comprising phosphate buffered saline (PBS) with 0.5% to 5% milk); (f) a vial containing a wash buffer (e.g., a wash buffer comprising phosphate buffered saline and 0.5% to 2% of a surfactant, such as polysorbate (e.g., polysorbate-20 or polysorbate-80) or Triton X-100). See Section 5.1 for examples of recombinant SARS-CoV-2 spike proteins comprising the amino acid sequence of a SARS-CoV-2 spike protein receptor binding domain or a derivative thereof that may be used as the first recombinant soluble SARS-CoV-2 spike protein. In some embodiments, the kit further comprises a second multi-well microtiter plate (e.g., a 96-well microtiter plate) coated with a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof. See Section 5.1 for examples of recombinant SARS-CoV-2 spike proteins comprising the amino acid sequence of a SARS-CoV-2 spike protein ectodomain or a derivative thereof that may be used as the second recombinant soluble SARS-CoV-2 spike protein. In some embodiments, the kit comprises 4 of the first multi-well microtiter plate and 5 of the second multi-well microtiter plate. In other embodiments, the kit comprises 7 of the first multi-well microtiter plate and 3 of the second multi-well microtiter plate. In a specific embodiment, the first recombinant SARS-CoV-2 spike protein comprises the amino acid sequence set forth in SEQ ID NO:2 or 10. In another specific embodiment, the second recombinant SARS-CoV-2 spike protein comprises the amino acid sequence set forth in SEQ ID NO:4 or 6. In another specific embodiment, the second recombinant SARS-CoV-2 spike protein comprises the amino acid sequence set forth in SEQ ID NO:4 or 6 without the first 14 amino acid residues. In some embodiments, the vial containing the concentrated monoclonal antibody specific to human IgG conjugated to horseradish peroxidase comprises 100 to 500 μl of the concentrated antibody. In another embodiment, the vial containing the concentrated monoclonal antibody specific to human IgG conjugated to horseradish peroxidase, alkaline phosphatase or another detectable moiety comprises 100 μl, 125 μl, 150 μl, 175 μl, 200 μl, 225 μl, 250 μl, 300 μl, 325 μl, 350 μl, 375 μl, 400 μl, 425 μl, 450 μl, 475 μl or 500 μl of the concentrated antibody. In certain embodiments, the concentrated monoclonal antibody specific to human IgG conjugated to horseradish peroxidase, alkaline phosphatase or another detectable moiety is 100× to 1000× concentrated monoclonal antibody. In some embodiments, the concentrated monoclonal antibody specific to human IgG conjugated to horseradish peroxidase, alkaline phosphatase or another detectable moiety is 100×, 200×, 300×, 400×, 500×, 600×, 700×, 800×, 900× or 1000× concentrated monoclonal antibody. In certain embodiments, the kit comprises a two or more vials containing concentrated monoclonal antibody specific to human IgG conjugated to horseradish peroxidase, alkaline phosphatase or another detectable moiety. In some embodiments, the stop solution comprises an acid such as, e.g., an alkylsuphonic acid (e.g., 1-5% methanesulfonic acid), 1 N HCl or 2 N H₂SO₄. In certain embodiments, the vial comprising the stop solution comprises 100 to 500 mL of the stop solution. In certain embodiments, the vial comprising the stop solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the stop solution. In certain embodiments, the vial comprising the substrate solution comprises 100 to 500 mL of the substrate solution. In certain embodiments, the vial comprising the substrate solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the substrate solution. In certain embodiments, the vial comprising the sample buffer comprises 100 to 500 mL of the sample buffer. In certain embodiments, the vial comprising the sample buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the sample buffer. In certain embodiments, the vial comprising the wash buffer comprises 100 to 500 mL of the wash buffer. In certain embodiments, the vial comprising the wash buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the wash buffer. In specific embodiments, the kit further comprises a vial containing a positive control monoclonal antibody specific for SARS-CoV-2 spike protein (e.g., a monoclonal antibody (e.g., an IgG) specific for SARS-CoV-2 spike protein binds to the receptor binding domain). In certain embodiments, the vial containing the positive control monoclonal antibody comprises 500 μl to 2 ml of the positive control monoclonal antibody. In some embodiments, the vial containing the positive control monoclonal antibody comprises 500 μl, 1 ml, 1.5 ml or 2 ml of the positive control monoclonal antibody. In certain embodiments, the positive control monoclonal antibody is diluted 1:2, 1:4, 1:5, 1:6, 1:8, 1:10 in the sample buffer. In certain embodiments, the kit further comprises a vial containing a negative control (e.g., a control buffer or a monoclonal antibody that does not bind to the SARS-CoV-2 spike protein). In some embodiments, the vial containing the negative control monoclonal antibody comprises 500 μl, 1 ml, 1.5 ml or 2 ml of the positive control monoclonal antibody. In certain embodiments, the negative control monoclonal antibody is diluted 1:2, 1:4, 1:5, 1:6, 1:8, 1:10 in the sample buffer. In some embodiments, the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml. In specific embodiments, the kit further comprises 7 calibrators, wherein each calibrator is a monoclonal antibody specific for SARS-CoV-2 spike protein and each calibrator has 0 to 200 AU/ml, and wherein each calibrator has a different AU/ml. In a specific embodiment, the calibrators are those in Example 11, infra. In specific embodiments, the kit comprises instructions for performing the immunoassay. In a specific embodiment, the kit is stored at 2° to 8° C.

In another embodiment, provided herein is a kit comprising: (a) a vial containing a first recombinant soluble SARS-CoV-2 spike protein, wherein the first recombinant soluble SARS-CoV-2 spike protein comprises a SARS-CoV-2 spike protein receptor binding domain (e.g., amino acid residues 319 to 541 of a SARS-CoV-2 spike protein) or a derivative thereof, and optionally a tag (e.g., histidine tag at the C-terminus); (b) a vial containing concentrated monoclonal antibody specific to human IgG or another immunoglobulin isotype or subtype, or an antibody pan-specific for human immunoglobulin conjugated to horseradish peroxidase, alkaline phosphatase or another detectable moiety; (c) a vial containing substrate solution (e.g., substrate solution comprising 3,3′,5,5′-Tetramethylbenzidine (TMB) substrate); (d) a vial containing a stop solution (e.g., a stop solution comprising an acid); (e) a vial containing a sample buffer (e.g., a sample buffer comprising phosphate buffered saline (PBS) with 0.5% to 5% milk); (f) a vial containing a wash buffer (e.g., a wash buffer comprising phosphate buffered saline and 0.5% to 2% of a surfactant, such as polysorbate (e.g., polysorbate-20 or polysorbate-80) or Triton X-100). See Section 5.1 for examples of recombinant SARS-CoV-2 spike proteins comprising the amino acid sequence of a SARS-CoV-2 spike protein receptor binding domain or a derivative thereof that may be used as the first recombinant soluble SARS-CoV-2 spike protein. In some embodiments, the kit further comprises a vial containing a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof. See Section 5.1 for examples of recombinant SARS-CoV-2 spike proteins comprising the amino acid sequence of a SARS-CoV-2 spike protein ectodomain or a derivative thereof that may be used as the second recombinant soluble SARS-CoV-2 spike protein. In a specific embodiment, the first recombinant SARS-CoV-2 spike protein comprises the amino acid sequence set forth in SEQ ID NO:2 or 10. In another specific embodiment, the second recombinant SARS-CoV-2 spike protein comprises the amino acid sequence set forth in SEQ ID NO:4 or 6. In some embodiments, the vial containing the concentrated monoclonal antibody specific to human IgG conjugated to horseradish peroxidase comprises 100 to 500 μl of the concentrated antibody. In another embodiment, the vial containing the concentrated monoclonal antibody specific to human IgG conjugated to horseradish peroxidase, alkaline phosphatase or another detectable moiety comprises 100 μl, 125 μl, 150 μl, 175 μl, 200 μl, 225 μl, 250 μl, 300 μl, 325 μl, 350 μl, 375 μl, 400 μl, 425 μl, 450 μl, 475 μl or 500 μl of the concentrated antibody. In certain embodiments, the concentrated monoclonal antibody specific to human IgG conjugated to horseradish peroxidase, alkaline phosphatase or another detectable moiety is 100× to 1000× concentrated monoclonal antibody. In some embodiments, the concentrated monoclonal antibody specific to human IgG conjugated to horseradish peroxidase, alkaline phosphatase or another detectable moiety is 100×, 200×, 300×, 400×, 500×, 600×, 700×, 800×, 900× or 1000× concentrated monoclonal antibody. In certain embodiments, the kit comprises a two or more vials containing concentrated monoclonal antibody specific to human IgG conjugated to horseradish peroxidase, alkaline phosphatase or another detectable moiety. In some embodiments, the stop solution comprises an acid such as, e.g., an alkylsuphonic acid (e.g., 1-5% methanesulfonic acid), 1 N HCl or 2 N H₂SO₄. In certain embodiments, the vial comprising the stop solution comprises 100 to 500 mL of the stop solution. In certain embodiments, the vial comprising the stop solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the stop solution. In certain embodiments, the vial comprising the substrate solution comprises 100 to 500 mL of the substrate solution. In certain embodiments, the vial comprising the substrate solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the substrate solution. In certain embodiments, the vial comprising the sample buffer comprises 100 to 500 mL of the sample buffer. In certain embodiments, the vial comprising the sample buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the sample buffer. In certain embodiments, the vial comprising the wash buffer comprises 100 to 500 mL of the wash buffer. In certain embodiments, the vial comprising the wash buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the wash buffer. In specific embodiments, the kit further comprises a vial containing a positive control monoclonal antibody specific for SARS-CoV-2 spike protein (e.g., a monoclonal antibody (e.g., an IgG) specific for SARS-CoV-2 spike protein binds to the receptor binding domain). In certain embodiments, the vial containing the positive control monoclonal antibody comprises 500 μl to 2 ml of the positive control monoclonal antibody. In some embodiments, the vial containing the positive control monoclonal antibody comprises 500 μl, 1 ml, 1.5 ml or 2 ml of the positive control monoclonal antibody. In certain embodiments, the positive control monoclonal antibody is diluted 1:2, 1:4, 1:5, 1:6, 1:8, 1:10 in the sample buffer. In certain embodiments, the kit further comprises a vial containing a negative control (e.g., a control buffer or a monoclonal antibody that does not bind to the SARS-CoV-2 spike protein). In some embodiments, the vial containing the negative control monoclonal antibody comprises 500 μl, 1 ml, 1.5 ml or 2 ml of the positive control monoclonal antibody. In certain embodiments, the negative control monoclonal antibody is diluted 1:2, 1:4, 1:5, 1:6, 1:8, 1:10 in the sample buffer. In some embodiments, the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml. In specific embodiments, the kit further comprises 7 calibrators, wherein each calibrator is a monoclonal antibody specific for SARS-CoV-2 spike protein and each calibrator has 0 to 200 AU/ml, and wherein each calibrator has a different AU/ml. In a specific embodiment, the calibrators are those in Example 11, infra. In another specific embodiment, the calibrators are those in Example 12, 13 or 14, infra. In specific embodiments, the kit comprises instructions for performing the immunoassay. In a specific embodiment, the kit is stored at 2° to 8° C.

In another embodiment, provided herein is a kit comprising: (a) a multi-well microtiter plate (e.g., a 96-well microtiter plate) coated with a recombinant soluble SARS-CoV-2 spike protein, wherein the recombinant soluble SARS-CoV-2 spike protein comprises a SARS-CoV-2 spike protein ectodomain or a derivative thereof; (b) a vial containing concentrated monoclonal antibody specific to human IgG or another immunoglobulin isotype or subtype, or an antibody pan-specific for human immunoglobulin conjugated to horseradish peroxidase, alkaline phosphatase or another detectable moiety; (c) a vial containing substrate solution (e.g., substrate solution comprising 3,3′,5,5′-Tetramethylbenzidine (TMB) substrate); (d) a vial containing a stop solution (e.g., a stop solution comprising an acid); (e) a vial containing a sample buffer (e.g., a sample buffer comprising phosphate buffered saline (PBS) with 0.5% to 5% milk); (f) a vial containing a wash buffer (e.g., a wash buffer comprising phosphate buffered saline and 0.5% to 2% of a surfactant, such as polysorbate (e.g., polysorbate-20 or polysorbate-80) or Triton X-100). See Section 5.1 for examples of recombinant SARS-CoV-2 spike proteins comprising the amino acid sequence of a SARS-CoV-2 spike protein ectodomain or a derivative thereof that may be used as the recombinant soluble SARS-CoV-2 spike protein. In another embodiment, provided herein is a kit comprising: (a) a vial containing a recombinant soluble SARS-CoV-2 spike protein, wherein the recombinant soluble SARS-CoV-2 spike protein comprises a SARS-CoV-2 spike protein ectodomain or a derivative thereof; (b) a vial containing concentrated monoclonal antibody specific to human IgG or another immunoglobulin isotype or subtype, or an antibody pan-specific for human immunoglobulin conjugated to horseradish peroxidase, alkaline phosphatase or another detectable moiety; (c) a vial containing solution (e.g., substrate solution comprising 3,3′,5,5′-Tetramethylbenzidine (TMB) substrate); (d) a vial containing a stop solution (e.g., a stop solution comprising an acid); (e) a vial containing a sample buffer (e.g., a sample buffer comprising phosphate buffered saline (PBS) with 0.5% to 5% milk); (f) a vial containing a wash buffer (e.g., a wash buffer comprising phosphate buffered saline and 0.5% to 2% of a surfactant, such as polysorbate (e.g., polysorbate-20 or polysorbate-80) or Triton X-100). See Section 5.1 for examples of recombinant SARS-CoV-2 spike proteins comprising the amino acid sequence of a SARS-CoV-2 spike protein ectodomain or a derivative thereof that may be used as the recombinant soluble SARS-CoV-2 spike protein. In a specific embodiment, the recombinant SARS-CoV-2 spike protein comprises the amino acid sequence set forth in SEQ ID NO:4 or 6. In some embodiments, the vial containing the concentrated monoclonal antibody specific to human IgG conjugated to horseradish peroxidase comprises 100 to 500 μl of the concentrated antibody. In another embodiment, the vial containing the concentrated monoclonal antibody specific to human IgG conjugated to horseradish peroxidase, alkaline phosphatase or another detectable moiety comprises 100 μl, 125 μl, 150 μl, 175 μl, 200 μl, 225 μl, 250 μl, 300 μl, 325 μl, 350 μl, 375 μl, 400 μl, 425 μl, 450 μl, 475 μl or 500 μl of the concentrated antibody. In certain embodiments, the concentrated monoclonal antibody specific to human IgG conjugated to horseradish peroxidase, alkaline phosphatase or another detectable moiety is 100× to 1000× concentrated monoclonal antibody. In some embodiments, the concentrated monoclonal antibody specific to human IgG conjugated to horseradish peroxidase, alkaline phosphatase or another detectable moiety is 100×, 200×, 300×, 400×, 500×, 600×, 700×, 800×, 900× or 1000× concentrated monoclonal antibody. In certain embodiments, the kit comprises a two or more vials containing concentrated monoclonal antibody specific to human IgG conjugated to horseradish peroxidase, alkaline phosphatase or another detectable moiety. In some embodiments, the stop solution comprises an acid such as, e.g., an alkylsuphonic acid (e.g., 1-5% methanesulfonic acid), 1 N HCl or 2 N H₂SO₄. In certain embodiments, the vial comprising the stop solution comprises 100 to 500 mL of the stop solution. In certain embodiments, the vial comprising the stop solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the stop solution. In certain embodiments, the vial comprising the substrate solution comprises 100 to 500 mL of the substrate solution. In certain embodiments, the vial comprising the substrate solution comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the substrate solution. In certain embodiments, the vial comprising the sample buffer comprises 100 to 500 mL of the sample buffer. In certain embodiments, the vial comprising the sample buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the sample buffer. In certain embodiments, the vial comprising the wash buffer comprises 100 to 500 mL of the wash buffer. In certain embodiments, the vial comprising the wash buffer comprises 100 mL, 110 mL, 115 mL, 116 mL, 117 mL, 118 mL, 120 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL or 500 mL of the wash buffer. In specific embodiments, the kit further comprises a vial containing a positive control monoclonal antibody specific for SARS-CoV-2 spike protein (e.g., a monoclonal antibody (e.g., an IgG) specific for SARS-CoV-2 spike protein binds to the receptor binding domain). In certain embodiments, the vial containing the positive control monoclonal antibody comprises 500 μl to 2 ml of the positive control monoclonal antibody. In some embodiments, the vial containing the positive control monoclonal antibody comprises 500 μl, 1 ml, 1.5 ml or 2 ml of the positive control monoclonal antibody. In certain embodiments, the positive control monoclonal antibody is diluted 1:2, 1:4, 1:5, 1:6, 1:8, 1:10 in the sample buffer. In certain embodiments, the kit further comprises a vial containing a negative control (e.g., a control buffer or a monoclonal antibody that does not bind to the SARS-CoV-2 spike protein). In some embodiments, the vial containing the negative control monoclonal antibody comprises 500 μl, 1 ml, 1.5 ml or 2 ml of the positive control monoclonal antibody. In certain embodiments, the negative control monoclonal antibody is diluted 1:2, 1:4, 1:5, 1:6, 1:8, 1:10 in the sample buffer. In some embodiments, the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml. In specific embodiments, the kit further comprises 7 calibrators, wherein each calibrator is a monoclonal antibody specific for SARS-CoV-2 spike protein and each calibrator has 0 to 200 AU/ml, and wherein each calibrator has a different AU/ml. In a specific embodiment, the calibrators are those in Example 11, infra. In a specific embodiment, the calibrators are those in Example 12, 13, or 14, infra. In specific embodiments, the kit comprises instructions for performing the immunoassay. In a specific embodiment, the kit is stored at 2° to 8° C.

In a specific embodiment, a kit described herein is one described in Section 6, infra (e.g., a kit described in Example 11, 12, 13, 14, or 16).

### 6. EXAMPLES

### 6.1 Example 1: A Detailed Protocol for a Serological Assay to Detect SARS-CoV-2 Seroconversion in Humans: Antigen Production and Test Set-Up

In late 2019, cases of atypical pneumonia were detected in China. The etiological agent was quickly identified as a betacoronavirus (named SARS-CoV-2) which has since caused a pandemic. Several methods allowing for the specific detection of viral nucleic acids have been established but only allow detection of the virus during a short period of time, generally during acute infection. Serological assays are urgently needed to conduct serosurveys, to understand the antibody responses mounted in response to the virus and to identify individuals who are potentially immune to re-infection. Here we describe a detailed protocol for expression of antigens derived from the spike protein of SARS-CoV-2 that can serve as a substrate for immunological assays as well as a two-step serological enzyme-linked immunosorbent assay (ELISA). These assays can be used for research studies and for testing in clinical laboratories.

### 6.1.1 Introduction:

Severe acute respiratory syndrome coronavirus 2 (SARS-CoV-2), the virus that causes COronaVIrus Disease 2019 (COVID19), emerged in late 2019 in Wuhan, China (Wu et al., 2020; Zhu et al., 2020). The virus spread globally causing a pandemic. Currently, no drugs or antivirals are available and countermeasures are limited to non-pharmaceutical interventions (NPIs). Nucleic acid-based tests for detection of the virus during acute disease are in use worldwide (Chu et al., 2020; Corman et al., 2020). However, the development of serological assays is lagging due to lack of suitable reagents. Serological assays are needed to perform serosurveys aimed at determining the real infection rate and infection fatality rate in a given population. Furthermore, they are useful to characterize the immune response to the virus in a detailed qualitative and quantitative manner. In addition, serological assays are also of immediate practical use. They can be used to identify individuals who were infected (including severe, mild and asymptomatic cases) and who are now potentially immune. A recent study in non-human primates showed that reinfection, at least in the small number of animals used in the study, does not occur (Bao et al., 2020) once antibody responses have been mounted. Infection with coronaviruses circulating in human populations such as HKU, NL63, etc., also leads to immunity that protects from re-infection for months to years (Callow, Parry, Sergeant, & Tyrrell, 1990). Therefore, individuals who have mounted an immune response to SARS-CoV-2 are likely immune, which means that they are unlikely to transmit the virus to others. As an example, healthcare workers who are immune could potentially care for COVID19 patients with minimal risk to themselves, their colleagues and other patients. In addition, the use of convalescent serum may serve as a valuable treatment option for patients with severe COVID19, especially in the absence of other options. A serological assay is critical for identifying potential blood donors.

The surface glycoprotein of the virus, termed the spike (S) protein, mediates attachment of the virus to human cells via its receptor-binding domain (RBD) (Wrapp et al., 2020) and mediates fusion of viral and cellular membranes. Antibodies binding to the spike protein, and especially to the RBD domain, can neutralize coronaviruses, including SARS-CoV-2. Therefore, we used different recombinant spike protein preparations as the antigens for our ELISA. We reported in our earlier work that individuals not exposed to SARS-CoV-2 are completely naïve to the spike protein and their serum samples show no reactivity in an ELISA (Amanat et al., 2020). It is, therefore, easy to distinguish between exposed/immune and naïve people.

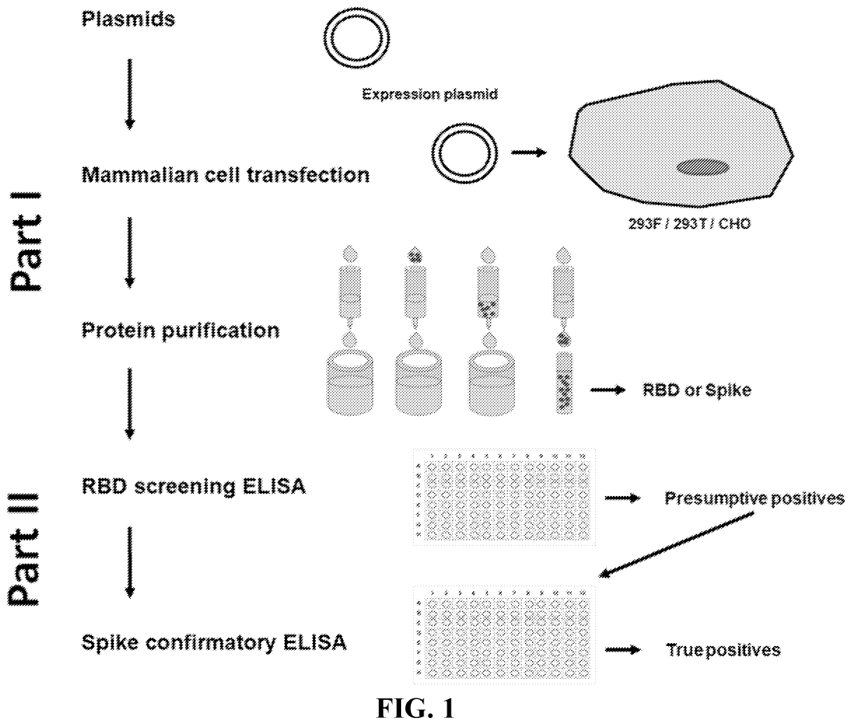

In this report, we provide detailed protocols for expressing the required antigen(s) (Part I, Basic Protocol 1: Mammalian cell transfection and protein purification protocol) as well as setting up the ELISA that we have developed (Part II, Basic Protocol 2: A two-step ELISA protocol for high-throughput screening of human serum samples for antibodies binding to the spike protein of SARS-CoV-2) (FIG. 1). We believe that this protocol will be useful not only for research laboratories around the globe, but also for testing in diagnostic/clinical laboratories. The described protocol setup works well for us but it can easily be modified, adapted to local needs and improved by the research community in the future. Mammalian expression plasmids for the generation of the recombinant proteins are available.

### 6.1.2 Part I: Mammalian Cell Transfection and Protein Purification Protocol

This protocol can be used for both expression vectors for the secreted RBD as well as a soluble, trimeric version of the SARS-CoV-2 spike protein. Expression levels of the RBD are very high in our hands (>20 mg/L culture) while expression levels for the full-length spike are lower (approximately 1 mg/L). Therefore, we use the recombinant RBD for initial screening ELISAs and the full-length spike or confirmatory ELISAs (as described in Part II). Preparation of plasmids for mammalian cell expression are not described here. The plasmids all carry a betalactamase (amp) resistance gene. They are grown in E. coli at 37° C. (or 30° C.) in shaker flasks over night. High-quality plasmid DNA can be obtained using commercially available maxiprep kits (ideally with an endotoxin removal step). Importantly, other cell lines (293T, CHO, etc.), other media, transfection reagents and more sophisticated protein purification methods might be used as alternatives if available. Of note, cells can be transfected in regular flasks in regular incubators without shaking.

### Materials:

Expi293 Expression Medium (Gibco #A1435102)
Opti-MEM™ I Reduced Serum Medium (Gibco #31985088)
ExpiFectamine™ 293 Transfection Kit (Gibco #A14524)
PBS (1×) (Gibco #10010-023 or equivalent)
Ni-NTA Agarose (Qiagen #30230 or equivalent)
SDS-PAGE gels (Bio-Rad #4561094 or equivalent)
SDS-PAGE cell and power supply
Sodium phosphate monobasic monohydrate NaH₂PO₄·H₂O (Sigma Aldrich #S3522 or equivalent)
Sodium Chloride NaCl (Sigma-Aldrich #S3014 or equivalent)
Imidazole (Sigma-Aldrich #15513 or equivalent)
Disposable Polycarbonate Erlenmeyer Flasks (Corning #431147)
Trypan blue solution, 0.4% (Gibco #15250-06 or equivalent)
Cell counting slides (Invitrogen #C10312 or equivalent)
5 mL Polypropylene columns (Qiagen #34964 or equivalent)
Amicon™ Ultra Centrifugal Filter Units 10 kDa (MilliporeSigma #UFC901024 or equivalent)
Amicon™ Ultra Centrifugal Filter Units 50 kDa (MilliporeSigma #UFC905024 or equivalent)
Polypropylene sterile conical tubes
   15 mL (Denville Scientific #C1018P or equivalent)
   50 mL (Fisher Denville Scientific #C1060P or equivalent)
Sterile, serological pipettes
   5 mL (Falcon #356543 or equivalent)
   10 mL (Falcon #357551 or equivalent)
   25 mL (Falcon #357535 or equivalent)
   50 mL (Falcon #356550 or equivalent)
Micropipette tips
   20 μL barrier tips (Denville Scientific #P1121 or equivalent)
   200 μL barrier tips (Denville Scientific #P1122 or equivalent)
   200 μL tips (USA Scientific #1111-1700 or equivalent)
   1000 μL barrier tips (Denville Scientific #P1126 or equivalent)
1.5 mL Eppendorf tubes (Denville #C2170 or equivalent)
Stericup Quick Release-GP Sterile Vacuum Filtration System (MilliporeSigma S2GPU05RE or equivalent)
Pipet-Aid
Micropipettes
Class II biological safety cabinet
Timer
Countess II cell counter or equivalent
CO₂ incubator with built in shaker (Eppendorf New Brunswick S41i or Equivalent)
Benchtop shaker (Benchmark #BT3000 or equivalent)
Cooling Centrifuge (Eppendorf 5810R or equivalent)
Refrigerator at 4° C. (+/−1° C.)
Ultra-low freezer (−80° C.)

### Definitions

RBD=Receptor-Binding Domain of SARS-CoV-2 (NR-52306)
PBS=Phosphate-Buffered Saline
RT=Room Temperature (18-25° C.)
MEM=Minimum Essential Medium
DNA=Deoxyribonucleic Acid
Ni-NTA=Nickel-Nitrilotriacetic acid

### Procedure

### Mammalian Cell Transfection:

HEK 293F cells are counted using an automated cell counter (or a regular counting chamber) and seeded at a density of 600,000 cells/ml in Expi293 expression medium. The viability of the cells must be greater than 90% at all times. Cells are passaged every 3-4 days and incubated in an orbital shaking incubator at 37° C. and 125 RPM with 8% CO₂. A maximum cell density of 4-5×10⁶ cells/ml is recommended and at this point, cells should be immediately passaged.

Transfections are performed according to manufacturer's instructions. 600×10⁶ cells are suspended in 200 ml of Expi293 expression media in a 1 L shaker flask. Twelve ml of Opti-MEM is added to two 50 ml falcon tubes: one tube receives 200 ug (1 ug/ul) of respective plasmid DNA (for RBD or full-length spike) while the other tube receives 640 μl of ExpiFectamine transfection reagent. The contents of both the 50 ml Falcon tubes are mixed together and incubated at RT for 10 minutes after which the transfection mixture is added dropwise to the cells. Cells are then returned to the shaking incubator. Sixteen hours post transfection, 1.2 ml of Expifectamine 293 Transfection Enhancer 1 and 12.1 ml of Expifectamine 293 Transfection Enhancer 2 is added to the culture and subsequently, the culture is returned to the shaking incubator.

Three days post-transfection, the cells are harvested and spun at 4,000 g for 20 minutes at 4° C. The supernatant is filtered using a 0.22 μm stericup filter, the cell pellet can be discarded. Alternately, cells can be spun at 200 g for 10 minutes, supernatant can be collected, and the same cells can be resuspended in 200 mls of fresh Expi293 expression medium and returned to the shaking incubator for another 3 days. This alternate strategy works well with the RBD but is less suitable for the full-length spike (we have detected protein degradation in that case).

Ideally is the supernatant containing the protein is further processed immediately. Alternatively, if it is stored, it must be kept at 4° C. (and for no longer than overnight/16h) in order to prevent denaturation of the protein at room temperature.

### Protein Purification Via Gravity Flow:

Note: This step can be substituted with more advanced purification methodology if, e.g., an Aekta purifier is available. The methods described below work, even in labs not geared towards protein purification.

Prior to use, Ni-NTA resin (6 ml per 200 ml culture) is washed with fresh PBS, then spun at 2000 g for 10 min in a centrifuge. Once the centrifugation is complete, PBS is discarded, and resin is resuspended with the supernatant from cells and inverted about two or three times. The resin is then incubated with the supernatant for 2 hours on a shaker at RT.

Two clean polypropylene columns are loaded with the supernatant-resin mixture and then washed with Wash Buffer four times. Columns are then eluted using the Elution Buffer. Which contains a high concentration of imidazole Four fractions are collected from each column by incubating the resin in the column with 3 ml of Elution Buffer for each fraction. Eluate is collected directly in a 50 ml falcon tube placed on ice. The total volume of eluate should be 24 ml from the two columns. More columns can be used to speed up the purification time depending on the volume of the culture.

Eluate is spun through 10 kDa Amicon Centrifugal Filter Units (for RBD) or 50 kDa Amicon Centrifugal Filter Units (for full-length spike) at 4000 g for 30 minutes (or longer if eluate takes longer to pass through the membrane) at 4° C. until only 200-300 μl remain in the unit. The Centrifugal Filter Unit is then washed with PBS twice by centrifugation at 4000 g for 30 minutes at 4° C. (washing means filling up with PBS and centrifugation until the volume in the unit is down to 200-300 ul again). Finally, the protein is collected from the Amicon centrifugal unit, concentration is measured (e.g., using Bradford reagent or similar methods), and a denaturing SDS-page is run to check integrity of the purified protein.

After the elution step, protein is always kept on ice. For storage longer than 24h it should be frozen to −80° C. to avoid degradation.

### Wash Buffer (4L):

NaH₂PO₄. H₂O 31.74 g
NaCl 70.16 g
Imidazole 5.44 g (final concentration is 20 mM)
Distilled water* 4L

### Elution Buffer (4L):

NaH₂PO4. H₂O 31.74 g
NaCl 70.16 g
Imidazole 64.0 g (final concentration is 235 mM)
Distilled water* 4L
*Use Distilled water filtered using a 0.22 um stericup vacuum filtration system.

### 6.1.3 Part II: A Two-Step ELISA Protocol for High Throughput Screening of Human Serum Samples for Antibodies Binding to the Spike Protein of SARS-CoV-2

The purpose of this part of the protocol is to describe the procedure for measuring human antibody responses to the recombinant receptor binding domain (RBD) of the spike protein or full-length spike protein of SARS-CoV-2 and to ensure the reproducibility and consistency of the obtained results.

We developed this as a two-step ELISA in which the first step (A) includes relatively high throughput screening of samples in a single serum dilution against the RBD (which expresses very well and therefore there is typically more protein available). This is followed by a second step (B) in which positive samples from the first step undergo a confirmatory ELISA against the full length spike protein (which is harder to purify, therefore there is usually less available). For the second step a dilution curve is performed. Typically, if only one operator is available, screening ELISAs can be run in the morning (760 samples/10 plates per run) and confirmatory ELISAs can be run in the afternoon (140 samples/10 plates per run). Of note, we describe the assay here as set up in our laboratory. We use a plate washer and a plate reader but no automated system. The protocol can be adapted to an automated liquid handler as well. In addition, one of the difficulties to set up the assay is the availability of appropriate negative and positive controls. Negative controls are easier to come by and can be serum pools of serum taken before 2020. Positive controls can be convalescent samples from COVID19 patients or monoclonal antibodies (mAbs) like CR3022 (Tian et al., 2020; ter Meulen et al., 2006). If no human sera or mAbs are available, mouse mAbs, mouse sera against SARS-CoV-2, order animal sera against SARS-CoVo2 or anti-his tag antibodies (the proteins are his-tagged) can be used. However, in this case a different secondary antibody for the species from which the primary antibody is derived is needed for the positive control. Also, we recommend generating large batches of positive controls, which can be used for many runs. The positive control should be selected to exceed an OD₄₉₀ of the negative control plus 3 standard deviations of the negative controls up to, at least, a 1:150 dilution. ELISAs can be run with both serum and plasma.

Of note: RBD or full length spike might be used for both ELISA steps if only one antigen is available. In addition, only step A (not recommended) or only step B might be performed if fewer resources are available.

### Materials

Recombinant RBD protein
Recombinant full-length spike protein
Flat-Bottom Immuno Nonsterile 96-Well Plates 4 HBX (Thermo Scientific #3855, or equivalent)
Flat Bottom Cell Culture Plates (Corning #3599 or equivalent)
Milk Powder (AmericanBio #AB10109-01000, or equivalent)
PBS (1×) (Gibco #10010-023 or equivalent)
Water For Injection (WFI) for Cell Culture (Gibco #A1287301 or equivalent)
Tween 20 (Fisher Bioreagents #BP337-500, or equivalent)
Phosphate Buffered Saline (10×) (Corning™ 46013CM or equivalent)
Polypropylene sterile conical tubes
   15 mL (Denville Scientific #C1018P or equivalent)
   50 mL (Fisher Denville Scientific #C1060P or equivalent)
Sterile, serological pipettes
   5 mL (Falcon #356543 or equivalent)
   10 mL (Falcon #357551 or equivalent)
   25 mL (Falcon #357535 or equivalent)
   50 mL (Falcon #356550 or equivalent)
Micropipette tips
   20 μL barrier tips (Denville Scientific #P1121 or equivalent)
   200 μL barrier tips (Denville Scientific #P1122 or equivalent)
   200 μL tips (USA Scientific #1111-1700 or equivalent)
   1000 μL barrier tips (Denville Scientific #P1126 or equivalent)
Sterile reservoirs (Fisher Scientific #07-200-127 or equivalent)
Anti-Human IgG (Fab specific)-Peroxidase antibody produced in goat (Sigma #A0293)
Hydrochloric Acid 3.0M (Fisher Scientific #S25856, or equivalent)
SIGMAFAST™ OPD (Sigma-Aldrich #P9187 or equivalent)
Kimberly-Clark Kimwipes (Kimberly-Clark Professional #34721 or equivalent)
Pipet-Aid
Micropipettes
Class II biological safety cabinet
Ultra-Low Freezer (−80° C.)
Refrigerator at 4° C. (+/−1° C.)
Multichannel pipette(s) capable of pipetting 50-250 μL
1.5 mL Eppendorf tubes (Denville #C2170 or equivalent)
Timer
Aquamax 2000 Plate Washer (Molecular Devices #AQUAMAX 2000 or equivalent)
Biotek SynergyH1 Microplate Reader or equivalent

### Definitions

RBD=Receptor Binding Domain
ELISA=Enzyme-Linked Immunosorbent Assay
PBS=Phosphate-Buffered Saline
PBS-T=Phosphate-Buffered Saline with 0.1% Tween 20
RT=Room Temperature (18-25° C.)
HRP=Horseradish Peroxidase
HCl=Hydrochloric Acid
OPD=o-phenylenediamine dihydrochloride

### A—RBD Screening ELISA

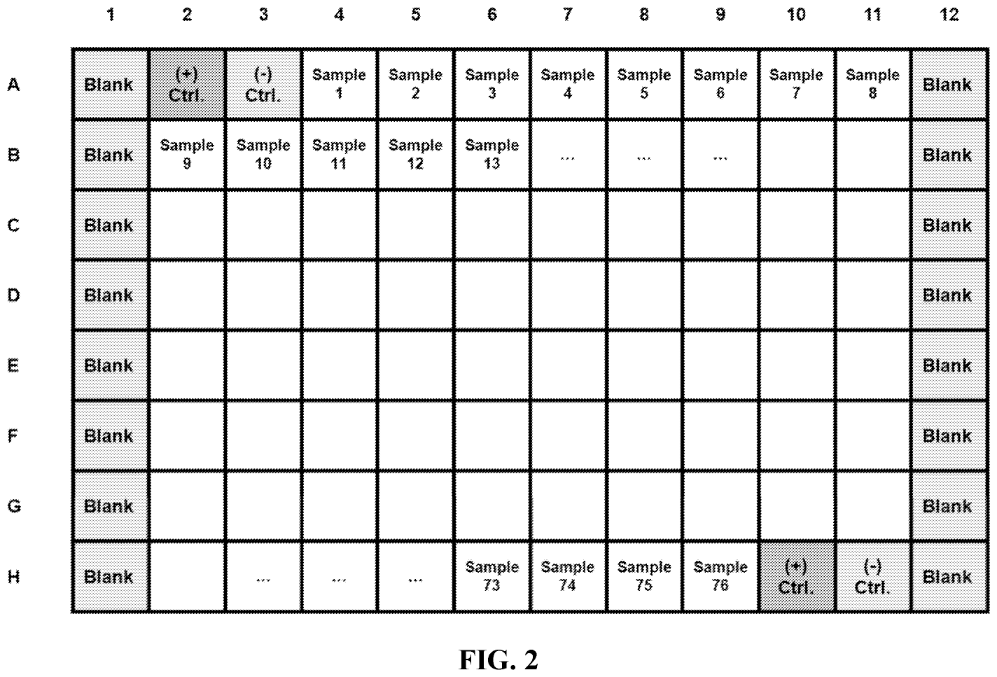

1. Coating ELISA Plates (Day 1)
   Thaw the required number of vials of antigen (SARS-CoV-2 RBD protein) to coat 96-well microtiter ELISA plates at a concentration of 2 ug/ml. Once thawed, mix by gently vortexing vial before diluting in 1×PBS.
   Prepare approximately 5 mL for each plate to be coated.
   Coat plates with 50 μl of diluted protein per well using a multichannel pipette and a reservoir. Lightly tap plates against surface to ensure protein is evenly coating the bottom of every well.
   Incubate at 4° C. overnight. Plates can be stored at 4° C. for up to 1 week.
   Always keep a cover plate on top of coated plates during all steps of the protocol!
2. Heat inactivation of samples (day 1, this is a safety precaution)
   Set the water bath to 56° C. Once temperature is reached, place the serum/plasma samples in and start the timer for 1h immediately.
   Remove samples when the timer goes off. Do not leave samples at 56° C. for longer than 1h. Store at 4° C. overnight or until use.
3. Block ELISA plate (day 2)
   Calculate to prepare at least 30 ml of blocking solution per plate.
   Blocking solution consists of PBS-T+3% milk powder (weight/volume).
   Using an automated plate washer, wash coated ELISA plates 3× with PBS-T.
   Add 200 μl blocking solution to all wells of the plates, starting the timer for 1h (do not exceed 4h) after completing the first plate. Place plates in a 20° C. (RT) incubator.
   Note: This step (and wherever a plate washer is needed below) can also be performed by washing plates with a multichannel pipette by hand if no plate washer is available.
4. Pre-diluting samples (day 2)
   In a biological safety cabinet, set up sterile Eppendorf tubes to pre-dilute serum samples 1:5.
   Add 40 μl of sterile 1×PBS to all tubes.
   Gently vortex serum sample to mix and add 10 μl to the Eppendorf tube, vortexing once more. Do this for all remaining samples including the positive and negative controls. Volume not needed in this part A will be stored and used for part B.
5. Dilution plate set-up (day 2)
   Calculate and prepare at least 30 ml of PBS-T+1% milk powder (weight/volume).
   Prepare 1 dilution plate (separate flat bottomed cell culture plate) per antigen coated plate prepared.
   Add 180 μl of PBS-T containing 1% milk to all wells of the dilution plate (including blank wells)
   Leaving Columns 1 and 12 as blanks, add 20 μl of sample (or control) into the designated well. This results in a final serum dilution of 1:50.
   Continue until all samples and controls have been added to designated wells. See FIG. 2 for reference plate layout below.
6. Transfer serum dilution (day 2)
   After blocking incubation, remove plates from the room temperature incubator and throw off the blocking solution. Tap the plates dry on a kimwipe.
   Using a multichannel pipette, pipette up and down 4-6 times in the first row of dilution plate to mix.
   Transfer 100 μl to the corresponding rows in the ELISA plate. Change tips and continue to transfer second row to the ELISA plate.
   Start the timer for 2h as soon as all the rows have been transferred to the first ELISA plate. (Do not exceed 4h)
   Place plates in a 20° C. (RT) incubator.
7. Secondary Antibody (day 2)
   After 2h, wash the plates 3× with PBS-T using the automated plate washer.
   Dilute anti-human IgG (Fab specific) HRP labeled secondary antibody 1:3000 in PBS-T containing 1% milk. Prepare at least 5 ml per plate.
   Add 50 μl to all wells of the plate using a multichannel pipette. Be sure to avoid touching the tips of the pipette to the walls of the well to avoid carry over and high background signals.
   Start the timer for 1h (stay in a range of 50 min to 65 min) as soon as the secondary antibody has been added to the first plate. Place plates in a 20° C. (RT) incubator.
8. Plate development and reading (day 2)
   After 1h, wash plates 3× with PBS-T using an automated plate washer.
   Prepare SigmaFast OPD solution and calculate amount needed. One set of tablets (1 gold+1 silver tablet) dissolved in 20 ml WFI can be used for 2 plates.
   Fully dissolve one gold tablet in 20 mL WFI. Do not add silver tablet to solution until ready to start adding to the plates (needs to be prepared fresh right before use).
   Add 100 μl to all wells of the plate. Begin timer for 10 minutes as soon as OPD has been added to the first row on the first plate. Do not exceed 10 minutes of developing before stopping the reaction.
   To stop the reaction after exactly 10 minutes, add 50 μl of 3M HCl to all wells.
   Read ELISA plates in plate reader at an absorbance of 490 nm (immediately after adding HCl) and record data.
   Samples that exceed certain OD₄₉₀ cutoff value (proposed cutoff: OD₄₉₀=0.15-0.2 or mean of negative controls plus 3 times the standard deviation of the negative controls) are assigned presumptive positive and will be tested in confirmatory ELISA using full-length spike protein.
   OD₄₉₀ cutoff has to be experimentally determined and depends on assay background and noise.

### B—Spike Confirmatory ELISA

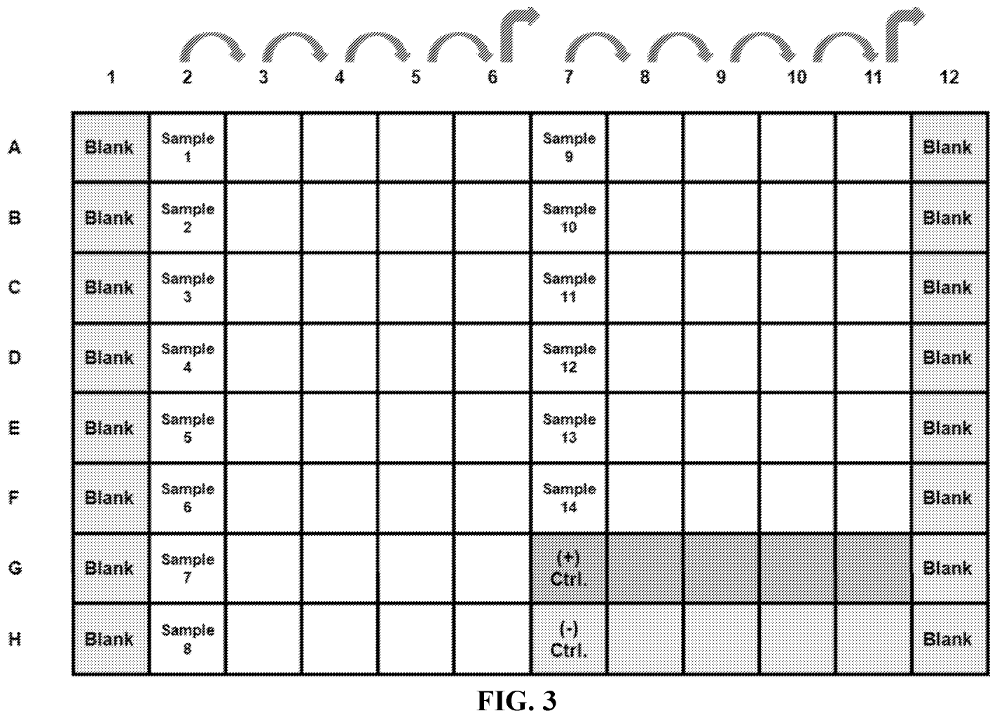

1. Coating ELISA plates (day 1)
   Thaw the required number of vials of antigen (SARS-CoV-2 Spike protein) to coat 96-well microtiter ELISA plates at a concentration of 2 μg/ml. Once thawed, mix by gently vortexing vial before diluting in 1×PBS.
   Prepare approximately 5 mL for each plate to be coated.
   Coat plates with 50 μl of diluted protein per well using a multichannel pipette and a reservoir. Lightly tap plates against surface to ensure protein is evenly coating the bottom of every well.
   Incubate at 4° C. overnight. Plates can likely be stored in 4° C. for up to 1 week but this needs to be validate locally to ascertain that it does not change the results.
2. Block ELISA plate (day 2)
   Calculate to prepare at least 30 ml of blocking solution per plate.
   Blocking solution consists of PBS-T+3% milk powder (weight/volume).
   Using an automated plate washer, wash coated ELISA plates 3× with PBS-T.
   Add 200 μl blocking solution to all wells of the plates, starting the timer for 1 h (do not exceed 4h) after completing the first plate. Place plates in a 20° C. (RT) incubator.
3. Pre-diluting samples (day 2)
   Retrieve 1:5 pre-diluted samples from Part A to be tested and confirmed (samples that are above certain threshold in RBD screening ELISA based on a set OD₄₉₀ value—see end of A).
4. Serial dilution (day 2)
   Calculate and prepare at least 20 ml of PBS-T+1% milk powder (weight/volume) per plate.
   After blocking incubation, remove plates from the room temperature incubator and throw off the blocking solution. Tap the plates dry on a kimwipe.
   Using a multichannel pipette, add 120 μl of PBS-T containing 1% milk to all wells of the plate.
   Leaving Columns 1 and 12 as BLANKS, add an extra 51 μl only to Columns 2 and 7 (=sample wells).
   Add 9 μl of 1:5 pre-diluted sample (final dilution 1:100 on the plate) to the first well in Column 2 and continue to add samples to all 8 wells. In Column 7, add samples to wells 1 through 6. Transfer positive and negative control into wells 7 and 8, respectively. See reference plate layout in FIG. 3.
   With the multichannel pipette, pipette up and down 4-6 times in Column 2 to mix. Discard these tips. With new tips, transfer 60 μl (3-fold dilution) from Column 2 to Column 3 and pipette up and down once 4-6 times to mix. Repeat this until Column 6; discard 60 μl before Column 7.
   Taking fresh tips mix Column 7 by pipetting. Repeat the same process of transferring, mixing, and discarding tips from Columns 7-11. Once Column 11 is reached, discard 60 μl.
   Start timer for 2h (do not exceed 4h) once the first ELISA plate has been serially diluted.
   Place plates in a 20° C. (RT) incubator.
5. Secondary Antibody (day 2)
   After 2h, wash the plates 3× with PBS-T using the automated plate washer.
   Dilute anti-human IgG (Fab specific) HRP labeled secondary antibody 1:3000 in PBS-T containing 1% milk. Prepare at least 5 ml per plate.
   Add 50 μl to all wells of the plate using a multichannel pipette. Be sure to avoid touching the tips of the pipette to the walls of the well.
   Start the timer for 1 h (50-65 min) as soon as the secondary antibody has been added to the first plate. Place plates in a 20° C. (RT) incubator.
6. Plate development and reading (day 2)
   After 1h, wash plates 3× with PBS-T using an automated plate washer.
   Prepare SigmaFast OPD solution and calculate amount needed. One set of tablets (1 gold+1 silver tablet) dissolved in 20 mL WFI can be used for 2 plates.
   Fully dissolve one gold tablet in 20 mL WFI. Do not add silver tablet to solution until ready to start adding to the plates.
   Add 100 μl to all wells of the plate. Begin timer for 10 minutes as soon as OPD has been added to the first row of the first plate. Do not exceed 10 minutes of developing before stopping the reaction.
   To stop the reaction after exactly 10 minutes, add 50 μl of 3M HCl to all wells.
   Read ELISA plates in plate reader at an absorbance of 490 nm (immediately after adding HCl) and record data.
   True positive samples will have a signal higher than the negative control plus 3 standard deviations of the negative controls in at least two consecutive dilutions.

### 6.1.4 REFERENCES CITED

1. Zhu, N., et al. A Novel Coronavirus from Patients with Pneumonia in China, 2019. N Engl J Med 382, 727-733 (2020).
2. Wu, F., et al. A new coronavirus associated with human respiratory disease in China. Nature (2020).
3. Corman, V. M., et al. Detection of 2019 novel coronavirus (2019-nCoV) by real-time RT-PCR. Euro Surveill 25(2020).
4. Chu, D. K. W., et al. Molecular Diagnosis of a Novel Coronavirus (2019-nCoV) Causing an Outbreak of Pneumonia. Clin Chem (2020).
5. Bao, L., et al. Reinfection could not occur in SARS-CoV-2 infected rhesus macaques. bioRxiv, 2020.2003.2013.990226 (2020).
6. Callow, K. A., Parry, H. F., Sergeant, M. & Tyrrell, D.A. The time course of the immune response to experimental coronavirus infection of man. Epidemiol Infect 105, 435-446 (1990).
7. Wrapp, D., et al. Cryo-EM structure of the 2019-nCoV spike in the prefusion conformation. Science (2020).
8. Amanat, F., et al. A serological assay to detect SARS-CoV-2 seroconversion in humans. medRxiv, 2020.2003.2017.20037713 (2020).
9. Tian, X., et al. Potent binding of 2019 novel coronavirus spike protein by a SARS coronavirus-specific human monoclonal antibody. Emerg Microbes Infect 9, 382-385 (2020).
10. ter Meulen, J., et al. Human monoclonal antibody combination against SARS coronavirus: synergy and coverage of escape mutants. PLOS Med 3, e237 (2006).

### 6.2 Example 2: SARS-CoV-2 Seroconversion in Humans: A Detailed Protocol for a Serological Assay, Antigen Production and Test Setup

This example provides a detailed protocol for expression of antigens derived from the spike protein of SARS-CoV-2 that can serve as a substrate for immunological assays as well as a two-step serological enzyme-linked immunosorbent assay (ELISA). These assays can be used for research studies and for testing in clinical laboratories. Not every aspect of this protocol has been tested in detail and we provided notes and comments whenever further optimizations and testing is recommended.

### 6.2.1 Basic Protocol 1

### Part I: Mammalian Cell Transfection and Protein Purification Protocol

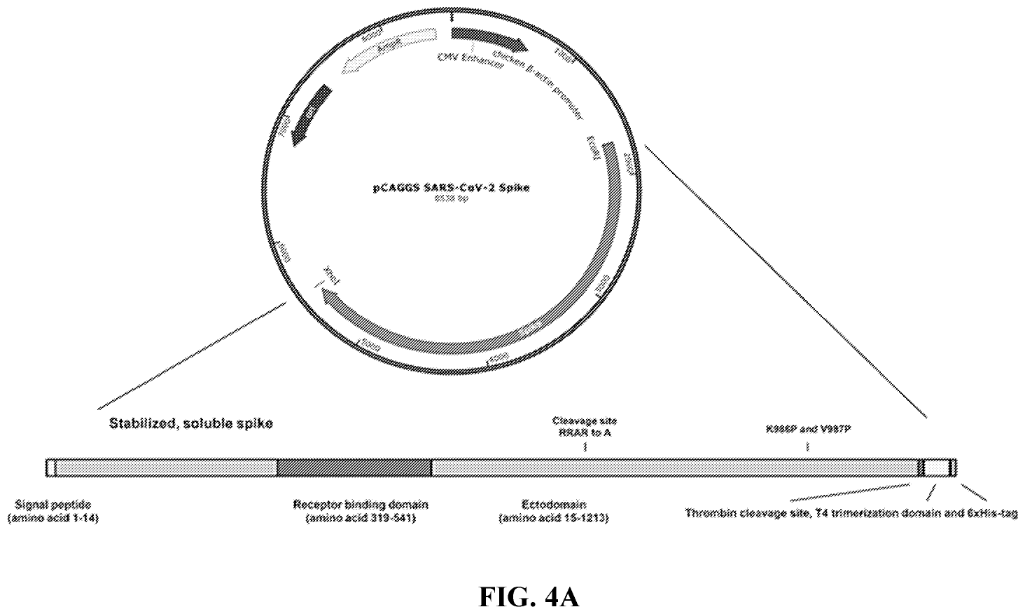

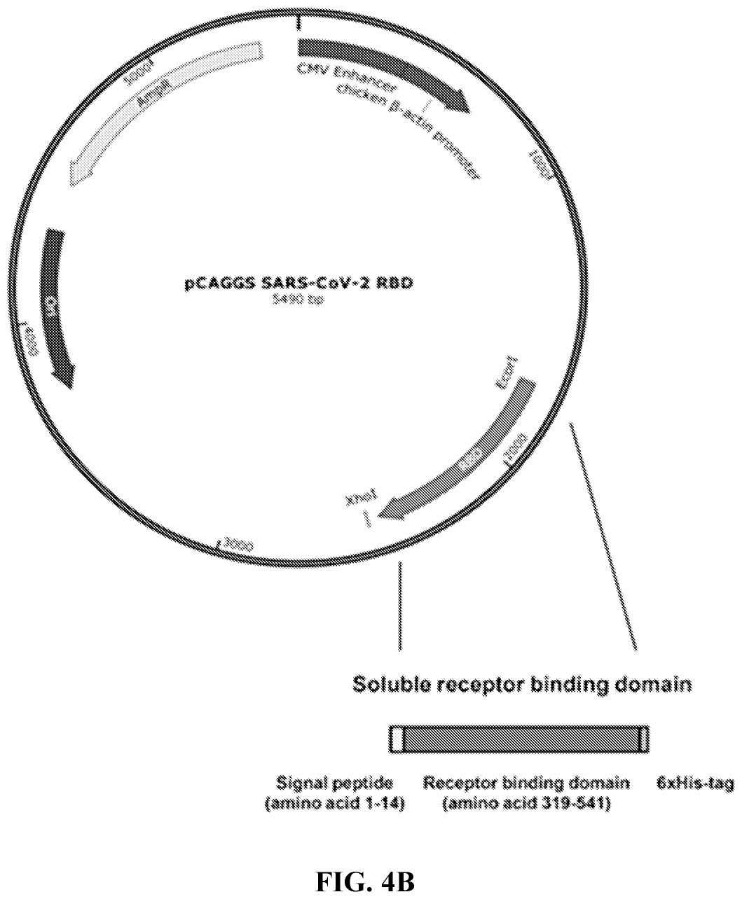

This protocol can be used for both expression vectors, the one expressing secreted RBD as well as the one expressing a soluble, trimeric version of the SARS-CoV-2 spike protein. Expression levels of the RBD are very high in our hands (>20 mg/L culture) while expression levels for the full-length spike are lower (approximately 1 mg/L). Therefore, we use the recombinant RBD (FIG. 4B) for initial screening ELISAs and the full-length spike (FIG. 4A) for confirmatory ELISAs (as described in Part II, Basic Protocol 2). Preparation of plasmids for mammalian cell expression are not described here. The expression vector constructs were described previously (Amanat, 2020). In brief, the sequences used for both proteins are based on the genomic sequence of the first isolate, Wuhan-Hu-1, which was released on January 10th 2020 (GenBank: MN908947.3). Sequences were codon optimized for mammalian cell expression. The full-length spike protein sequence was modified to remove the polybasic cleavage site, which is recognized by furin and to add a pair of stabilizing mutations (FIG. 4A). These two modifications were included to enhance the stability of the protein based on published literature (Amanat et al., 2020). The plasmids are grown in E. coli at 37° C. (or 30° C.) at 225 rpm in Luria-Bertani broth with ampicillin (LB-amp) in shaker flasks overnight. High-quality plasmid DNA can be obtained using commercially available maxiprep kits (ideally with an endotoxin removal step). Importantly, other cell lines (293T, CHO, etc.), other media, transfection reagents and more sophisticated protein purification methods might be used as alternatives if available.

Materials: See the materials below and those in Example 1, supra, under Part I.
Expi293F cells (Gibco #A14527)
pCAGGS containing the SARS-Related Coronavirus 2, Wuhan-Hu-1 Spike Glycoprotein Gene RBD with C-Terminal Hexa-Histidine Tag (available at BEI resources, NR-52309, https: www.beiresources.org Catalog BEIPlasmidVectors NR-52309.aspx)
pCAGGS containing the SARS-Related Coronavirus 2, Wuhan-Hu-1 full length Spike Glycoprotein Gene with C-Terminal Hexa-Histidine Tag (will be made available at BEI resources and can be requested)
   DEFINITIONS: See Example 1, supra, under Part I for definitions.

### Procedure

Mammalian cell transfection:

1. HEK 293F cells are counted using an automated cell counter (or a regular counting chamber) and seeded at a density of 600,000 cells/mL in Expi293 expression medium.

The viability of the cells must be greater than 90% at all times.

2. Cells are passaged every 3-4 days and incubated in an orbital shaking incubator at 37° C. and 125 RPM with 8% CO₂.

A maximum cell density of 4-5×10⁶ cells ml, is recommended at which point, cells should be immediately passaged.

3. 600×10⁶ cells are suspended in 200 mL (3×10⁶ cell/mL) of Expi293 expression media in a 1 L shaker flask.

Transfections are performed according to manufacturer's instructions.

4. Twelve mL of Opti-MEM is added to two 50 mL Falcon tubes: one tube receives 200 μg (1 μg/μL final dilution in the total volume of culture) of respective plasmid DNA (for RBD or full-length spike) while the other tube receives 640 μL of ExpiFectamine transfection reagent.

5. The contents of both 50 mL Falcon tubes are mixed together and incubated at RT for 10 minutes after which the transfection mixture is added dropwise to the cells.

6. Cells are then returned to the shaking incubator.

7. Sixteen hours post-transfection, 1.2 mL of Expifectamine 293 Transfection Enhancer 1 and 12.0 mL of Expifectamine 293 Transfection Enhancer 2 is added to the culture and subsequently, the culture is returned to the shaking incubator.

8. Three days post-transfection, the cells are harvested and spun at 4,000 g for 20 minutes at 4° C.

9. The supernatant is filtered using a 0.22 μm Stericup filter; the cell pellet can be discarded. Alternately, cells can be spun at 200 g for 10 minutes, supernatant can be collected, and the same cells can be resuspended in 200 mL of fresh Expi293 expression medium and returned to the shaking incubator for another 3 days. This alternate strategy works well with the RBD but is less suitable for the full-length spike (we have detected protein degradation in that case).

10. Continue to process the supernatant and purify protein immediately.

Alternatively, if the supernatant is stored, it must be kept at 4° C. (and for no longer than overnight 16 hours) in order to prevent denaturation of the protein at room temperature.

### Protein Purification Via Gravity Flow

Note: This step can be substituted with more advanced purification methodology, for example, if an Äkta purifier is available. The methods described below work even in labs not geared towards protein purification.

11. Prior to use, Ni-NTA resin (6 mL per 200 mL culture) is washed once with fresh PBS (transfer resin into 50 ml tube and fill up with PBS), then spun at 2000 g for 10 minutes in a centrifuge.

12. Once the centrifugation is complete, PBS is discarded, and resin is resuspended with the cell culture supernatant and inverted two or three times.

13. The resin is then incubated with the supernatant for 2 hours on a shaker (65 rpm) at RT.

14. Two clean polypropylene columns are loaded with the supernatant-resin mixture and then washed with one column volume of Wash Buffer four times.

15. Columns are then eluted using the Elution Buffer.

16. Four fractions are collected from each column by incubating the resin in the column with 3 mL of Elution Buffer for each fraction.

Incubate resin with elution buffer for 5 minutes after each addition of elution buffer.

17. Eluate is collected directly in a 50 mL Falcon tube placed on ice.

The total volume of eluate should be 24 mL from the two columns. More columns can be used to speed up the purification time depending on the volume of the culture.

18. Eluate is spun through 10 kDa Amicon Ultra Centrifugal Filter Units (for RBD) or 50 kDa Amicon Ultra Centrifugal Filter Units (for full-length spike) at 4000 g for 30 minutes (or longer if eluate takes longer to pass through the membrane) at 4° C. until only 200-300 μL remain in the unit.

Amicon Filter Units should be equilibrated with PBS before use.

19. PBS is added twice to the Amicon Ultra Centrifugal Filter Unit and spun at 4000 g for 30 minutes at 4° C. or until only 200-300 μL remain in the unit.

This step exchanges the buffer to PBS.

20. Finally, the protein is collected from the Amicon Ultra Centrifugal Filter Unit, its concentration is measured (e.g., using the Bradford protein assay or similar methods), and a denaturing SDS-page (4 to 20% gradient) is run to check the integrity of the purified protein. The size of the expected bands is 30 kDa for the RBD and about 140 kDa for the full-length spike.

21. After the elution step, protein should always be kept on ice or stored at 4° C.

For storage longer than 24 hours, protein should be frozen to −80° C. to avoid degradation. A concentration of 2 mg/mL and a volume of 50-200 μL is recommended as an aliquot size.

### 6.2.2 Basic Protocol 2

### Part II: A Two-Step ELISA Protocol for High-Throughput Screening of Human Serum Samples for Antibodies Binding to the Spike Protein of SARS-CoV-2

The purpose of this part of the protocol is to describe the procedure for measuring human antibody responses to the recombinant receptor-binding domain (RBD) of the spike protein or full-length spike protein of SARS-CoV-2 and to ensure the reproducibility and consistency of the obtained results.

We developed this as a two-step ELISA in which the first step (A) includes relatively high-throughput screening of samples in a single serum dilution against the RBD (which expresses very well and therefore can be produced in greater quantities). This is followed by a second step (B) in which positive samples from the first step undergo a confirmatory ELISA against the full-length spike protein (which is harder to express, therefore there is usually less available). For the second step, a dilution curve is performed. Typically, if only one operator is available, screening ELISAs can be run in the morning (760 samples/10 plates per run) and confirmatory ELISAs can be run in the afternoon (140 samples/10 plates per run). Of note, we describe the assay here as it is set up in our laboratory. We use a plate washer and a plate reader but no automated system. The protocol can be adapted to use with an automated liquid handler. In addition, one of the difficulties to set up the assay is the availability of appropriate negative and positive controls. Negative controls are easier to come by and can be serum pools taken before 2020. Positive controls can be convalescent samples from COVID19 patients or monoclonal antibodies (mAbs) like CR3022 (ter Meulen et al., 2006; Tian et al., 2020). If no human sera or mAbs are available, mouse mAbs, mouse sera against SARS-CoV-2, other animal sera against SARS-CoV-2 or anti-His tag antibodies (the proteins are His-tagged) can be used. However, in this case a different secondary antibody for the species from which the primary antibody is derived is needed for the positive control. Also, we recommend generating large batches of positive controls, which can be used for many runs. The positive control should be selected to result in a strong signal (recommend OD₄₉₀ of about 2.0) and should be clearly distinguishable from the negative controls. ELISAs can be run with either serum or plasma.

Of note: RBD or full-length spike might be used for both ELISA steps if only one antigen is available. In addition, only step A (not recommended) or only step B might be performed if fewer resources are available.

Materials: See Example 1, supra, under Part II for the materials.

DEFINITIONS: See the definitions under Part II of Example 1, supra.

### A—RBD Screening ELISA

1. Coating ELISA plates (day 1)
   Thaw the required number of vials of antigen (SARS-CoV-2 RBD protein) to coat 96-well microtiter ELISA plates at a concentration of 2 μg/mL. Once thawed, mix by gently vortexing vials before diluting in 1×PBS.
      Prepare approximately 5 mL for each plate to be coated.
   Coat plates with 50 μl of diluted protein per well using a multichannel pipette and a reservoir. Lightly tap plates against surface to ensure protein is evenly coating the bottom of every well.
   Incubate at 4° C. overnight.
      Always keep a cover plate on top of coated plates during all steps of the protocol! Plates can likely be stored in 4° C.′ for up to 1 week but this needs to be validated locally to ascertain that it does not change the results.
2. Heat inactivation of samples (day 1, this is a general safety precaution for work with human serum, we have not tested if this procedure inactivates SARS-CoV-2, please consult with your local biosafety officer to discuss proper safety precautions)
   Set the water bath to 56° C. Once temperature is reached, place the serum/plasma samples in and start the timer for 1 hour immediately.
   Remove samples when the timer goes off. Do not leave samples at 56° C. for longer than 1 hour.
      Store at 4° C. overnight or until use.
3. Blocking ELISA plates (day 2)
   Calculate to prepare at least 30 mL of blocking solution per plate.
   Blocking solution consists of PBS-T+3% milk powder (weight/volume).
   Using an automated plate washer, wash coated ELISA plates 3× with PBS-T.
   Add 200 μL blocking solution to all wells of the plates, starting the timer for 1 hour (do not exceed 4 hours) after completing the first plate. Place plates in a 20° C. (RT) incubator.
      Note: This step (and wherever a plate washer is needed below) can also be performed by washing plates with a multichannel pipette by hand if no plate washer is available.
4. Pre-diluting samples (day 2)
   In a biological safety cabinet, set up sterile Eppendorf tubes to pre-dilute serum samples with a 1:5 ratio.
   Add 40 μL of sterile 1×PBS to all tubes.
   Gently vortex each serum sample to mix and add 10 μL to the Eppendorf tube, vortexing once more. Do this for all remaining samples including the positive and negative controls.
      Volume not needed in this part A will be stored and used for part B.
5. Setting up dilution plates (day 2)
   Calculate and prepare at least 30 mL of PBS-T+1% milk powder (weight/volume).
   Prepare 1 dilution plate (separate flat-bottomed cell culture plate) per antigen-coated plate prepared.
   Add 180 μL of PBS-T containing 1% milk to all wells of the dilution plate (including blank wells).
   Leaving Columns 1 and 12 as blanks, add 20 μL of pre-diluted sample (or control) into the designated well.
      This results in a final serum dilution of 1:50.
   Continue until all samples and controls have been added to the designated wells. See reference plate layout below (FIG. 2).
6. Transferring serum dilution (day 2)
   After the blocking incubation, remove plates from the RT incubator and throw off the blocking solution. Tap the plates dry on a Kimwipe or other absorbent material.
   Using a multichannel pipette, pipette up and down 4-6 times in the first row of the dilution plate to mix.
   Transfer 100 μL to the corresponding rows in the ELISA plate. Change tips and continue to transfer the second row to the ELISA plate.
   Start the timer for 2 hours as soon as all of the rows' contents have been transferred to the first ELISA plate.
      Do not exceed 4 hours
   Place plates in a 20° C. (RT) incubator.
7. Incubating with secondary antibody (day 2)
   After 2 hours, wash the plates 3× with PBS-T using an automated plate washer.
   Dilute anti-human IgG (Fab specific) HRP labeled secondary antibody 1:3000 in PBS-T containing 1% milk.
      Prepare at least 5 mL per plate.
   Add 50 μL to all wells of the plate using a multichannel pipette.
      Be sure to avoid touching the tips of the pipette to the walls of the well to avoid carry over and high background signals.
   Start the timer for 1 hour (stay in a range of 50 minutes to 65 minutes) as soon as the secondary antibody has been added to the first plate.
   Place plates in a 20° C. (RT) incubator.
8. Developing and reading plates (day 2)
   After 1 hour, wash plates 3× with PBS-T using an automated plate washer.
   Prepare SigmaFast OPD solution and calculate amount needed.
      One set of tablets (1 gold+1 silver tablet) dissolved in 20 mL WFI can be used for 2 plates.
   Fully dissolve one gold tablet in 20 mL WFI.
      Do not add silver tablet to solution until ready to start adding to the plates. This needs to be prepared immediately before use.
   Add 100 μL OPD solution to all wells of the plate. Begin the timer for 10 minutes as soon as OPD has been added to the first row on the first plate.
      Do not exceed 10 minutes of developing before stopping the reaction.
   To stop the reaction after exactly 10 minutes, add 50 μL of 3M HCl to all wells.
   Read ELISA plates in plate reader at an absorbance of 490 nm (immediately after adding HCl) and record data.
      Samples that exceed a certain OD₄₉₀ cutoff value (proposed cutoff: OD₄₉₀=0.15−0.2 or mean of negative controls plus 3 times the standard deviation of the negative controls) are assigned as presumptive positives and will be tested in the confirmatory ELISA using full-length spike protein.
      OD₄₉₀ cutoff has to be experimentally determined and depends on assay background and noise.
         B—Spike confirmatory ELISA
1. Coating ELISA plates (day 1)
   Thaw the required number of vials of antigen (SARS-CoV-2 Spike protein) to coat 96-well microtiter ELISA plates at a concentration of 2 ug/mL. Once thawed, mix by gently vortexing vial before diluting in 1×PBS.
      Prepare approximately 5 mL for each plate to be coated.
   Coat plates with 50 μL of diluted protein per well using a multichannel pipette and a reservoir. Lightly tap plates against surface to ensure protein is evenly coating the bottom of every well.
   Incubate at 4° C. overnight.
      Plates can likely be stored in 4° C. for up to 1 week but this needs to be validated locally to ascertain that it does not change the results.
2. Blocking ELISA plates (day 2)
   Calculate to prepare at least 30 mL of blocking solution per plate.
   Blocking solution consists of PBS-T+3% milk powder (weight/volume).
   Using an automated plate washer, wash coated ELISA plates 3× with PBS-T.
   Add 200 μL blocking solution to all wells of the plates, starting the timer for 1 hour after completing the first plate.
      Do not exceed 4 hours.
   Place plates in a 20° C. (RT) incubator.
3. Pre-diluting samples (day 2)
   Retrieve 1:5 pre-diluted samples from Part A to be tested and confirmed (samples that are above certain threshold in RBD screening ELISA based on a set OD₄₉₀ value—see end of A).
4. Performing serial dilutions (day 2)
   Calculate and prepare at least 20 mL of PBS-T+1% milk powder (weight/volume) per plate.
   After the blocking incubation, remove plates from the RT incubator and throw off the blocking solution. Tap the plates dry on a Kimwipe or other absorbent material.
   Using a multichannel pipette, add 120 μL of PBS-T containing 1% milk to all wells of each plate.
   Leaving Columns 1 and 12 as blanks, add an extra 51 μL of PBS-T containing 1% milk to wells only in Columns 2 and 7.
      Wells of column 2 and 7 will be the sample wells.
   Add 9 μL of 1:5 pre-diluted sample (final dilution 1:100 on the plate) to the first well in Column 2 and continue to add samples to all 8 wells.
   In Column 7, add samples to wells A through F.
   Transfer positive and negative control into wells G and H respectively. See reference plate layout below (FIG. 3).
   With the multichannel pipette, pipette up and down 4-6 times in Column 2 to mix. Discard these tips. With new tips, transfer 60 μL (3-fold dilution) from Column 2 to Column 3 and pipette up and down 4-6 times to mix. Repeat this until Column 6; discard 60 μL before Column 7.
   Taking fresh tips, mix Column 7 by pipetting up and down 4-6 times. Repeat the same process of transferring, mixing, and discarding tips from Columns 7-11. Once Column 11 is reached, discard 60 μL.
   Start the timer for 2 hours once the first ELISA plate has been serially diluted.
      Do not exceed 4 h.
   Place plates in a 20° C. (RT) incubator.
5. Incubating with secondary antibody (day 2)
   After 2 hours, wash the plates 3× with PBS-T using the automated plate washer.
   Dilute anti-human IgG (Fab specific) HRP labeled secondary antibody 1:3000 in PBS-T containing 1% milk.
      Prepare at least 5 mL per plate.
   Add 50 μL to all wells of the plate using a multichannel pipette.
      Be sure to avoid touching the tips of the pipette to the walls of the well.
   Start the timer for 1 hour as soon as the secondary antibody has been added to the first plate.
      Incubation time should be in the range of 50 to 65 minutes.
   Place plates in a 20° C. (RT) incubator.
6. Developing and reading plates (day 2)
After 1 hour, wash plates 3× with PBS-T using an automated plate washer.
   Prepare SigmaFast OPD solution and calculate amount needed.
   One set of tablets (1 gold+1 silver tablet) dissolved in 20 mL WFI can be used for 2 plates.
   Fully dissolve one gold tablet in 20 mL WFI.
   Do not add silver tablet to solution until ready to start adding to the plates.
   Add 100 μl to all wells of the plate. Begin timer for 10 minutes as soon as OPD has been added to the first row of the first plate.
   Do not exceed 10 minutes of developing before stopping the reaction.
   To stop the reaction after exactly 10 minutes, add 50 μl of 3M HCl to all wells.
   Read ELISA plates in plate reader at an absorbance of 490 nm and record data.
      Ideally, read plates immediately after adding HCl.
   True positive samples will have a signal higher than the negative control plus 3 standard deviations of the negative controls in at least two consecutive dilutions.

### Reagents and Solutions:

### Wash Buffer (4 L):

See Example 1, supra, under Part I for the formula for the Wash buffer.

Can be stored at room temperature for up to 4 months.

### Elution Buffer (4 L):

See Example 1, supra, under Part I for the formula for the Wash buffer.

Can be stored at room temperature for up to 4 months.

Phosphate-Buffered Saline with 0.1% Tween 20 (50 L):

| | | |
|---|---|---|
| Distilled water | 45 | L |
| PBS 10X | 5 | L |
| Tween 20 | 50 | mL |

Can be stored at room temperature for up to 4 months.

### Commentary

BACKGROUND INFORMATION: The protein expression and purification methods (Basic Protocol 1) described in this protocol are based on well-established techniques. The expression plasmids and protein sequences have been optimized to increase protein stability and yield (Amanat et al., 2020). Plasmids can be requested from the Krammer lab or can be found on BEI resources. The ELISA protocol (Basic Protocol 2) has been designed to allow for a high-throughput screening of many samples per day, followed by a confirmatory step to verify presumptive positive results. The ELISA assay itself is based on well-established protocols and has been optimized for the use of SARS-CoV-2 antigens.

CRITICAL PARAMETERS and TROUBLESHOOTING: The most common problem for the transfection (Basic Protocol 1) is low cell viability before performing the transfection. The cells need to be 90-95% viable. The absence of antibiotics/antifungals requires good sterile techniques to prevent contamination. Sterile plasmid preparations are also recommended and it is important to add the enhancer to the shaking flasks sixteen hours post-transfection.

For the protein purification, we recommend always using fresh Ni-NTA resin to prevent cross-contamination with other proteins. Harvested supernatant should be ideally processed immediately to ensure protein integrity. To make filtering of the supernatant easier, an additional centrifugation step (after pelleting the cells) is recommended to pellet residual cells and other particles. When performing the buffer exchange using the Amicon Ultra Centrifugal Filter Units, make sure to use the right sized cut-off (use smaller cut-off for RBD). It is recommended that purified protein is diluted to a concentration of about 2 mg/mL. Storage at higher concentrations may result in aggregation of protein.

For the ELISA (Basic Protocol 2), performing all of the washing steps and adhering to the incubation times are important to achieve low background reactivity. Most critical are the incubation times for the secondary antibody and the substrate (OPD and HCl for stopping the reaction). In addition, touching wells with tips when transferring secondary antibody and substrate can result in higher background and possibly false positive wells and needs to be avoided. In preparing the OPD, it is also important to dissolve the gold tablet fully and only add the silver tablet right before the substrate is added to the ELISA plate.

UNDERSTANDING RESULTS: We expect expressions levels of the RBD to be around 20 mg/L culture cells and expression of the full-length spike protein to be around approximately 1 mg/L in 293Fs using a gravity flow protein purification strategy. When running the SDS-PAGE to confirm protein integrity, clear single bands are expected for the RBD and full-length spike at around 25-30 kDa and ˜140 kDa, respectively. Additionally, ELISAs with positive and negative controls (e.g., monoclonal antibodies) are performed to confirm correct protein folding. We expect a good binding profile for the positive control and low-to-no background reactivity for the negative control.

TIME CONSIDERATIONS: Basic protocol 1 and 2 can be completed in about 6 days. Basic protocol 1 takes about 4 days. Growing up a cryostock of 293F cells, bringing them to passage four (recommended before transfection) and obtaining a sufficient cell number would take another few days and are not taken into account in this protocol. Basic protocol 2 takes at least 2 days (antigen coating on day 1 and running the ELISA on day 2). The screening ELISA could be performed in the morning and the confirmatory ELISA in the afternoon, or the assays can be done on consecutive days.

### 6.2.3 LITERATURE CITED

Amanat, F., Nguyen, T., Chromikova, V., Strohmeier, S., Stadlbauer, D., Javier, A., . . . Krammer, F. (2020). A serological assay to detect SARS-CoV-2 seroconversion in humans. medRxiv, 2020.2003.2017.20037713. doi:10.1101/2020.03.17.20037713
ter Meulen, J., van den Brink, E. N., Poon, L. L., Marissen, W. E., Leung, C. S., Cox, F., Goudsmit, J. (2006). Human monoclonal antibody combination against SARS coronavirus: synergy and coverage of escape mutants. PLOS Med, 3(7), e237.
doi:10.1371/journal.pmed.0030237
Tian, X., Li, C., Huang, A., Xia, S., Lu, S., Shi, Z., . . . Ying, T. (2020). Potent binding of 2019 novel coronavirus spike protein by a SARS coronavirus-specific human monoclonal antibody. Emerg Microbes Infect, 9(1), 382-385. doi: 10.1080/22221751.2020.1729069

### 6.3 Example 3: A Serological Assay to Detect SARS-CoV-2 Seroconversion in Humans

Introduction: SARS-Cov-2 (severe acute respiratory disease coronavirus 2), which causes Coronavirus Disease 2019 (COVID19), was first detected in China in late 2019 and has since then caused a global pandemic. While molecular assays to directly detect the viral genetic material are available for the diagnosis of acute infection, we currently lack serological assays suitable to specifically detect SARS-CoV-2 antibodies.

Methods: Here we describe serological enzyme-linked immunosorbent assays (ELISA) that we developed using recombinant antigens derived from the spike protein of SARS-CoV-2. These assays were developed with negative control samples representing pre-COVID 19 background immunity in the general population and samples from COVID19 patients.

Results: The assays are sensitive and specific, allowing for screening and identification of COVID 19 seroconverters using human plasma/serum as early as 3 days post symptom onset. Importantly, these assays do not require handling of infectious virus, can be adjusted to detect different antibody types and are amendable to scaling.

Conclusion: Serological assays are of critical importance to determine seroprevalence in a given population, define previous exposure and identify highly reactive human donors for the generation of convalescent serum as therapeutic. Sensitive and specific identification of Coronavirus SARS-Cov-2 antibody titers will also support screening of health care workers to identify those who are already immune and can be deployed to care for infected patients minimizing the risk of viral spread to colleagues and other patients.

### 6.3.1 Introduction

On Dec. 31, 2019 China reported first cases of atypical pneumonia in Wuhan, the capital of Hubei province. The causative virus was found to be a betacoronavirus, closely related to the severe acute respiratory syndrome coronavirus (SARS-CoV-1) from 2003 and similar to Sarbecoviruses isolated from bats. 1.2 It was therefore termed SARS-CoV-2 and the disease it causes was named COVID19 (COrona VIrus Disease 2019).3 The outbreak in Wuhan expanded quickly and led to the lockdown of Wuhan, the Hubei province and other parts of China. While the lockdown, at least temporarily, brought the situation under control in China, SARS-CoV-2 spread globally causing a pandemic with, so far, 150,000 infections and 5,500 fatalities (as of Mar. 16, 2020).

Nucleic acid tests that detect the SARS-CoV-2 RNA genome were quickly developed and are now widely employed to diagnose COVID19 disease.4.5 However, there remains a great need for laboratory assays that measure antibody responses and determine seroconversion. While such serological assays are not well suited to detect acute infections, they support a number of highly relevant applications. First, serological assays allow us to study the immune response(s) to SARS-CoV-2 in dynamic qualitative and quantitative manner. Second, serosurveys are needed to determine the precise rate of infection in an affected area, which is an essential variable to accurately determine the infection fatality rate. Third, serological assays will allow for the identification of individuals who mounted strong antibody responses and who could serve as donors for the generation of convalescent serum therapeutics. Lastly, serological assays will permit the determination of who is immune and who is not. This would be very useful for deploying immune healthcare workers in a strategic manner as to limit the risk of exposure and avoid spreading the virus inadvertently.

Sarbecoviruses express a large (approximately 140 kDa) glycoprotein termed spike protein (S, a homotrimer), which mediates binding to host cells via interactions with the human receptor angiotensin converting enzyme 2 (ACE2).6′8 The S protein is very immunogenic with the receptor-binding domain (RBD) being the target of many neutralizing antibodies.′ Individuals infected with coronaviruses typically mount neutralizing antibodies, which might be associated with some level of protection for a period of months to years, and neutralizing response has been demonstrated for SARS-CoV-2 in an individual case from day 9 onwards.^{10,13} Serum neutralization can be measured using live virus but the process requires several days and must be conducted under biosafety level 3 laboratory containment for SARS-CoV-2. Potentially, pseudotyped viral particle entry assays based on lentiviruses or vesicular stomatitis virus can be used as well, but these reagents are not trivial to produce. A simple solution is the use of a binding assay, e.g., an enzyme linked immunosorbent assays (ELISA), with recombinantly expressed antigen as substrate. Here we report the development of such an assay and provide a protocol for both recombinant antigen production as well as the ELISA.

### 6.3.2 Methods

### Recombinant Proteins

The mammalian cell codon optimized nucleotide sequence coding for the spike protein of SARS-CoV-2 isolate (GenBank: MN908947.3) was synthesized commercially (GeneWiz). The receptor binding domain (RBD, amino acid 319 to 541, RVQP . . . CVNF) along with the signal peptide (amino acid 1-14, MFIF . . . TSGS) plus a hexahisitidine tag was cloned into mammalian expression vector pCAGGS as well as in a modified pFastBacDual vectors for expression in baculovirus system. The soluble version of the spike protein (amino acids 1-1213, MFIF . . . IKWP) including a C-terminal thrombin cleavage site, T4 foldon trimerization domain and hexahistidine tag was also cloned into pCAGGS. The protein sequence was modified to remove the polybasic cleavage site (RRAR to A) and two stabilizing mutations were introduced as well (K986P and V987P, wild type numbering). Recombinant proteins were produced using the well-established baculovirus expression system and this system has been published in great detail in 16.24.25 including a video guide. Recombinant proteins were also produced in Expi293F cells (ThermoFisher) by transfections of these cells with purified DNA using ExpiFectamine 293 Transfection Kit (ThermoFisher). Supernatants from transfected cells were harvested on day 3 post-transfection by centrifugation of the culture at 4000 g for 20 minutes. Supernatant was then incubated with 6 mis Ni-NTA agarose (Qiagen) for 1-2 hours at room temperature. Next, gravity flow columns were used to collect the Ni-NTA agarose and the protein was eluted. Each protein was concentrated in Amicon centrifugal units (EMD Millipore) and re-suspended in phosphate buffered saline (PBS). Proteins were analyzed on reducing SDS-PAGE. The DNA sequence for all constructs is available from the Krammer laboratory. Several of the expression plasmids and proteins have also been submitted to BEI Resources and can be requested from their web page for free (https://www.beiresources.org/).

### SDS-PAGE

Recombinant proteins were analyzed via a standard SDS-PAGE gel to check protein integrity. One ug of protein was mixed with 2× Laemmli buffer containing 5% beta-mercaptoethanol (BME) at a ratio of 1:1. Samples were heated at 100° Celsius for 15 minutes and then loaded onto a polyacrylamide gel (5% to 20% gradient; Bio-Rad). Gels were stained with SimplyBlue SafeStain (Invitrogen) for 1-2 hours and then de-stained in distilled water overnight.

### Human Samples

Banked human samples were collected from study participants enrolled in ongoing IRB approved longitudinal observational study protocols of the Mount Sinai Personalized Virology Initiative. Samples were selected based on the date of collection (2019, early 2020) and whether participants had a documented history of viral infection. Samples were collected in the Clinical Research Unit at the Icahn School of Medicine at Mount Sinai after obtaining written consent and all participants agreed to sample banking and future research use. Self-reported ethnicities of the individuals from which samples were tested included Caucasian, Asian, African American and Hispanic. Samples included sera from a participant with acute NL63 infection as determined by the Biofire Respiratory panel. We included serum collected at day 3 post symptom onset as well as convalescent serum from the same person (day 30 post symptom onset). In addition, we tested convalescent sera from individuals with dengue, chikungunya and hantavirus infections. These samples served as negative controls given that they were collect prior to SARS-Cov-2 spread in the US. Six subjects were 20-29, 19 were 30-39, 13 were 40-49, 7 were 50-59 years old and six were 60 or older. For the mRBD ELISAs sera from additional nine t subjects were tested (30-39: 2; 40-49: 4; 50-59: 2; 60+: 1).

De-identified samples from the University of Melbourne and University of Helsinki were used as positive controls. For those, human experimental work was conducted according to the Declaration of Helsinki Principles and according to the Australian National Health and Medical Research Council Code of Practice. All participants provided written informed consent prior to the study. The studies were approved by the Alfred Hospital (ID #280/14) and University of Melbourne (ID #1442952.1, 1955465.2) Human Research Ethics Committees, and under research permit for project TYH2018322 of Helsinki University Hospital Laboratory.

### ELISA

The ELISA protocol was adapted from previously established protocols^{26,27}. Ninety-six well plates (Immulon 4 HBX; Thermo Scientific) were coated overnight at 4° Celsius with 50 μl per well of a 2 ug/ml solution of each respective protein suspended in PBS (Gibco). The next morning, the coating solution was removed and 100 ul per well of 3% non-fat milk prepared in PBS with 0.1% Tween 20 (TPBS) was added to the plates at room temperature (RT) for 1 hour as blocking solution. Serum samples were heated at 56° C. for 1 hour before use to reduce risk from any potential residual virus in serum. Serial dilutions of serum and antibody samples were prepared in 1% non-fat milk prepared in TPBS. The blocking solution was removed and 100 μl of each serial dilution was added to the plates for 2 hours at RT. Next, the plates were washed thrice with 250 ul per well of 0.1% TPBS. Next, a 1:3000 dilution of goat anti-human IgG-horseradish peroxidase (HRP) conjugated secondary antibody (ThermoFisher Scientific) prepared in 0.1% TPBS and 100 ul of this secondary antibody was added to each well for 1 hour. Plates were again washed thrice with 0.1% TBS. Once completely dry, 100 μl of SigmaFast OPD (o-phenylenediamine dihydrochloride; Sigma-Aldrich) solution was added to each well. This substrate was left on the plates for 10 minutes and then the reaction was stopped by addition of 50 μL per well of 3 M hydrochloric acid (HCl). The optical density at 490 nanometers was measured via a Synergy 4 (BioTek) plate reader. The background value was set at and optical density 490 nm of 0.11 and area under the curve (AUC) was calculated. AUC values below 1 were assigned a value of 0.5 for graphing and calculation purposes. Data was analyzed in Prism 7 (Graphpad).

To assess the distribution of the different antibody isotypes/subclasses in the samples that reacted well in our standard ELISA, another ELISA was performed with different secondary antibodies 23. These antibodies include anti-human IgA (a-chain-specific) HRP antibody (Sigma A0295) (1:3,000), anti-human IgM (p-chain-specific) HRP antibody (Sigma A6907) (1:3,000), anti-human IgG1 Fc-HRP (Southern Biotech 9054-05) (1:3,000), anti-human lgG3hinge-HRP (Southern Biotech 9210-05) (1:3,000), and anti-human lgG4 Fc-HRP (Southern Biotech 9200-05).

### 6.3.3 Results

### Expression Constructs and Generation of Recombinant SARS-Cov-2 Proteins

We generated two different versions of the spike protein. The first construct expresses a full length trimeric and stabilized version of the spike protein and the second only the much smaller receptor binding domain (RBD). The sequence used for both proteins is based on the genomic sequence of the first virus isolate, Wuhan-Hu-1, which was released on January 10th 2020.1 Sequences were codon optimized for mammalian cell expression. The full-length spike protein sequence was modified to remove the polybasic cleavage site, which is recognized by furin and to add a pair of stabilizing mutations (FIGS. 5B-5C).^{7,14,15} These two modifications were included to enhance the stability of the protein based on published literature.^{7,14} At amino acid P1213 the sequence was fused to a thrombin cleavage site, a T4 foldon sequence for proper trimerization and a C-terminal hexahistidine tag for purification (FIG. 5C).^{16,17} The sequence was cloned into a pCAGGS vector for expression in mammalian cells and into a modified pFastBac Dual vector for generation of baculoviruses and expression in insect cells. For expression of the RBD, the natural N-terminal signal peptide of S was fused to the RBD sequence (amino acid 419 to 541) and joined with a C-terminal hexahistidine tag (FIG. 5D).¹⁸ The same vectors as for the full length S protein were used to express the RBD. In mammalian cells, the RBD domain gave outstanding yields (approximately 25 mg/liter culture), but expression was lower in insect cells (approximately 0.4 mg/liter culture). Clear single bands were visible when the recombinant RBD proteins were analyzed on a reducing sodium dodecyl sulfate-polyacrylamide gel electrophoresis (SDS-PAGE), with the insect cell derived protein (iRBD) running slightly lower than the mammalian cell derived protein (mRBD) (FIGS. 5E-5F). The size difference likely reflects differences in glycan sizes between insect cells and mammalian cells. The full-length S protein was expressed in both systems with slightly higher yields in mammalian cells (mSpike) than in insect cells (iSpike) (approximately 2 mg/liter cultures versus 0.5 mg/liter culture). The full-length protein appeared as a double band on a reducing SDS-PAGE, the higher species likely being the full-length protein and the slightly lower species likely a cleavage product.

### ELISA Development

We used a panel of 59 banked human serum samples collected from study participants including participants with confirmed previous viral infections (e.g., hantavirus, dengue virus, coronavirus NL63—samples taken 30 days post symptom onset) to establish an ELISA with these proteins. These human sera were used to test the background reactivity to the SARS-CoV-2 spike in the general US population covering an age range from approximately 20 to 65+ years. Four plasma/serum samples from three COVID19 patients were used to determine the reactivity of SARS-CoV-2 infected individuals to the RBD and the full length spike.

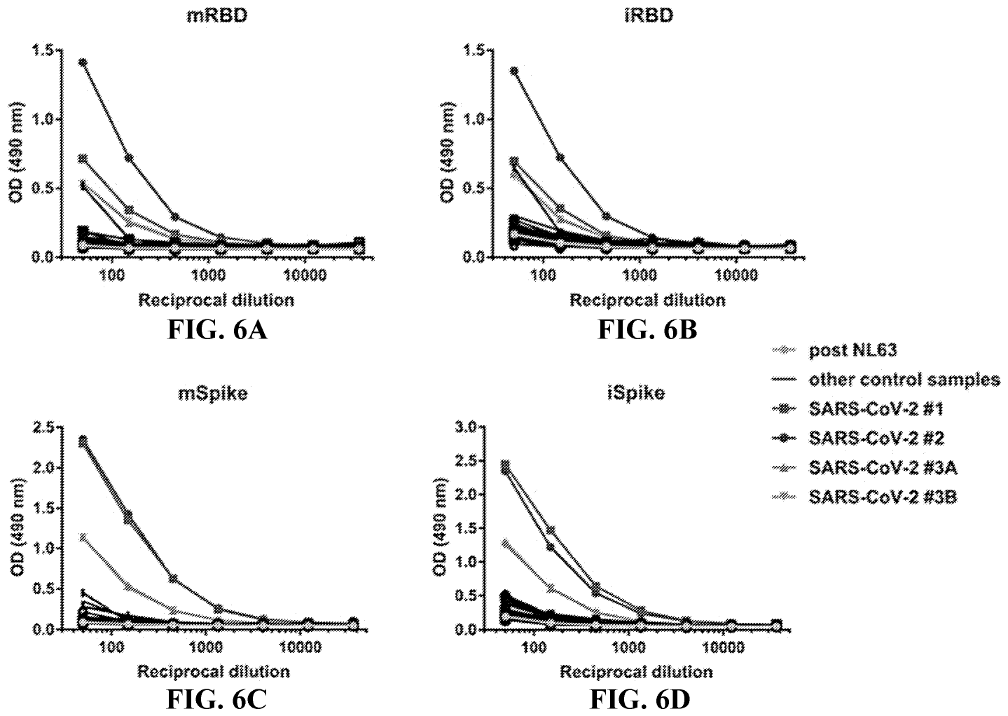

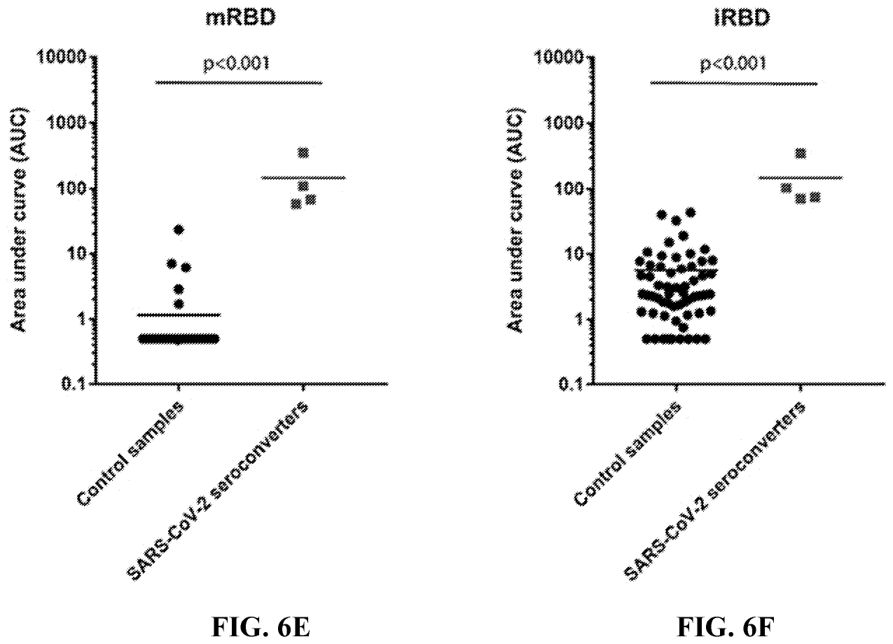

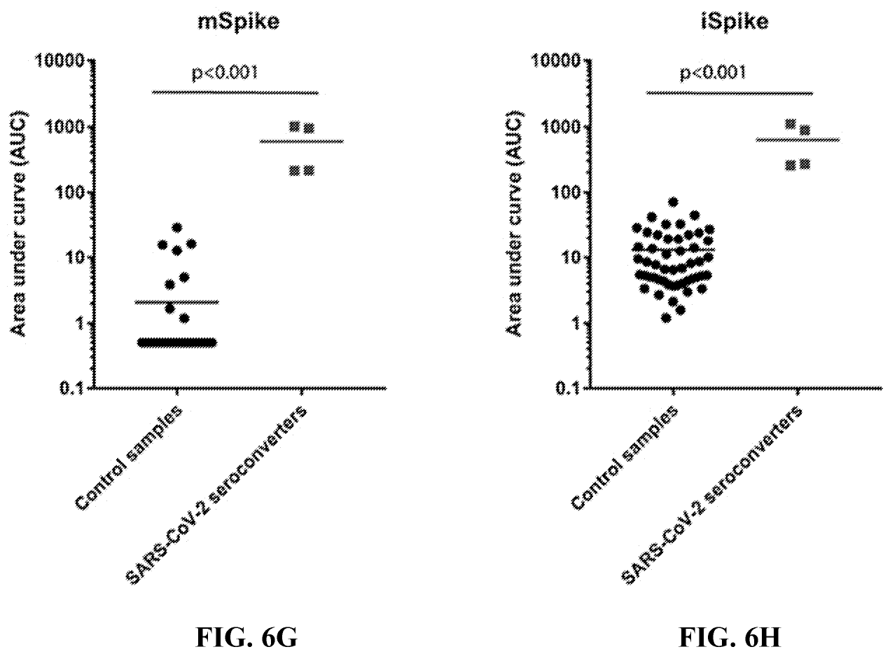

ELISAs were performed by doing serial dilution of the individual serum samples. Values from the dilution curves were used to determine the area under the curve (AUC), which was graphed. All COVIDIO plasma/serum samples reacted strongly to both RBD and full-length spike protein while reactivity of the other serum samples only yielded background reactivity (FIGS. 6A-6H). Reactivity of COVID 19 sera was, in general, stronger against the full-length S protein than against the RBD, likely reflecting the higher number of epitopes found on the much larger spike protein. For the RBD the difference between control sera and convalescent sera was larger when the insect cell derived protein was used as compared to the mammalian cell derived RBD. The same was true for the full-length spike protein. The assay allowed to clearly distinguish the convalescent sera from the banked control sera.

### Antibody Isotyping and Subtyping

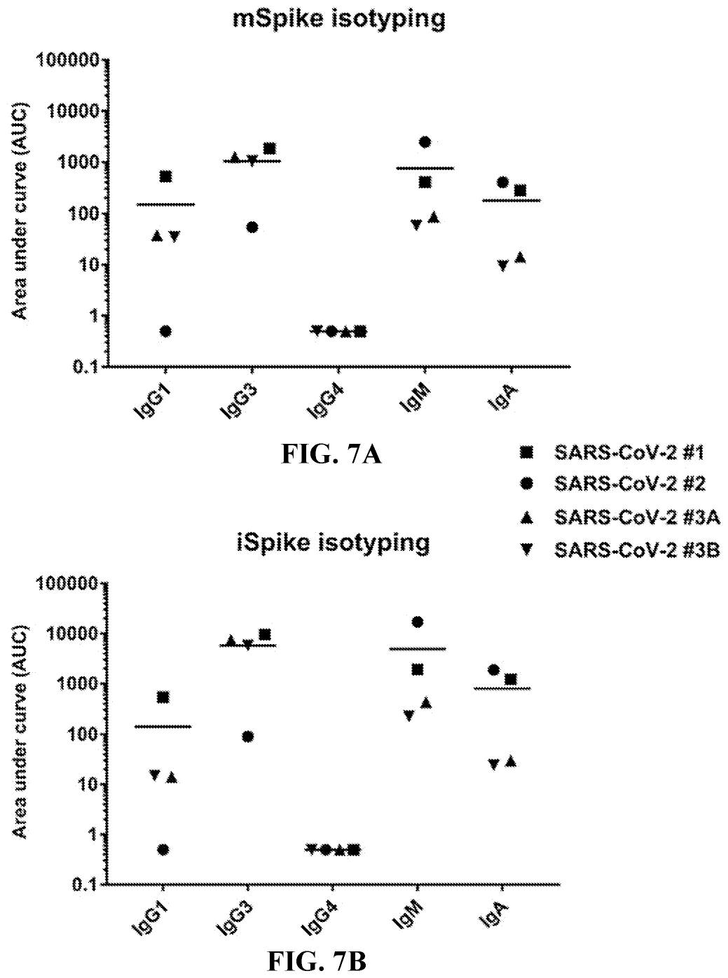

For the four COVID19 patient plasma/sera, we also performed an isotyping and subtyping ELISA using the insect cell and mammalian cell expressed S proteins. Strong reactivity was found for all samples for lgG3, IgM and IgA (FIGS. 7A-7B). An IgG1 signal was also detected for three out of the four samples, while one sample showed no reactivity. No signal was detected for lgG4 and reagents for lgG2 were unavailable.

### 6.3.4 Discussion

Here we describe a serological method to detect seroconversion upon SARS-CoV-2 infection. The method is based on reactivity to the immunogenic S protein of the virus. It is relatively simple and quick in its execution and can be performed at biosafety level 2 as it does not involve life virus. We have tested these methods using banked serum samples obtained from study participants in 2019 and early 2020 when this virus was not widely circulating in the US. These serum samples produced low, close to baseline signals in our ELISA. Since the age range of the participants was broad, ranging from 20 to 65+ years of age, it is likely that most had experienced infections with human coronaviruses including the alphacoronaviruses NL63 and 229E as well as the betacoronaviruses OC43 and HKU1. We included paired serum samples (acute and convalescent) from a participant with a laboratory confirmed coronavirus NL63 infection. Our data show that there is no or only negligible cross-reactivity from human coronaviruses to SARS-CoV-2. Of note, even infection with the human alphacoronavirus NL63, which also uses ACE2 as receptor¹⁹, did not induce cross-reactivity. This is of great importance because it suggests that humans are completely naive to SARS-CoV-2, which may explain the relatively high Ro of SARS-CoV-2 compared to other respiratory viruses such as influenza virus.²⁰ It might also suggest that antibody-dependent enhancement from human coronavirus induced cross-reactive antibodies targeted at the S protein is unlikely to be the cause of the high pathogenicity of the virus in humans.²¹ The plasma/sera used in this study from patients with COVID 19 were obtained at day 20 (SARS-CoV-2 #1), at day 4 (SARS-CoV-2 #2), days 2 and 6 (SARS-CoV-2 #3A and B) post symptom onset. Our data shows significant seroconversion after natural infection with SARS-CoV-2. Our results suggest that antibodies mounted upon infection target the full length S protein as well as the RBD, which is the major target for neutralizing antibodies for related viruses and coronaviruses. In fact, sample SARS-CoV-2 #1 was tested in another study in neutralization assays and showed a neutralizing titer of 1:160.¹³ Thus, seroconversion may lead to protection at a minimum for a limited time. Interestingly, the lgG3 response was stronger than the IgG1 response which is in contrast to, e.g., the immune response to influenza where usually IgG1 responses dominates.^{22,23} Lastly, we also detected strong IgA and IgM responses in the blood compartment. Of note, level of reactivity and antibody isotypes matched expected patterns based on time since symptom onset very well.

We believe that our ELISA method will be key for serosurveys aimed at determining the real attack rate and infection fatality rate in different human populations and to map the kinetics of the antibody response to SARS-CoV-2. In addition, clinical trials with convalescent serum as therapeutic have been initiated in China (e.g., NCT04264858) and anecdotal evidence from the epidemic in Wuhan suggests that compassionate use of these interventions was successful. China has recently shipped convalescent sera to Italy for use in patients and efforts to produce convalescent serum batches are ongoing in the US as well. Screening sera using our assay would be faster and easier than performing standard neutralization assays in BSL3 containment laboratories. Patients recovering from COVID19 disease could be screened for strong antibody responses using the assays described here, especially the one using the RBD as substrate since anti-RBD antibodies likely correlate with virus neutralization. In addition, the assay could be used to screen health care workers to allow selective deployment of immune medical personnel to care for patients with COVID19. Such a strategy would likely limit nosocomial spread of the virus. Of course, the generated recombinant proteins are also excellent reagents for vaccine development and can serve as baits for sorting B cells for monoclonal antibody generation. We are making the methods and laboratory reagents widely available to the research community in order to support the global effort to limit and mitigate spread of SARS-CoV-2.

### 6.3.5 References

1. Wu F, Zhao S, Yu B, et al. A new coronavirus associated with human respiratory disease in China. Nature 2020.
2. Zhou P, Yang X L, Wang X G, et al. A pneumonia outbreak associated with a new coronavirus of probable bat origin. Nature 2020.
3. Gorbalenya A E, Baker S C, Baric R S, et al. The species Severe acute respiratory syndrome-related coronavirus: classifying 2019-nCoV and naming it SARS-CoV-2. Nature Microbiology 2020.
4. Chu D K W, Pan Y, Cheng S M S, et al. Molecular Diagnosis of a Novel Coronavirus (2019-nCoV) Causing an Outbreak of Pneumonia. Clin Chem 2020.
5. Corman V M, Landt O, Kaiser M, et al. Detection of 2019 novel coronavirus (2019-nCoV) by real-time R T-PCR. Euro Surveill 2020; 25.
6. Letko M, Marzi A, Munster V. Functional assessment of cell entry and receptor usage for SARS-CoV-2 and other lineage B betacoronaviruses. Nat Microbiol 2020.
7. Wrapp D, Wang N, Corbett K S, et al. Cryo-E M structure of the 2019-nCoV spike in the prefusion conformation. Science 2020.
8. Walls A C, Park Y J, Tortorici M A, Wall A, McGuire A T, Veesler D. Structure, Function, and Antigenicity of the SARS-CoV-2 Spike Glycoprotein. Cell 2020.
9. Berry J D, Hay K, Rini J M, et al. Neutralizing epitopes of the SARS-CoV S-protein cluster independent of repertoire, antigen structure or mAb technology. MAbs 2010; 2:53-66.
10. Liu W, Fontanet A, Zhang P H, et al. Two-year prospective study of the humoral immune response of patients with severe acute respiratory syndrome. J Infect Dis 2006; 193:792-5.
11. Callow K A, Parry H F, Sergeant M, Tyrrell D A. The time course of the immune response to experimental coronavirus infection of man. Epidemiol Infect 1990; 105:435-46.
12. Choe P G, Perera R A P M, Park W B, et al. MERS-COV Antibody Responses 1 Year after Symptom Onset, South Korea, 2015. Emerg Infect Dis 2017; 23:1079-84.
13. Haveri A, Smura T, Kuivanen S, et al. Serological and molecular findings during SARS-CoV-2 infection: the first case study in Finland, January to February 2020. Eurosurveillance2020.
14. Pallesen J, Wang N, Corbett K S, et al. Immunogenicity and structures of a rationally designed prefusion MERS-COV spike antigen. Proc Natl Acad Sci USA 2017; 114:E7348-E57.
15. Kirchdoerfer R N, Cottrell C A, Wang N, et al. Pre-fusion structure of a human coronavirus spike protein. Nature 2016; 531:118-21.
16. Krammer F, Margine I, Tan G S, Pica N, Krause J C, Palese P. A carboxy-terminal trimerization domain stabilizes conformational epitopes on the stalk domain of soluble recombinant hemagglutinin substrates. PLOSOne 2012; 7:e43603.
17. Margine I, Palese P, Krammer F. Expression of Functional Recombinant Hemagglutinin and Neuraminidase Proteins from the Novel H7N9 Influenza Virus Using the Baculovirus Expression System. J Vis Exp 2013.
18. Li F, Li W, Farzan M, Harrison S C. Structure of SARS coronavirus spike receptor-binding domain complexed with receptor. Science 2005; 309:1864-8.
19. Wu K, Li W, Peng G, Li F. Crystal structure of NL63 respiratory coronavirus receptor-binding domain complexed with its human receptor. Proc Natl Acad Sci USA 2009; 106:19970-4.
20. Li Q, Guan X, Wu P, et al. Early Transmission Dynamics in Wuhan, China, of Novel Coronavirus-Infected Pneumonia. N Engl J Med 2020.
21. Tseng C T, Sbrana E, Iwata-Yoshikawa N, et al. Immunization with SARS coronavirus vaccines leads to pulmonary immunopathology on challenge with the SARS virus. PLOS One 2012; 7:e35421.
22. Nachbagauer R, Choi A, Izikson R, Cox M M, Palese P, Krammer F. Age Dependence and Isotype Specificity of Influenza Virus Hemagglutinin Stalk-Reactive Antibodies in Humans. MBio 2016; 7.
23. Rajendran M, Nachbagauer R, Ermler M E, et al. Analysis of Anti-Influenza Virus Neuraminidase Antibodies in Children, Adults, and the Elderly by ELISA and Enzyme Inhibition: Evidence for Original Antigenic Sin. MBio 2017; 8.
24. Amanat F, DuehrJ, Oestereich L, Hastie K M, Ollmann Saphire E, Krammer F. Antibodies to the Glycoprotein GP2 Subunit Cross-React between Old and New World Arenaviruses. mSphere 2018; 3.
25. Margine I, Palese P, Krammer F. Expression of functional recombinant hemagglutinin and neuraminidase proteins from the novel H7N9 influenza virus using the baculovirus expression system. J Vis Exp 2013:e51112.
26. Amanat F, Meade P, Strohmeier S, Krammer F. Cross-reactive antibodies binding to H4 hemagglutinin protect against a lethal H4N6 influenza virus challenge in the mouse model. Emerg Microbes Infect 2019; 8:155-68.
27. Wohlbold T J, Podolsky K A, Chromikova V, et al. Broadly protective murine monoclonal antibodies against influenza B virus target highly conserved neuraminidase epitopes. Nat Microbiol 2017; 2:1415-24.

### 6.4. Example 4: A Serological Assay to Detect SARS-CoV-2 Seroconversion in Humans

This example reports the development of an enzyme linked immunosorbent assay (ELISA) provide a protocol for both recombinant antigen production as well as the ELISA methodology.⁷ In particular, this example describes serological enzyme-linked immunosorbent assays for screening and identification of human SARS-CoV-2 seroconverters. The assays do not require handling of infectious virus, can be adjusted to detect different antibody types in serum and plasma and are amenable to scaling. Serological assays are of critical importance to help define previous exposure to SARS-CoV-2 in populations, identify highly reactive human donors for convalescent plasma therapy and investigate correlates of protection.

### 6.4.1 Methods

### Recombinant Proteins

The mammalian cell codon optimized nucleotide sequence coding for the spike protein of SARS-CoV-2 isolate (GenBank: MN908947.3) was synthesized commercially (GeneWiz). The receptor binding domain (RBD, amino acid 319 to 541, RVQP . . . CVNF) along with the signal peptide (amino acid 1-14, MFVF . . . VSSQ) plus a hexahistidine tag was cloned into mammalian expression vector pCAGGS as well as in a modified pFastBacDual vectors for expression in baculovirus system. The soluble version of the spike protein (amino acids 1-1213, MFVF . . . IKWP) including a C-terminal thrombin cleavage site, T4 foldon trimerization domain and hexahistidine tag was also cloned into pCAGGS. The protein sequence was modified to remove the polybasic cleavage site (RRAR to A) and two stabilizing mutations were introduced as well (K986P and V987P, wild type numbering). Recombinant proteins were produced using the well-established baculovirus expression system and this system has been published in great detail in 21-23 including a video guide. Recombinant proteins were also produced in Expi293F cells (ThermoFisher) by transfections of these cells with purified DNA using ExpiFectamine 293 Transfection Kit (ThermoFisher) Supernatants from transfected cells were harvested on day 3 post-transfection by centrifugation of the culture at 4000 g for 20 minutes. Supernatant was then incubated with 6 mls Ni-NTA agarose (Qiagen) for 1-2 hours at room temperature Next, gravity flow columns were used to collect the Ni-NTA agarose and the protein was eluted. Each protein was concentrated in Amicon centrifugal units (EMD Millipore) and re-suspended in phosphate buffered saline (PBS). Proteins were analyzed on reducing SDS-PAGE. The DNA sequence for all constructs is available from the Krammer laboratory Several of the expression plasmids and proteins have also been submitted to BEI Resources and can be requested from their web page for free (www.beiresources.org). S1 proteins of NL63 and 229E were obtained from Sino Biologics (produced in 293HEK cells, hexa-histidine tagged). A detailed protocol for protein expression of RBD and spike in mammalian cells is also available.⁷

### SDS-PAGE

Recombinant proteins were analyzed via a standard SDS-PAGE gel to check protein integrity. One ug of protein was mixed with 2× Laemmli buffer containing 5% beta-mercaptoethanol (BME) at a ratio of 1:1. Samples were heated at 100° Celsius for 15 minutes and then loaded onto a polyacrylamide gel (5% to 20% gradient; Bio-Rad). Gels were stained with SimplyBlue SafeStain (Invitrogen) for 1-2 hours and then de-stained in distilled water overnight.

### Human Samples

Human plasma and serum samples were obtained from a number of different sources. First, de-identified samples from the University of Melbourne (n=3, taken on day 2, 4 and 6 after symptom onset) and University of Helsinki (n=1, day 20 after symptom onset, neutralizing titers 1:160)⁶ were used as positive controls. For those, human experimental work was conducted according to the Declaration of Helsinki Principles and according to the Australian National Health and Medical Research Council Code of Practice. All participants provided written informed consent prior to the study. The studies were approved by the Alfred Hospital (ID #280/14) and University of Melbourne (ID #1442952.1, 1955465.2) Human Research Ethics Committees, and under research permit for project TYH2018322 of Helsinki University Hospital Laboratory.

Second, banked human samples were collected from study participants enrolled in several ongoing IRB approved longitudinal observational study protocols of the Mount Sinai Personalized Virology Initiative. The pre-pandemic serum panel comprised samples selected based on the date of collection (e.g., fall 2019) and whether participants had a documented history of viral infection (e.g., dengue virus, hantavirus, Chikungunya virus, coronavirus NL63). All participants agreed to sample banking and future research use. Self-reported ethnicities of the individuals from which samples were tested included Caucasian, Asian, African American and Hispanic. Samples included convalescent sera from a participant with an NL63 infection as determined by the Biofire Respiratory panel. We included serum collected at day 3 post symptom onset as well as convalescent serum from the same person (day 30 post symptom onset). These samples served as negative controls given that they were collect prior to SARS-CoV-2 spread in the US. Six subjects were 20-29, 19 were 30-39, 13 were 40-49, 7 were 50-59 years old and six were 60 or older. For the mRBD ELISAs sera from additional nine subjects were tested (30-39: 2; 40-49: 4; 50-59: 2; 60+: 1). The pre-pandemic panel was complemented by a collection of plasma samples collected from 50 HIV-1 infected individuals between 2008 and 2011.

Third, the Mount Sinai COVID19 panel comprised serum (n=12) and plasma samples from individuals with severe, mild or asymptomatic SARS-CoV-2 infections (see Supplementary Table 1). Seven paired serum and plasma samples from patients with COVID19 were used for comparison purposes. These samples were collected between 7 and 30 days post symptom onset. These samples were collected from study participants enrolled in an ongoing IRB approved study protocols of the Mount Sinai Personalized Virology Initiative.

SUPPLEMENTRAY TABLE 1

| | Sample taken post day | |
|---|---|---|
| Sample # | symptom onset | Severity |
| 1 | 20 | ND |
| 2 | 4 | ND |
| 3 | 3 | ND |
| 4 | 6 | ND |
| 5 | 8 | Severe |
| 6 | 11 | Severe |
| 7 | 15 | Severe |
| 8 | 7 | Severe |
| 9 | 10 | Severe |
| 10 | 13 | Severe |
| 11 | 17 | Severe |
| 12 | 20 | Mild |
| 13 | 20 | Mild |
| 14 | 30 | Mild |
| 15 | 21 | Mild |
| 16 | ND | Asymptomatic |

### Normal Human Immunoglobulin (NHIG)

The following NHIG preparations, each prepared from >1000 blood/plasma donors and intended for intravenous use for medical conditions, were tested in an ELISA to determine if they have reactivity against SARS-CoV-2 spike or RBD: Octagam (M934A8541), Gamunex-c (B2GMD00943, A1GLD01882, B3GLD01223, A1GLD01902, B2GLD01972, B3GGD00143, A1GKE00012 (2 different vials), B2GKD00863, B2GJE00033 (3 different vials)), Gammagard liquid (LE12T292AB, LE12V238AB, LE12V278AD), Gammagard S/D (LE08V027AB, 4 different vials), Gammagard liquid (C19G080AAA, LE12V071AD, LE12V230AB, LE12V115AC, LE12V205AB, LE12VE25AB, LE12V115AC).

### ELISA

The ELISA protocol was adapted from previously established protocols^{24,25} Ninety-six well plates (Immulon 4 HBX; Thermo Scientific) were coated overnight at 4° Celsius with 50 μl per well of a 2 ug/ml solution of each respective protein suspended in PBS (Gibco). The next morning, the coating solution was removed and 100 ul per well of 3% non-fat milk prepared in PBS with 0.1% Tween 20 (TPBS) was added to the plates at room temperature (RT) for 1 hour as blocking solution. Serum samples were heated at 56° C. for 1 hour before use to reduce risk from any potential residual virus in serum. Serial dilutions of serum and antibody samples were prepared in 1% non-fat milk prepared in TPBS. The blocking solution was removed and 100 ul of each serial dilution was added to the plates for 2 hours at RT. Next, the plates were washed thrice with 250 ul per well of 0.1% TPBS. Next, a 1:3000 dilution of goat anti-human IgG-horseradish peroxidase (HRP) conjugated secondary antibody (ThermoFisher Scientific) was prepared in 0.1% TPBS and 100 ul of this secondary antibody was added to each well for 1 hour. Plates were again washed thrice with 0.1% TBS. Once completely dry, 100 ul of SigmaFast OPD (o-phenylenediamine dihydrochloride; Sigma-Aldrich) solution was added to each well. This substrate was left on the plates for 10 minutes and then the reaction was stopped by addition of 50 μL per well of 3 M hydrochloric acid (HCl). The optical density at 490 nanometers was measured via a Synergy 4 (BioTek) plate reader. The background value was set at and optical density (OD) 490 nm of 0.11 and area under the curve (AUC) was calculated. AUC values below 1 were assigned a value of 0.5 for graphing and calculation purposes. Data were analyzed using Prism 7 (Graphpad). In some cases endpoint titers were calculated, the endpoint titer being the last dilution before reactivity dropped below and OD 490 nm of below 0.11. To determine the impact of heat treatments, paired samples that were heat treated or not treated were analyzed. NHIGs were run similar as serum/plasma samples but with a starting dilution at a concentration of 100 ug/ml. Three non-SARS-CoV-2 reactive human mAbs and CR302212-14 a human mAb reactive to the RBD of both SARS-CoV-1 and SARS-CoV-2 were used as controls.

To assess the distribution of the different antibody isotypes/subclasses in the samples that reacted well in our standard ELISA, another ELISA was performed with different secondary antibodies²⁶. These antibodies include anti-human IgA (a-chain-specific) HRP antibody (Sigma A0295) (1:3,000), anti-human IgM (μ-chain-specific) HRP antibody (Sigma A6907) (1:3,000), anti-human IgG1 Fc-HRP (Southern Biotech 9054-05) (1:3,000), anti-human IgG2 Fc-HRP (Southern Biotech #9060-05) (1:3,000), anti-human IgG3hinge-HRP (Southern Biotech 9210-05) (1:3,000), and anti-human IgG4 Fc-HRP (Southern Biotech 9200-05).

Of note, different ELISA substrates and stopping solutions that are less hazardous might be used in order to comply to local guidelines if appropriate.

### Microneutralization Assay

Vero. E6 cells were seeded at a density of 20,000 cells per well in a 96-well cell culture plate in cDMEM. The following day, heat inactivated serum samples (dilution of 1:10) were serially diluted 3-fold in 2×MEM (20% 10× minimal essential medium (Gibco), 4 mM L-glutamine, 0.2% of sodium bicarbonate [wt/vol; Gibco], 20 mM 4-(2-hydroxyethyl)-1-piperazineethanesulfonic acid (HEPES, Gibco), 200 U/ml penicillin-200 μ/ml streptomycin (Gibco), and 0.4% bovine serum albumin (MP Biomedical)). The authentic SARS-CoV-2 virus (USA-WA1/2020, GenBank: MT020880) was diluted to a concentration of 100 50% cell culture infectious doses (TCID₅₀) in 2×MEM. Eighty μL of each serum dilution and 80 μL of the virus dilution were added to a 96-well cell culture plate and allowed to incubate for 1 hr at room temperature. cDMEM was removed from Vero.E6 cells and 120 μL of the virus-serum mixture was added to the cells and the cells were incubated at 37° C. for 1 hr. After the 1 hr incubation, the virus-serum mixture was removed from the cells and 100 μL of each corresponding serum dilution and 100 μL of 2×MEM containing 2% FBS (Corning) was added to the cells. The cells were incubated for 48 hr at 37° C. and then fixed with 10% paraformaldehyde (PFA) (Polysciences, Inc) for 24 hr at 4° C. Following fixation, the PFA was removed and the cells were washed with 200 μL of PBS. The cells were then permeabilized by the addition of 150 μL of PBS containing 0.1% Triton X-100 for 15 minutes at room temperature. The plates were then washed three times with PBS containing 0.1% Tween 20 (PBS-T) and blocked in blocking solution (3% milk [American Bio] in PBS-T) for 1 h at room temperature. After blocking, 100 μL of 1C7 (anti-SARS NP antibody generated in house) at a dilution of 1:1000 was added to all wells and the plates were allowed to incubate for 1 hr at room temperature. Plates were then washed three times with PBS-T before the addition of goat anti-mouse IgG-horseradish peroxidase (IgG-HRP; Rockland Immunochemicals) (diluted 1:3000) in blocking solution for 1 hr at room temperature. Plates were then washed three times with PBS-T and the ( )phenylenediamine dihydrochloride (OPD) substrate (SigmaFast OPD; Sigma-Aldrich) was added. After a 10-minute room temperature incubation, the reaction was stopped by adding 50 μL of 3 M HCl to the mixture. The optical density (OD) was measured at 490 nm on a Synergy 4 plate reader (BioTek). A cutoff value of the average of the OD values of blank wells plus three standard deviations was established for each plate and used for calculating the microneutralization titer. Microneutralization assays were performed in a biosafety level 3 facility.

### Statistical Analysis

Differences between negative controls and positive controls were analyzed using an unpaired t-test. Differences between paired non-treated and heat-treated samples as well as paired serum and plasma samples were analyzed using a paired t-test. Correlation between ELISA titers and neutralization titers were analyzed using Spearman's rank test. Analyses were performed in GraphPad Prism.

### 6.4.2 Results

### Expression Constructs and Generation of Recombinant SARS-CoV-2 Proteins

We generated two different versions of the SARS-CoV-2 spike protein, based on the genomic sequence of the first virus isolate, Wuhan-Hu-1⁸. The first construct encodes a full length trimeric and stabilized version of the spike protein and the second one produces only the much smaller receptor binding domain (RBD). Sequences were codon optimized for mammalian cell expression. The full-length spike protein sequence was modified to remove the polybasic cleavage site, which is recognized by furin and to add a pair of stabilizing mutations (FIGS. 5B-5C).^{2,9,10} These two modifications were included to enhance the stability of the protein based on published literature.^{2,9} At amino acid P1213 the sequence was fused to a thrombin cleavage site, a T4 foldon sequence for proper trimerization and a C-terminal hexahistidine tag for purification (FIG. 5C). The sequence was cloned into a pCAGGS vector for expression in mammalian cells and into a modified pFastBac Dual vector for generation of baculoviruses and expression in insect cells. For expression of the RBD, the natural N-terminal signal peptide of S was fused to the RBD sequence (amino acid 319 to 541) and joined with a C-terminal hexahistidine tag (FIG. 5D).¹¹ The same vectors as for the full length S protein were used to express the RBD. In mammalian cells (Expi293F), the RBD domain gave high yields (approximately 25-50 mg/liter culture), but expression was lower in insect cells (approximately 1.5 mg/liter culture). Clear single bands were visible when the recombinant RBD proteins were analyzed on a reducing sodium dodecyl sulfate-polyacrylamide gel electrophoresis (SDS-PAGE), with the insect cell derived protein (iRBD) running slightly lower than the mammalian cell derived protein (mRBD) (FIGS. 5E-5F). The size difference likely reflects differences in glycan sizes between insect cells and mammalian cells. The full-length S protein was also expressed in both systems with higher yields in mammalian cells (mSpike) than in insect cells (iSpike) (approximately 5 mg/liter cultures versus 0.5 mg/liter culture). The full-length protein appeared as a prominent band between 135 and 190 kDa followed by a faint, second band slightly below on a reducing SDS-PAGE, which may be a cleavage product.

### ELISA Development

ELISAs were performed by doing serial dilution of the individual serum samples. Values from the dilution curves were used to determine the area under the curve (AUC), which was graphed. Initially, we tested a panel of 50 (59 for mRBD) banked human serum samples collected from study participants without and with confirmed previous viral infections (but otherwise healthy) to establish an ELISA with our proteins. These human sera were used to test the background reactivity to the SARS-CoV-2 spike in samples representative of the general US population from individuals ranging from 20 to 65+ years. An initial set of four plasma/serum samples from three COVID19 survivors were used to determine the reactivity of SARS-CoV-2 infected individuals to the RBD and the full length spike (FIGS. 8A-8H).

All COVID19 plasma/serum samples reacted strongly to both RBD and full-length spike protein while reactivity of the other serum samples only yielded background reactivity (FIGS. 8A-8H). Reactivity of COVID19 sera was, in general, stronger against the full-length S protein than against the RBD both for raw optical density and AUC values, which may reflect the higher number of epitopes found on the much larger spike protein. For the RBD, the difference between control sera and convalescent samples (initial n=4) was larger when the mammalian cell derived protein was used as compared to the insect cell derived RBD. The same was true for the full-length spike protein. We tested an additional 12 serum samples from patients with acute COVID19 disease as well as convalescent participants for reactivity to mRBD and mSpike (FIGS. 8A-8H). All 12 samples reacted with both RBD and spike protein. Thus, our assays distinguished sera from participants diagnosed with COVID19 from those collected prior to the pandemic (e.g., collected in the fall of 2019).

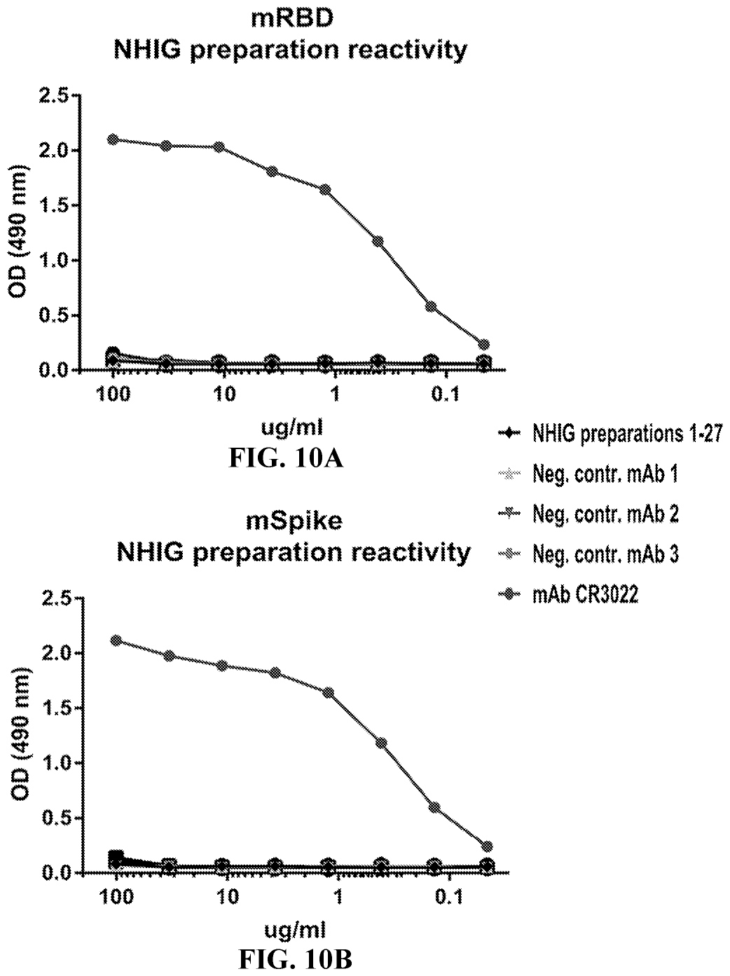

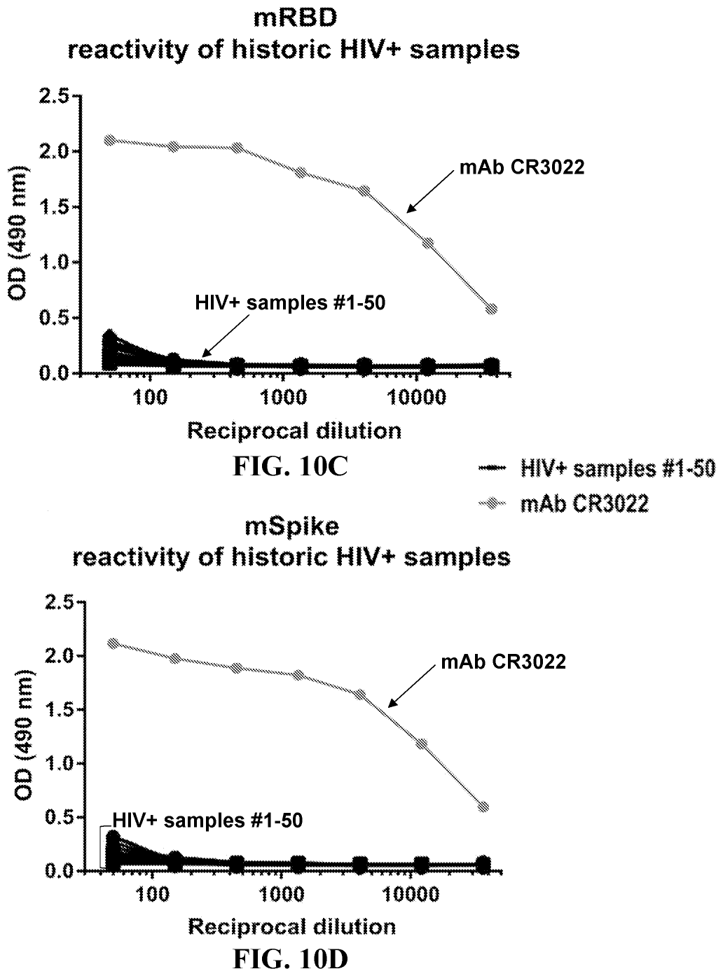

Our initial set of negative controls included convalescent serum from a participant with a confirmed NL63 infection. Importantly, this sample did not produce a signal against the SARS-CoV-2 RBD or spike. Since human coronaviruses OC43, 229E, NL63 and/or HKU1 are responsible for a large proportion of common colds every year, cross-reactivity between SARS-CoV-2 and these seasonal coronaviruses is of particular importance and warrants further investigation. To test how common antibodies to human coronaviruses other than SARS-CoV are in our “pre-pandemic serum panel”, we performed ELISAs coated with spike protein of coronaviruses 229E and NL63. While none of the negative control sera reacted to SARS-CoV-2 RBD and spike, the majority of samples yielded strong signals to the spike proteins of these two human coronaviruses (FIGS. 8I-8J). In addition, we tested 21 different batches (27 vials) of pools of different products of normal human immune globulin (NHIG), intended for intravenous use and derived from >1000 donors each. None of the NHIG preparations reacted with SARS-CoV-2 RBD or spike protein and the signal obtained was similar to that of the three irrelevant human monoclonal antibodies (mAbs). The RBD-binding mAb CR3022 produced, in contrast, a strong signal in the ELISA (FIGS. 10A-10B). 12-14 Lastly, we tested a panel of fifty plasma samples collected from HIV positive patients and banked from 2008 and 2011. Again, none of the samples reacted with the SARS-CoV-2 RBD or spike (FIGS. 10C-10D).

### Antibody Isotyping, Subtyping and Neutralizing Activity

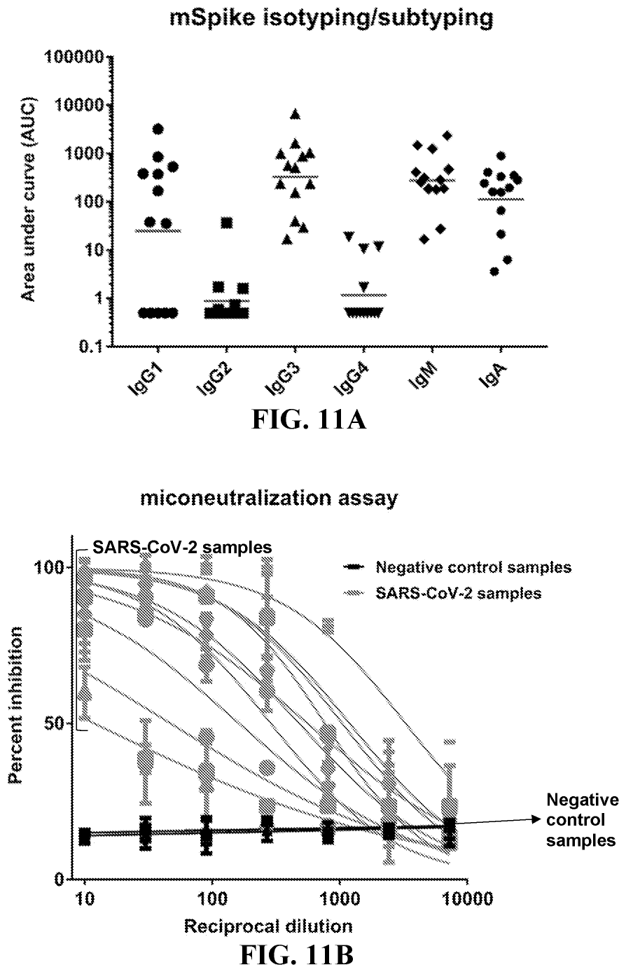

For the COVID19 patient plasma/sera from our initial panel, we performed an isotyping and subtyping ELISA using the mammalian cell expressed S proteins. Strong reactivity was found for all samples for IgG3, IgM and IgA (FIG. 11A). An IgG1 signal was detected for the majority of samples; low reactivity for IgG2 (in five of the COVID19 samples) and IgG4 (in four samples) was also detected. Furthermore, we correlated the ELISA reactivity with the neutralizing activity of sera against the USA-WA1/2020 isolate. ELISA titers and microneutralization titers correlated significantly (FIGS. 8K AND 11B) with a Spearman r of 0.9279.

### Reactivity of Serum, Plasma and Heat-Inactivated Samples

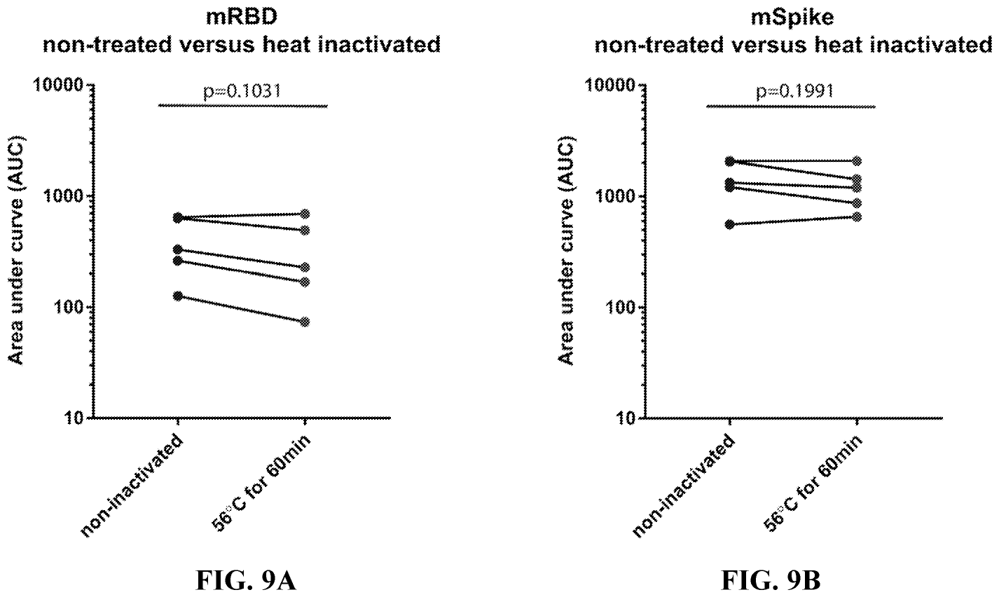

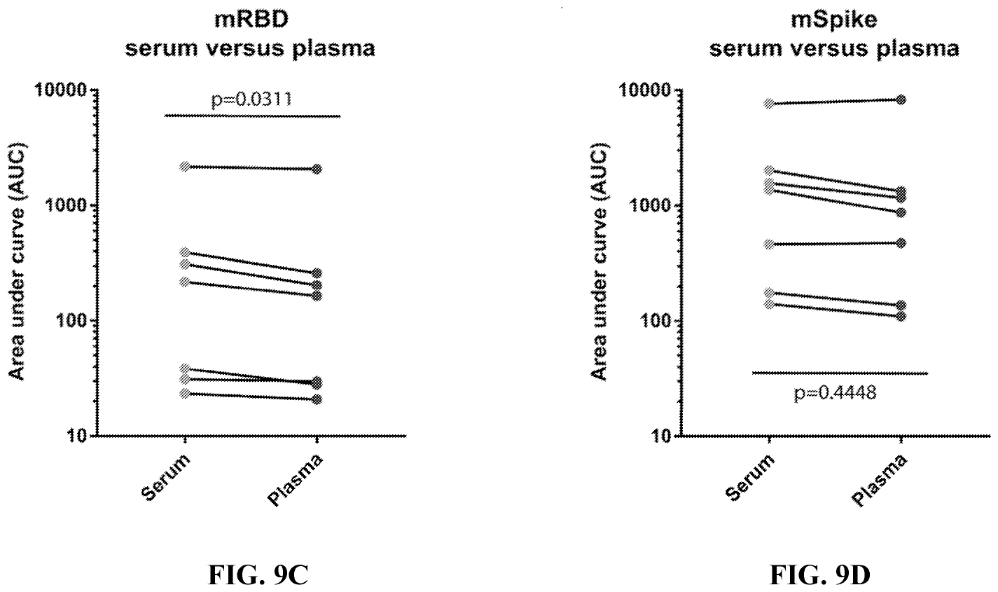

One complexity with measuring antibodies in bodily fluids of COVID 19 patients is, that infectious virus could be present in the biospecimen. To limit this risk, serum or plasma is heat inactivated for 1 hour at 56° C. To test if such a heat treatment has an effect on detecting antibodies to the SARS-CoV2 RBD and spike, we compared reactivity of matched non-treated and heat-treated serum samples from COVID19 patients. While slight differences were observed, they were minimal suggesting that heat treatment may have no negative impact on assay performance (FIGS. 9A-9B). Similarly, we tested matched serum and plasma samples from patients with COVID19 and found negligible differences suggesting that both types of specimens can be used in the assay interchangeably (FIGS. 9C-9D).

### 6.4.3 Discussion

Here we describe a serological method to detect seroconversion upon SARS-CoV-2 infection. The method is based on reactivity to the immunogenic S protein of the virus, is relatively simple and quick in its execution and can be performed at biosafety level 2 level as it does not involve live virus. We have tested these methods using banked serum samples and NHIG preparations obtained from individuals before SARS-CoV-2 started to widely circulate in the US. These serum samples produced low, close to baseline signals in our ELISAs. The age of the participants ranged from 20 to 65+ years of age and it is likely that most of these individuals had experienced infections with human coronaviruses including the alphacoronaviruses NL63 and 229E as well as the betacoronaviruses OC43 and HKU1.5 In fact, the majority of our negative control subjects had strong reactivity to the spike protein of NL63 and 229E, but showed no cross-reactivity to SARS-CoV-2 RBD and spike. We also included a convalescent serum sample from a participant with a laboratory confirmed coronavirus NL63 infection. Our data show that there is no or only negligible cross-reactivity from human coronaviruses to SARS-CoV-2 in these individuals. Similar findings were reported in a recent preprint where sera from negative control subjects reacted well with spike proteins from human coronavirus but not with SARS-CoV-2.¹⁵ This is notable because it suggests that humans are serologically naïve to SARS-CoV-2, which may explain the relatively high Ro of SARS-CoV-2 compared to other respiratory viruses, such as influenza virus.¹⁶ As a caveat, reactivity of samples from SARS-CoV-1 or Middle Eastern Respiratory Syndrome CoV infected individuals were not tested and might cross-react in this assay. Another caveat is of course the relatively small number of samples tested.

Our data show strong seroconversion with ELISA AUC values in the 1:1000 range after natural infection with SARS-CoV-2. Results from our assays suggest that antibodies mounted upon infection target the full length S protein as well as the RBD, which is the major target for neutralizing antibodies for related viruses coronaviruses.⁴ In fact, one of the samples SARS-CoV2 was previously tested in another study in neutralization assays and showed a neutralizing titer of 1:160.6 In addition, we performed microneutralization assays with a subset of our samples and found excellent correlation between our ELISA titers against the spike protein and virus neutralization, with several samples showing strong neutralizing activity with 50% inhibitory concentrations in the hundreds and thousands. This is in line with findings by Okbar and colleagues who also found a strong correlation between ELISA and neutralization.¹⁷ Of note, the ELISA reagents used are derived from the original sequence from Wuhan, the neutralization assays were performed with USA-WA1/2020 (an Asian lineage strain) while the majority of sera were obtained from subjects infected with European-lineage viruses.¹⁸ The observed correlation between ELISA and neutralization assays hints at minimal antigenic changes.

We believe that our ELISA method will be very useful for serosurveys aimed at determining the real attack rate and infection fatality rate in different human populations and to map the kinetics of the antibody response to SARS-CoV-2. While we found seroconversion in severe, mild and asymptomatic cases, it is possible that some individuals do not seroconvert or that antibody titers wane within short periods of time. To be able to interpret serosurveys correctly, studies to assess the kinetics of the antibody response and the rate of non-responders are urgently needed. Clinical trials with convalescent serum as therapeutic have been initiated in China (e.g. NCT04264858). In addition, a recent report suggests that compassionate use of these interventions could be successful.¹⁹ Screening potential plasma donors for high antibody titers using our assay is faster and easier than performing standard neutralization assays in BSL3 containment laboratories. Our assay has already been implemented for this purpose in Mount Sinai's Clinical Laboratory Improvement Amendments (CLIA) regulated clinical laboratory and has received emergency use authorization from New York State and from the FDA. Indeed, more than 120 patients with COVID 19 have been compassionately treated at Mount Sinai Hospital with antibody-rich plasma from convalescent donors identified with our assays.²⁰ Importantly, the assumption that antibodies to SARS-CoV-2 confer protection from reinfection needs to be confirmed and studies to investigate antibody titer as correlate of protection should be started as soon as possible. We are making the methods and laboratory reagents widely available to the research community in order to support the global effort to limit and mitigate spread of SARS-CoV-2. A detailed protocol⁷ for antigen expression and ELISA set up is available from the corresponding author and plasmids and proteins have been deposited at BEI Resources.

### 6.4.4 References

1 Letko, M., Marzi, A. & Munster, V. Functional assessment of cell entry and receptor usage for SARS-CoV-2 and other lineage B betacoronaviruses. Nat Microbiol (2020).
2. Wrapp, D., et al. Cryo-EM structure of the 2019-nCoV spike in the prefusion conformation. Science (2020).
3. Walls, A. C., et al. Structure, Function, and Antigenicity of the SARS-CoV-2 Spike Glycoprotein. Cell (2020).
4. Berry, J. D., et al. Neutralizing epitopes of the SARS-CoV S-protein cluster independent of repertoire, antigen structure or mAb technology. MAbs 2, 53-66 (2010).
5. Huang, A. T., et al. A systematic review of antibody mediated immunity to coronaviruses: antibody kinetics, correlates of protection, and association of antibody responses with severity of disease. medRxiv, 2020.2004.2014.20065771 (2020).
6. Haveri, A., et al. Serological and molecular findings during SARS-CoV-2 infection: the first case study in Finland, January to February 2020. (Eurosurveillance, 2020).
7. Stadlbauer, D., et al. SARS-CoV-2 Seroconversion in Humans: A Detailed Protocol for a Serological Assay, Antigen Production, and Test Setup. Curr Protoc Microbiol 57, e100 (2020).
8. Wu, F., et al. A new coronavirus associated with human respiratory disease in China. Nature (2020).
9. Pallesen, J., et al. Immunogenicity and structures of a rationally designed prefusion MERS-COV spike antigen. Proc Natl Acad Sci USA 114, E7348-E7357 (2017).
10. Kirchdoerfer, R. N., et al. Pre-fusion structure of a human coronavirus spike protein. Nature 531, 118-121 (2016).
11. Li, F., Li, W., Farzan, M. & Harrison, S.C. Structure of SARS coronavirus spike receptor-binding domain complexed with receptor. Science 309, 1864-1868 (2005).
12. Tian, X., et al. Potent binding of 2019 novel coronavirus spike protein by a SARS coronavirus-specific human monoclonal antibody. Emerg Microbes Infect 9, 382-385 (2020).
13. ter Meulen, J., et al. Human monoclonal antibody combination against SARS coronavirus: synergy and coverage of escape mutants. PLOS Med 3, e237 (2006).
14. Yuan, M., et al. A highly conserved cryptic epitope in the receptor-binding domains of SARS-CoV-2 and SARS-CoV. Science (2020).
15. Khan, S., et al. Analysis of Serologic Cross-Reactivity Between Common Human Coronaviruses and SARS-CoV-2 Using Coronavirus Antigen Microarray. bioRxiv, 2020.2003.2024.006544 (2020).
16. Li, Q., et al. Early Transmission Dynamics in Wuhan, China, of Novel Coronavirus-Infected Pneumonia. N Engl J Med (2020).
17. Okba, N. M. A., et al. Severe Acute Respiratory Syndrome Coronavirus 2-Specific Antibody Responses in Coronavirus Disease 2019 Patients. Emerg Infect Dis 26(2020).
18. Gonzalez-Reiche, A. S., et al. Introductions and early spread of SARS-CoV-2 in the New York City area. medRxiv, 2020.2004.2008.20056929 (2020).
19. Shen, C., et al. Treatment of 5 Critically Ill Patients With COVID-19 With Convalescent Plasma. JAMA (2020).
20. personal communication, Dr. Nicole Bouvier, Mount Sinai Hospital.
21. Amanat, F., et al. Antibodies to the Glycoprotein GP2 Subunit Cross-React between Old and New World Arenaviruses. mSphere 3(2018).
22. Krammer, F., et al. A carboxy-terminal trimerization domain stabilizes conformational epitopes on the stalk domain of soluble recombinant hemagglutinin substrates. PLOS One 7, e43603 (2012).
23. Margine, I., Palese, P. & Krammer, F. Expression of functional recombinant hemagglutinin and neuraminidase proteins from the novel H7N9 influenza virus using the baculovirus expression system. J Vis Exp, e51112 (2013).
24. Amanat, F., Meade, P., Strohmeier, S. & Krammer, F. Cross-reactive antibodies binding to H4 hemagglutinin protect against a lethal H4N6 influenza virus challenge in the mouse model. Emerg Microbes Infect 8, 155-168 (2019).
25. Wohlbold, T. J., et al. Broadly protective murine monoclonal antibodies against influenza B virus target highly conserved neuraminidase epitopes. Nat Microbiol 2, 1415-1424 (2017).
26. Rajendran, M., et al. Analysis of Anti-Influenza Virus Neuraminidase Antibodies in Children, Adults, and the Elderly by ELISA and Enzyme Inhibition: Evidence for Original Antigenic Sin. mBio 8(2017).

### 6.5 Example 5: Production of Cell Lines

The current pandemic of COVID-19 spiked an urgent need for SARS-CoV-2 research and diagnostic reagents of reliable quality in high amounts. The fastest approach adopted so far by our laboratory and others to accommodate this need is a series of transient transfections using serum-free high-expression cell lines, such as 293F cells (1). Several drawbacks accompany this approach, such as the cost of proprietary transfection reagents, cytotoxicity of transfection reagents, transfection efficiency, protein batch to batch variability and quality of expressed protein being dependent on passage number of the cell line at the time of transfection. Therefore, we decided to proceed with establishment of three cell lines stably expressing 1) SARS-CoV-2 Spike protein attached to the cell membrane via its transmembrane domain (Spike™ CHO DHFR), 2) SARS-CoV-2 Spike protein ectodomain (Spike CHO DHFR) and 3) SARS-CoV-2 receptor binding domain (RBD) of Spike protein (RBD CHO SHFR). Out of the possible parental cell lines to be used for this task, Chinese hamster ovary (CHO) DG44 cell line (2), which was previously adapted to serum-free conditions in-house (3), was selected. Different CHO strains have been a power horse of biotechnology industry for production of different therapeutic glycoproteins for years thanks to their human-like glycosylation, safety profile, transfectability and scalability, reaching high yields of produced proteins, surpassing many other systems (4). Among the mammalian-based expression systems, CHO cells are involved in the production of over 70% of recombinant biopharmaceutical proteins (5). Additionally, the CHO DG44 cell line is dihydrofolate reductase (DHFR) deficient (2). DHFR is an enzyme playing a crucial role in de novo synthesis of purines, thymidilic acid and certain amino acids, and thus the hypoxanthine and thymidine (HT) supplement must be added to the cultivation media to compensate for this deficiency. This feature can be exploited by co-transfecting an exogenous DHER together with the gene of interest (GOI) into the parental cell line. Upon removal of HT supplement from the cultivation media, only the clones with exogenous DHFR (and GOI) will be selected for. Furthermore, upon an amplification pressure provided by methotrexate (MTX), an inhibitor of DHFR, the gene amplification process starts in the positive clones, contributing to an additional increase in cell specific productivity and final protein yield.

### Methods

pCAGGs expression plasmids coding for SARS-CoV-2 Spike protein including the transmembrane domain, Spike protein ectodomain or receptor binding domain (RBD) only (1) were co-transfected together with pIRES_DHFR vector into CHO DG44 SF cells (3) using Lipofectamine 2000 (Invitrogen). Briefly, 1×10⁶ cells/well were transfected by an equal amount (4 ug) of each of the plasmids (in total volume of 250 μL OptiMEM media (Gibco)) and 5 μL Lipofectamine 2000 (in total volume of 250 μL of OptiMEM media) according to manufacturer's instructions in the 6-well plate format.

24 hours post transfection the transfected cells were moved to 96-well plates into the selection media (cultivation media without HT supplement, containing G418 (Thermo Fisher Scientific) and 50 nM MTX (Sigma Aldrich)) at the seeding density of 5×10\hat{ }3 cells/well and the selection process began. At two weeks post transfection, when the viable clones could be clearly distinguished under the microscope, the cells were fed again and the cycle of screening, expanding the viable clones into bigger cell culture volumes and increasing amplification pressure commenced.

For constructs expressing soluble protein into the supernatant (Spike ectodomain and RBD), the screening process consists of rounds of qualitative and later, quantitative ELISA assays. Since both recombinantly expressed proteins carry a hexahistidine tag, Ni-NTA 96-well ELISA plates (Qiagen) can be used. Briefly, for qualitative ELISA, 50 μL of cell culture supernatant is added onto the Ni-NTA 96-well ELISA plates and incubated for 2 hours at room temperature (RT) shaking. Human CR3022 monoclonal antibody against SARS-CoV-1, shown to cross-react with SARS-CoV-2 (6) is then used as a primary antibody (incubation 1 hr at RT, shaking) and goat anti-human IgG conjugated with horseradish peroxidase (HRP) (Sigma Aldrich) is used for detection (incubation 1 hr at RT, shaking). SigmaFAST™ OPD (O-phenylenediamine dihydrochloride; Sigma Aldrich) serves as a substrate for HRP and the reaction is stopped by 3M HCl before the plates are measured using Synergy H1 (BioTek) plate reader at 490 nm.

Constructs expressing the Spike protein attached to the cell membrane via the transmembrane domain of the protein are screened using immunofluorescence (IF). Either poly-D-lysine coating of plates is used or addition of low percentage FBS is added into the cell culture media, to help cells attach to the bottom of the plate. The clones are subsequently incubated in the presence of suitable primary antibody (e.g. CR3022) followed by detection with suitable secondary antibody (e.g anti-human IgG—Alexa 488). The plates are then analyzed using EVOS fluorescent microscope (Thermo Fisher Scientific).

### Results

Spike™ CHO DHFR: Spike™ construct was the first one transfected. After the first round of selection, only 24 viable clones were observed with cell numbers too low in the 96-well format to perform the immunofluorescence screening. The cells were therefore expanded gradually to 24-well plates and subsequently to 6-well plates without increasing the amplification pressure (staying at 50 nM MTX). Individual clones further stratified based on growth rate and it is now clearly possible to distinguish between the ones which seem to be growing steadily versus some which had to be downscaled back to 12-well plates at a higher seeding density in an attempt to rescue them. The IF screening on the best growing cultures is scheduled and based on the result the MTX pressure will be adjusted. Some of the clones are ready to be moved to T25 flasks, after which the next step will be to expand them to 30 ml shaking flasks.

RBD CHO DHFR: Following the transfection of the RBD construct, the first qualitative ELISA was performed right before the expansion of clones into 24-well plates. All the clones manifesting clearly distinguishable signal (28) were then expanded into 24-well plates and the amplification pressure was increased to 100 nM MTX. Another screen of qualitative ELISA is scheduled before the next expansion step and depending on the signals observed, MTX pressure will be increased to 200 nM. We do not expect major problems with scalability of these clones.

Spike CHO DHFR: Spike ectodomain construct was transfected last. Perhaps because of this additional time, the parental cell line had much longer time to recover from being frozen, and consequently many promising viable clones were observed already at 96-well plate stage. Therefore, MTX pressure from 50 nM to 100 nM right early at this stage was performed, before the initial ELISA screening round, which is now scheduled.

### References Cited in Example 5

1. Amanat F, Stadlbauer D, Strohmeier S, H. O N T, Chromikova V, McMahon M, Jiang K, Asthagiri Arunkumar G, Jurczyszak D, Polanco J, Bermudez-Gonzalez M, Kleiner G, Aydillo T, Miorin L, Fierer D, Lugo L A, Kojic E M, Stoever J, Liu S, Cunningham-Rundles C, Felgner P L, Moran T, Garcia-Sastre A, Caplivski D, Zheng A, Kedzierska K, Vapalathi O, Hepojoki J M, Simon V, Krammer F. 2020. A serological assay to detect SARS-CoV-2 seroconversion in humans, Nature Medicine.
2. Urlaub G, Kas E, Carothers A M, Chasin L A. 1983. Deletion of the diploid dihydrofolate reductase locus from cultured mammalian cells. Cell 33:405-12.
3. Chromikova V, Zaragoza M A, Krammer F. 2017. Generation of a serum free CHO DG44 cell line stably producing a broadly protective anti-influenza virus monoclonal antibody. PLOS One 12:e0183315.
4. Wurm F M, Hacker D. 2011. First CHO genome. Nat Biotechnol 29:718-20.
5. Lalonde M E, Durocher Y. 2017. Therapeutic glycoprotein production in mammalian cells. J Biotechnol 251:128-140.
6. Tian X, Li C, Huang A, Xia S, Lu S, Shi Z, Lu L, Jiang S, Yang Z, Wu Y, Ying T. 2020. Potent binding of 2019 novel coronavirus spike protein by a SARS coronavirus-specific human monoclonal antibody. Emerg Microbes Infect 9:382-385.

### 6.6 Example 6: Quantitative Determination of SARS-CoV-2 IgG

Technical Data

| | |
|---|---|
| Type of Assay: | ELISA |
| Measurand: | Severe acute respiratory syndrome coronavirus 2 |
| | (SARS-CoV-2) total IgG |
| Antigen: | Recombinant Sars Cov-2 Structural protein or spike |
| | (S) glycoprotein coated 96 well plates |
| Calibration: | Quantitative, in Arbitary Units/mL; 10 point |
| | calibration 4 PL Curve |
| Controls: | Negative and Low. Medium and High Controls |
| Reagents: | Buffer. Anti-Human IgG (Fab specific) - Peroxidase |
| | antibody, Substrate |
| Measurement: | 490 nm |
| Statistical Analysis: | GraphPad Prism. ® version 8 (GraphPad Software, |
| | La Jolla, CA) |

### Clinical Significance

Since December 2019, a pandemic of coronavirus disease 2019 (COVID-19) is caused by SARS-CoV-2 with a spectrum of symptoms that include fever, fatigue, dry cough, myalgia, and dyspnea. It is vital to evaluate the prevalence of antibody measurement quantitatively in both asymptomatic and symptomatic of SARS-CoV-2 patients. For SARS-CoV-2, neutral methods are ideal for detection, however, they were poorly characterized in symptomatic and asymptomatic patient's sera. Quantification of SARS-CoV-2 antibodies (IgG) correlates well with immune responses, disease severity and viral replication rates.

### Diagnostic Application

SARS-CoV-2 IgG is an enzyme-linked immunosorbent assay (ELISA) used for the quantitative detection of IgG antibodies to SARS-CoV-2 in human serum, and plasma (Gold-top Serum Separator Tube, Sodium Heparin, Lithium Heparin, and Potassium EDTA tubes).

Rationale: ELISA is widely used for several Clinical Immunology/Serological assays. These assays' lack of reference material, calibrators and controls are prepared from serum pool (to avoid inter-individual variability). Arbitary units are assigned to calibrators and test samples are reported in same units and this is a common practice in CLIA/CAP laboratories. The current assay quantitatively measures SARS-CoV-2 IgG antibodies using similar strategy. The advantages of quantitative determination is 3 fold: to provide more reliable values for antibody levels that correlates with neutralization titers and efficacy of future vaccine studies (quantitative ELISA that measures IgG levels is used in the evaluation of vaccines as a surrogate assay) and finally establishing a gold standard assay that is traceable globally (like WHO assays)

Methodology: MSHS lab resulted 13657 patient Covid specimens by FDA approved EUA assay and created a biobank for future studies. From this repository. very high titer (≥2880) specimens were pooled (to avoid inter-individual) and established a reference calibration/standard serum. In an identical fashion negative control serum (from naive patients) and low, medium and high level control materials were prepared.

The quantitate ELISA methodology was identical to FDA approved EUA method that is currently in use at Mount Sinai. In brief, 96 well plates were coated with recombinant SARS COV-2 spike protein (2 ug/mL) Plates were incubated overnight at 2-8° C. and then washed, blocked and finally pre-diluted (1.80) patient specimens, controls were added to respective assigned wells. A 10-point calibration curve was generated by serially diluting reference calibration standard pool. Following incubation, the plate was washed three times with Wash Buffer. Horseradish peroxidase (HRP)-conjugated goat anti-human IgG. was added to all wells, then the plate was incubated and washed followed by the addition of o-phenylenediamine dihydrocloride (OPD) substrate. Within 30 minutes the reaction was suspended by adding 3 M HCl Stop Solution. Optical density (OD) readings for each well were obtained at 490 nm.

### Data Analysis:

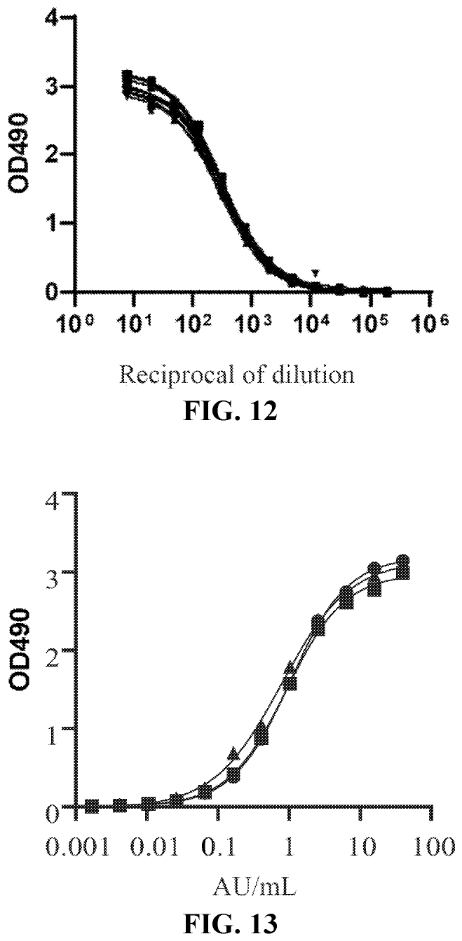

Calculation of Arbitrary units for reference standard serum: A 10 point calibration curve was generated by serial dilution and optical density (OD 490) values were plotted against reciprocal of dilution by 4-Parameter Logistic (4PL) curve. The four parameters of the 4PL model are the lower asymptote of the sigmoidal curve (a), a curvature parameter related to the slope of the curve (b), a parameter related to the dilution at the midpoint of the curve (c), the upper asymptote of the curve (d). The constants are calculated using GraphPad Prism.® version 8 (GraphPad Software, La Jolla. CA) and IC50 value was assigned as an arbitrary baseline antibody concertation (AU/mL) for the reference standard (FIG. 12). Thereafter, Arbitrary unit were used to create a stand curve (X axis=AU/mL and Y axis=OD 490 nm) using 4 PL curve (FIG. 13), unknown specimens OD value was used to calculate concentration by following equation.

$$x = {c ( \frac{a - x}{y - d} )}^{( \frac{1}{b} )}$$

X=AU/mL, y=OD490, a=lower asymptote, b=hill slope, c=IC50, d=upper asymptote of 4PL curve.

TABLE 1
The constants were calculated using Graph Pad
Prism for the reference calibration curve.

| Best-fit values | Reference Standard |
|---|---|
| Bottom | −0.01028 |
| Top | 3.066 |
| IC50 | 331.4 |
| Hill Slope | −0.9449 |
| logIC50 | 2.52 |
| Span | 3.076 |
| R squared | 0.9991 |

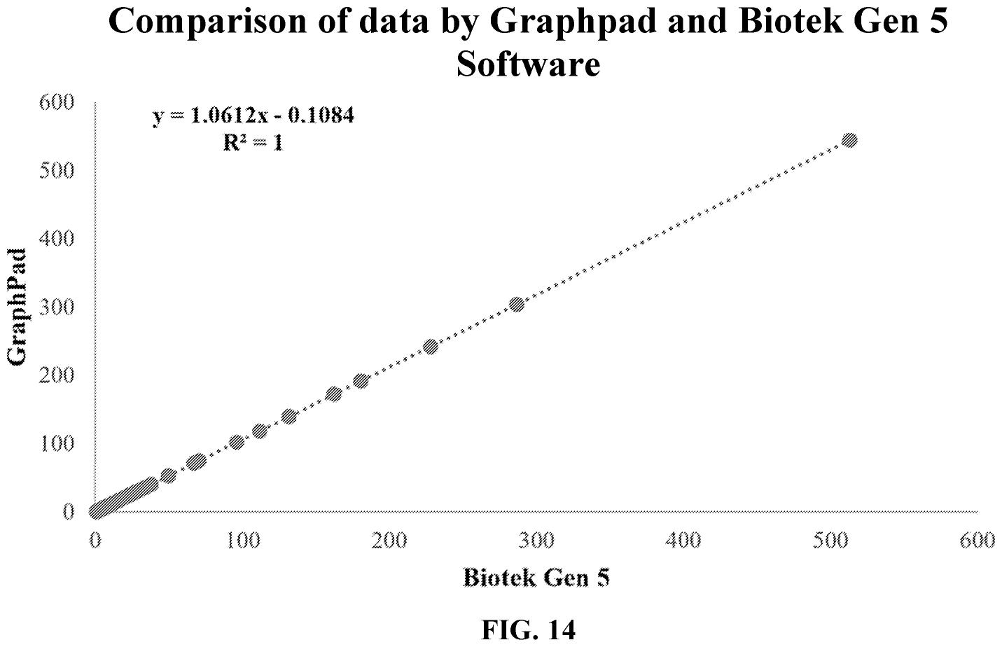

Preliminary data
1. Accuracy of the assay:
   a. Sixty patient specimens were analyzed by our standard procedure and the raw data was analyzed by GraphPad Prism and Biotek Gen 5 software (Winooski, VT). Both statistical programs used 4 PL calibration curve to determine unknowns. The acceptable criteria is +/−20% and MSHS quantitative assay values are truly agreeable on both statistical packages (FIG. 14).

### 6.7 Example 7: ELISA IoG Antibody Test

A. PURPOSE: Use of a COVID-19 ELISA IgG Antibody Test for the in vitro quantification of IgG antibody in serum and plasma from individuals suspected of previous COVID-19 infection by their healthcare provider, for the assessment of seroconversion from an antibody negative status to an antibody positive status in acutely infected patients.

B. MEASURAND: Anti-SARS COV-2 IgG antibodies

C. PROPOSED INTENDED USE

Intended use: A SARS-CoV-2 assay is an enzyme-linked immunosorbent assay (ELISA) is intended for the quantitative measurement of IgG antibodies to SARS-CoV-2 spike protein in human serum and plasma. The SARS-CoV-2 assay is read on an IVD microplate reader at 490 nm. The SARS COV-2 quantitative assay also aids in correlating the disease severity, viral replication rates or immune outcome patients with severity.

Indications For Use: The ELISA for SARS COV-2 IgG assay is intended for the quantitative detection of IgG antibodies to SARS COV-2 antigen from SARS coronavirus in human serum/plasma. The assay is intended for use as an aid in identifying individuals with an adaptive immune response to SARS-CoV-2, indicating recent or prior infection (in serum/plasma samples from asmptomatic/symptomatic patients or those suspected of having SARS Cov-19 symptoms).

Instruments Used with Test:
BioTek (Winooski, VT) Energy Neo 2 Multi-Mode Reader coupled with BioTek (Winooski, VT) Biostack Neo
BioTek (Winooski, VT) EL406 Washer Dispenser coupled with BioTek (Winooski, VT) Biostack 4 Microplate Stacker
Labconco™ Purifier™ Logic™+ Class II A2 Biosafety Cabinets, Fisher Scientific, Cat #30-248-1001, or equivalent

D. DEVICE DESCRIPTION AND TEST PRINCIPLE

The quantitative ELISA for SARS COV-2 IgG assay is designed to detect IgG class antibodies in human sera/plasma to corona virus spike antigen.
I. Purified recombinant Spike antigen is immobilized on a plastic matrix in a 96-well plate format and the antibodies present in diluted human serum/plasma are bound to the antigen. Levels of IgG antibodies are then determined using a horseradish peroxidase (HRP)-labeled anti-human IgG (fab specific) secondary antibody produced in goat.
II. Horseradish peroxidase (HRP) conjugated goat anti-human IgG (Human Fab specific IgG) is added to the wells and the plate is incubated. The conjugate will react with anti-SARS COV-2 IgG bound to antigen and the wells are washed to remove unreacted conjugate
III. The microwells containing HRP conjugate are incubated with peroxidase substrate (o-Phenylenediamine) solution. Hydrolysis of the substrate by peroxidase produces a color change. After a period of time the reaction is stopped by adding 3 M Hydrochloric acid and the color intensity of the solution is measured by spectrophotometer with the wavelength set at 490 nm.
IV. Antibody levels are reported in an arbitrary unit value based upon a four parameter logistic curve generated from a serial dilution of a strong positive (high titer ≥2880 using our qualitative assay) patient serum pool that is included on each ELISA plate. This assay measures only the level of antibodies present in a given serum specimen.

### Description of Test Steps:

### Full Length Spike Protein by ELISA

1. Coating ELISA plates
   Thaw the required number of vials of antigen (SARS-CoV-2 Spike protein) to coat 96-well microtiter ELISA plates at a concentration of 2 ug/mL. Once thawed, mix by gently vortexing vial before diluting in 1×PBS.
   Prepare approximately 6 mL for each plate to be coated.
   Coat plates with 50 μL of diluted protein per well using a multichannel pipettor and a reservoir. Lightly tap plates against surface to ensure protein is completely coated on the bottom of every well.
   Incubate at 4° C. overnight. Plates can be stored at 4° C. for up to 1 week.
   Always keep a cover plate on top of coated plates during all steps of the protocol.
2. Block ELISA plate
   Prepare at least 25 mL of blocking solution per plate
   Blocking solution consists of PBS-T+3% non-fat milk powder (weight/volume).
   Using an automated plate washer, wash coated ELISA plates 3× with PBS-T.
   Add 200 μL blocking solution to all wells of the plates, incubate at room temperature for 1 hour, but do not exceed 4 hours, after completing the first plate.
3. Pre-diluting samples
   In a biological safety cabinet, set up microcentrifuge tubes to pre-dilute samples 1:5.
   Add 40 μL of 1×PBS to all tubes.
   Add 10 μL of controls and patient specimens to each tube containing 40 μL of 1×PBS, vortex to mix.
4. Standards preparation
   Standard curve is generated by serial dilutions of a high titer patient serum pool in 0.05% Tween-20 PBS. The dilutions ranges from 8-4883.
5. Controls preparation
   Control (Low, Medium an High Quality Control) sera are stable up to one month when refrigerated at 2-8° C.
6. Calibration and calibration verification procedures
   Not required for this procedure
7. Sample Preparation
   Specimens are diluted to 1:80 with 0.05% Tween-20 PBS, vortexed briefly, and stored at 4° C. until assayed.
8. Detailed Sample Preparation
   The plates were washed in Tween-20 Phosphate Buffered Saline and blocked with 100 μL per well of 0.3% Tween-20 PBS for 2 h at 4° C.
   After a series of three washes (subsequent washes were all with 0.05% Tween-20 PBS), 100 μL aliquots of serum diluted 1:80 with wash buffer were added to each well. A serial dilution (1:50 to 1:6400) of a strong positive serum pool was used to generate a standard curve on each individual plate. A buffer blank, three levels of control sera, and a negative for SARS COV-2 IgG specimen (that were previously known by our qualitative ELISA assay) were also included on each plate.
   Plates were incubated 2 h at room temperature, and then washed four times with wash buffer
   A secondary antibody against human IgG fab specific conjugate (Horseradish peroxidase (HRP) was added to each well and incubated for 1 h at room temperature.
   After three washes the microwells containing HRP conjugate are incubated with peroxidase substrate (o-Phenylenediamine) solution. Hydrolysis of the substrate by peroxidase produces a color change. After a period of time the reaction is stopped by adding 3 M Hydrochloric acid and the color intensity of the solution is measured by spectrophotometer with the wavelength set at 490 nm.
   The absorbance's were measured at 490 nm using a Biotek microplate reader. Antibody levels of the unknown samples were assigned a unit value based on the 8-point standard curve with a four-parameter curve fit.
   Unknown specimens with absorbance values above the standard curve were diluted further and reassayed. Arbitrary unit values were expressed per mL of serum.
9. Recording of Data
   Biotek Gen 5 Analysis Software or by Graphpad prism Software
10.Calculations
   Optical densities at 490 nm were converted to arbitrary unit values per mL of serum using the Gen 5 Analysis Software.

Control Material(s) to be Used with the Quantitative COVID-19 ELISA IgG Assay: Three known Low, Medium and high titered sera and negative serum were included on each ELISA plate along with an eight point standard curve. The four-parameter curve that is fitted to the standard curve values must have a correlation coefficient of >0.98 or the run is rejected. As with the unknowns, the values for the positive control standards must have a CV of less than 20% from the established value. See Table 2, infra.

TABLE 2
Assay results and interpretation (preliminary)

| | Result | | |
|---|---|---|---|
| Test | (AU/mL) | Final Result | |
| Quantitative | <5 | NEGATIVE | A result of indicates that the result is below the test's |
| IgG | | | Limit of Detection (LoQ), the sample is negative |
| measurement | >5 | POSITIVE | This result is consistent with levels of anti-SARS |
| | | | CoV-2 IgG at >5 AU/mL, indicates that anti-SARS |
| | | | CoV-2 IgG has been detected at levels consistent with |
| | | | protective immunity against SARS CoV-2 infection. |

### Note:

The quantitative must be used in conjunction with immune status of the individual should be further assessed by considering other factors, such as clinical status, follow-up testing, associated risk factors.

E. PRODUCT MANUFACTURING

The COVID-19 ELISA IgG Quantitative measurement assay/test has been validated using only the components referenced in this submission and shall not be changed without prior concurrence from the FDA.

### 1) Overview of Manufacturing and Distribution:

The product and components are all developed at the Mount Sinai Laboratory (MSL), Center for Clinical Laboratories, a division of the Department of Pathology, Molecular, and Cell-Based Medicine, New York, NY.
   Components Included with the Test
Recombinant full-length Spike protein
   2) Components Required but Not Included with the Test
Flat-Bottom Immuno Nonsterile 96-Well Plates 4 HBX (Thermo Scientific #3855, or equivalent
Thermo Scientific™ (Waltham, MA) Nunc™ MicroWell™ 96-Well Microplates, Non-Treated, Cat #260895, or equivalent
Milk Powder, Fisher Scientific, Cat #NC0115668, or equivalent. Store at room temperature, stable until expiration date
PBS (1×) (Gibco), ThermoFisher, Cat #100010023, or equivalent. Store at room temperature, stable 24 months from date of manufacture
Water for Injection (WFI) for Cell Culture (Gibco #A1287301 or equivalent)
Tween™ 20 (C58H114026; MW: 1227.5), Fisher BioReagents™, Fisher Scientific, Cat #BP337-500, CAS #9005-64-5, or equivalent, store at room temperature, stable until expiration date.
Phosphate Buffered Saline (10×), Fisher Scientific, Cat #MT-46013CM, or equivalent, store at room temperature, stable until manufacturers expiration date.
Eppendorf (Hamburg, Germany) Repeater M4 pipettor; 10 mL Eppendorf tips (Cat #30089820), 5 mL Eppendorf tips (Cat #30089812) and 1 mL Eppendorf tips (Cat #30089790)
Micropipette tips
   Fisher Scientific™ (Hampton, NH) Fisherbrand™ SureOne™ Micropoint Pipette Tips, Universal Fit, Non-Filtered 0.1-10 μL pipette tips (Cat. #02-707-420), or equivalent
   Fisher Scientific™ (Hampton, NH) Fisher Brand 1-200 μL pipette tips (Cat. #02-707-420), or equivalent
   USA Scientific, 200 μL TIPONE PIPETTE TIP STACKS, Cat #1111-0240, or equivalent
   Fisher Scientific™ (Hampton, NH) Fisher Brand 100-1250 μL pipette tips (Cat. #02-707-403), or equivalent
Sterile, serological pipettes
   5 mL (Falcon #356543 or equivalent)
   10 mL (Falcon #357551 or equivalent)
   25 mL (Falcon #357535 or equivalent)
   50 mL (Falcon #356550 or equivalent)
Polypropylene sterile conical tubes
   Fisher Scientific Centrifuge Tubes, 15 mL (17×120 mm), Molded with Graduations, Polypropylene with screw cap, Fisher Scientific, Cat #50-145-8829, or equivalent.
   Fisher Scientific Tubes, 50 mL (30×115 mm), Molded with Graduations, Polypropylene with screw cap, Fisher Scientific, Cat #50-145-8841, or equivalent.
Anti-Human IgG (Fab specific, in 0.01 M phosphate buffered saline, pH 7.4, containing 0.01% thimerosal)-Peroxidase antibody produced in goat, Sigma Aldrich® (St. Louis, MO), Cat #A-0293. Store at −20° C. freezer, stable until manufacturer's expiration date.
Hydrochloric Acid 3.0M (HCl; MW:36.4), Fisher Scientific, Cat #S25856, CAS #7647-01-0, or equivalent. Store at room temperature in flammable safety cabinet.
SIGMAFAST™ OPD (o-Phenylenediamine dihydrochloride), Sigma-Aldrich (St. Louis, MO), #P9187 or equivalent, storage temperature 2-8 C, stable until manufacturer's expiration date.
Precision pipette capable of delivering 0-20 μL, Eppendorf Research Plus or equivalent, (San Diego, CA) Cat #3120000810)
Precision pipette capable of delivering 0-2.5 μL, Eppendorf Research Plus or equivalent, (San Diego, CA) Cat #3120000011)
Precision pipette capable of delivering 0-200 μL, Eppendorf Research Plus or equivalent, (San Diego, CA) Cat #3123000055)

### Assay Performance Characteristics

### Precision/Reproducibility:

Limit of Blank (LoB), Limit of Detection (LoD), and Limit of Quantitation (LoQ):

The limits of detection (LoD) was determined by running 13 replicates of the assay buffer (treated as patient specimen) on one assay plate. Two standard deviations were added to the mean OD reading (Limit of Blank), and this value was read using calibration curve to calculate the LoD. Limits of Quantification (LoQ) was calculated by adding 10 standard deviation to Limits of Detection. See Table 3, infra.

TABLE 3

| Blank | AU/mL |
|---|---|
| Blank 1 | 2.10 |
| Blank 2 | 2.10 |
| Blank 3 | 2.14 |
| Blank 4 | 2.62 |
| Blank 5 | 2.43 |
| Blank 6 | 2.85 |
| Blank 7 | 2.66 |
| Blank 8 | 2.48 |
| Blank 9 | 2.71 |
| Blank 10 | 2.38 |
| Blank 11 | 2.14 |
| Blank 12 | 2.10 |
| Blank 13 | 2.24 |
| Mean | 2.38 |
| SD | 0.27 |
| CV | 11.18 |
| LoD | 3.18 |
| LoQ | 5.04 |

### Analytical Specificity:

### Reactivity/Inclusivity

Although mutations in the SARS-CoV-2 genome have been identified as the virus has spread, no serologically unique strains have been described relative to the originally isolated virus (this research is limited at present).

### Analytical Specificity:

A study was conducted to assess cross reactivity with the Mount Sinai IgG Test System using patient sera that was seropositive to different microorganisms and samples from patients diagnosed with certain disease conditions. See Table 4, infra.

TABLE 4
Cross Reactivity Study

| Antibody positive sera | N | COVID-19 IgG (AU/mL) |
|---|---|---|
| Varicella | 5 | Validation in progress |
| Influenza | 4 | |
| HSV | 7 | |
| Rubella | 5 | |
| Hepatitis | 4 | |
| HIV | 4 | |
| Elevated IgG | 5 | |
| Elevated IgM | 4 | |
| CMV | 2 | |

### Precision

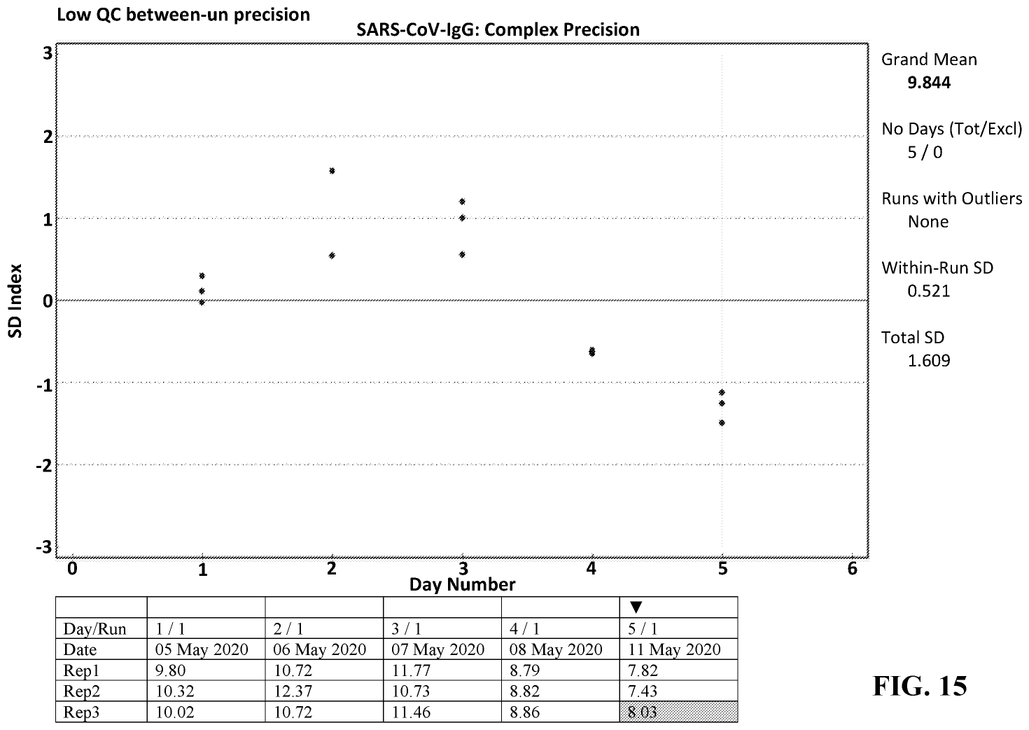

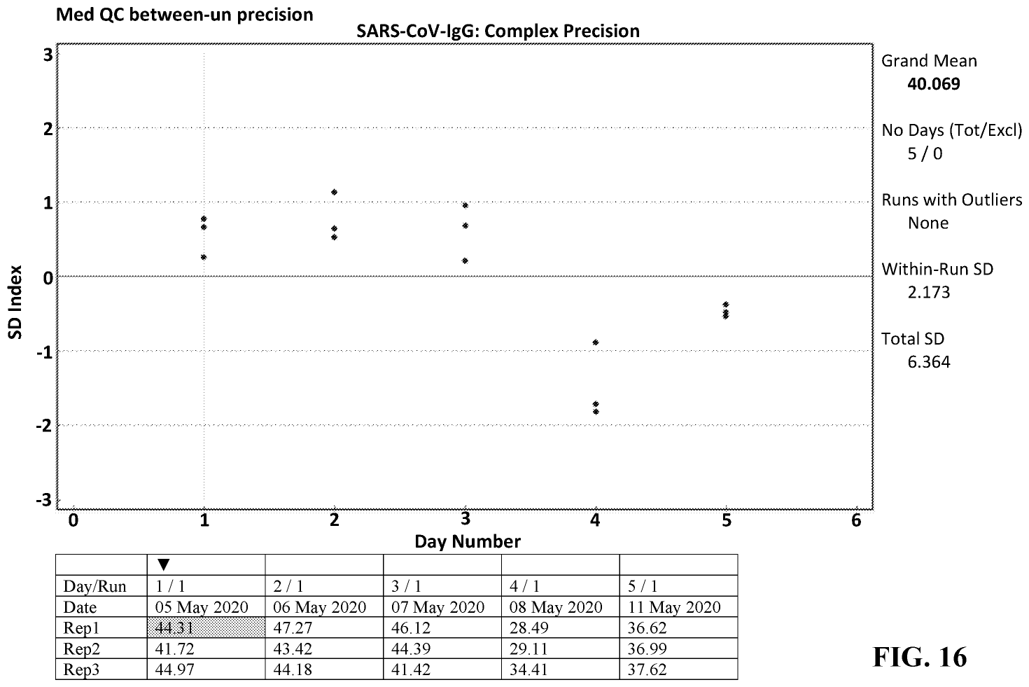

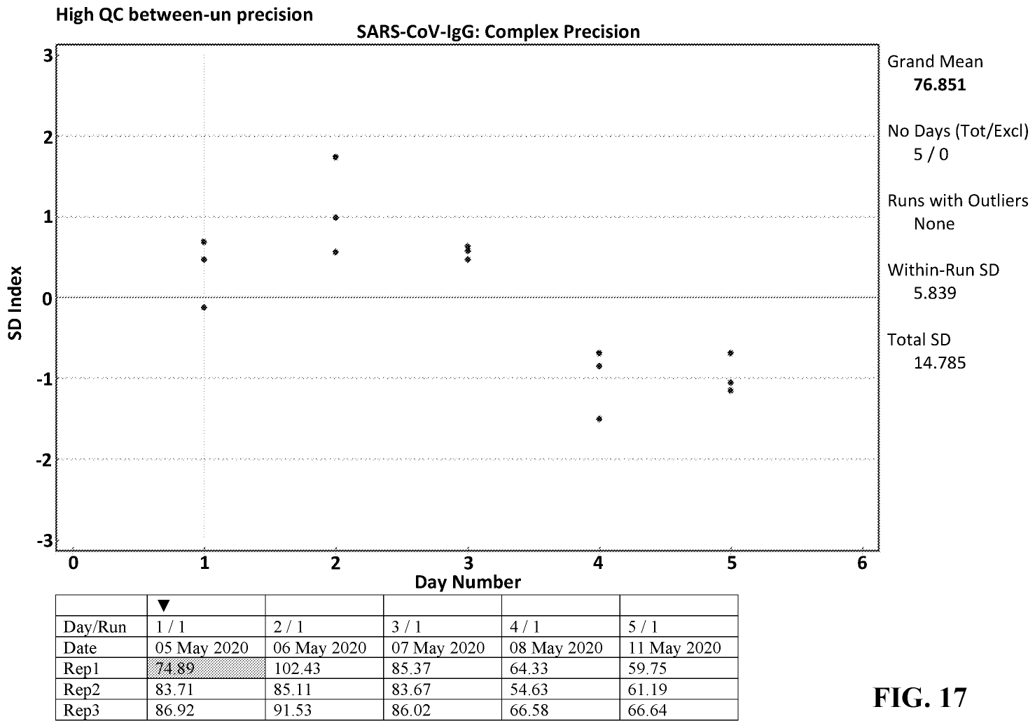

Assay precision within day and between-day precision was evaluated using Low, Medium and High controls for quantitative ELISA. Precision was reported as the coefficient of variation (CV). Within day precision was calculated by analyzing 10 replicates of negative, Low, Medium and High controls on one day. Between day precision was calculated by analyzing negative, Low, Medium and High controls for 5 different days. The acceptance criterion for within day and between day precision (% CV) was ≤20% for each control tested. The between-run precision data was analyzed by EP Evaluator (South Burlington, VT, USA). See FIGS. 15-17 and Table 5, infra.

TABLE 5
With-run precision:

| | Neg (AU/mL) | Low QC | Med QC | High QC |
|---|---|---|---|---|
| Replicate 1 | 1.12 | 8.66 | 26.77 | 69.78 |
| Replicate 2 | 0.99 | 8.79 | 28.64 | 56.50 |
| Replicate 3 | 1.06 | 8.82 | 29.16 | 59.01 |
| Replicate 4 | 0.91 | 8.86 | 28.49 | 58.00 |
| Replicate 5 | 0.86 | 10.29 | 29.11 | 60.57 |
| Replicate 6 | 0.91 | 10.08 | 34.41 | 45.88 |
| Replicate 7 | 1.09 | 9.33 | 36.82 | 66.67 |
| Replicate 8 | 0.93 | 10.36 | 35.11 | 64.33 |
| Replicate 9 | 0.99 | 10.53 | 36.88 | 54.63 |
| Replicate 10 | 0.99 | 9.77 | 35.69 | 66.58 |
| Mean | 0.98 | 9.55 | 32.11 | 60.20 |
| SD | 0.09 | 0.74 | 3.99 | 7.05 |
| % CV | 8.71 | 7.74 | 12.43 | 11.72 |

Interfering Substances: The following potentially interfering substances were evaluated in serum to establish their effect on the investigational device: cholesterol, hemoglobin.

The concentration of analyte of each potential interfering substance is in process: Hemoglobin: 10 g/dL (low), 15 g/dL (High); Intralipid: 300 mg/dL (low), 750 mg/dL (high).

Three clinical samples exhibiting differing reactivities with the Mount Sinai IgG Test System were tested for interference with above substances: a low QC specimen, and high QC specimen. All specimens exhibited a change of signal less than % when tested with each potential interferant.

### Dilutional Linearity

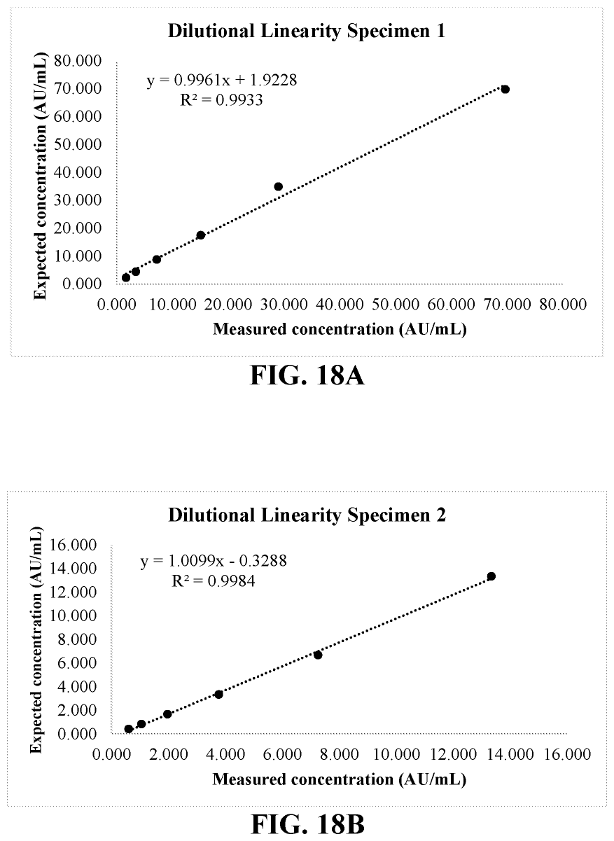

Acceptability criteria for dilution integrity experiments was +20% from the expected values. The linearity of the analytical measurement range (AMR) was evaluated by two patients specimens serum with various SARS COV-2 IgG antibody concentrations were diluted with Phosphate Buffered Saline serially (1:2, 1:4, 1:8, 1:16 and 1:32) to obtain values that cover the AMR and clinically reportable range (CRR). Acceptability criteria for dilution integrity experiments was ±20% from the expected values. All two specimens fulfilled the acceptance criteria, showing good linearity in their respective ranges. See FIGS. 18A-18B.

### Matrix Comparison: N/A

Sample Stability and Handling: Three samples were tested in duplicates for up to 7 days while stored at 2-8° C., and after repeated freeze/thaw cycles up to 3 cycles (−80° C.). Results were compared to those obtained on same specimens (day zero, stored at 2-8° C. and −80° C.).

### 6.8 Example 8: ELISA IgG

### A. Proposed Intended Use

### 1) Intended Use (IU):

The Mount Sinai Quantitative SARS-CoV-2 assay is an Enzyme-Linked Immunosorbent Assay (ELISA) that is intended for the quantitative measurement of IgG antibodies to SARS-CoV-2 spike protein in human serum and plasma. The Mount Sinai Quantitative SARS-CoV-2 assay is read on an IVD microplate reader at 490 nm. The Mount Sinai Quantitative SARS-CoV-2 assay is intended for use as an aid in identifying individuals with an adaptive immune response to SARS-CoV-2, indicating significant exposure or prior infection of the SARS-Cov-2 virus. The SARS-Cov-2 quantitative assay also aids in establishing the functional serum antibody titer as an objective relative measure of the strength of the immune response to SARS-Cov-2, irrespective of reported clinical illness or not, among significantly exposed or infected individuals. The quantitative assay provides a tool for the objective assessment of seroconversion among members of any defined group or community and the assessment of an attained level of herd immunity in a population. The quantitative assay will also allow monitoring the level of protective immunity among exposed individuals including those who receive vaccination when it becomes available.

Negative results do not preclude acute SARS-CoV-2 infection. If acute infection is suspected, direct testing for SARS-CoV-2 is necessary.

False positive results for The Mount Sinai SARS-CoV-2 assay may occur due to cross-reactivity from pre-existing antibodies or other possible causes.

The Mount Sinai SARS-CoV-2 assay is only for use under the Food and Drug Administration's Emergency Use Authorization.

### 2) Special Conditions for Use Statements:

For prescription use only

For in vitro diagnostic use only

For Emergency Use Authorization only

### 3) Instruments Used:

The Mount Sinai Quantitative SARS-CoV-2 assay is to be used with the instruments described in Example 7, supra, under C.

### 4) Overview of Manufacturing and Distribution:

The product and components are all developed at the Mount Sinai Laboratory (MSL), Center for Clinical Laboratories, a division of the Department of Pathology, Molecular and Cell Based Medicine, New York, NY.

5) Components Included with the Test
Recombinant full-length spike protein
   6) Components Required but Not Included with the Test
Flat-Bottom Immuno Nonsterile 96-Well Plates 4 HBX (Thermo Scientific #3855, or equivalent
Thermo Scientific™ (Waltham, MA) Nunc™ MicroWell™ 96-Well Microplates, Non-Treated, Cat #260895, or equivalent
Milk Powder, Fisher Scientific, Cat #NC0115668, or equivalent. Store at room temperature, stable until expiration date
PBS (1λ) (Gibco), ThermoFisher, Cat #100010023, or equivalent. Store at room temperature, stable 24 months from date of manufacture
Water for Injection (WFI) for Cell Culture (Gibco #A1287301 or equivalent)
Tween™ 20 (C58H114O26; MW: 1227.5), Fisher BioReagents™, Fisher Scientific, Cat #BP337-500, CAS #9005-64-5, or equivalent, store at room temperature, stable until expiration date
Phosphate Buffered Saline (10×), Fisher Scientific, Cat #MT-46013CM, or equivalent, store at room temperature, stable until manufacturers expiration date
Eppendorf (Hamburg, Germany) Repeater M4 pipettor; 10 mL Eppendorf tips (Cat #30089820), 5 mL Eppendorf tips (Cat #30089812) and 1 mL Eppendorf tips (Cat #30089790)
Micropipette tips o Fisher Scientific™ (Hampton, NH) Fisherbrand™ SureOne™ Micropoint Pipette Tips, Universal Fit, Non-Filtered 0.1-10 μL pipette tips (Cat. #02-707-420), or equivalent
   Fisher Scientific™ (Hampton, NH) Fisher Brand 1-200 μL pipette tips (Cat. #02-707-420), or equivalent
   USA Scientific, 200 μL TIPONE PIPETTE TIP STACKS, Cat #1111-0240, or equivalent
   Fisher Scientific™ (Hampton, NH) Fisher Brand 100-1250 μL pipette tips (Cat. #02707-403), or equivalent
Sterile, serological pipettes o 5 mL (Falcon #356543 or equivalent) o 10 mL (Falcon #357551 or equivalent) o 25 mL (Falcon #357535 or equivalent) o 50 mL (Falcon #356550 or equivalent)
Polypropylene sterile conical tubes o Fisher Scientific Centrifuge Tubes, 15 mL (17×120 mm), Molded with Graduations, Polypropylene with screw cap, Fisher Scientific, Cat #50-145-8829, or equivalent.
   Fisher Scientific Tubes, 50 mL (30×115 mm), Molded with Graduations, Polypropylene with screw cap, Fisher Scientific, Cat #50-145-8841, or equivalent.
Anti-Human IgG (Fab specific, in 0.01 M phosphate buffered saline, pH 7.4, containing 0.01% thimerosal)-Peroxidase antibody produced in goat, Sigma Aldrich® (St. Louis, MO), Cat #A-0293. Store at −20° C. freezer, stable until manufacturer's expiration date.
Hydrochloric Acid 3.0M (HCl; MW:36.4), Fisher Scientific, Cat #S25856, CAS #764701-0, or equivalent. Store at room temperature in flammable safety cabinet.
SIGMAFAST™ OPD (o-Phenylenediamine dihydrochloride), Sigma-Aldrich (St. Louis, MO), #P9187 or equivalent, storage temperature 2-8 C, stable until manufacturer's expiration date.
Precision pipette capable of delivering 0-20 μL, Eppendorf Research Plus or equivalent, (San Diego, CA) Cat #3120000810)
Precision pipette capable of delivering 0-2.5 μL, Eppendorf Research Plus or equivalent, (San Diego, CA) Cat #3120000011)
Precision pipette capable of delivering 0-200 μL, Eppendorf Research Plus or equivalent, (San Diego, CA) Cat #3123000055)

### B. Device Description and Test Principle

### 1) Product Overview/Test Principle:

The Mount Sinai Quantitative ELISA for SARS-Cov-2 IgG assay is designed to detect IgG class antibodies in human sera/plasma to corona virus spike antigen.
I. Purified recombinant Spike antigen is immobilized on a plastic matrix in a 96-well plate format and the antibodies present in diluted human serum/plasma are bound to the antigen. Levels of IgG antibodies are then determined using a horseradish peroxidase (HRP) labeled anti-human IgG (fab specific) secondary antibody produced in goat.
II. Horseradish peroxidase (HRP) conjugated goat anti-human IgG (Human Fab specific IgG) is added to the wells and the plate is incubated. The conjugate will react with anti-SARS-Cov-2 IgG bound to antigen and the wells are washed to remove unreacted conjugate
III. The microwells containing HRP conjugate are incubated with peroxidase substrate (Phenylenediamine) solution. Hydrolysis of the substrate by peroxidase produces a color change. After a period of time the reaction is stopped by adding 3 M Hydrochloric acid and the color intensity of the solution is measured by spectrophotometer with the wavelength set at 490 nm.
IV. Antibody levels are reported in an arbitrary unit value based upon a four parameter logistic curve generated from a serial dilution of a strong positive (high titer ≥2880 using our qualitative assay) patient serum pool that is included on each ELISA plate. This assay measures only the level of antibodies present in a given serum or plasma specimen.

### 2) Description of Test Steps:

### Description of Test Steps:

Full Length Spike Protein by ELISA
1. Coating ELISA plates
   Thaw the required number of vials of antigen (SARS-CoV-2 Spike protein) to coat 96-well microtiter ELISA plates at a concentration of 2 μg/mL. Once thawed, mix by gently vortexing vial before diluting in 1×PBS.
   Prepare approximately 6 mL for each plate to be coated.
   Coat plates with 50 μL of diluted protein per well using a multichannel pipettor and a reservoir. Lightly tap plates against surface to ensure protein is completely coated on the bottom of every well.
   Incubate at 4° C. overnight. Plates can be stored at 4° C. for up to 1 week.
   Always keep a cover plate on top of coated plates during all steps of the protocol.
2. Block ELISA plate
   Prepare at least 25 mL of blocking solution per plate.
   Blocking solution consists of PBS-T+3% non-fat milk powder (weight/volume).
   Using an automated plate washer, wash coated ELISA plates 3× with PBS-T.
   Add 200 μL blocking solution to all wells of the plates, incubate at room temperature for 1 hour, but do not exceed 4 hours, after completing the first plate.
3. Pre-diluting samples
   In a biological safety cabinet, set up microcentrifuge tubes to pre-dilute samples 1:5.
   Add 40 μL of 1×PBS to all tubes.
   Add 10 μL of controls and patient specimens to each tube containing 40 μL of 1×PBS, vortex to mix.
4. Standards preparation
   Standard curve is generated by serial dilutions of a high titer patient serum pool in 0.05% Tween-20 PBS. The dilutions ranges from 8 to 4883.
5. Controls preparation
   Control (Low, Medium and High Quality Control) sera are stable up to one month when refrigerated at 2-8° C.
6. Calibration and calibration verification procedures
   Not required for this procedure
7. Sample Preparation
   Specimens are diluted to 1:80 with 0.05% Tween-20 PBS, vortexed briefly, and stored at 4° C. until assayed.
8. Detailed Sample Preparation
   The plates were washed in Tween-20 Phosphate Buffered Saline and blocked with 100 μL per well of 0.3% Tween-20 PBS for 2 h at 4° C.
   After a series of three washes (subsequent washes were all with 0.05% Tween-20 PBS), 100 μL aliquots of serum diluted 1:80 with wash buffer were added to each well. A serial dilution (1:50 to 1:6400) of a strong positive serum pool was used to generate a standard curve on each individual plate. A buffer blank, three levels of control sera, and a negative for SARS-Cov-2 IgG specimen (that were previously known by our qualitative ELISA assay) were also included on each plate.
   Plates were incubated 2 h at room temperature, and then washed four times with wash buffer.
   A secondary antibody against human IgG fab specific conjugate (Horseradish peroxidase (HRP) was added to each well and incubated for 1 h at room temperature.
   After three washes the microwells containing HRP conjugate are incubated with peroxidase substrate (o-Phenylenediamine) solution.
   Hydrolysis of the substrate by peroxidase produces a color change. After a period of time the reaction is stopped by adding 3 M Hydrochloric acid and the color intensity of the solution is measured by spectrophotometer with the wavelength set at 490 nm.
   The absorbances were measured at 490 nm using a Biotek microplate reader. Antibody levels of the unknown samples were assigned a unit value based on the 8-point standard curve with a four-parameter curve fit.
   Unknown specimens with absorbance values above the standard curve were diluted further and re-assayed. Arbitrary unit values were expressed per mL of serum.
9. Recording of Data
   Biotek Gen 5 Analysis Software or by Graphpad Prism Software
10. Calculations

Optical densities at 490 nm were converted to arbitrary unit values per mL of serum using the Gen 5 Analysis Software.

### Control Materials

Three known Low, Medium and High titered sera and negative serum were included on each ELISA plate along with an eight-point standard curve. The four-parameter curve that is fitted to the standard curve values must have a correlation coefficient of >0.98 or the run is rejected. As with the unknowns, the values for the positive control standards must have a CV of less than 20% from the established value.

### C. Interpretation of Results

Assessment of The Mount Sinai SARS-CoV-2 assay results should be performed after the positive and negative controls have been examined and determined to be valid and acceptable. If the controls are not valid, the patient results cannot be interpreted. See Table 2, supra, for assay results and interpretation (preliminary). The quantitative result must be used in conjunction with immune status of the individual and should be further assessed by considering other factors, such as clinical status, follow-up testing, and associated risk factors.

### D. Performance Evaluation

### 1) Analytical Sensitivity and Specificity

a) Reactivity inclusivity:
   Although mutations in the SARS-CoV-2 genome have been identified as the virus has spread, no serologically unique strains have been described relative to the originally isolated virus (this research is exceptionally limited at present).
b) Cross-Reactivity:
   A study is currently underway to assess cross reactivity with the Mount Sinai IgG Test System using patient sera that was seropositive to different microorganisms and samples from patients diagnosed with certain disease conditions. This study is currently in process. See Table 4, supra.

### Precision/Reproducibility:

### Limit of Blank (LoB), Limit of Detection (LoD), and Limit of Quantitation (LoQ):

The limits of detection (LoD) was determined by running 13 replicates of the assay buffer (treated as patient specimen) on one assay plate. Two standard deviations were added to the mean OD reading (Limit of Blank), and this value was read using calibration curve to calculate the LoD. Limits of Quantification (LoQ) was calculated by adding 10 standard deviation to Limits of Detection. See Table 3, supra.

### Precision

Assay precision within day and between-day precision was evaluated using Low, Medium and High controls for quantitative ELISA. Precision was reported as the coefficient of variation (CV). Within-day precision was calculated by analyzing 10 replicates of negative, Low, Medium and High controls on one day. Between-day precision was calculated by analyzing negative, Low, Medium and High controls for 5 different days. The acceptance criterion for within-day and between-day precision (% CV) was ≤20% for each control tested. The between-run precision data was analyzed by EP Evaluator (South Burlington, VT, USA). See Table 5, supra, and FIGS. 15-17.

### Interfering Substances:

This study is currently in process. The following potentially interfering substances were evaluated in serum to establish their effect on the investigational device: cholesterol, hemoglobin. The concentration of analyte of each potential interfering substance was as follows:
Hemoglobin: 10 g/dL (low), 15 g/dL (high) and Intralipid: 300 mg/dL (low), 750 mg/dL (high)

Three clinical samples exhibiting differing reactivities with the Mount Sinai IgG Test System were tested for interference with above substances: a low QC specimen, and high QC specimen. All specimens exhibited a change of signal less than (TBD) when tested with each potential interferant.

Hook Effect: This study is currently in process.

### Dilutional Linearity

Acceptability criteria for dilution integrity experiments was +20% from the expected values. The linearity of the analytical measurement range (AMR) was evaluated by two patients specimens serum with various SARS-Cov-2 IgG antibody concentrations were diluted with Phosphate Buffered Saline serially (1:2, 1:4, 1:8, 1:16 and 1:32) to obtain values that cover the AMR and clinically reportable range (CRR). Acceptability criteria for dilution integrity experiments was +20% from the expected values. All two specimens fulfilled the acceptance criteria, showing good linearity in their respective ranges. See FIGS. 18A-18B.

### Sample Stability and Handling:

Three samples will be tested in duplicates for up to 7 days while stored at 2-8° C., and after repeated freeze/thaw cycles up to 3 cycles (−80° C.). Results will be compared to those obtained on same specimens (day zero, stored at 2-8° C. and −80° C. This testing is currently under way.

### 2) Clinical Agreement Study:

MSHS lab has tested 13657 patient COVID-19 specimens by FDA-approved EUA 200282 (Section 6.8.1) assay and created a biobank for future studies. From this repository, very high titer (>2880) specimens were pooled (to avoid inter-individual variability) and a reference calibration/standard serum was established. Negative control serum (from naïve patients) and low, medium and high-level control materials were prepared, in an identical fashion.

The quantitative ELISA methodology is identical to the qualitative FDA-approved EUA 200282 (Section 6.8.1) method that is currently in use at Mount Sinai. In brief, 96-well plates are coated with recombinant SARS-Cov-2 spike protein (2 ug/mL). Plates are incubated overnight at 2-8° C. and then washed, blocked and finally pre-diluted (1:80). Patient specimens, controls are added to respective assigned wells. A 10-point calibration curve is generated by serially diluting a reference calibration standard pool. Following incubation, the plate is washed three times with Wash Buffer. Horseradish peroxidase (HRP)-conjugated goat anti-human IgG, is added to all wells, then the plate is incubated and washed, followed by the addition of o-phenylenediamine dihydrocloride (OPD) substrate. Within 30 minutes the reaction is suspended by the addition of 3 M HCl Stop Solution. Optical density (OD) readings for each well are obtained at 490 nm.

### Data Analysis:

Method for calculation of Arbitrary Units for reference standard serum: A 10 point calibration curve is generated by serial dilution, and optical density (OD 490) values are plotted against the reciprocal of the dilution factor on a 4-Parameter Logistic (4PL) curve. The four parameters of the 4PL model are the lower asymptote of the sigmoidal curve (a), a curvature parameter related to the slope of the curve (b), a parameter related to the dilution at the midpoint of the curve (c), and the upper asymptote of the curve (d). The constants are calculated using GraphPad Prism® version 8 (GraphPad Software, La Jolla, CA) and an IC50 value is assigned as an arbitrary baseline antibody concentration (AU/mL) for the reference standard (FIG. 12). Thereafter, Arbitrary Units are used to create a standard curve (X axis=AU/mL; Y axis=OD 490 nm) using the 4PL curve (FIG. 13), The measured OD value of unknown specimens is used to calculate concentration by the following equation:

$$x = {c ( \frac{a - x}{y - d} )}^{( 1 / b )}$$

x=AU/mL, y=OD490, a=lower asymptote, b=hill slope, c=IC50, d=upper asymptote of 4PL curve.

See Table 1, supra, for the constants were calculated using GraphPad Prism for the reference calibration curve.

Preliminary Data

I. Accuracy of the Assay:

a. Sixty patient specimens were analyzed by our standard procedure and the raw data was analyzed by GraphPad Prism and Biotek Gen 5 software (Winooski, VT). Both statistical programs used 4 PL calibration curves to determine unknowns. The acceptable criteria is +/−20% and MSHS quantitative assay values are truly agreeable on both statistical packages (FIG. 14).

### Approved/Cleared Alternative Products

Currently no methods for the qualitative detection of SARS-CoV-2 IgM or IgG antibodies have been approved or cleared by FDA.

### E. Record Keeping and Reporting Information to FDA:

The laboratory will track adverse events and report to FDA under 21 CFR Part 803. A website is available to report on adverse events, and this website is referenced in the Fact Sheet for Health Care providers. The laboratory will maintain will information on the performance of the test, and report to FDA any suspected change in performance of which they become aware. The laboratory will maintain records associated with this EUA and ensure these records are maintained until notified by FDA. Such records will be made available to FDA for inspection upon request.

### 6.8.1 Qualitative ELISA

### A. Proposed Intended Use

### 1) Intended Use:

The COVID-19 ELISA IgG Antibody Test is a direct Enzyme-Linked Immunosorbent Assay (ELISA) for the qualitative detection of IgG antibodies against recombinant Receptor Binding Domain of SARS-CoV-2 in serum followed by confirmatory ELISA of positive specimen against full length SARS-CoV-2 Spike protein in serum. The COVID-19 ELISA IgG Antibody Test detects IgG antibodies as indicative of an immune response to SARS-CoV-2 in patients suspected of previous SARS-CoV-2 infection, or for the detection of IgG seroconversion in patients following known recent SARS-CoV-2 infection. The COVID-19 ELISA IgG Antibody Test should not be used for the diagnosis of patients with acute COVID-19 infection. The COVID-19 ELISA IgG Antibody Test may also be used to identify positive specimens with an antibody titer up to a dilution of 1:2880 for the detection of individuals with higher antibody titers. Testing is limited to the Mount Sinai Laboratory (MSL), Center for Clinical Laboratories), Department of Pathology, Molecular, and Cell-Based Medicine, New York, NY-10029, certified under the Clinical Laboratory Improvement Amendments of 1988 (CLIA), 42 U.S.C. § 263a, to perform high complexity tests.

IgG antibodies to SARS-CoV-2 generally become detectable beginning 10-14 days following infection but may occur later. The presence of IgG antibodies following previously negative testing defines IgG antibody seroconversion following SARS-CoV-2 infection.

Laboratories within the United States and its territories are required to report all positive results to the appropriate public health authorities.

Negative results do not preclude acute SARS-CoV-2 infection and should not be used as the basis for patient management decisions. IgG antibodies as IgG antibodies may not be present for more than two weeks following infection, and patients may remain infectious during acute infection even if IgG antibody is present. The sensitivity of the COVID-19 ELISA Antibody IgG Test early after infection in unknown.

False positive results for IgG antibodies may occur due to cross-reactivity from pre-existing antibodies or other possible causes. Prevalence of SARS-CoV-2 infection in the area where testing has occurred should be considered when interpreting positive test results.

At this time, it is unknown for how IgG antibodies may persist following infection.

2) Instruments Used with Test:
BioTek (Winooski, VT) Energy Neo 2 Multi-Mode Reader coupled with BioTek (Winooski, VT) Biostack Neo
BioTek (Winooski, VT) EL406 Washer Dispenser coupled with BioTek (Winooski, VT) Biostack 4 Microplate Stacker
Labconco™ Purifier™ Logic™+ Class II A2 Biosafety Cabinets, Fisher Scientific, Cat #30-248-1001, or equivalent

### B. Description and Test Principle

### 1) Product Overview/Test Principle:

The COVID-19 Elisa IgG Antibody Test has been developed for the qualitative detection of human SARS-CoV-2 IgG antibody and measurement of circulating antibody in serum via direct Enzyme-Linked ImmunoSorbent Assay (ELISA). Assay controls and patient serum samples are diluted 1:50 and added to Thermo Scientific Immulon™ 96-wells microtiter plate that was coated with SARS-CoV-2 recombinant Receptor Binding Domain protein (RBD). The coated RBD protein combine with patient's SARS-CoV-2 IgG antibodies. The subsequent addition of a secondary anti-human IgG (Fab specific) HRP labeled antibody creates a specific complex of antigen-antibody bound to the plate surface. The binding reaction is then enhanced visually with Sigma-Aldrich SIGMAFAST™ OPD (o-Phenylenediamine dihydrochloride) substrate generating a yellow color for positive specimens. After application of the stop solution (3M Hydrochloric acid), the color changes to orange and optical density is monitored at 490 nm. The depth of color is relative with the content of the SARS-CoV-2 IgG antibodies. When the value of color is greater than the cut-off value (OD490=0.15), the specimens are reported as positive screen. Positive screen specimens are serially diluted for assessment of total circulating antibody titer (80×, 160×, 320×, 960×, 2800×) using SARS-CoV-2 Spike protein coated Thermo Scientific Immulon™ 96-wells microtiter plate. There is a stronger reaction against the full-length Spike protein than against the RBD, likely reflecting the higher numbers of epitopes found on the much larger Spike protein.

### 2) Description of Test Steps:

a. Overview of the ELISA assay:

### PART A: Screening by Receptor Binding Domain Protein

SARS-CoV-2 antigen coated Microplate
Heat inactivation of samples
Block ELISA Plate
Pre-Diluting Samples
Dilution Plate Set-Up
Transfer serum dilution
Addition of Secondary Antibody
Plate development and reading

### PART B: Spike Titration by ELISA

Coating ELISA plates
Block ELISA plate
Pre-Diluting samples
Serial Dilution
Addition of Secondary Antibody
Plate development and reading

### Detailed Assay Procedure:

### PART A: Screening by Receptor Binding Domain (RBD) Protein by ELISA

### 1. SARS-CoV-2 Antigen Coated Microtiter Plate

Thaw the required number of vials of antigen (SARS-CoV-2 RBD) to coat 96-well microtiter ELISA plates at a concentration of 2 ug/mL. Once thawed, mix by gently vortexing vial before diluting in 1×PBS.
Prepare approximately 6 mL for each plate to be coated.
Coat plates with 50 μL of diluted protein per well using a multichannel pipettor and a reservoir. Lightly tap plates against surface to ensure protein is completely coated on the bottom of every well.
Incubate at 4° C. overnight. Plates can be stored at 4° C. for up to 1 week.
Always keep a cover plate on top of coated plates during all steps of the protocol.

### 2. Heat Inactivation of Samples

Set the water bath to 56° C. Once temperature is reached, place the specimens in and start the timer for 1 hour.
Remove samples when the timer goes off. Do not leave samples at 56° C. for longer than 1 hour. Specimens are stable for 15 days when refrigerated at 4° C. Samples frozen at −80° C. have remained stable for up to 6 months.

### 3. Block ELISA Plate

Prepare at least 25 mL of blocking solution per plate.
Blocking solution consists of PBS-T+3% non-fat milk powder (weight/volume).
Using an automated plate washer, wash coated ELISA plates 3× with PBS-T.
Add 200 μL blocking solution to all wells of the plates, incubate at room temperature for 1 hour, but do not exceed 4 hours, after completing the first plate. Note: This step (and wherever a plate washer is needed below) can also be performed by washing plates with a multichannel pipette by hand if no plate washer is available.

### 4. Pre-Diluting Samples

In a biological safety cabinet, set up microcentrifuge tubes to pre-dilute samples 1:5.
Add 40 μL of 1×PBS to all tubes.
Add 10 μL of controls and patient specimens to each tube containing 40 μL of 1×PBS, vortex to mix.

### 5. Dilution Plate Set-Up

Prepare at least 25 mL of PBS-T+1% non-fat milk powder (weight/volume). This will also be used to dilute the secondary antibody.
Prepare 1 dilution plate (separate flat bottomed 95-wells cell culture plate) per antigen coated plate.
Add 180 μL of PBS-T containing 1% non-fat milk to all wells of the dilution plate.
Leaving columns 1 and 12 as Blanks, add 20 μL of pre-diluted controls and patient specimens into the designated wells. This results in a final dilution of 1:50.
Continue until all samples and controls have been added to designated wells. See reference plate layout in FIG. 2.

### 6. Transfer Dilution Plate to ELISA Plate

After blocking incubation, throw off the blocking solution. Tap the plates dry on a tissue, e.g., Kimwipe.
Using a multichannel pipettor, pipette up and down 4-6 times in the first row of dilution plate to mix.
Transfer 100 μL to the corresponding rows in the ELISA plate. Change tips and continue to transfer second row to the ELISA plate.
Incubate plates at room temperature for 2 hours as soon as all the rows have been transferred to the first ELISA plate. Do not exceed 4 hours.

### 7. Secondary Antibody

After 2 hours, wash the plates 3× with PBS-T using the automated plate washer.
Dilute anti-human IgG (Fab specific) HRP labeled secondary antibody 1:3000 in PBS-T containing 1% non-fat milk. Prepare at least 6 mL per plate.
Add 50 μL to all wells of the plate using a multichannel pipettor. Be sure to avoid touching the tips of the pipette to the walls of the well to avoid carry over and high background signals.
Incubate at room temperature for 1 hour as soon as the secondary antibody has been added to the first plate.

### 8. Plate Development and Reading

After 1 hour, wash plates 3× with PBS-T using an automated plate washer.
Prepare SigmaFast OPD solution and calculate amount needed. One set of tablets (1 gold+1 silver tablet) dissolved in 20 mL WFI for Cell Culture can be used for 2 plates. If necessary, multiple sets of substrate can be prepared in the same container.
Fully dissolve 1 gold tablet. Do not add silver tablet to solution until ready to start adding to the plates (needs to be prepared fresh right before use).
Add 100 μL to all wells of the plate. Begin timer for 10 minutes as soon as SigmaFast OPD solution has been added to the first row on the first plate. Do not exceed 10 minutes of developing before stopping the reaction.
To stop the reaction after exactly 10 minutes, add 50 μL of 3M HCl to all wells.
Read ELISA plates in plate reader at an absorbance of 490 nm (immediately after adding HCl) and record data.
Samples that exceed certain OD490 cutoff value (OD490=0.15) are assigned presumptive positive and will be tested by a confirmatory ELISA using full-length Spike protein.

### PART B: Titration by Full Length Spike Protein by ELISA

### 1. Coating ELISA Plates

Thaw the required number of vials of antigen (SARS-CoV-2 Spike protein) to coat 96-well microtiter ELISA plates at a concentration of 2 ug/mL. Once thawed, mix by gently vortexing vial before diluting in 1×PBS.
Prepare approximately 6 mL for each plate to be coated.
Coat plates with 50 μL of diluted protein per well using a multichannel pipettor and a reservoir. Lightly tap plates against surface to ensure protein is completely coated on the bottom of every well.
Incubate at 4° C. overnight. Plates can be stored at 4° C. for up to 1 week.
Always keep a cover plate on top of coated plates during all steps of the protocol.

### 2. Block ELISA Plate

Prepare at least 25 mL of blocking solution per plate
Blocking solution consists of PBS-T+3% non-fat milk powder (weight/volume).
Using an automated plate washer, wash coated ELISA plates 3× with PBS-T.
Add 200 μL blocking solution to all wells of the plates, incubate at room temperature for 1 hour, but do not exceed 4 hours, after completing the first plate.

### 3. Pre-Diluting Samples

In a biological safety cabinet, set up microcentrifuge tubes to pre-dilute samples 1:5.
Add 40 μL of 1×PBS to all tubes.
Add 10 μL of controls and patient specimens to each tube containing 40 μL of 1×PBS, vortex to mix.

### 4. Serial Dilution Plate Set Up

Calculate and prepare at least 25 mL of PBS-T+1% non-fat milk powder (weight/volume) per plate. This will also be used to dilute the secondary antibody.
Prepare 1 dilution plate (separate flat bottomed 95-wells cell culture plate) per antigen coated plate.
Using a multichannel pipettor, dispense 285 μL of PBS-T containing 1% milk to columns 2 and 7 of the plate.
Using a multichannel pipettor, dispense 150 μL of PBS-T containing 1% milk to columns 3, 4, 8 and 9 of the plate.
Using a multichannel pipettor, dispense 140 μL of PBS-T containing 1% milk to columns 1, 5, 6, 10, 11 and 12 of the plate.
Leaving columns 1 and 12 as Blanks, add 19 μL of 1:5 pre-diluted controls and patient specimens (final dilution 1:80 on the plate) to the designated wells in columns 2 and 7.
With the multichannel pipettor, pipette up and down 4-6 times in column 2/7 to mix. Transfer 150 μL (2-fold dilution) from column 2/7 to column 3/8, respectively, and pipette up and down 4-6 times to mix.
Transfer 150 μL (2-fold dilution) from column 3/8 to column 4/9, respectively, and pipette up and down 4-6 times to mix.
Transfer 70 μL (3-fold dilution) from column 4/9 to column 5/10, respectively, and pipette up and down 4-6 times to mix.
Transfer 70 μL (3-fold dilution) from column 5/10 to column 6/11, respectively, and pipette up and down 4-6 times to mix.
See reference plate layout below.
   3) Control Material(s) to be Used with the COVID-19 ELISA IgG Antibody Test

To assure validity of the specimen results each 96 well plate includes both negative and positive controls. The average value of the absorbance (optical density of 490 nm) of the negative control must be less than 0.15, and the absorbance (optical density of 490 nm) of the positive control must be ≥than 0.15. The negative and positive controls are run in duplicate, and controls and samples are assayed at the same time for every 96 well ELISA plate.

### Negative Control:

Negative control material does not contain any serum from patients with SARS-CoV-2 coronavirus infection. Negative Controls are prepared using remnant pooled serum that was tested negative for SARS-CoV-2 antibodies by direct ELISA. The negative control absorbance at 490 nm must be less than 0.15.

### Positive Control:

Positive control material contains serum from patients with new type of SARS-CoV-2 coronavirus infection. Positive Controls are prepared using remnant pooled serum that was tested positive for SARS-CoV-2 antibodies by direct ELISA. The positive control absorbance at 490 nm must be >0.15.

### 4) Assay Results and Interpretation

Controls Performed with the Assay:

QC serum is human serum with known acceptable results for RBD screen and Titer. To assure the validity of the results each assay must include both negative and positive controls. The average value of the absorbance of the negative control is less than 0.1, and the absorbance of the positive control is greater than 0.15.
Pooled Serum Negative and Positive controls aliquots are stable for 6 months when frozen at −20° C. or lower
Pooled Serum Negative and Positive controls are stable for 1 week when refrigerated

Note: Controls of human origin have tested negative for HBsAg, anti-HCV, anti-HIV-1 and anti-HIV-2. All reagents should be treated as being a potential infection hazard and should be handled with care.

### Test Result and Interpretation:

The cut-off for screening by receptor binding domain protein (RBD) and titer analysis by spike protein is >0.15 at an absorbance of 490 nm.

Negative result: A negative antibody screen result indicates that a serum dilution of 1/50 showed no specific antibodies to SARS-CoV-2 virus in RBD ELISA.

Positive result: The specimen is classified (reported) as POSITIVE for SARS-CoV-2 IgG antibody when both the RBD screen and Spike protein testing are positive. A positive antibody screen result indicates antibodies to SARS-CoV-2 virus for antibodies at a dilution greater than 1/50. Subsequent testing by the spike protein is considered positive if antibody is detected at a titer of 1:320 or greater. The test result is considered overall positive only if positive results are seen for the RBD screen ELISA and subsequent spike protein ELISA. See Table 6 below.

TABLE 6
Interpretation of Results

| Test | Result | Final Result | |
|---|---|---|---|
| RBD screen | NEGATIVE | NEGATIVE | No further testing |
| (Part A) | POSITIVE | | Presumptive positive; test |
| | | | by spike protein |
| Spike protein | NEGATIVE | NEGATIVE | |
| (Part B) | POSITIVE | POSITIVE | Confirmed positive for IgG |
| | | | antibody |

### C. Product Manufacturing

The COVID-19 ELISA IgG Antibody Test has been validated using only the components referenced in this submission and shall not be changed without prior concurrence from the FDA.

### 1) Overview of Manufacturing and Distribution:

The product and components are all developed at the Mount Sinai Laboratory (MSL), Center for Clinical Laboratories, a division of the Department of Pathology, Molecular, and Cell-Based Medicine, New York, NY (CLIA #33D1051889).

Components Included with the Test
Recombinant RBD protein
Recombinant full-length Spike protein
   2) Components Required but not Included with the Test
Flat-Bottom Immuno Nonsterile 96-Well Plates 4 HBX (Thermo Scientific #3855, or equivalent
Thermo Scientific™ (Waltham, MA) Nunc™ MicroWell™ 96-Well Microplates, Non-Treated, Cat #260895, or equivalent
Milk Powder, Fisher Scientific, Cat #NC0115668, or equivalent. Store at room temperature, stable until expiration date
PBS (1×) (Gibco), ThermoFisher, Cat #100010023, or equivalent. Store at room temperature, stable 24 months from date of manufacture
Water for Injection (WFI) for Cell Culture (Gibco #A1287301 or equivalent)
Tween™ 20 (C58H114026; MW: 1227.5), Fisher BioReagents™, Fisher Scientific, Cat #BP337-500, CAS #9005-64-5, or equivalent, store at room temperature, stable until expiration date.
Phosphate Buffered Saline (10×), Fisher Scientific, Cat #MT-46013CM, or equivalent, store at room temperature, stable until manufacturers expiration date.
Eppendorf (Hamburg, Germany) Repeater M4 pipettor; 10 mL Eppendorf tips (Cat #30089820), 5 mL Eppendorf tips (Cat #30089812) and 1 mL Eppendorf tips (Cat #30089790)
Micropipette tips
   Fisher Scientific™ (Hampton, NH) Fisherbrand™ SureOne™ Micropoint Pipette Tips, Universal Fit, Non-Filtered 0.1-10 μL pipette tips (Cat. #02-707-420), or equivalent
   Fisher Scientific™ (Hampton, NH) Fisher Brand 1-200 μL pipette tips (Cat. #02-707-420), or equivalent
   USA Scientific, 200 μL TIPONE PIPETTE TIP STACKS, Cat #1111-0240, or equivalent
   Fisher Scientific™ (Hampton, NH) Fisher Brand 100-1250 μL pipette tips (Cat. #02-707-403), or equivalent
Sterile, serological pipettes
   5 mL (Falcon #356543 or equivalent)
   10 mL (Falcon #357551 or equivalent)
   25 mL (Falcon #357535 or equivalent)
   50 mL (Falcon #356550 or equivalent)
Polypropylene sterile conical tubes
   Fisher Scientific Centrifuge Tubes, 15 mL (17×120 mm), Molded with Graduations, Polypropylene with screw cap, Fisher Scientific, Cat #50-145-8829, or equivalent.
   Fisher Scientific Tubes, 50 mL (30×115 mm), Molded with Graduations, Polypropylene with screw cap, Fisher Scientific, Cat #50-145-8841, or equivalent.
Anti-Human IgG (Fab specific, in 0.01 M phosphate buffered saline, pH 7.4, containing 0.01% thimerosal)-Peroxidase antibody produced in goat, Sigma Aldrich® (St. Louis, MO), Cat #A-0293. Store at −20° C. freezer, stable until manufacturer's expiration date.
Hydrochloric Acid 3.0M (HCl; MW:36.4), Fisher Scientific, Cat #S25856, CAS #7647-01-0, or equivalent. Store at room temperature in flammable safety cabinet.
SIGMAFAST™ OPD (o-Phenylenediamine dihydrochloride), Sigma-Aldrich (St. Louis, MO), #P9187 or equivalent, storage temperature 2-8 C, stable until manufacturer's expiration date.
Precision pipette capable of delivering 0-20 μL, Eppendorf Research Plus or equivalent, (San Diego, CA) Cat #3120000810)
Precision pipette capable of delivering 0-2.5 μL, Eppendorf Research Plus or equivalent, (San Diego, CA) Cat #3120000011)
Precision pipette capable of delivering 0-200 μL, Eppendorf Research Plus or equivalent, (San Diego, CA) Cat #3123000055)

### 3) Testing Capabilities

Qualitative Screening Assay: A total of 380-760 samples can be processed at one time through parts A and B described above by use of 96-well microtiter ELISA plates. The entire process takes 12-24 hours from sample preparation to final reading of test results. The Mount Sinai Laboratory anticipates performing approximately 500-2500 patient samples per day.

### D. Performance Evaluation

### 1) Analytical Sensitivity:

There is no standard reference SARS-CoV-2 antigen material available; accordingly, absolute analytical sensitivity cannot be calculated.

### 2) Analytical Specificity:

### Reactivity Inclusivity

Although mutations in the SARS-CoV-2 genome have been identified as the virus has spread, no serologically unique strains have been described relative to the originally isolated virus (this research is limited at present).

### Analytical Specificity and Microbial Interference:

Cross-reactivity of non SARS-CoV-2 specific Ab against RBD protein was examined using sera with known antibodies against confirmed past infections or elevated gamma globulins described below. No interference was observed. Negative screen results using RBD protein have been shown to be negative when tested at 1:80 dilution using Spike protein as antigen. See Table 7.

TABLE 7

| | | COVID-19 ELISA IgG Antibody |
|---|---|---|
| Antibody positive sera | N | Test Result |
| Varicella | 5 | NEGATIVE |
| Influenza | 4 | NEGATIVE |
| HSV | 7 | NEGATIVE |
| Rubella | 5 | NEGATIVE |
| Hepatitis | 4 | Not Detected |
| HIV | 4 | NEGATIVE |
| Elevated IgG | 5 | NEGATIVE |
| Elevated IgM | 4 | NEGATIVE |
| CMV | 2 | NEGATIVE |

### 3) Precision

Assay precision within day and between-day precision was evaluated using negative and positive controls for both the RBD and spike protein confirmation ELISAs. Precision was reported as the coefficient of variation (CV). Within day precision was calculated by analyzing 10 replicates of negative and positive controls on one day. Between day precision was calculated by analyzing negative and positive controls for 21 different days. The acceptance criterion for within day and between day precision (% CV) was ≤20% for each control tested. The cut-off for positive control was >0.15 at OD490 nm and for the negative control was <0.15.

The precision for within day and between day precision measurements for both the RBD screen and spike protein confirmation met the specified acceptance criteria. For the RBD screen, precision testing was performed over 22 days with a single run per day; for the spike protein within-run testing was assessed using 10 replicates per day and between-run testing with a single run over 22 days. For spike protein, 5 dilutions of the positive and negative controls were tested in a similarly to the RBD protein.

### 4) Class Specificity:

Not applicable.

### 5) High-Dose Hook Effect

The hook effect is based on the saturation of antigen with antibody. The COVID-19 ELISA IgG Antibody Test assay hook effect was studied by diluting every specimen/control by 1:50 and including a high titered positive control specimen in every 96 well plate. No Hook effect was observed by either RBD or Spike protein at a reciprocal of 4050 and 8100 dilutions.

### 6) Matrix Equivalency

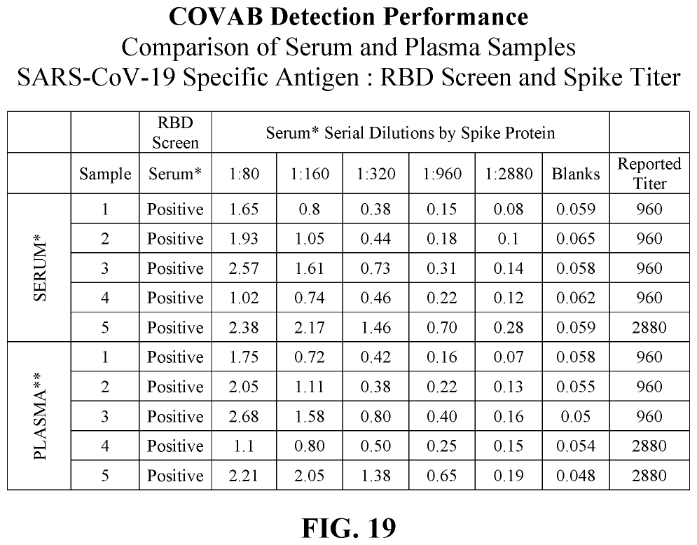

Matrix was evaluated by a limited study comparing paired plasma and serum specimens from five SARS-CoV-2 antibody titer positive patients. Results are shown in FIG. 19 for the RBD screen and titers to 1:2880.

### 7) Specimen Stability

The stability of one negative and one positive control was evaluated after 7 days storage under refrigeration. The screen was performed with 1:50 dilution on the RBD protein; serial dilutions were used for testing using the Spike protein as per assay protocol. Specimens were refrigerated at (2-8° C.) and tested at Days 0 and at Day 7, on 3 replicates. For Spike protein tested specimen, 5 separate dilutions up to 1:2880 were used. Accuracy acceptable criteria was set at a difference of +20%.

Results shown in the table below met acceptance criteria, with mean extraction efficiency at day 7 of 95.7% for the negative control and 88% for the positive control. For the spike protein, mean efficiency differed by titer, as shown in Table 8.

TABLE 8
Mean % Extraction Efficiency

| Dilution | Negative QC by RBD protein | Titer | Positive QC by S-protein |
|---|---|---|---|
| 1:50 | 95.7 | 80 | 87.7 |
| | | 160 | 89.4 |
| | | 320 | 88.9 |
| | | 960 | 82.3 |
| | | 2880 | 78.3 |

### 8) Fresh/Frozen Testing

One negative control and one positive control serum sample with elevated levels of SARS-CoV-2 antibodies was frozen at −20 C degrees. For both the RBD and spike proteins, the sample was tested in three replicates at time 0 and at 72 hours; the spike protein was also tested at 5 separate dilutions up to 1:2880. For the RBD protein extraction efficiency was 103.8% (negative) and for Spike protein ranged from 87.0-95.5% (positive control). For the spike protein, mean efficiency differed by titer, as shown in the table below; however, the sponsor notes that samples are not frozen for routine clinical testing. See Table 9.

TABLE 9
Mean % Extraction Efficiency

| Dilution | Negative QC | Titer | Positive QC |
|---|---|---|---|
| 1:50 | 103.8 | 80 | 95.5 |
| | | 160 | 93.8 |
| | | 320 | 89.0 |
| | | 960 | 85.4 |
| | | 2880 | 87.0 |

### 9) Clinical Evaluation:

Test performance was assessed for samples obtained from patients with clinical findings suggestive of COVID-19 infection; patients were subsequently identified to include both PCR+ or PCR− negative patients. Initially 58 serum samples were collected between 7 and 14 days after onset of symptoms. Fifty five presumed true negative samples that had been collected prior to the onset of the emergence of COVID-19 pandemic were also randomized before sending to the clinical laboratory for testing. The COVID-19 Elisa IgG Antibody Test on samples at or before 14 days were positive in 67% of cases. PCR status of patients was subsequently confirmed, and all PCR+ patients retested at 21 days and results reanalyzed for positive and negative agreement. PCR+ samples were 92.5% positive and all negative samples returned a negative result for 100% specificity. Results are shown in the Table 10:

TABLE 10

SARS-CoV-2 PCR Status

| | | Positive | Negative | Total |
|---|---|---|---|---|
| COVID-19 | Positive | 37 | 0 | 37 |
| Elisa | Negative | 3 | 74 | 77 |
| IgGAntibody | Total | 40 | 74 | 114 |
| Test | | | | |

Sensitivity: 0.925 (95% CI: 0.785239, 0.980428)

Specificity: 1.00 (95% CI: 0.93851, 1)

For all positive samples, the sponsor has identified samples with titers to 1:2880 by dilution. Samples with titers to 1:2880 represent approximately 44% of all samples positive above the threshold titer of 1:320 for the spike protein confirmatory assay.

### E. Unmet Need Addressed by the Product

Reports of undiagnosed pneumonia cases linked to a seafood market in Wuhan City, Hubei Province, China first appeared in early December 2019. The cause of the respiratory illness was determined in early January 2020 to be a novel (new) coronavirus (originally named “2019-nCoV”—later renamed to SARS-CoV-2), that has continued to expand both within China and Internationally. Cases of COVID-19 have now been identified in over 60 international locations. There also are reports of human to human transmission through close contact with an individual confirmed to be ill with COVID-19, in the United States and globally.

On Jan. 30, 2020, the International Health Regulations Emergency Committee of the World Health Organization declared the outbreak a “public health emergency of international concern” (PHEIC). On Jan. 31, 2020, Health and Human Services Secretary Alex M. Azar II declared a public health emergency (PHE) for the United States to aid the nation's healthcare community in responding to 2019-nCoV. Also, on January 31, the President of the United States signed a presidential “Proclamation on Suspension of Entry as Immigrants and Nonimmigrants of Persons who Pose a Risk of Transmitting 2019 Novel Coronavirus”.

Most patients with confirmed 2019-nCoV infection appear to develop fever and/or symptoms of acute respiratory illness (e.g., cough, difficulty breathing). However, limited information is currently available to characterize the full spectrum of clinical illness associated with 2019-nCoV infection. Signs and symptoms may appear any time from 2 to 14 days after exposure to SARS-CoV-2. Based on preliminary data, the median incubation period is approximately 4 days.

The WHO characterized it as a pandemic on Mar. 11, 2020. The WHO COVID-19 Situation report #52 (Mar. 12, 2020) reports a total of 125,048 globally confirmed cases, with 44,067 now confirmed outside of China in 117 countries, with 987 total cases in the United States. Laboratories in the United States are in need of diagnostic tools for use in the COVID-19 emergency for the identification of antibodies to SARS-CoV-2 to assess prior infection.

On Feb. 4, 2020, pursuant to section 564(b)(1)(C) of the Act, the Secretary of the Department of Health and Human Services (HHS) determined that there is a public health emergency that has a significant potential to affect national security or the health and security of United States citizens living abroad, and that involves the COVID-19. Pursuant to section 564 of the Act, and on the basis of such determination, the Secretary of HHS then declared that circumstances exist justifying the authorization of the emergency use of in vitro diagnostics for detection and/or diagnosis of COVID-19 subject to the terms of any authorization issued under section 564(a) of the Act.

The COVID-19 ELISA IgG Antibody Test can augment important clinical and laboratory evidence for individuals who meet clinical and/or epidemiological criteria for SARS-CoV-2 IgG or IgM testing.

Under emergency use authorization (EUA), the Mount Sinai Laboratory intends to perform the COVID-19 ELISA IgG Antibody Test for the detection of total antibody as indicative of an immune response to SARS-CoV-2 infection in patients suspected of SARS-CoV-2 infection, or for the detection of seroconversion in following known recent SARS-CoV-2 infection. This EUA request for the COVID-19 ELISA IgG Antibody Test is intended to expand domestic readiness within the United States and its territories by expanding diagnostic testing capabilities for COVID-19 during public health emergency.

FDA consulted with subject matter experts within HHS on the public health needs for diagnostic devices to detect SARS-CoV-2 IgG in response to infection. It is FDA's conclusion that there currently exists a public health need for such devices, i.e., that there is no adequate, approved (cleared), and available alternative to the COVID-19 ELISA IgG Antibody Test for qualitative detection of IgG antibodies to SARS-CoV-2 and for identifying samples with titers up to 1:2880 during the public health emergency.

### F. Approved/Cleared Alternative Products

Currently no methods for the qualitative detection of SARS-CoV-2 IgG antibody have been approved or cleared by FDA.

### G. Risks and Benefits:

### Risks

The COVID-19 ELISA IgG Antibody Test has been designed to minimize the likelihood of inaccurate test results, both false positive and false negative results, and the risk of misinterpretation of results. The well-recognized delay in the appearance of IgG antibodies following infection may yield a false negative result with use of the COVID-19 ELISA IgG Antibody Test if patients are tested prior to 14-21 days after infection. Positive results approximately may suggest that a patient is immune to SARS-CoV-2 reinfection, although it has not been established whether an IgG antibody response to confers immunity to reinfection, potentially contributing to SARS-CoV-2 re-exposure. In addition, high performance observed in this study may be due to unrecognized biases in the selection of patients for testing or other factors.

### False Positive Results:

A false positive result in the context of the current public health emergency could lead to misallocation of resources used for surveillance and prevention.
A false positive could be wrongly interpreted as conveying immunity to infection, leading to increased risk of infection if there is concomitant increased exposure to infected patients.

### False Negative Result:

A false negative result may be misinterpreted as ruling out previous SARS-CoV-2 infection, with a possible restriction of activities.
Testing during the period of acute infection may be mistakenly interpreted as indicating that a patient is not infected.

### True Positive or False Positive Result:

A positive result during acute infection may be misinterpreted as indicative of the patient being non-infectious, with the potential for infecting non-immune contacts.

### False High Titer Result:

Patients may be incorrectly considered for plasma donations intended for therapeutic use.

### Benefits

The primary benefit of distributing the COVID-19 ELISA IgG Antibody Test for clinical use is that it provides a means to identify patients who have IgG antibodies to SARS-CoV-2, indicating previous infection. The test also identifies patients with antibody titers above 1:2880 dilution, and serial sampling can document patient seroconversion from an antibody negative to an antibody positive status.
True positive test results provide support for the diagnosis of previous SARS-CoV-2 infection.
Results can be used identify higher titer patients that may be considered for plasma donation.

### Risk Assessment

The risks posed by use of the COVID-19 ELISA IgG Antibody Test are mitigated by:
Negative results do not preclude SARS-CoV-2 infection. The FACT sheets for health care practitioners and patients emphasizes that the COVID-19 ELISA IgG Antibody Test should not be used for the diagnosis of acute SARS-CoV-2 infection, and that samples should not be obtained in the first two weeks following infection unless used to document seroconversion.
Healthcare provider and patient fact sheets, which inform users that the test risk of patient harm during sample collection is minimal.
Performance at a single laboratory eliminates interlaboratory variability in test performance and reduces errors due to variation in reagents and performing the assay procedure.

### Risk-Benefit Assessment

Based on these factors, the potential benefits from the use of the COVID-19

ELISA IgG Antibody Test are expected to outweigh the risks.

### H. Approved/Cleared Alternative Products

Currently no methods for the detection of the COVID-19 have been approved/cleared by FDA.

### I. Fact Sheet for Healthcare Providers and Patients:

Appendix A COVID-19 ELISA IgG Antibody Test Fact Sheet for Healthcare Providers
Appendix B COVID-19 ELISA IgG Antibody Test Fact Sheet for Patients

### J. Instructions for Use/Proposed Labeling/Package Insert:

We have all the necessary scientific data for the test and an adequate description of procedures to run the test to conclude the criteria for issuance of an EUA are met.

### K. Record Keeping and Reporting Information to FDA:

The Mount Sinai Laboratory will track adverse events and report to FDA under 21 CFR Part 803. Each report of an adverse event will be processed the Mount Sinai Laboratory internal processes, and Medical Device Reports will be filed with the FDA as required. Through a process of inventory control, the Mount Sinai Laboratory will also maintain records of test volume. The Mount Sinai Laboratory will collect information on the performance of the test, and report to FDA any suspected occurrence of false positive or false negative results of which the Mount Sinai Laboratory becomes aware. The Mount Sinai Laboratory will maintain records associated with this EUA and ensure these records are maintained until notified by FDA. Such records will be made available to FDA for inspection upon request.

Final Conclusions for Interactive Review—It is FDA's conclusion that there currently exists a public health need for such a device, i.e., that there is no adequate, approved (cleared), and available alternative to the Mount Sinai Laboratory COVID-19 IgG ELISA Antibody Test for the in vitro qualitative detection of SARS-CoV-2 specific IgG antibody as indicative of an immune response to SARS-CoV-2 infection, for the detection of seroconversion in patients following known recent SARS-CoV-2 infection, and the identification of patients with titers above 1:2880 dilution during the public health emergency.

### L. FDA Administrative Information:

### Product Code:

### QKO: Reagent, Coronavirus Serological

### 6.8.2 References

1. Amanat, Fatima & Nguyen, Thi & Chromikova, Veronika & Strohmeier, Shirin & Stadlbauer, Daniel & Javier, Andres & Jiang, Kaijun & Asthagiri-Arunkumar, Guha & Polanco, Jose & Bermudez-Gonzalez, Maria & Caplivski, Daniel & Cheng, Allen & Kedzierska, Katherine & Vapalahti, Olli & Hepojoki, Jussi & Simon, Viviana & Krammer, Florian. (2020). A serological assay to detect SARS-Cov-2 seroconversion in humans. 10.1101/2020.03.17.20037713.
2. Wölfel R, Corman V M, Guggemos W, et al. Virological assessment of hospitalized patients with COVID-2019. Nature 2020.
3. Zhao J, Yuan Q, Wang H, et al. Antibody responses to SARS-CoV-2 in patients of novel coronavirus disease 2019. Clin Infect Dis 2020.

### 6.9 Example 9: Microneutralization Assay

Vero. E6 cells were seeded at a density of 20,000 cells per well in a 96-well cell culture plate in cDMEM. The following day, heat inactivated serum samples (dilution of 1:10) were serially diluted 3-fold in 2×MEM (20% 10× minimal essential medium (Gibco), 4 mM L-glutamine, 0.2% of sodium bicarbonate [wt/vol; Gibco], 20 mM 4-(2-hydroxyethyl)-1-piperazineethanesulfonic acid (HEPES, Gibco), 200 U/ml penicillin-200 μ/ml streptomycin (Gibco), and 0.4% bovine serum albumin (MP Biomedical)). The authentic SARS-CoV-2 virus (USA-WA1/2020, GenBank: MT020880) was diluted to a concentration of 100 50% cell culture infectious doses (TCID₅₀) in 2×MEM. Eighty μL of each serum dilution and 80 μL of the virus dilution were added to a 96-well cell culture plate and allowed to incubate for 1 hr at room temperature. cDMEM was removed from Vero.E6 cells and 120 μL of the virus-serum mixture was added to the cells and the cells were incubated at 37° C. for 1 hr. After the 1 hr incubation, the virus-serum mixture was removed from the cells and 100 μL of each corresponding serum dilution and 100 μL of 2×MEM containing 2% FBS (Corning) was added to the cells. The cells were incubated for 48 hr at 37° C. and then fixed with 10% paraformaldehyde (PFA) (Polysciences, Inc) for 24 hr at 4° C. Following fixation, the PFA was removed and the cells were washed with 200 μL of PBS. The cells were then permeabilized by the addition of 150 μL of PBS containing 0.1% Triton X-100 for 15 minutes at room temperature. The plates were then washed three times with PBS containing 0.1% Tween 20 (PBS-T) and blocked in blocking solution (3% milk [American Bio] in PBS-T) for 1 h at room temperature. After blocking, 100 μL of 1C7 (anti-SARS NP antibody generated in house) at a dilution of 1:1000 was added to all wells and the plates were allowed to incubate for 1 hr at room temperature. Plates were then washed three times with PBS-T before the addition of goat anti-mouse IgG-horseradish peroxidase (IgG-HRP; Rockland Immunochemicals) (diluted 1:3000) in blocking solution for 1 hr at room temperature. Plates were then washed three times with PBS-T and the ( )phenylenediamine dihydrochloride (OPD) substrate (SigmaFast OPD; Sigma-Aldrich) was added. After a 10-minute room temperature incubation, the reaction was stopped by adding 50 μL of 3 M HCl to the mixture. The optical density (OD) was measured at 490 nm on a Synergy 4 plate reader (BioTek). A cutoff value of the average of the OD values of blank wells plus three standard deviations was established for each plate and used for calculating the microneutralization titer. Microneutralization assays were performed in a biosafety level 3 facility.

FIGS. 20A-20C demonstrate the correlation between microneutralization IC₅₀ and ELISA IgG titers.

### 6.10 Example 10: SARS-CoV-2 Infection Induces Robust, Neutralizing Antibody Responses that are Stable for at Least Three Months

SARS-CoV-2 has caused a global pandemic with millions infected and numerous fatalities. Questions regarding the robustness, functionality and longevity of the antibody response to the virus remain unanswered. This example reports that the vast majority of infected individuals with mild-to-moderate COVID-19 experience robust IgG antibody responses against the viral spike protein, based on a dataset of 19,860 individuals screened at Mount Sinai Health System in New York City. The data described in this example shows that titers are stable for at least a period approximating three months, and that anti-spike binding titers significantly correlate with neutralization of authentic SARS-CoV-2. The data suggests that more than 90% of seroconverters make detectible neutralizing antibody responses and that these titers are stable for at least the near-term future.

### 6.10.1 Materials & Methods

Study participants, human samples and study design. Starting in late March, 2020, we conducted an outreach program in the New York City area, including parts of New York State, Connecticut, and New Jersey, to identify people recovered from SARS-CoV-2. Participants were tested for SARS-CoV-2 antibodies and, if positive, obtained a titer level (1:80, 160, 320, 960 or ≥2880) using the Mount Sinai ELISA described here. Participants were recruited via REDCap® (Vanderbilt University, Tennessee) online survey response which was advertised on the hospital website, and subsequently shared by multiple news organizations and public officials in New York. REDCap respondents' were deemed eligible if they had previously tested positive for SARS-CoV-2 via nasopharyngeal PCR (Cobas® SARS-CoV-2, Roche Diagnostics, Indiana). Due to the lack of PCR testing in the New York area prior to mid-March, 2020, we also included people for antibody testing and screening if they were symptomatic with suspected SARS-CoV-2 symptoms after Feb. 1, 2020, if they had a high risk exposure to someone with a positive SARS-CoV-2 PCR test, or were healthcare workers. Additionally, only participants who were asymptomatic at time of survey were eligible to come in for antibody testing. Respondents self-reported date of symptom onset, date of positive SARS-CoV-2 test (if applicable), and last date of symptoms.

During the first week of serum antibody testing, participants were brought in ten or more days after they had a confirmed/suspected diagnosis and had been asymptomatic for at least three days. In week 2, as more potential donors were identified and more was learned about our antibody assay, the timeline was extended to 14 days after symptoms onset, with at least three days asymptomatic. Quickly it was seen that this was a little early for serum conversion, and by week 3, participants 21 days or more after symptom onset, who had been completely asymptomatic for at least 14 days, were included. Donors for plasma donation were referred to the New York Blood Center if their antibodies were ≥1:320. All interested participants with antibody titers >1:320 and negative SARS-CoV-2 PCR swabs were screened by the New York Blood Center using standard criteria for plasma donation and included as donors in our convalescent plasma study if eligible as per CFR Title 21. Studies were reviewed and approved by our institutional review board.

It is important to note that as greater numbers of potential donors were identified and built capacity for testing in our clinical lab, testing was offered to wider groups including family and friends of individuals with positive PCR or antibodies, essential workers, patients in our health system, members of our community, and finally to all employees of the Mount Sinai Health System. Testing for employees was voluntary, and included both front line health care workers and administrative support staff, faculty and students, regardless of their clinical assignments during the COVID-19 pandemic. This broadening likely explains why our antibody testing yielded lower positive rates over time, not because of characteristics of the test or of the population curve.

Enzyme-linked immunosorbent assay (ELISA). The Mount Sinai Hospital (MSH) enzyme linked immunosorbent assay (ELISA) is an orthogonal immune assay specific for IgG anti SARS-CoV-2 spike protein in serum or plasma and measures the relative concentration of IgG as the highest dilution of serum giving a positive signal (OD₄₉₀≥0.15) after exactly 5 minutes of color development. The result is reported as reciprocal value of the sample's highest dilution producing a signal. The assay received FDA authorization for clinical use on 4/15/202 validating the safe application on the original research assay developed and described by the Krammer laboratory team in the CLIA environment. The MSH-ELISA Anti IgG COVID-19 assay was also independently authorized as a laboratory developed test (LDT) for clinical application by the NYSDOH at the Mount Sinai Laboratory (MSL), Center for Clinical Laboratories, a division of the Department of Pathology, Molecular, and Cell-Based Medicine, New York, NY (CLIA #33D1051889) from individuals suspected of previous COVID-19 infection by their healthcare provider, for the assessment of seroconversion from an antibody negative status to an antibody positive status in acutely infected patients, and for identification of individuals with SARS-Cov-2 IgG antibodies titers of up to 1:2880. A detailed description of the assay can be found here (8, 13).

Neutralization assay. Human samples were heat-inactivated at 56° C. for an hour prior to use. Vero.E6 cells (ATCC #CRL-1586) were seeded at a density of 20,000 cells per well in a 96-well cell culture plate (Corning, cat. no. 3595) one day before the assay was performed. Cells were maintained in culture in complete Dulbecco's Modified Eagle's Medium and the media used for the neutralization assay was 1× Minimal Essential Medium supplemented with 2% fetal bovine serum (FBS; Corning). The details of the protocol have been described in detail earlier (18).

Starting with 1:10, serial dilutions of each sample was prepared in a 96-well plate in duplicates. Six wells in each plate were used as “no virus” controls and 6 wells were used as “virus only” controls. Next, 80 uls of each respective dilution was mixed with 600TCID₅₀ of SARS-CoV-2 isolate USA-WA1/2020, (BEI Resources NR-52281) in 80 μls. The virus-serum mixture was incubated for an hour. Next, media from cells was removed and 120 μls of virus-serum mixture was added onto the cells. The cells were incubated for an hour at 37° C. After an hour, the virus-serum mixture was removed and 100 μls of 1×MEM and 100 uls of each respective serum dilution was added to the cells. The cells were kept in the 37° C. incubator for 2 days. After 48 hours, the media from the cells was removed and 150 μls of 10% formaldehyde (Polysciences) was added to the cells to inactivate the virus for 24 hours. The next day, cells were permeabilized and stained using an anti-NP antibody. Percent inhibition of virus was calculated for each dilution. This protocol has been earlier published in much greater detail (18).

Statistical analysis. Correlation analysis was performed using Spearman's rank test. A paired t-test was used to compare longitudinal titers. A p>0.05 was considered significant. Analysis was performed in GraphPad Prism.

### 6.10.2 Results

Severe acute respiratory syndrome coronavirus 2 (SARS-CoV-2) has infected millions of individuals globally and, as of July 2020, has led to the death of more than 500,000 individuals. Despite the global spread of the virus, there is still a lack understanding of many aspects of the humoral immune response that natural infection with SARS-CoV-2 induces (1). Many SARS-CoV-2 infections are mild or even asymptomatic. While the antibody responses to severe COVID-19 are relatively well characterized (2, 3), understanding the response in mild COVID-19 cases is of high importance, since mild and asymptomatic cases constitute the majority of infections. It will be critical to understand the robustness of the antibody response in mild cases, including its longevity and its functionality, so as to inform serosurveys, as well as to determine levels and duration of antibody titers that may be protective from reinfection

Antibodies to SARS-CoV-2 can target many of its encoded proteins, including structural and non-structural antigens. So far, two structural proteins have been utilized as target antigens for serological assays. One of them is the abundant nucleoprotein (NP), which is found inside the virus or inside infected cells. A number of antibody-based assays targeting NP have been developed, and are being used for serological studies. However, due to the biological function of NP and the fact that it is shielded from antibodies by viral or cellular membranes, it is unlikely that NP antibodies can directly neutralize SARS-CoV-2. For SARS-CoV-1, it has been shown that vaccination with NP can induce strong antibody responses; however, they were found to be non-neutralizing (4) While non-neutralizing antibodies might still exert antiviral activity, for example via Fe-Fc receptor-based effector function, non-neutralizing NP antibodies have led to enhanced disease for some vaccine candidates in animal models when neutralizing antibodies were absent (4). The second structural protein often used as target for characterizing the immune response to SARS-CoV-2 is the spike protein. The spike is a large trimeric glycoprotein that contains the receptor binding domain (RBD), which the virus uses to dock to its cellular receptor angiotensin converting enzyme 2 (ACE2), in addition to possessing the machinery that allows fusion of viral and cellular membranes (5, 6). It is known from other coronaviruses as well as for SARS-CoV-2 that the spike is the main, and potentially the only target for neutralizing antibodies (7). Therefore, the assay used in this study to characterize the antibody response to SARS-CoV-2 is based on the trimerized, stabilized ectodomain of the spike protein (8). An enzyme-linked immunosorbent assay (ELISA) was initially developed in early 2020, has been extensively used in research (9-12), and was established in Mount Sinai's CLIA laboratory where it received New York State Department of Health (NYSDOH) and FDA emergency use authorization (EUA) (8, 13). The so-called Mount Sinai ELISA antibody test has high sensitivity (92.5%) and specificity (100%) as determined with an initial validation panel of samples (Table 11); and a positive predictive value (PPV) of 100%, with a negative predictive value (NPV) of 99.6%.

TABLE 11
Contingency table to calculate sensitivity and specificity
of the Mount Sinai ELISA SARS-CoV-2 antibody test

COVID-19

| | | positive | negative | total |
|---|---|---|---|---|
| Test | Positive | 37 | 0 | 37 |
| | Negative | 3 | 74 | 77 |
| | Total | 40 | 74 | 114 |

| | Value | 95% CI |
|---|---|---|
| Sensitivity | 0.925 | 0.8014 to 0.9742 |
| Specificity | 1 | 0.9507 to 1 |

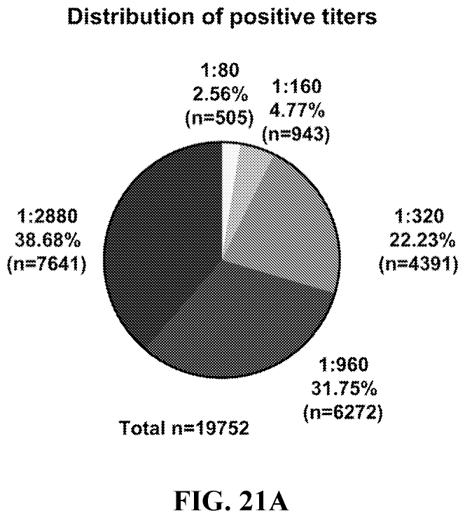

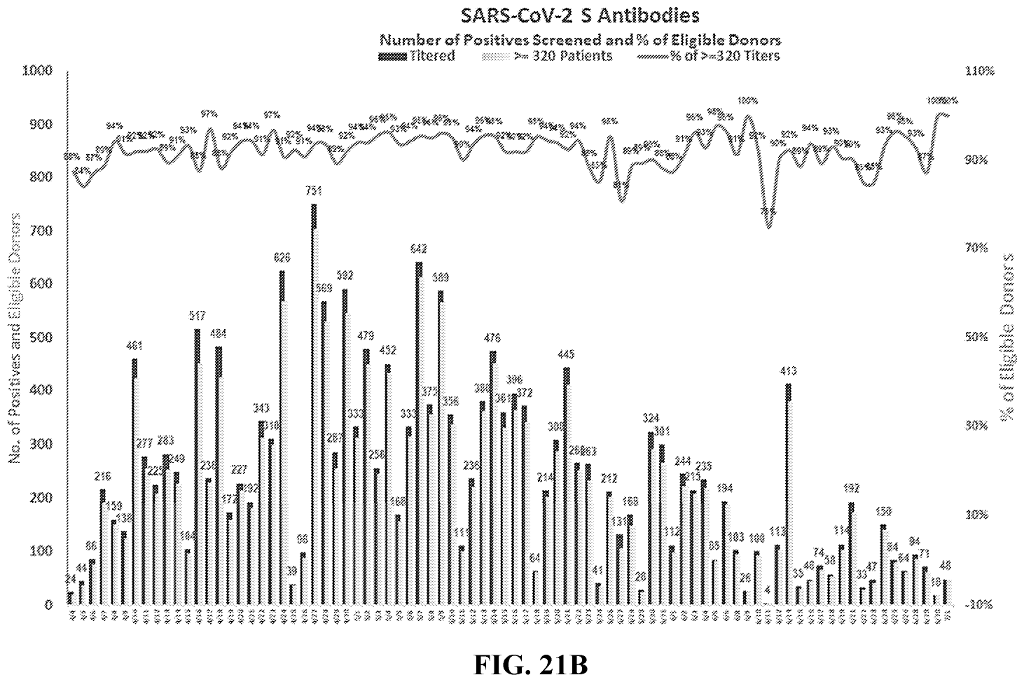

In March 2020, Mount Sinai Health System started to screen individuals for antibodies to SARS-CoV-2 to recruit volunteers as donors for convalescent plasma therapy (14). Screened patients either had confirmed SARS-CoV-2 infections by PCR, or suspected disease, defined as being told by a physician that symptoms may be related to SARS-Cov-2 or exposure to someone with confirmed SARS-CoV-2 infection. The vast majority of symptomatic cases that were screened experienced mild-to-moderate disease, with less than 5% requiring emergency department evaluation or hospitalization. In addition to screening potential donors, Mount Sinai also offered the Mount Sinai ELISA antibody test to all employees within our health system on a voluntary basis. By July 2^{nd}, Mount Sinai bad screened 51,829 individuals using ELISAs with 19,763 individuals being positive (defined as detectable antibodies to the spike protein at a titer of 1:80 or higher) and 32,063 individuals being negative. The CLIA ELISA set up results in a discrete titer at either 1:80, 1:160, 1:320, 1:960 or ≥1:2880. The titers of 1:80 and 1:160 were characterized as low titers, titers of 1:320 were characterized as moderate, and titers of 1:960 and ≥1:2880 were characterized as high titers. For plasma therapy, titers of 1:320 or higher were initially deemed eligible. Of the 19,763 positive samples 505 (2.56%) had a titer of 1.80, 943 (4.77%) of 1.160, 4391 (22.23%) of 1:320, 6272 (31.75%) of 1:960 and 7641 (38.68%) of 1:2880 (FIG. 21). Therefore, the vast majority of positive individuals have moderate to high titers of anti-spike antibodies. Of course, the argument could be made that the authors could be missing a number of individuals that had been infected with SARS-CoV-2 and did not produce antibodies, since many individuals included in the data set had never been tested by a nucleic acid amplification test (NAAT) for the virus. An earlier analysis performed with a smaller subset of 568 PCR-confirmed individuals using the same ELISA showed that >99% of them developed an anti-spike antibody response (9). In a later dataset of 2,347 patients who self-reported positive PCR, 95% of them had positive antibody titers, indicating the authors are not missing large numbers of patients and confirming our prior sensitivity findings. Therefore, this example reports here that the rate of individuals who do not seroconvert after SARS-CoV-2 infection is low, although such individuals may exist, and the majority of responders mount titers of 1:320 or higher

Binding antibody titers tell us how robust the immune response to a certain virus or antigen is. However, it does not necessarily tell us anything about functionality of the antibody response, and this has been an open question related to SARS-CoV-2. Determining the neutralizing effects of SARS-CoV-2 spike antibodies is critical to understanding possible protective effects of the innate immune response Antibodies can exert antiviral actions via many different pathways, including Fc-Fc receptor mediated effector functions and Fc-complement interactions (75-17) However, the antiviral activity that correlates with protection for most viruses is in vitro neutralization activity. In order to determine if antibodies induced against the spike protein exert neutralizing activity, a well-established. quantitative microneutralization assay (78) was performed based on authentic SARS-Cov-2 with 120 samples of known ELISA titers ranging from ‘negative’ to 1:2880. Neutralization titers significantly correlated (Spearman r=0.87, p<0 0001) with spike-binding titers (FIG. 22A). While there was some variability, sera with 1:320 ELISA titers had a geometric mean 50% inhibitory dilution (IDs) of approximately 1:30, for the 1:960 titer, it was approximately 1:75 and for the 1:2880 titer it was approximately 1:550. Considering any neutralizing activity above background, approximately 50% of sera in the 1:80-1.160 titer range had neutralizing activity, 90% in the 1:320 range had neutralizing activity, and all sera in the 1:960-1:2880 range had neutralizing activity (FIG. 22B). This is encouraging, and further validates the use of antibody titers to refer for plasma donation and for further investigation into protective effects at the various titer levels.

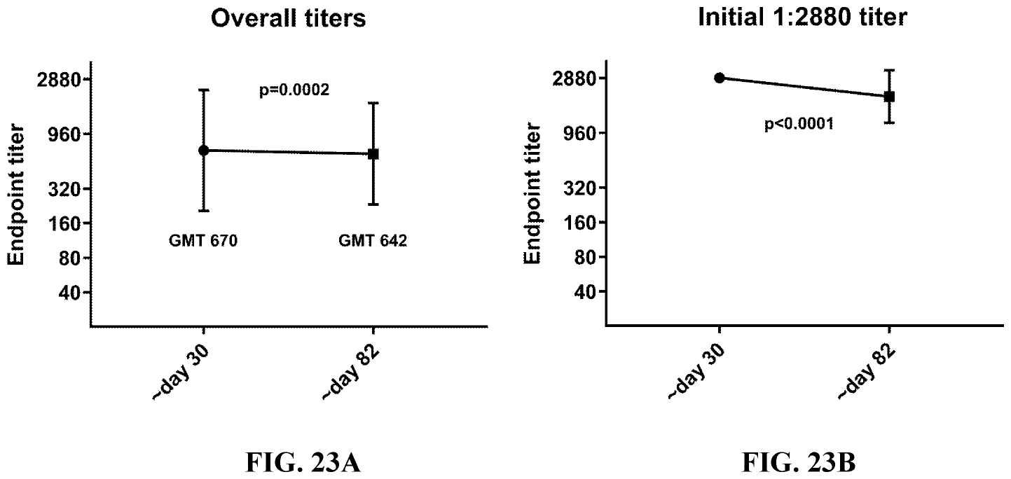

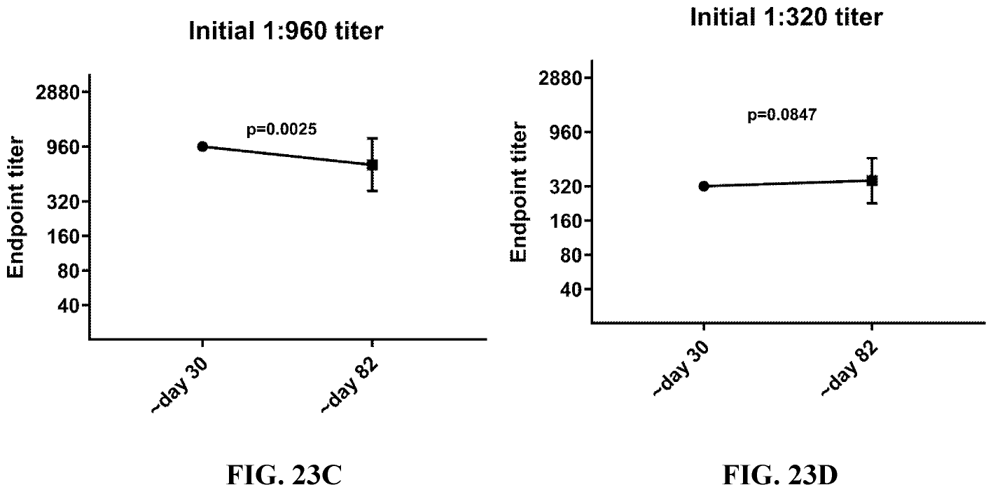

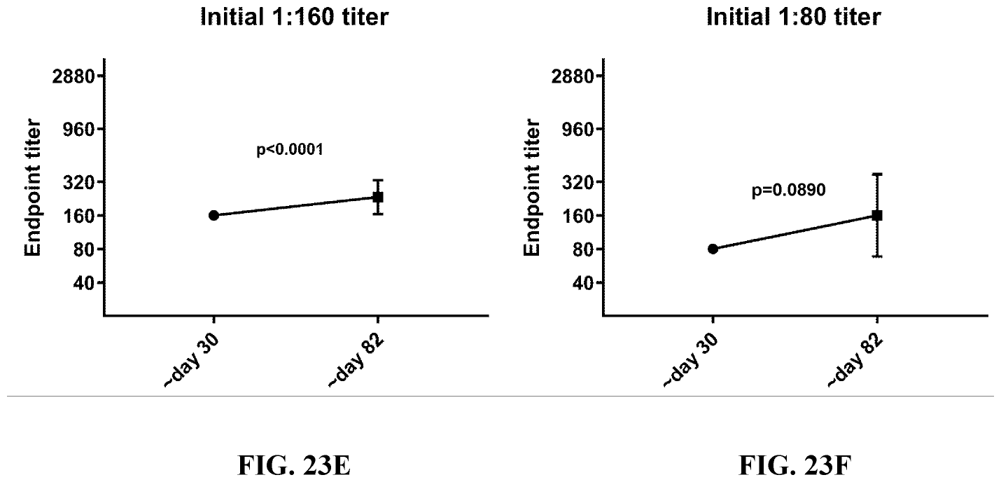

Another important question is longevity of the antibody response to the spike To assess the medium-range stability of serum antibody titers against the spike protein, 121 plasma donors at a variety of titer levels that had initially been screened at approximately day 30 post symptom onset were recalled. The mean interval between the initial titer measurement and the second was 52 days (range 33-67 days), setting the second time point at a mean of 82 days post symptom onset (range 52-104 days). When the overall titers were compared a slight drop from a geometric mean titer (GMT) of 670 to a GMT of 642 was seen (FIG. 23A). In the higher titer range of 1:2880 and 1:960, a slight drop in titer between the two time points was observed (FIGS. 23B and 23C). Surprisingly, but in agreement with earlier observations from our group that seroconversion in mild COVID-19 cases might take longer time to mount (9), an increase in individuals who had an initial titer of 1:320, 1:160 or 1:80 was seen (FIGS. 23D-23F). The exception was one individual that dropped from a 1:80 titer to being negative. The initial serum antibody titer was likely produced by plasmablasts, and plasmablast-produced antibody peaks 2-3 weeks post symptom onset. Given an IgG half-life of approximately 21 days, the sustained or increasing antibody titers observed are likely produced by long-lived plasma cells in the bone marrow. Of note, the observations are in contrast to a recent report by Long et al. that found waning titers 8 weeks post virus infection as compared to acute responses (19). Especially in asymptomatic cases, antibody responses disappeared after 8 weeks in 40% of individuals in the Long et al. study. However, the antibodies measured in their paper were targeting the NP plus a single linear spike epitope. Much more in agreement with the data here, the same paper also reports relatively stable (slightly declining) neutralizing antibody titers. The stability of the antibody response over time might therefore also depend on the target antigen. The titers measured here do correlate with neutralization as discussed above.

For many different viral infections correlates of protection have been established. These correlates are usually based on a specific level of antibody acquired through vaccination or natural infection that significantly reduces the risk of (re-)infection. Examples are the hemagglutination inhibition titer for influenza virus, where a 1:40 titer reduces the risk of getting infected by 50% (20). Similar titers have been established for measles virus (an ID₅₀ titer of 1.120), hepatitis A virus, hepatitis B virus and many others (27). These titers have facilitated vaccine development significantly. For some viruses/vaccines, the kinetics of the antibody response is also known, allowing for an accurate prediction of how long protection will last (22).

It is still not clear if infection with SARS-CoV-2 in humans protects from re-infection and for how long. It is known from work with common human coronaviruses that neutralizing antibodies are induced and these antibodies can last for years and provide protection from re-infection or attenuate disease, even if individuals get re-infected (23). Furthermore, it is now known from non-human primate models that infection with SARS-CoV-2 does protect from re-infection for at least some time (24, 25). This protection is, in some cases, more pronounced in the lower respiratory tract than in the upper respiratory tract (25). It is also know that transferring serum, for example from infected hamsters into naïve hamsters, reduces virus replication significantly when the naïve hamsters are challenged (26). Similarly, non-human primates can be protected by prophylactic treatment with neutralizing monoclonal antibodies (27). Finally, vaccine induced neutralizing antibody titers have been established as correlate of protection in non-human primates (28). Of note, these titers were relatively low and in the lower range of the titers observed here. The data here reveals that individuals who have recovered from mild COVID-19 experience robust antibody responses. Antibody binding titers to the spike protein correlate significantly with neutralization with authentic SARS-CoV-2 virus, and the vast majority of individuals with antibody titers of 1:320 or higher show neutralizing activity in their serum. Consistent with data for human coronaviruses, SARS-CoV-1 and Middle Eastern respiratory syndrome-CoV (23), the data show stable antibody titers over a period of at least 3 months, and this cohort will be followed over longer intervals of time. While this cannot provide conclusive evidence that these antibody responses protect from re-infection, the authors believe it is very likely that they will decrease the odds ratio of getting re-infected, and may attenuate disease in the case of breakthrough infection. The authors believe it is imperative to swiftly perform studies to investigate and establish a correlate of protection from infection with SARS-CoV-2. A correlate of protection, combined with a better understanding of antibody kinetics to the spike protein, would inform policy regarding the COVID-19 pandemic and would be of benefit to vaccine development.

### 6.10.3 References Cited in Example 10

1. F. Krammer, V. Simon, Serology assays to manage COVID-19. Science 368, 1060-1061 (2020).
2. L. Liu et al., High neutralizing antibody titer in intensive care unit patients with COVID-19. Emerg Microbes Infect, 1-30 (2020).
3. Q. X. Long et al., Antibody responses to SARS-CoV-2 in patients with COVID-19. Nat Med 26, 845-848 (2020).
4. D. Deming et al., Vaccine efficacy in senescent mice challenged with recombinant SARS-CoV bearing epidemic and zoonotic spike variants. PLOS Med 3, e525 (2006).
5. D. Wrapp et al., Cryo-EM structure of the 2019-nCoV spike in the prefusion conformation. Science, (2020).
6. M. Letko, A. Marzi, V. Munster, Functional assessment of cell entry and receptor usage for SARS-CoV-2 and other lineage B betacoronaviruses. Nat Microbiol 5, 562-569 (2020).
7. F. Amanat, F. Krammer, SARS-CoV-2 Vaccines: Status Report. Immunity 52, 583-589 (2020).
8. F. Amanat et al., A serological assay to detect SARS-CoV-2 seroconversion in humans. Nat Med, (2020).
9. A. Wajnberg et al., Humoral immune response and prolonged PCR positivity in a cohort of 1343 SARS-CoV 2 patients in the New York City region. medRxiv, 2020.2004.2030.20085613 (2020).
10. A. S. Dingens et al., Seroprevalence of SARS-CoV-2 among children visiting a hospital during the initial Seattle outbreak. medRxiv, (2020).
11. D. S. Hains et al., Asymptomatic Seroconversion of Immunoglobulins to SARS-CoV-2 in a Pediatric Dialysis Unit. JAMA, (2020).
12. D. Stadlbauer et al., Seroconversion of a city: Longitudinal monitoring of SARS-CoV-2 seroprevalence in New York City. medRxiv, 2020.2006.2028.20142190 (2020).
13. D. Stadlbauer et al., SARS-CoV-2 Seroconversion in Humans: A Detailed Protocol for a Serological Assay, Antigen Production, and Test Setup. Curr Protoc Microbiol 57, e100 (2020).
14. S. T. H. Liu et al., Convalescent plasma treatment of severe COVID-19: A matched control study. medRxiv, 2020.2005.2020.20102236 (2020).
15. N. K. Thulin, T. T. Wang, The Role of Fc Gamma Receptors in Broad Protection against Influenza Viruses. Vaccines (Basel) 6, (2018).
16. B. M. Gunn et al., A Role for Fc Function in Therapeutic Monoclonal Antibody-Mediated Protection against Ebola Virus. Cell Host Microbe 24, 221-233.e225 (2018).
17. E. O. Saphire, S. L. Schendel, B. M. Gunn, J. C. Milligan, G. Alter, Antibody-mediated protection against Ebola virus. Nat Immunol 19, 1169-1178 (2018).
18. F. Amanat et al., An In Vitro Microneutralization Assay for SARS-CoV-2 Serology and Drug Screening. Curr Protoc Microbiol 58, e108 (2020).
19. Q. X. Long et al., Clinical and immunological assessment of asymptomatic SARS-CoV-2 infections. Nat Med, (2020).
20. F. Krammer, J. P. Weir, O. Engelhardt, J. M. Katz, R. J. Cox, Meeting report and review: Immunological assays and correlates of protection for next-generation influenza vaccines. Influenza Other Respir Viruses, (2019).
21. S. A. Plotkin, Correlates of protection induced by vaccination. Clin Vaccine Immunol 17, 1055-1065 (2010).
22. K. Van Herck, P. Van Damme, Inactivated hepatitis A vaccine-induced antibodies: follow-up and estimates of long-term persistence. J Med Virol 63, 1-7 (2001).
23. A. T. Huang et al., A systematic review of antibody mediated immunity to coronaviruses: antibody kinetics, correlates of protection, and association of antibody responses with severity of disease. medRxiv, 2020.2004.2014.20065771 (2020).
24. W. Deng et al., Primary exposure to SARS-CoV-2 protects against reinfection in rhesus macaques. Science, (2020).
25. A. Chandrashekar et al., SARS-CoV-2 infection protects against rechallenge in rhesus macaques. Science, (2020).
26. M. Imai et al., Syrian hamsters as a small animal model for SARS-CoV-2 infection and countermeasure development. Proc Natl Acad Sci USA, (2020).
27. R. Shi et al., A human neutralizing antibody targets the receptor binding site of SARS-CoV-2. Nature, (2020).
28. J. Yu et al., DNA vaccine protection against SARS-CoV-2 in rhesus macaques. Science, (2020).

### 6.11 Example 11: Quantitative SARS-CoV-2 IgG Antibody Research Kit

This example describes a Quantitative SARS-CoV-2 IgG Antibody Research Kit consisting of two serial direct Enzyme-Linked Immunosorbent Assays (ELISA) intended for quantitative detection of human IgG antibodies to the SARS-CoV-2 virus in serum and plasma (Heparin and EDTA) samples. An initial qualitative ELISA is performed against recombinant Receptor Binding Domain of SARS-CoV-2, followed for positive specimens by a quantitative ELISA against full length SARS-CoV-2 Spike protein.

The two-step assay described in this Example is an antigen-down enzyme immunoassay where a recombinant SARS-CoV-2 Receptor Binding Domain protein (RBD) is pre-coated onto a 96-well microplate in step one. When the sample is added, antibodies found in the sample that recognize SARS-CoV-2 RBD bind the antigen coated plate and are retained in the well. After washing away unbound substances, an enzyme-linked monoclonal antibody specific for human IgG is added to the wells. Following a wash to remove any unbound enzyme-linked antibody, a substrate is added to the wells and color develops in proportion to the amount of IgG antibodies in the sample bound to the SARS-CoV-2 RBD antigen. The color development is stopped, and the intensity of the color is measured. Samples that have a measured value above a pre-determined cutoff are determined to be positive and tested in the second-step ELISA.

Positive samples are evaluated on a second orthogonal ELISA step to quantify the levels of IgG antibodies to the SARS-CoV-2 Spike protein. For this assay, a recombinant SARS-CoV-2 Spike Protein is pre-coated onto a 96-well microplate and used to bind antibodies found in the sample. When the sample is added, antibodies found in the sample that recognize SARS-CoV-2 Spike Protein bind the antigen coated plate and are retained in the well. After washing away unbound substances, an enzyme-linked monoclonal antibody specific for human IgG is added to the wells. Following a wash to remove any unbound enzyme-linked antibody, a substrate is added to the wells and color develops in proportion to the amount of IgG antibodies in the sample bound to the SARS-CoV-2 Spike antigen. The color development is stopped, and the intensity of the color is measured. The signal from unknown samples is compared to a calibration curve to generate a final result in arbitrary units per milliliter (AU/mL).

### 6.11.1 Kit Components and Storage Conditions

| | Quantity | | Storage Of Opened/ |
|---|---|---|---|
| Part | (10 Pack) | Description | Reconstituted Material |
| RBD Antigen | 4 plates | 96 well polystyrene microplate coated | Reseal along entire edge |
| Microplate | | with recombinant SARS-CoV-2 Spike | of the zip-seal. May be |
| | | protein RBD antigen. | stored for up to 1 month |
| Spike Protein | 5 plates | 96 well polystyrene microplate coated | at 2-8° C.* |
| Microplate | | with full length recombinant SARS- | |
| | | CoV-2 Spike protein. | |
| RBD | 1 vial | 125 μL of 1000X concentrated | May be stored for up to 1 |
| Conjugate | | monoclonal antibody specific to | month at 2-8° C. |
| Concentrate | | human IgG conjugated to horseradish | |
| (1000X) | | peroxidase. | |
| Spike | 1 vial | 125 μL of 1000X concentrated | |
| Conjugate | | monoclonal antibody specific to | |
| Concentrate | | human IgG conjugated to horseradish | |
| (1000X) | | peroxidase. | |
| Conjugate | 1 bottle | 120 mL monoclonal antibody specific | |
| Buffer - IgG | | to human IgG conjugated to | |
| ELISA | | horseradish peroxidase with | |
| | | preservatives. | |
| Sample | 3 bottles | 91 mL of a buffered protein base with | |
| Buffer - IgG | | preservatives. Sample buffer | |
| ELISA | | comprises, inter alia, phosphate buffer | |
| | | and 1.25% milk. | |
| TMB | 1 bottle | 116 mL of stabilized hydrogen | |
| Substrate - | | peroxide and chromogen | |
| IgG ELISA | | (tetramethylbenzidine). | |
| Stop Solution - | 1 bottle | 116 mL of acidic solution. | |
| IgG ELISA | | | |
| Wash Buffer - | 2 bottles | 101 mL of a 25-fold concentrated | |
| IgG ELISA | | solution of buffered surfactant with | |
| | | preservative. | |
| RBD Positive | 1 vial | 1.0 mL of monoclonal antibody in a | May be stored for up to 1 |
| Control | | protein buffered base with | month at 2-8° C. |
| | | preservatives. | |
| RBD | 1 vial | 1.0 mL of monoclonal antibody in a | |
| Negative | | protein buffered base with | |
| Control | | preservatives. | |
| Spike Low | 1 vial | 1.0 mL of monoclonal antibody in a | |
| Control | | protein buffered base with | |
| | | preservatives. | |
| Spike Mid | 1 vial | 1.0 mL of monoclonal antibody in a | |
| Control | | protein buffered base with | |
| | | preservatives. | |
| Spike High | 1 vial | 1.0 mL of monoclonal antibody in a | |
| Control | | protein buffered base with | |
| | | preservatives. | |
| Spike | 1 vial | 1.25 mL of a monoclonal antibody in a | |
| Calibrator 1 | | buffered base with preservatives. | |
| (200 AU/mL) | | | |
| Spike | 1 vial | 1.25 mL of a monoclonal antibody in a | |
| Calibrator 2 | | buffered base with preservatives. | |
| (66.7 AU/mL) | | | |
| Spike | 1 vial | 1.25 mL of a monoclonal antibody in a | |
| Calibrator 3 | | buffered base with preservatives. | |
| (22.2 AU/mL) | | | |
| Spike | 1 vial | 1.25 mL of a monoclonal antibody in a | |
| Calibrator 4 | | buffered base with preservatives. | |
| (7.41 AU/mL) | | | |
| Spike | 1 vial | 1.25 mL of a monoclonal antibody in a | |
| Calibrator 5 | | buffered base with preservatives. | |
| (2.47 AU/mL) | | | |
| Spike | 1 vial | 1.25 mL of a monoclonal antibody in a | |
| Calibrator 6 | | buffered base with preservatives. | |
| (0.82 AU/mL) | | | |
| Spike | 1 vial | 1.25 mL of a monoclonal antibody in a | |
| Calibrator 7 | | buffered base with preservatives. | |
| (0 AU/mL) | | | |

The unopened kit is stored at 2-8° C.

### 6.11.2 Sample Collection and Storage

Serum—Use a serum separator tube (SST) and allow samples to clot for 30 minutes at room temperature before centrifugation for 15 minutes at 1000×g. Remove serum and assay immediately or aliquot and store samples at ≤−20° C. Avoid repeated freeze-thaw cycles.

Plasma—Collect plasma using EDTA, heparin, or citrate as an anticoagulant. Centrifuge for 15 minutes at 1000×g within 30 minutes of collection. Assay immediately or aliquot and store samples at ≤−20° C. Avoid repeated freeze-thaw cycles. Grossly hemolyzed or lipemic samples are not acceptable in this assay.

### 6.11.3 Reagent Preparation

RBD Conjugate—Add 11 μL of RBD Conjugate Concentrate (1000×) to 11 mL of Conjugate Buffer. Mix well.

Spike Conjugate—Add 11 μL of Spike Conjugate Concentrate (1000×) to 11 mL of Conjugate Buffer. Mix well.

Wash Buffer—If crystals have formed in the concentrate, warm to room temperature and mix gently until the crystals have completely dissolved. Add 20 mL of Wash Buffer Concentrate to 480 mL of deionized or distilled water to prepare 500 mL of Wash Buffer.

Control Preparation—Dilute each control 1:5 by pipetting 2 mL of Sample Buffer into a tube. Add 0.5 mL of the control. Repeat for all 5 controls (RBD Positive, RBD Negative, Spike Low, Spike Mid, and Spike High).

Calibrators—No preparation is required.

### 6.11.4 Sample Preparation

### RBD Assay:

1. Heat inactivate samples by placing in a water bath or heat block at 56° C. for 1 hour. Do not leave samples at 56° C. for longer than 1 hour.
2. Dilute samples 1:5 in microcentrifuge tubes by adding 10 μL of sample to 40 μL of Sample Buffer.
3. Further dilute samples 1:20 (final dilution of 1:100) by adding 10 μL of sample (1:5) to 190 μL of Sample Buffer.

### Spike Assay:

1. Heat inactivate samples by placing in a water bath or heat block at 56° C. for 1 hour. Do not leave samples at 56° C. for longer than 1 hour.
2. Dilute samples 1:5 in microcentrifuge tubes by adding 10 μL of sample to 40 μL of Sample Buffer.
3. Further dilute samples 1:40 (final dilution of 1:200) by adding 10 μL of sample (1:5) to 390 μL of Sample Buffer.

### 6.11.5 Assay Procedure

### 6.11.5.1 Phase 1 RBD ELISA:

| | 1 | 2 | 3 | 4 | 5 | 6 | 7 | 8 | 9 | 10 | 11 | 12 |
|---|---|---|---|---|---|---|---|---|---|---|---|---|
| A | Blank | S6 | S14 | S22 | S30 | S38 | S46 | S54 | S62 | S70 | S78 | S86 |
| B | Pos | S7 | S15 | S23 | S31 | S39 | S47 | S55 | S63 | S71 | S79 | S87 |
| | Control | | | | | | | | | | | |
| C | Neg | S8 | S16 | S24 | S32 | S40 | S48 | S56 | S64 | S72 | S80 | S88 |
| | Control | | | | | | | | | | | |
| D | S1 | S9 | S17 | S25 | S33 | S41 | S49 | S57 | S65 | S73 | S81 | S89 |
| E | S2 | S10 | S18 | S26 | S34 | S42 | S50 | S58 | S66 | S74 | S82 | S90 |
| F | S3 | S11 | S19 | S27 | S35 | S43 | S51 | S59 | S67 | S75 | S83 | Blank |
| G | S4 | S12 | S20 | S28 | S36 | S44 | S52 | S60 | S68 | S76 | S84 | Pos |
| | | | | | | | | | | | | Control |
| H | S5 | S13 | S21 | S29 | S37 | S45 | S53 | S61 | S69 | S77 | S85 | Neg |
| | | | | | | | | | | | | Control |

1. Add 100 μL of control (1:5) or sample (1:100) per well. Incubate for 2 hours at room temperature on benchtop.

2. Aspirate each well and wash, repeating the process two times for a total of three washes. Wash by filling each well with Wash Buffer (400 μL) using a squirt bottle, manifold dispenser, or autowasher. Complete removal of liquid at each step is essential to good performance. After the last wash, remove any remaining Wash Buffer by aspirating or decanting. Invert the plate and blot it against clean paper towels.

3. Add 100 μL of 1× Conjugate to each well. Incubate for one hour at room temperature.

4. Repeat the aspiration/wash as in step 2.

5. Add 100 μL of Substrate Solution to each well. Incubate for 20 minutes at room temperature and protect from light.

6. Add 100 μL of Stop Solution to each well. The color in the well should change from blue to yellow. If the color in the well is green or if the color change does not appear uniform, gently tap the plate to ensure thorough mixing.

7. Determine the optical density of each well within 30 minutes, using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

RBD ELISA Calculation of Results: The RBD Positive Control (1:5) is used for normalization. Corrected sample OD values (see RBD ELISA step 7) are divided by the corrected RBD Positive Control (1:5) OD value to calculate a CI value. If the calculated CI value is >0.5, the sample is considered RBD positive and requires testing in Phase 2, the Spike ELISA. If the CI value is <0.5, the sample is negative for SARS-CoV-2 RBD antigen antibodies and is not tested in Phase 2.

### 6.11.5.2 Phase 2 Spike ELISA:

| | 1 | 2 | 3 | 4 | 5 | 6 | 7 | 8 | 9 | 10 | 11 | 12 |
|---|---|---|---|---|---|---|---|---|---|---|---|---|
| A | Cal 1 | Cal 1 | S2 | S2 | S10 | S10 | S18 | S18 | S26 | S26 | S34 | S34 |
| B | Cal 2 | Cal 2 | S3 | S3 | S11 | S11 | S19 | S19 | S27 | S27 | S35 | S35 |
| C | Cal 3 | Cal 3 | S4 | S4 | S12 | S12 | S20 | S20 | S28 | S28 | S36 | S36 |
| D | Cal 4 | Cal 4 | S5 | S5 | S13 | S13 | S21 | S21 | S29 | S29 | S37 | S37 |
| E | Cal 5 | Cal 5 | S6 | S6 | S14 | S14 | S22 | S22 | S30 | S30 | S38 | S38 |
| F | Cal 6 | Cal 6 | S7 | S7 | S15 | S15 | S23 | S23 | S31 | S31 | Low | Low |
| G | Cal 7 | Cal 7 | S8 | S8 | S16 | S16 | S24 | S24 | S32 | S32 | Medium | Medium |
| H | S1 | S1 | S9 | S9 | S17 | S17 | S25 | S25 | S33 | S33 | High | High |

1. Add 100 μL of control (1:5), calibrator (neat), or sample (1:200) per well transferring directly from dilution plate. Incubate for 2 hours at room temperature on benchtop.

2. Aspirate each well and wash, repeating the process two times for a total of three washes. Wash by filling each well with Wash Buffer (400 μL) using a squirt bottle, manifold dispenser, or autowasher. Complete removal of liquid at each step is essential to good performance. After the last wash, remove any remaining Wash Buffer by aspirating or decanting. Invert the plate and blot it against clean paper towels.

3. Add 100 μL of 1× Conjugate to each well. Incubate for one hour at room temperature.

4. Repeat the aspiration/wash as in step 2.

5. Add 100 μL of Substrate Solution to each well. Incubate for 20 minutes at room temperature and protect from light.

6. Add 100 μL of Stop Solution to each well. The color in the well should change from blue to yellow. If the color in the well is green or if the color change does not appear uniform, gently tap the plate to ensure thorough mixing.

7. Determine the optical density of each well within 30 minutes, using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

Spike ELISA Calculation of Results: Create a standard curve by reducing the data using computer software capable of generating a four parameter logistic (4-PL) curve fit. As an alternative, construct a standard curve by plotting the mean absorbance for each standard on the y-axis against the concentration on the x-axis and draw a best ft curve through the points on the graph. The data may be linearized be plotting the log of the concentration versus the log of the OD add the best fit line can be determined by regression analysis. This procedure will produce an adequate but less precise fit of the data.

If samples have been diluted, the concentration read from the standard curve must be multiplied by the dilution factor.

### 6.11.6. Additional Considerations

In order to achieve optimal performance, the pipette tip should not touch the inside of the well while loading standards, controls, samples, or blanks.
When mixing or reconstituting protein solutions, always avoid foaming.
To avoid cross-contamination, change pipette tips between additions of each standard level, between sample additions, and between reagent additions. Also, use separate reservoirs for each reagent.
To ensure accurate results, proper adhesion of plate sealers during incubation steps is necessary.
When using an automated plate washer, adding a 30 second soak period following the addition of Wash Buffer, and/or rotating the plate 180 degrees between wash steps may improve assay precision.
Substrate Solution should remain colorless until added to the plate. Keep Substrate Solution protected from light. Substrate Solution should change from colorless to gradations of blue.
Stop Solution should be added to the plate in the same order as the Substrate Solution. The color developed in the wells will turn from blue to yellow upon addition of the Stop Solution. Wells that are green in color indicate that the Stop Solution has not mixed thoroughly with the Substrate Solution.

### 6.12 Example 12: Semi-Quantitative SARS-CoV-2 IgG Antibody kit

### A. Intended Use

This example describes a Semi-Quantitative SARS-CoV-2 IgG Antibody Kit, which is a two-step Enzyme-Linked Immunosorbent Assay (ELISA) intended for qualitative and semi-quantitative detection of human IgG antibodies to the SARS-CoV-2 virus in serum and Li-Heparin plasma. An initial qualitative ELISA step is performed against recombinant Receptor Binding Domain of SARS-CoV-2, followed, for positive specimens, by a confirmatory semi-quantitative ELISA step against full-length SARS-CoV-2 Spike protein. The Semi-Quantitative SARS-CoV-2 IgG Antibody Kit aids in identifying individuals with an adaptive immune response to SARS-CoV-2, indicating recent or prior infection.

Results are for the qualitative and semi-quantitative detection of SARS-CoV-2 IgG antibodies. IgG antibodies to SARS-CoV-2 are generally detectable in blood several days after initial infection, although the duration of time antibodies are present post-infection is not well characterized. Individuals may have detectable virus present for several weeks following seroconversion.

Negative results do not preclude acute SARS-CoV-2 infection. If acute infection is suspected, direct testing for SARS-CoV-2 is necessary.

A negative result for an individual subject indicates the absence of detectable anti-SARS-CoV-2 antibodies. Negative results do not preclude SARS-CoV-2 infection and should not be used as the sole basis for patient management decisions. A negative result can occur if the quantity of the anti-SARS-CoV-2 antibodies that are detected and are present in the specimen is below the detection limits of the assay, or the antibodies that are detected are not present during the stage of disease in which a sample is collected.

False positive results for the Semi-Quantitative SARS-CoV-2 IgG Antibody Kit may occur due to cross-reactivity from pre-existing antibodies or other possible causes.

A positive result may not indicate previous SARS-CoV-2 infection. Consider other information, including clinical history and local disease prevalence, in assessing the need for a second but different serology test to confirm an immune response.

The test samples are generally collected from individuals that 15 days or more post symptom onset.

The Semi-Quantitative SARS-CoV-2 IgG Antibody Kit is only for use under the Food and Drug Administration's Emergency Use Authorization.

### B. Description and Test Principle

### 1) Test Principle:

The 2-step assay is an antigen-down enzyme immunoassay which utilizes a recombinant SARS-CoV-2 Receptor Binding Domain (RBD) antigen pre-coated onto a 96-well microplate in step 1. When the sample is added, antibodies found in the sample that recognize SARS-CoV-2 RBD antigen bind the antigen-coated plate and are retained in the well. After washing away unbound substances, an enzyme-linked monoclonal antibody specific for human IgG is added to the wells. Following a wash to remove any unbound enzyme-linked antibody, a substrate is added to the wells and color develops. The color development is stopped and the intensity of the color is measured. Samples that have a measured value above a pre-determined cutoff are determined to be positive in the first step ELISA and tested in the 2nd step ELISA, the Spike ELISA.

Positive samples from step 1 are evaluated on a second orthogonal ELISA that provides semi-quantitative measurement of IgG antibodies to the full-length SARS-CoV-2 Spike protein. For this assay, a recombinant SARS-CoV-2 Spike protein is pre-coated onto a 96-well microplate and used to bind antibodies found in the sample. When the sample is added, antibodies found in the sample that recognize SARS-CoV-2 Spike protein bind the antigen-coated plate and are retained in the well. After washing away unbound substances, an enzyme-linked monoclonal antibody specific for human IgG is added to the wells. Following a wash to remove any unbound enzyme-linked antibody, a substrate is added to the wells and color develops in proportion to the amount of IgG antibodies in the sample bound to the SARS-CoV-2 Spike protein. The color development is stopped and the intensity of the color is measured. The signal from unknown samples is compared to a calibration curve to generate a final result in arbitrary units per milliliter (AU/mL).

### 2) Kit Components and Storage Conditions:

TABLE 12
Materials included in Kit

| | | | Storage of |
|---|---|---|---|
| Part | Quantity | Description | Opened Material |
| RBD Antigen | 7 plates | 96 well polystyrene microplate coated | Use a new plate |
| Microplate | | with recombinant SARS-CoV-2 | for each assay. |
| | | Spike protein RBD antigen. | Discard after use. |
| Spike Protein | 3 plates | 96 well polystyrene microplate coated | Unused plates |
| Microplate | | with full length recombinant SARS- | from open kits |
| | | CoV-2 Spike protein. | may be stored for |
| | | | up to 1 month at |
| | | | 2-8° C. |
| RBD Conjugate | 1 vial | 125 μL of 1000X concentrated | May be stored for |
| Concentrate | | monoclonal antibody specific to | up to 1 month at |
| (1000X) | | human IgG conjugated to horseradish | 2-8° C.* |
| | | peroxidase. | Discard diluted |
| Spike | 1 vial | 125 μL of 1000X concentrated | solutions after |
| Conjugate | | monoclonal antibody specific to | use. |
| Concentrate | | human IgG conjugated to horseradish | |
| (1000X) | | peroxidase. | |
| Conjugate | 1 bottle | 120 mL of a buffered protein base | |
| Buffer - IgG | | with preservatives. | |
| ELISA | | | |
| Sample Buffer - | 3 bottles | 91 mL of a buffered protein base with | |
| IgG ELISA | | preservatives. | |
| TMB Substrate - | 1 bottle | 116 mL of stabilized hydrogen | |
| IgG ELISA | | peroxide and chromogen | |
| | | (tetramethylbenzidine). | |
| Stop Solution - | 1 bottle | 116 mL of acidic solution. | |
| IgG ELISA | | | |
| Wash Buffer - | 2 bottles | 101 mL of a 25-fold concentrated | |
| IgG ELISA | | solution of buffered surfactant with | |
| | | preservative. | |
| RBD Positive | 1 vial | 1.0 mL of monoclonal antibody in a | Store at 2-8° C. |
| Control | | protein buffered base with | Refer to vial label |
| | | preservatives. | for expiration |
| RBD Negative | 1 vial | 1.0 mL of a buffered protein base | date.* |
| Control | | with preservatives. | Discard diluted |
| Spike Low | 1 vial | 1.0 mL of monoclonal antibody in a | solutions after |
| Control | | protein buffered base with | use. |
| | | preservatives. | |
| Spike Mid | 1 vial | 1.0 mL of monoclonal antibody in a | |
| Control | | protein buffered base with | |
| | | preservatives. | |
| Spike High | 1 vial | 1.0 mL of monoclonal antibody in a | |
| Control | | protein buffered base with | |
| | | preservatives. | |
| Spike | 1 vial | 1.25 mL of a monoclonal antibody in | |
| Calibrator 1 | | a buffered base with preservatives. | |
| (0 AU/mL) | | | |
| Spike | 1 vial | 1.25 mL of a monoclonal antibody in | |
| Calibrator 2 | | a buffered base with preservatives. | |
| (0.82 AU/mL) | | | |
| Spike | 1 vial | 1.25 mL of a monoclonal antibody in | |
| Calibrator 3 | | a buffered base with preservatives. | |
| (2.47 AU/mL) | | | |
| Spike | 1 vial | 1.25 mL of a monoclonal antibody in | |
| Calibrator 4 | | a buffered base with preservatives. | |
| (7.41 AU/mL) | | | |
| Spike | 1 vial | 1.25 mL of a monoclonal antibody in | |
| Calibrator 5 | | a buffered base with preservatives. | |
| (22.2 AU/mL) | | | |
| Spike | 1 vial | 1.25 mL of a monoclonal antibody in | |
| Calibrator 6 | | a buffered base with preservatives. | |
| (66.7 AU/mL) | | | |
| Spike | 1 vial | 1.25 mL of a monoclonal antibody in | |
| Calibrator 7 | | a buffered base with preservatives. | |
| (200 AU/mL) | | | |

*The unopened kit is stored at 2-8° C.

### C. Description and Steps

### 1) Sample Collection and Storage:

Serum—Use a serum separator tube (SST) and allow samples to clot for 30 minutes at room temperature before centrifugation for 15 minutes at 1000×g. Remove serum and assay immediately or aliquot and store samples at 2-8° C. or ≤−20° C.

Plasma—Collect plasma using Li-Heparin as an anticoagulant. Centrifuge for 15 minutes at 1000×g within 30 minutes of collection. Assay immediately or aliquot and store samples at 2-8° C. or ≤−20° C.

Serum and Li-Heparin plasma samples are stable for 7 days at 2-8° C. and 3 weeks at ≤−20° C. Do not freeze samples more than one time prior to use.

### 2) Reagent Preparation

1×RBD Conjugate—For each plate, add 11 μL of RBD Conjugate Concentrate (1000λ) to 11 mL of Conjugate Buffer. Mix well.

1× Spike Conjugate—For each plate, add 11 μL of Spike Conjugate Concentrate (1000×) to 11 mL of Conjugate Buffer. Mix well.

Wash Buffer—If crystals have formed in the concentrate, warm to room

temperature and mix gently until the crystals have completely dissolved. For one plate, add 20 mL of Wash Buffer Concentrate to 480 mL of deionized or distilled water to prepare 500 mL of Wash Buffer.

Control Preparation—PDilute each control 5-fold by pipetting 0.4 mL of Sample Buffer into a tube. Add 0.1 mL of the control. Repeat for all 5 controls (RBD Positive, RBD Negative, Spike Low, Spike Mid, and Spike High). Make fresh for each plate.

Calibrators—No preparation required.

### 3) Sample Preparation:

### Heat Inactivation:

1. Heat inactivate samples by placing in a water bath or heat block at 56° C. for 1 hour. Do not leave samples at 56° C. for longer than 1 hour.

2. Aliquot and store samples at 2-8° C. or ≤−20° C. until use.

### RBD ELISA Step:

1. Dilute heat inactivated samples 5-fold in microcentrifuge tubes by adding 10 μL of sample to 40 μL of Sample Buffer.

2. Further dilute samples 20-fold (final 100-fold dilution) by adding 10 μL of diluted sample from step 1 (diluted 5-fold) to 190 μL of Sample Buffer.

### Spike Assay:

1. Dilute heat inactivated samples 5-fold in microcentrifuge tubes by adding 10 μL of sample to 40 μL of Sample Buffer.

2. Further dilute samples 40-fold (final 200-fold dilution) by adding 10 μL of diluted sample from step 1 (diluted 5-fold) to 390 μL of Sample Buffer.

### 4) Interpretation of Results:

### Semi-Quantitative SARS-CoV-2 IgG Antibody Kit Interpretation of Results:

TABLE 13
Semi-Quantitative SARS-CoV-2 IgG Antibody Kit
Semi-Quantitative SARS-CoV-2 IgG Antibody Kit

| RBD ELISA | | | | | |
|---|---|---|---|---|---|
| (Qualitative) | | Spike ELISA (Semi- | | | |
| CI | | Quantitative) | | | |
| Val- | | AU/mL | | Final | |
| ue | Result | Value | Result | Result | Description |
| <0.7 | Negative | N/A | N/A | Nega- | IgG antibodies |
| ≥0.7 | Positive. | <3.2 | Negative | tive | to SARS-CoV-2 |
| | Additional | Between | Positive | Posi- | are NOT |
| | Test is | 3.2 and 125 | | tive | detected. |
| | Required | | | | IgG antibodies |
| | with Spike | | | | to SARS-CoV-2 |
| | ELISA. | | | | ARE detected. |
| | | | | | The measured |
| | | | | | value in AU/mL |
| | | | | | is reported. |
| | | >125 | Positive | | IgG antibodies |
| | | | | | to SARS-CoV-2 |
| | | | | | ARE detected. |
| | | | | | The measured |
| | | | | | value is |
| | | | | | reported as |
| | | | | | “>125 AU/mL”. |

In the RBD ELISA step, if the calculated CI value is ≥0.70, the sample is considered RBD positive and requires confirmation using the Spike ELISA. If the CI value is <0.70, the sample is determined to be negative. See Interpretation of Results table above.

The Spike ELISA step reports results in arbitrary units per milliliter (AU/mL). A value less than the Limit of Quantitation (LoQ) is considered to be negative. The LoQ is 3.2 AU/mL. A value greater than 3.2 AU/mL is considered positive. In addition to reporting a value greater than 3.2 AU/mL as positive, the measured AU/mL can be reported. Values greater than the analytical measuring range should be reported as >125 AU/mL. See Table 13 above for Interpretation of Results.

### D. Semi-Quantitative Sars-Cov-2 IGG Antibody Kit Assay Procedure

Step 1: RBD ELISA Procedure:

CHART 1

| | 1 | 2 | 3 | 4 | 5 | 6 | 7 | 8 | 9 | 10 | 11 | 12 |
|---|---|---|---|---|---|---|---|---|---|---|---|---|
| A | Blank | S6 | S14 | S22 | S30 | S38 | S46 | S54 | S62 | S70 | S78 | S86 |
| B | Pos | S7 | S15 | S23 | S31 | S39 | S47 | S55 | S63 | S71 | S79 | S87 |
| | Control | | | | | | | | | | | |
| C | Neg | S8 | S16 | S24 | S32 | S40 | S48 | S56 | S64 | S72 | S80 | S88 |
| | Control | | | | | | | | | | | |
| D | S1 | S9 | S17 | S25 | S33 | S41 | S49 | S57 | S65 | S73 | S81 | S89 |
| E | S2 | S10 | S18 | S26 | S34 | S42 | S50 | S58 | S66 | S74 | S82 | S90 |
| F | S3 | S11 | S19 | S27 | S35 | S43 | S51 | S59 | S67 | S75 | S83 | Blank |
| G | S4 | S12 | S20 | S28 | S36 | S44 | S52 | S60 | S68 | S76 | S84 | Pos |
| | | | | | | | | | | | | Control |
| H | S5 | S13 | S21 | S29 | S37 | S45 | S53 | S61 | S69 | S77 | S85 | Neg |
| | | | | | | | | | | | | Control |

1. Add 100 μL of control (diluted 5-fold), heat inactivated sample (diluted 100-fold, tested in singlets), or sample buffer (blank) per well. Incubate for 2 hours at room temperature on benchtop. Cover with an adhesive strip if needed.

2. Aspirate each well and wash, repeating the process two times for a total of three washes. Wash by filling each well with Wash Buffer (400 μL) using a squirt bottle, manifold dispenser, or automated plate washer. Complete removal of liquid at each step is essential for good performance. After the last wash, remove any remaining Wash Buffer by aspirating or decanting. Invert the plate and blot it against clean paper towels.

3. Add 100 μL of 1×RBD Conjugate to each well. Incubate for one hour at room temperature. Cover with an adhesive strip if needed.

4. Repeat the aspiration/wash as in step 2.

5. Add 100 μL of Substrate Solution to each well. Incubate for 20 minutes at room temperature. Protect from light.

6. Add 100 μL of Stop Solution to each well. The color in the well should change from blue to yellow. If the color in the well is green or if the color change does not appear uniform, gently tap the plate to ensure thorough mixing.

7. Determine the optical density of each well within 30 minutes (minimum 0 minutes, maximum 30 minutes), using a microplate reader set to 450 nm. If wavelength correction is available, set to 540 nm or 570 nm. If wavelength correction is not available, subtract readings at 540 nm or 570 nm from the readings at 450 nm. This subtraction is required to correct for optical imperfections in the plate.

### RBD ELISA Calculation of Results:

Read the absorbance of each well on a microplate reader using 450 nm as the primary wavelength and 540 nm or 570 nm as the reference wavelength. Average the duplicate readings for each control.

The RBD Positive Control (diluted 5-fold) is used for normalization. Corrected sample OD values are divided by the corrected RBD Positive Control (diluted 5-fold) OD value to calculate a cutoff index (CI) value.

$$\frac{Corrected OD of the sample}{Mean of Corrected OD of RBD Positive Control} = Cutoff Index ( CI )$$

### RBD Quality Control:

Controls with known anti-SARS-CoV-2 IgG concentrations (provided) should be tested in each plate. Satisfactory performance is obtained when controls fall within the established ranges provided in the Certificate of Analysis.

The corrected OD of the blank should be <0.03 OD.

If the results obtained for the controls and blank do not fall within the acceptable limits, the assay results are invalid and samples should be run again.

### Step 2: Spike ELISA Procedure

CHART 2

| | 1 | 2 | 3 | 4 | 5 | 6 | 7 | 8 | 9 | 10 | 11 | 12 |
|---|---|---|---|---|---|---|---|---|---|---|---|---|
| A | Cal 1 | Cal 1 | S2 | S2 | S10 | S10 | S18 | S18 | S26 | S26 | S34 | S34 |
| B | Cal 2 | Cal 2 | S3 | S3 | S11 | S11 | S19 | S19 | S27 | S27 | S35 | S35 |
| C | Cal 3 | Cal 3 | S4 | S4 | S12 | S12 | S20 | S20 | S28 | S28 | S36 | S36 |
| D | Cal 4 | Cal 4 | S5 | S5 | S13 | S13 | S21 | S21 | S29 | S29 | S37 | S37 |
| E | Cal 5 | Cal 5 | S6 | S6 | S14 | S14 | S22 | S22 | S30 | S30 | S38 | S38 |
| F | Cal 6 | Cal 6 | S7 | S7 | S15 | S15 | S23 | S23 | S31 | S31 | Low | Low |
| G | Cal 7 | Cal 7 | S8 | S8 | S16 | S16 | S24 | S24 | S32 | S32 | Mid | Mid |
| H | S1 | S1 | S9 | S9 | S17 | S17 | S25 | S25 | S33 | S33 | High | High |

1. Add 100 μL of control (diluted 5-fold), calibrator (undiluted), or RBD positive heat inactivated sample (diluted 200-fold, tested in duplicates) per well. Incubate for 2 hours at room temperature on benchtop. Cover with an adhesive strip if needed.

2. Aspirate each well and wash, repeating the process two times for a total of three washes. Wash by filling each well with Wash Buffer (400 μL) using a squirt bottle, manifold dispenser, or automated plate washer. Complete removal of liquid at each step is essential to good performance. After the last wash, remove any remaining Wash Buffer by aspirating or decanting. Invert the plate and blot it against clean paper towels.

3. Add 100 μL of 1× Spike Conjugate to each well. Incubate for one hour at room temperature. Cover with an adhesive strip if needed.

4. Repeat the aspiration/wash as in step 2.

5. Add 100 μL of Substrate Solution to each well. Incubate for 20 minutes at room temperature. Protect from light.

6. Add 100 μL of Stop Solution to each well. The color in the well should change from blue to yellow. If the color in the well is green or if the color change does not appear uniform, gently tap the plate to ensure thorough mixing.

7. Determine the optical density of each well within 30 minutes (minimum 0 minutes, maximum 30 minutes), using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate.

### Spike ELISA Calculation of Results:

Read the absorbance of each well on a microplate reader using 450 nm as the primary wavelength and 540 nm or 570 nm as the reference wavelength. Average the duplicate readings for each calibrator, control, and sample.

The calibrators are provided as individual vials and are ready to use. 100 μL of each calibrator is added to the calibrator wells and processed along with the unknown samples and controls.

A monoclonal antibody that recognizes the SARS-CoV-2 RBD of the Spike protein is used as a calibrator. This is used to generate a calibration curve to convert corrected OD units into arbitrary units per milliliter (AU/mL) in the Spike ELISA.

Seven discrete calibrators are provided with the concentrations listed in the following table. Calibration of the Semi-Quantitative SARS-CoV-2 IgG Antibody Kit is traceable to an in-house value assigned reference calibrator.

### Assigned Calibrator Values:

Each calibrator is measured in duplicate and the average of the two measurements is used to generate the calibration curve. A four-parameter logistic (4-PL) curve is fitted to the calibrator values to convert corrected OD units to AU/mL. The calibration curve is generated by the end-user for every plate using appropriate software (e.g., SoftMax Pro, Prism).

The four-parameter curve must have a correlation coefficient of >0.98 or the assay is rejected.

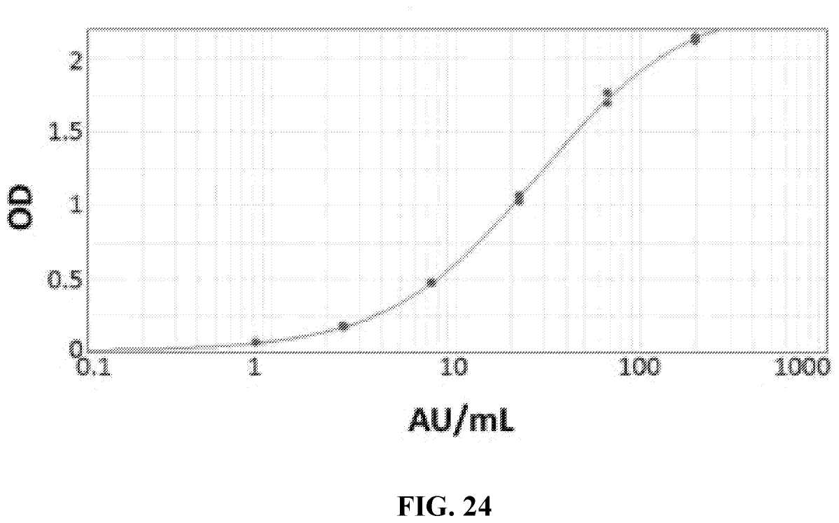

Typical calibration curve: A 7-point standard curve is generated by reducing the data, such as the exemplary data shown in Table 14 below, using computer software capable of generating a four-parameter logistic (4-PL) curve ft (FIG. 24).

TABLE 14
Exemplary Data:

| Calibrator | AU/mL | Average OD |
|---|---|---|
| 1 | 0 | 0.003 |
| 2 | 0.82 | 0.062 |
| 3 | 2.47 | 0.175 |
| 4 | 7.41 | 0.471 |
| 5 | 22.2 | 1.048 |
| 6 | 66.7 | 1.726 |
| 7 | 200 | 2.132 |

The mean value of the duplicate measured OD value of unknown specimens is used to calculate concentration by the following equation where X=AU/mL, Y=OD450, A=lower asymptote, B=hill slope, C=IC50, D=upper asymptote of 4PL curve. These values and the conversion of unknown samples values from the corrected OD to AU/mL are calculated with appropriate software (e.g., SoftMax Pro, Prism GraphPad).

$$x = {c ( \frac{a - x}{y - d} )}^{( 1 / b )}$$

### Spike Quality Control:

Satisfactory performance is obtained when controls fall within the established ranges provided in the Certificate of Analysis. If the results obtained do not fall within the acceptable limits, the assay results are invalid and samples should be retested.

### E. Performance Characteristics

### Analytical Measuring Range

The RBD ELISA step is a qualitative ELISA and there is no defined analytical measuring range (AMR). The output of this step is given in CI values. CI values are calculated by dividing the corrected OD value for unknown samples by the corrected OD value for the positive control. Patient samples with a CI≥0.70 for the RBD ELISA step are tested in the Spike ELISA step. Patient samples with a CI<0.70 for the RBD ELISA are considered negative and not tested in the Spike ELISA step.

The AMR for the Spike protein ELISA step is based on the Limit of Blank, Limit of Detection, Limit of Quantitation (LoQ), Linearity, and Precision studies conducted. This range is based on the LoQ for the lower limit of the measuring interval, the determination of the linear range as described in the linearity section, and the high calibrator, which is set at 200 AU/mL. Results of the semi-quantitative Spike ELISA step are reported in arbitrary units per milliliter (AU/mL).

The claimed AMR for the Spike ELISA is 3.2-125 AU/mL.

### Within-Site Imprecision

### 1) RBD ELISA

Within Laboratory Precision for the RBD ELISA step was determined by measuring four serum samples in two tests per day, three replicates per test for three days. Positive and negative controls were also measured in two replicates per test, two tests per day for three days.

Lot-to-lot imprecision for the RBD ELISA step was determined by measuring four serum samples in two runs per day, three replicates per run for three days using two different lots of reagents. Positive and negative controls were also measured in two replicates per run, two runs per day for three days with two lots of reagents. See Table 15, infra.

TABLE 15

| | | Mean | Within-Run | | Between-Run | | Between-Day | | Between-Lot | | Total | |
|---|---|---|---|---|---|---|---|---|---|---|---|---|
| Sample | n | (CI) | SD | % CV | SD | % CV | SD | % CV | SD | % CV | SD | % CV |
| Negative | 24 | 0.041 | 0 | 9.40% | 0 | 8.10% | 0 | 0.00% | 0 | 0.00% | 0.01 | 12.40% |
| Control | | | | | | | | | | | | |
| Sample 1 | 36 | 0.143 | 0.01 | 4.40% | 0.01 | 4.20% | 0.01 | 4.40% | 0.01 | 9.50% | 0.02 | 12.10% |
| (Negative) | | | | | | | | | | | | |
| Sample 5 | 36 | 0.173 | 0.01 | 5.50% | 0.01 | 8.00% | 0.01 | 7.30% | 0.03 | 17.80% | 0.04 | 21.50% |
| (Negative) | | | | | | | | | | | | |
| Sample 6 | 36 | 0.597 | 0.03 | 4.70% | 0.06 | 10.90% | 0.01 | 1.80% | 0.08 | 12.90% | 0.11 | 17.60% |
| (Near | | | | | | | | | | | | |
| Cutoff) | | | | | | | | | | | | |
| Sample 2 | 36 | 0.686 | 0.03 | 4.70% | 0.04 | 6.50% | 0.03 | 4.40% | 0.1 | 13.90% | 0.11 | 16.70% |
| (Near | | | | | | | | | | | | |
| Cutoff) | | | | | | | | | | | | |
| Sample 7 | 36 | 0.75 | 0.04 | 5.60% | 0.04 | 4.70% | 0.03 | 4.60% | 0.13 | 16.90% | 0.14 | 19.00% |
| (Near LoD) | | | | | | | | | | | | |
| Sample 3 | 36 | 0.963 | 0.05 | 5.40% | 0.03 | 3.10% | 0.04 | 4.10% | 0.14 | 14.40% | 0.16 | 16.20% |
| (Near LoD) | | | | | | | | | | | | |
| Positive | 24 | 1 | 0.04 | 3.90% | 0 | 0.00% | 0 | 0.00% | 0 | 0.00% | 0.04 | 3.90% |
| Control | | | | | | | | | | | | |
| Sample 4 | 36 | 1.704 | 0.06 | 3.50% | 0.1 | 6.00% | 0.03 | 1.70% | 0.24 | 14.10% | 0.27 | 15.80% |
| (Positive) | | | | | | | | | | | | |
| Sample 8 | 36 | 1.864 | 0.07 | 3.80% | 0.04 | 2.20% | 0.1 | 5.20% | 0.27 | 14.30% | 0.29 | 15.80% |
| Positive) | | | | | | | | | | | | |

### 2) Spike ELISA

Within Laboratory Precision for the Spike ELISA step was determined by measuring three serum samples in two tests per day, three replicates per test for three days. The Low, Mid, and High controls were also measured in two replicates per test, two tests per day for three days.

Lot-to-lot imprecision for the Spike ELISA step was determined by measuring three serum samples in two runs per day, three replicates per run for three days with two different lots of reagents. The Low, Mid, and High controls were also measured in two replicates per run, two runs per day for three days with two lots of reagents. See Table 16, infra.

TABLE 16

| | | Mean | Within-Run | | Between-Run | | Between-Day | | Between-Lot | | Total | |
|---|---|---|---|---|---|---|---|---|---|---|---|---|
| Sample | n | (AU/mL) | SD | % CV | SD | % CV | SD | % CV | SD | % CV | SD | % CV |
| High Ctrl | 66 | 38.28 | 3.13 | 8.2% | 0.79 | 2.1% | 1.50 | 3.9% | 0.00 | 0.0% | 3.56 | 9.3% |
| Low Ctrl | 66 | 2.23 | 0.14 | 6.2% | 0.08 | 3.4% | 0.00 | 0.0% | 0.06 | 2.8% | 0.17 | 7.6% |
| Mid Ctrl | 66 | 9.80 | 0.51 | 5.2% | 0.30 | 3.0% | 0.00 | 0.0% | 0.10 | 1.0% | 0.60 | 6.1% |
| Sample | 36 | 3.46 | 0.13 | 3.6% | 0.12 | 3.5% | 0.00 | 0.0% | 0.00 | 0.0% | 0.17 | 5.0% |
| 353 | | | | | | | | | | | | |
| Sample | 36 | 4.29 | 0.13 | 3.0% | 0.13 | 3.0% | 0.02 | 0.5% | 0.05 | 1.1% | 0.19 | 4.4% |
| 534 | | | | | | | | | | | | |
| Sample | 36 | 41.80 | 3.07 | 7.3% | 1.09 | 2.6% | 3.41 | 8.1% | 0.00 | 0.0% | 4.71 | 11.3% |
| 735 | | | | | | | | | | | | |
| Sample | 36 | 58.47 | 4.55 | 7.8% | 1.03 | 1.8% | 3.68 | 6.3% | 0.00 | 0.0% | 5.95 | 10.2% |
| 744 | | | | | | | | | | | | |
| Sample | 36 | 121.83 | 12.64 | 10.4% | 8.64 | 7.1% | 7.58 | 6.2% | 2.77 | 2.3% | 17.31 | 14.2% |
| 820 | | | | | | | | | | | | |
| Sample | 36 | 104.19 | 10.32 | 9.9% | 5.13 | 4.9% | 9.65 | 9.3% | 1.25 | 1.2% | 15.08 | 14.5% |
| 850 | | | | | | | | | | | | |

### Site-To-Site Reproducibility

### 1) RBD ELISA

Site-to-site reproducibility for RBD ELISA step was determined by measuring four serum samples in two tests per day, three replicates per test for three days using at two different sites. Positive and negative controls were also measured in two replicates per test, two tests per day for three days at the two sites. See Table 17, infra.

TABLE 17

| | | Mean | Within-Run | | Between-Run | | Between-Day | | Between-Site | | Total | |
|---|---|---|---|---|---|---|---|---|---|---|---|---|
| Sample | n | (CI) | SD | % CV | SD | % CV | SD | % CV | SD | % CV | SD | % CV |
| Negative | 24 | 0.092 | 0.01 | 5.7% | 0.01 | 7.2% | 0.00 | 0.0% | 0.07 | 79.0% | 0.07 | — |
| Control | | | | | | | | | | | | |
| Positive | 24 | 1.000 | 0.03 | 3.3% | 0.00 | 0.0% | 0.00 | 0.0% | 0.00 | 0.0% | 0.03 | 3.3% |
| Control | | | | | | | | | | | | |
| Sample 1 | 36 | 0.228 | 0.01 | 5.2% | 0.02 | 10.9% | 0.00 | 0.0% | 0.11 | 46.2% | 0.11 | 47.8% |
| (Negative) | | | | | | | | | | | | |
| Sample 2 | 36 | 0.718 | 0.03 | 4.2% | 0.07 | 10.2% | 0.00 | 0.0% | 0.05 | 6.4% | 0.09 | 12.8% |
| (Near | | | | | | | | | | | | |
| Cutoff) | | | | | | | | | | | | |
| Sample 3 | 36 | 1.044 | 0.06 | 6.2% | 0.05 | 5.0% | 0.08 | 7.6% | 0.00 | 0.0% | 0.11 | 11.0% |
| (Near LoD) | | | | | | | | | | | | |
| Sample 4 | 36 | 1.811 | 0.10 | 5.6% | 0.06 | 3.2% | 0.06 | 3.5% | 0.08 | 4.4% | 0.16 | 8.6% |
| (Positive) | | | | | | | | | | | | |
| Sample 5 | 36 | 0.299 | 0.02 | 7.9% | 0.02 | 8.2% | 0.01 | 2.9% | 0.15 | 48.6% | 0.15 | 50.0% |
| (Negative) | | | | | | | | | | | | |
| Sample 7 | 36 | 0.832 | 0.05 | 6.0% | 0.02 | 2.7% | 0.06 | 7.2% | 0.00 | 0.0% | 0.08 | 9.8% |
| (Near LoD) | | | | | | | | | | | | |
| Sample 8 | 36 | 2.032 | 0.08 | 3.8% | 0.07 | 3.5% | 0.09 | 4.5% | 0.00 | 0.0% | 0.14 | 6.9% |
| (Positive) | | | | | | | | | | | | |
| Sample 8 | 36 | 1.864 | 0.07 | 3.8% | 0.04 | 2.2% | 0.10 | 5.2% | 0.27 | 14.3% | 0.29 | 15.8% |
| (Positive) | | | | | | | | | | | | |

### 2) Spike ELISA

Site-to-site reproducibility for the Spike ELISA step was determined by measuring three serum samples in two tests per day, three replicates per test for three days at two different sites. The Low, Mid, and High controls were also measured in two replicates per test, two tests per day for three days at the two sites. See Table 18, infra.

TABLE 18

| | | Mean | Within-Run | | Between-Run | | Between-Day | | Between-Site | | Total | |
|---|---|---|---|---|---|---|---|---|---|---|---|---|
| Sample | n | (AU/mL) | SD | % CV | SD | % CV | SD | % CV | SD | % CV | SD | % CV |
| High Ctrl | 42 | 38.79 | 3.86 | 10.0% | 0.72 | 1.8% | 1.23 | 3.2% | 1.36 | 3.5% | 4.33 | 11.2% |
| Low Ctrl | 42 | 2.23 | 0.15 | 6.9% | 0.08 | 3.8% | 0.00 | 0.0% | 0.15 | 6.7% | 0.23 | 10.4% |
| Mid Ctrl | 42 | 9.54 | 0.55 | 5.7% | 0.35 | 3.7% | 0.00 | 0.0% | 0.31 | 3.2% | 0.72 | 7.5% |
| Sample | 36 | 3.54 | 0.16 | 4.6% | 0.27 | 7.6% | 0.00 | 0.0% | 0.00 | 0.0% | 0.31 | 8.9% |
| 353 | | | | | | | | | | | | |
| Sample | 36 | 4.52 | 0.15 | 3.4% | 0.45 | 9.9% | 0.00 | 0.0% | 0.19 | 4.1% | 0.51 | 11.3% |
| 534 | | | | | | | | | | | | |
| Sample | 33 | 42.45 | 2.54 | 6.0% | 1.28 | 3.0% | 2.04 | 4.8% | 0.00 | 0.0% | 3.50 | 8.2% |
| 735 | | | | | | | | | | | | |
| Sample | 36 | 63.67 | 7.37 | 11.6% | 0.00 | 0.0% | 2.35 | 3.7% | 7.04 | 11.1% | 10.46 | 16.4% |
| 744 | | | | | | | | | | | | |
| Sample | 35 | 137.71 | 13.58 | 9.9% | 15.09 | 11.0% | 0.00 | 0.0% | 15.12 | 11.0% | 25.31 | 18.4% |
| 820 | | | | | | | | | | | | |
| Sample | 36 | 99.59 | 12.96 | 13.0% | 0.00 | 0.0% | 2.37 | 2.4% | 12.65 | 12.7% | 18.26 | 18.3% |
| 850 | | | | | | | | | | | | |

### Analytical Sensitivity

The analytical sensitivity—Limit of Blank (LoB), Limit of Detection (LoD), and Limit of Quantitation (LoQ) were established according to the recommendations in CLSI guideline EP17-A2. The summary data for the RBD and Spike ELISA steps is presented in Table 19 below.

TABLE 19
Summary data for the RBD and Spike ELISA steps:

| | RBD ELISA | Spike ELISA |
|---|---|---|
| Sensitivity | (CI) | (AU/mL) |
| LoB | 0.70 | 1.98 |
| LoD | 0.82 | 2.61 |
| LoQ | — | 3.20 |

### Linearity

Linearity was demonstrated according to recommendations in CLSI guideline EP06-A. Three individual serum samples were proportionally diluted with negative serum samples. The negative serum samples used to make the dilutions were pre-COVID samples collected prior to September 2019. Each dilution was measured with four replicates.

The linear range was demonstrated to be 1.89-125.7 AU/mL with a deviation from linearity≤15%. The Analytical Measuring Range (AMR) is 3.2-125 AU/mL.

### Clinical Agreement Study

To evaluate clinical performance of the Semi-Quantitative SARS-CoV-2 IgG Antibody Kit a clinical study using retrospective samples was conducted. Positive Percent Agreement (PPA) was determined by evaluating 251 unique serum samples collected from SARS-CoV-2 positive subjects confirmed by RT-PCR. Negative Percent Agreement (NPA) was determined by evaluating 284 negative serum samples. The 284 negative serum samples were collected prior to December 2019 (Pre-COVID) and 11 of the samples were collected from HIV-positive subjects.

### Positive Percent Agreement:

Out of the 251 PCR positive subjects, 235 subjects had the days from symptoms onset information available. The PPA was calculated considering days from PCR result and also from symptoms onset, as indicated in the Tables 20 and 21.

TABLE 20
Positive Percent Agreement in PCR-Confirmed Positive
Subjects from Days from Positive PCR Test

| Days between Positive | | | | 95% |
|---|---|---|---|---|
| PCR and Sample | Total | Number | | Confident |
| Collection | Samples | Positive | PPA | Interval |
| ≤7 | 3 | 3 | 100% | 43.85%-100% |
| 8-14 | 3 | 3 | 100% | 43.85%-100% |
| ≥15 | 245 | 242 | 98.78% | 96.46%-99.58% |
| Total | 251 | | | |

TABLE 21
Positive Percent Agreement in PCR-Confirmed Positive
Subjects from Days from Symptom Onset

| Days between | | | | |
|---|---|---|---|---|
| Symptom Onset | | | | |
| and Sample | | Number | | 95% Confident |
| Collection | Total Samples | Positive | PPA | Interval |
| ≤7 | 0 | 0 | N/A | N/A |
| 8-14 | 0 | 0 | N/A | N/A |
| ≥15 | 235 | 233 | 99.15% | 96.95%-99.77% |
| Total | 235 | | | |

### Negative Percent Agreement:

For the negative samples, the NPA was 99.6% (283/284, 95% CI: 98.0%-100%).

All 11 of the HIV positive samples tested negative with the Semi-Quantitative SARS-CoV-2 IgG Antibody Kit. See Table 22, infra.

TABLE 22

| | | | | | 95% |
|---|---|---|---|---|---|
| | Total | Number | | | Confidence |
| | Samples | Negative | NPA | | Interval |
| PreCOVID-19 | 273 | 272 | 99.6% | (272/273) | 97.5% to |
| | | | | | 99.9% |
| HIV Positive | 11 | 11 | 100.0% | (11/11) | 74.12% to |
| | | | | | 100% |
| Total | 284 | 283 | 99.6% | (283/284) | 98.0% to |
| | | | | | 99.9% |

### F. Analytical Specificity

### Cross-Reactivity

The Semi-Quantitative SARS-CoV-2 IgG Antibody Kit was evaluated for potential cross-reactivity. Eighty-six (86) serum samples with antibodies to the diseases included in the Table 23 below were tested with the Semi-Quantitative SARS-CoV-2 IgG Antibody Kit. The samples evaluated were all collected prior to August 2019. No cross-reactivity was observed.

TABLE 23
Cross-Reactivity Results for the Semi-Quantitative
SARS-CoV-2 IgG Antibody Kit

| Disease | n | % Negative |
|---|---|---|
| Epstein-Barr Virus | 5 | 100% |
| Rheumatoid Arthritis | 5 | 100% |
| Rheumatoid Factor | 5 | 100% |
| Cytomegalovirus | 5 | 100% |
| Human anti-Mouse Antibody | 5 | 100% |
| Varicella Zoster Virus | 5 | 100% |
| Rubella | 5 | 100% |
| Antinuclear Antibody | 5 | 100% |
| Influenza B | 5 | 100% |
| Herpes Simplex Virus | 5 | 100% |
| Hepatitis C Virus | 5 | 100% |
| Hepatitis B Virus | 5 | 100% |
| Lupus | 5 | 100% |
| HIV | 11 | 100% |
| Common Coronavirus | 10 | 100% |
| Total | 86 | 100% |

### Potential Interfering Substances

Potential interference with the Semi-Quantitative SARS-CoV-2 IgG Antibody Kit test was evaluated by assessing potential interference with the RBD ELISA step as well as the Spike ELISA step.

### RBD ELISA Step:

Interference testing was conducted following recommendations in CLSI guideline EP07-A3. Four serum samples were used to evaluate potential endogenous interferents. Data was evaluated quantitatively by comparing the percent difference between the mean CI value of the sample without the potential interferent substance and the mean CI value of the samples containing the potential interferent substance. All samples demonstrated a difference of ≤15% at the specified concentrations in the table below.

### Spike ELISA Step:

Interference testing was conducted following recommendations in CLSI guideline EP07-A3. Two serum samples were used to evaluate potential endogenous interferents for the Spike ELISA, one at approximately 5.0 AU/mL, and one at approximately 50 AU/mL. Data was evaluated by comparing the percent difference between the mean AU/mL value of the sample without the potential interferent substance and the mean AU/mL value of the samples containing the potential interferent substance. All samples demonstrated a difference of ≤15% at the specified concentration.

The following potential interferent were evaluated at the indicated concentrations:

TABLE 24

| Interferent | Highest Concentration | |
|---|---|---|
| Conjugated Bilirubin | 104 | mg/dL |
| Unconjugated Bilirubin | 96.6 | mg/dL |
| Hemoglobin | 10.6 | g/dL |
| Total Protein | 8.6 | g/dL |
| Cholesterol | 315 | mg/dL |
| Triglycerides | 6710 | mg/dL |

### Matrix Equivalency

Matrix equivalency between serum and Lithium Heparin plasma specimens was evaluated with the Semi-Quantitative SARS-CoV-2 IgG Antibody Kit by assessing equivalence between serum and lithium heparin plasma for the RBD ELISA step as well as the Spike ELISA step.

### RBD ELISA Step:

Matrix equivalency for the RBD ELISA step was demonstrated by testing 36 matched serum and Li-Heparin plasma samples that spanned the range of expected CI values. Passing-Bablok regression analysis was used to compare the results from the serum sample measurement to the results from the Li-Heparin plasma sample measurements. The Table 25 below summarizes the results of this study.

TABLE 25

| | | Range | Slope | Intercept | |
|---|---|---|---|---|---|
| Comparison | n | (CI) | (95% CI) | (95% CI) | r |
| Serum vs | 36 | 0.13-5.1 | 1.02 | −0.01 | 0.99 |
| Li-Heparin Plasma | | | (0.97-1.07) | (−0.03-−0.05) | |

### Spike ELISA Step:

Matrix equivalency for the Spike ELISA step was demonstrated by testing 18 matched serum and Li-Heparin plasma samples that spanned the analytical measuring range of the assay. Passing-Bablok regression analysis was used to compare the results from the serum sample measurement to the results from the Li-Heparin plasma sample measurements. Table 26 below summarizes the results of this study.

TABLE 26

| | | Range | Slope | Intercept | |
|---|---|---|---|---|---|
| Comparison | n | (CI) | (95% CI) | (95% CI) | r |
| Serum vs | 18 | 6.1-96.4 | 0.99 | −0.04 | 0.99 |
| Li-Heparin Plasma | | | (0.96-1.06) | (−1.8-0.88) | |

### 6.13 Example 13: Quantitative SARS-CoV-2 IgG Antibody Kit

The 2-phase assay is an antigen-down enzyme immunoassay which utilizes a recombinant SARS-CoV-2 Spike protein RBD antigen pre-coated onto a 96-well microplate in phase 1. When the sample is added, antibodies found in the sample that recognize SARS-CoV-2 RBD antigen bind to the antigen coated plate and are retained in the well. After washing away unbound substances, an enzyme-linked monoclonal antibody specific for human IgG is added to the wells. Following a wash to remove any unbound enzyme-linked antibody, a substrate is added to the wells and color develops in proportion to the amount of IgG antibodies in the sample bound to the SARS-CoV-2 RBD antigen. The color development is stopped, and the intensity of the color is measured. Samples that have a measured value above a pre-determined cutoff are determined to be positive and tested in the 2nd phase ELISA.

Positive samples from phase 1 are evaluated on a second orthogonal ELISA to quantify the levels of IgG antibodies to the SARS-CoV-2 Spike protein. For this assay, a recombinant SARS-CoV-2 Spike protein is pre-coated onto a 96-well microplate and used to bind antibodies found in the sample. When the sample is added, antibodies found in the sample that recognize SARS-CoV-2 Spike protein bind the antigen coated plate and are retained in the well. After washing away unbound substances, an enzyme-linked monoclonal antibody specific for human IgG is added to the wells. Following a wash to remove any unbound enzyme-linked antibody, a substrate is added to the wells and color develops in proportion to the amount of IgG antibodies in the sample bound to the SARS-CoV-2 Spike protein. The color development is stopped, and the intensity of the color is measured. The signal from unknown samples is compared to a calibration curve to generate a final result in arbitrary units per milliliter (AU/mL).

Materials Provided & Storage Conditions

The materials and storage conditions are the same as described in Table 12, supra, in Example 12 with the exception that (i) 4 RBD Antigen Microplates and 5 Spike Protein Plates are provided, and (ii) Conjugate buffer IgG ELISA is supplied as 1 bottle of 120 mL of a protein based solution with preservatives.

### Sample Collection and Storage

Serum—Use a serum separator tube (SST) and allow samples to clot for 30 minutes at room temperature before centrifugation for 15 minutes at 1000×g. Remove serum and assay immediately or aliquot and store samples at ≤−20° C. Avoid repeated freeze-thaw cycles.

Plasma—Collect plasma using EDTA or heparin as an anticoagulant. Centrifuge for 15 minutes at 1000×g within 30 minutes of collection. Assay immediately or aliquot and store samples at ≤−20° C. Avoid repeated freeze-thaw cycles.

Note: Citrate plasma has not been validated for use in this assay. Grossly hemolyzed or lipemic samples are not acceptable in this assay.

### Reagent Preparation

See Reagent Preparation in Example 12 for Reagent Preparation.

### Sample Preparation

See Sample Preparation in Example 12 for Sample Preparation—with the exception that aliquots of heat inactivated samples should be stored at ≤20° C. until use.

### RBD ELISA Procedure

See Step 1: RBD ELISA Procedure in Example 12, supra (including Chart 1), for the RBD ELISA Procedure. With respect to optical density readings, readings made directly at 450 nm without correction may be higher and less accurate.

### RBD ELISA Calculation Results

The RBD Positive Control (diluted 5-fold) is used for normalization. Corrected sample OD values (see RBD ELISA step 7) are divided by the corrected RBD Positive Control (diluted 5-fold) OD value to calculate a cutoff index (CI) value.

$$\frac{Corrected OD of the Sample}{Corrected OD of RBD Positive Control} = Cutoff Index ( CI )$$

If the calculated CI value is >0.7, the sample is considered RBD positive and requires confirmation using the Spike ELISA. If the CI value is <0.7, the sample is negative and contained no detectable levels of antibodies to the RBD protein fragment of SARS-CoV2 Spike protein.

### Spike ELISA Procedure

CHART 3

| | 1 | 2 | 3 | 4 | 5 | 6 | 7 | 8 | 9 | 10 | 11 | 12 |
|---|---|---|---|---|---|---|---|---|---|---|---|---|
| A | Cal 1 | Cal 1 | S3 | S11 | S19 | S27 | S35 | S43 | S51 | S59 | S67 | S72 |
| B | Cal 2 | Cal 2 | S4 | S12 | S20 | S28 | S36 | S44 | S52 | S60 | S68 | S73 |
| C | Cal 3 | Cal 3 | S5 | S13 | S21 | S29 | S37 | S45 | S53 | S61 | S69 | S74 |
| D | Cal 4 | Cal 4 | S6 | S14 | S22 | S30 | S38 | S46 | S54 | S62 | S70 | S75 |
| E | Cal 5 | Cal 5 | S7 | S15 | S23 | S31 | S39 | S47 | S55 | S63 | S71 | S76 |
| F | Cal 6 | Cal 6 | S8 | S16 | S24 | S32 | S40 | S48 | S56 | S64 | Low | Low |
| G | Cal 7 | Cal 7 | S9 | S17 | S25 | S33 | S41 | S49 | S57 | S65 | Medium | Medium |
| H | S1 | S2 | S10 | S18 | S26 | S34 | S42 | S50 | S58 | S66 | High | High |

1. Add 100 μL of control (diluted 5-fold), calibrator (undiluted), or RBD positive heat inactivated sample (diluted 200-fold, run in singlets) per well. Incubate for 2 hours at room temperature on benchtop. Cover with an adhesive strip if needed.

2. Aspirate each well and wash, repeating the process two times for a total of three washes. Wash by filling each well with Wash Buffer (400 μL) using a squirt bottle, manifold dispenser, or autowasher. Complete removal of liquid at each step is essential to good performance. After the last wash, remove any remaining Wash Buffer by aspirating or decanting. Invert the plate and blot it against clean paper towels.

3. Add 100 μL of 1× Spike Conjugate to each well. Incubate for one hour at room temperature. Cover with an adhesive strip if needed.

4. Repeat the aspiration/wash as in step 2.

5. Add 100 μL of Substrate Solution to each well. Incubate for 20 minutes at room temperature. Protect from light.

6. Add 100 μL of Stop Solution to each well. The color in the well should change from blue to yellow. If the color in the well is green or if the color change does not appear uniform, gently tap the plate to ensure thorough mixing.

7. Determine the optical density of each well within 30 minutes, using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

### Spike ELISA Calculation of Results

Create a standard curve by reducing the calibrator values using computer software capable of generating a four parameter logistic (4-PL) curve fit. As an alternative, construct a standard curve by plotting the mean absorbance for each standard on the y-axis against the concentration on the x-axis and draw a best fit curve through the points on the graph. The data may be linearized by plotting the log of the concentration versus the log of the OD and the best fit line can be determined by regression analysis. This procedure will produce an adequate but less precise fit of the data.

Samples falling below the Limit of Quantification (LoQ) of 3.20 AU/mL are considered negative. Values above the analytical measuring range should be reported as >160 AU/mL.

See Table 14 for exemplary data and FIG. 24 for an exemplary standard curve. A standard curve should be generated for each Spike plate.

### Precision

Intra-Assay Precision (Precision within an assay): Three samples of known concentration were tested on one plate to assess intra-assay precision.

Inter-Assay Precision (Precision between assays): Three samples of known concentration were tested in separate assays to assess inter-assay precision.

TABLE 27
RBD ELISA

| | Intra-Assay Precision | | | Inter-Assay Precision | | |
|---|---|---|---|---|---|---|
| Sample | 1 | 2 | 3 | 1 | 2 | 3 |
| n | 3 | 3 | 3 | 18 | 18 | 18 |
| Mean (CI) | 0.565 | 0.914 | 1.58 | 0.616 | 0.862 | 1.53 |
| Standard deviation | 0.029 | 0.011 | 0.020 | 0.047 | 0.039 | 0.123 |
| CV (%) | 5.1 | 1.2 | 1.3 | 7.6 | 4.5 | 8.0 |

TABLE 28
Spike ELISA

| | Intra-Assay Precision | | | Inter-Assay Precision | | |
|---|---|---|---|---|---|---|
| Sample | 1 | 2 | 3 | 1 | 2 | 3 |
| n | 3 | 3 | 3 | 18 | 18 | 18 |
| Mean (AU/mL)* | 3.44 | 57.2 | 109 | 3.47 | 58.5 | 109 |
| Standard deviation | 0.080 | 3.03 | 7.49 | 0.093 | 5.09 | 12.7 |
| CV (%) | 2.3 | 5.3 | 6.9 | 2.7 | 8.7 | 11.7 |

### Analytical Sensitivity

The analytical sensitivity—limit of blank (LoB), limit of detection (LoD), and limit of quantitation (LoQ) were established according to the recommendations in CLSI guideline EP17-A2. RBD and Spike ELISAs summary data is presented below.

TABLE 29

| | RBD ELISA | Spike ELISA |
|---|---|---|
| Sensitivity | (CI) | (AU/mL) |
| LoB | 0.70 | 1.98 |
| LoD | 0.82 | 2.61 |
| LoQ | — | 3.20 |

### Linearity

Linearity was demonstrated according to recommendations in CLSI guideline EP06-A. Three individual samples were proportionally diluted with blank serum samples. The blank serum samples used to make the dilutions were preCOVID-19 samples collected prior to September 2019.

The linear range is 3.1-160 AU/mL and the Analytical Measuring Range (AMR) is 3.2-161 AU/mL.

TABLE 30

| | # Dilution Levels in | Linear Range |
|---|---|---|
| Sample | the Linear Range | (AU/mL) |
| 1 | 10 | 4.16-161 |
| 2 | 10 | 8.19-145 |
| 3 | 9 | 3.08-75.1 |

### Sample Results

Samples known to be PCR positive for COVID-19 and samples obtained prior to September 2019 (PreCOVID-19) were tested according to the protocol on one lot of materials.

TABLE 31

| Sample Type | Total Samples | Number Negative | Number Positive |
|---|---|---|---|
| PreCOVID-19 | 470 | 469 | 1 |
| PCR Positive | 359 | 8 | 351 |

### Calibration

A monoclonal antibody with apparent viral neutralizing properties specific to the SARS-CoV-2 receptor binding domain of the spike protein is used as a calibrator. This is used to generate a standard curve to convert OD units into arbitrary units per milliliter (AU/mL) in the Spike ELISA.

NIBSC Anti-SARS-CoV-2 Diagnostic Calibrant (20/162) approximate value (U)=0.007× Kantaro ELISA value (AU/mL)

### Class Specificity

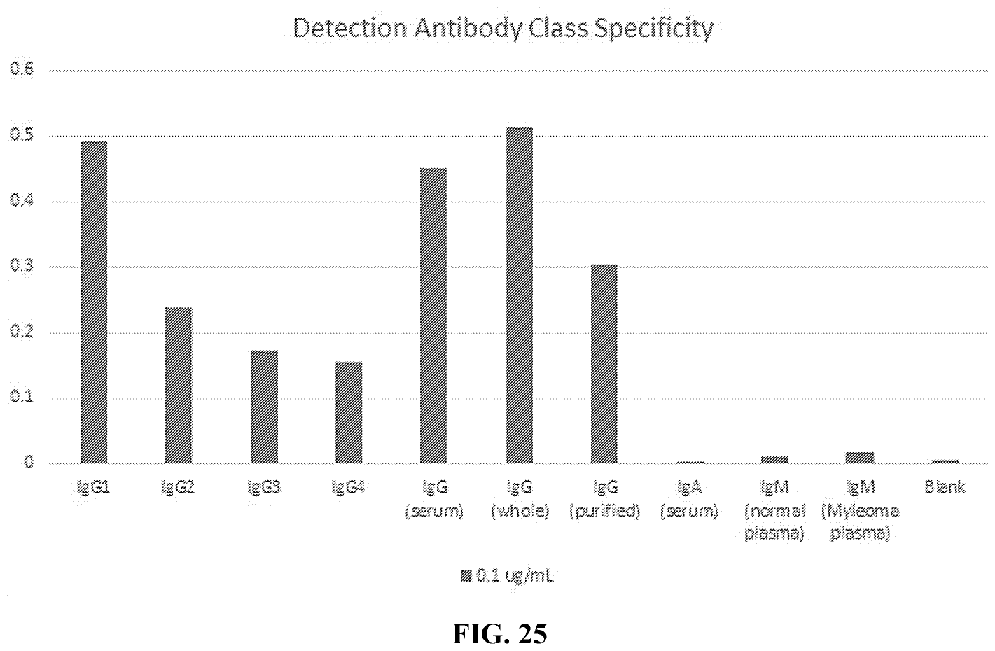

Class specificity of the monoclonal detection antibody was evaluated in an antigen-down ELISA study. Ten antigens, including seven different human IgG samples, were diluted to 25 ng/ml or 100 ng/ml (not shown) and coated on a plate. A dilution series of the monoclonal detection antibody was incubated on the plate prior to detection. Summary data indicates that the monoclonal detection antibody detects human IgG isotypes (FIG. 25) and has minimal detection of human IgA or IgM that approaches level of the blank with titration. Specificity

Disease state (Antinuclear Antibody, Cold coronaviruses, Cytomegalovirus, Epstein-Barr Virus, Hepatitis B Virus, Hepatitis C Virus, Herpes Simplex Virus, HIV, Human anti-Mouse Antibody Influenza virus, Lupus, Rheumatoid Arthritis, Rheumatoid Factor, Rubella, and Varicella Zoster Virus) samples collected prior to August 2019 were tested in this assay for cross-reactivity. No cross-reactivity was observed.

### Microneutralization

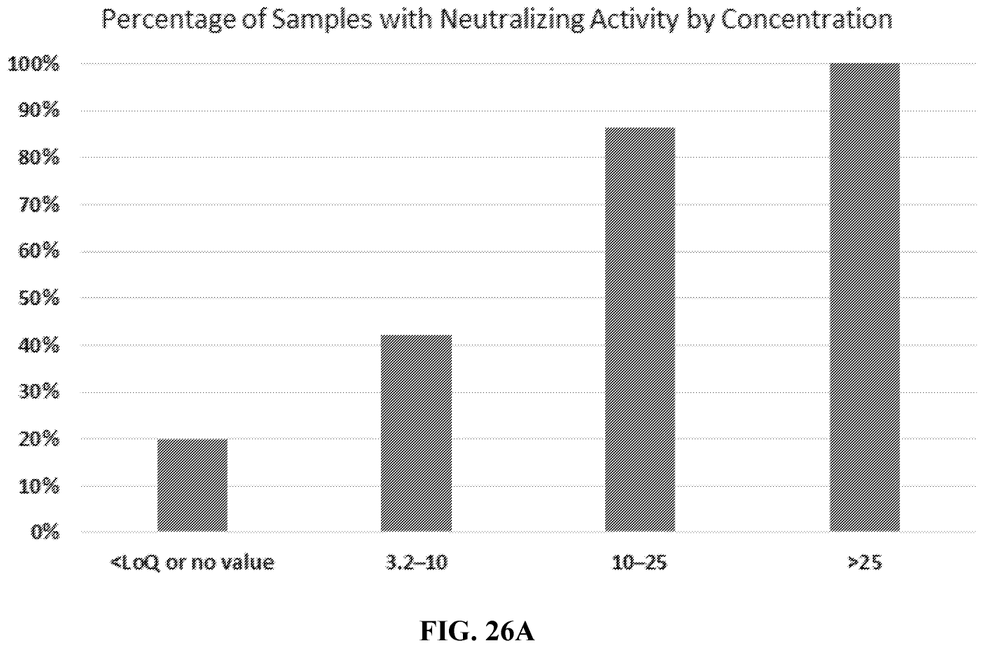

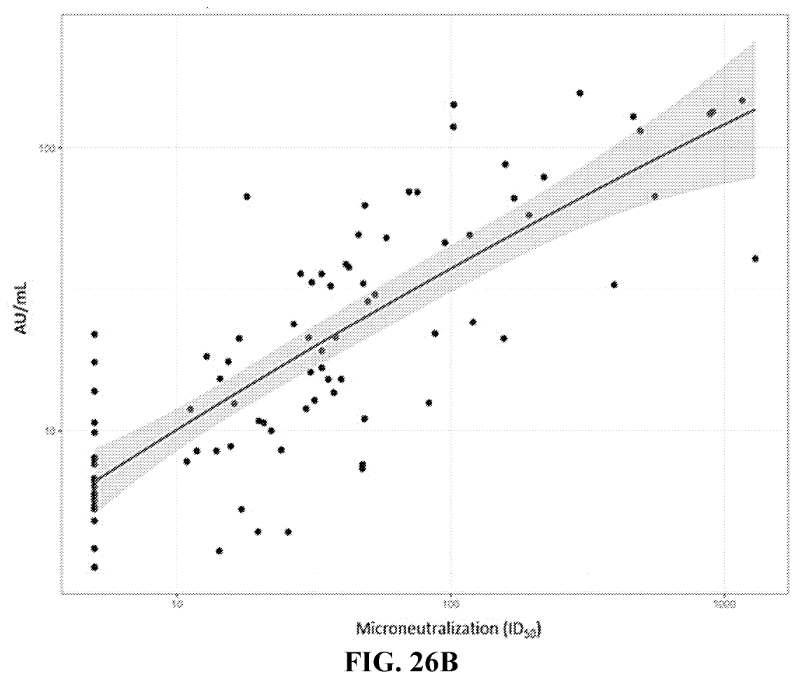

A study was conducted to correlate the quantitative levels of anti-Spike protein IgG antibodies to viral neutralization in a microneutralization (MN) assay. 120 patient samples with levels of antibodies across the AMR of the assay were evaluated in a MN assay. See FIGS. 26A-26B. Values shown below are not multiplied by the dilution factor. Information on the format and interpretation of the MN assay can be found in the following reference: Amanat, F., et. al., “A Serological Assay to Detect SARS-CoV-2 Seroconversion in Humans”; Nature Medicine. 2020 May 12. PMID: 32398876. See also Example 4, supra.

### 6.14. Example 14: Quantitative SARS-CoV-2 IgG Antibody Kit

This example describes a Quantitative SARS-CoV-2 IgG Antibody Kit, which consists of two serial direct Enzyme-Linked Immunosorbent Assays (ELISA) intended for quantitative detection of human IgG antibodies to the SARS-CoV-2 virus in serum and plasma (Li-Heparin and K2-EDTA) specimens collected from individuals suspected by their healthcare provider of prior infection with the SARS-CoV-2 virus that causes COVID-19.

An initial qualitative ELISA is performed against recombinant Receptor Binding Domain of SARS-CoV-2, followed for positive specimens by a quantitative ELISA against full length SARS-CoV-2 Spike protein. The assay aids in establishing the quantitative levels of neutralizing antibodies indicative of an adaptive immune response to SARS-CoV-2 in patients suspected of previous SARS-CoV-2 infection, or for the detection of IgG seroconversion in patients following known recent SARS-CoV-2 infection.

Determination of the number of individuals who are demonstrated to have developed specific antibodies to SARS-CoV-2 aids in the determination of seroprevalence in any geographic region or group of exposed individuals and may be indicative of the potential risk of reinfection. The results of the assay correlate with the neutralization of SARS-CoV-2 virus in vitro.

Generally, results from the Quantitative SARS-CoV-2 IgG Antibody IVD Kit should not be used as the sole basis for diagnosis and should not be used for the diagnosis of patients with acute COVID-19 infection.

Results are for the detection of SARS-CoV-2 IgG antibodies. IgG antibodies to SARS-CoV-2 generally become detectable beginning 10-14 days following infection but may occur later. The presence of IgG antibodies, following previously negative testing, defines IgG antibody seroconversion following SARS-CoV-2 infection.

Negative results do not preclude acute SARS-CoV-2 infection and should not be used as the sole basis for patient management decisions. IgG antibodies may not be present for more than two weeks following infection, and patients may remain infectious during acute infection even if IgG antibody is present. Results must be combined with clinical observations, patient history, and epidemiological information. The sensitivity of the Quantitative SARS-CoV-2 IgG Antibody IVD Kit early after infection is unknown.

False positive results for IgG antibodies may occur due to cross-reactivity from pre-existing antibodies or other possible causes. Prevalence of SARS-CoV-2 infection in the area where testing has occurred should be considered when interpreting positive test results.

At this time, it is unknown how long SARS-CoV-2 IgG antibodies may persist following infection.

a. Samples from pregnant women, especially multipara (women who had more than one pregnancy).

Samples from patients previously infected with the closely related virus strains SARS-CoV and MERS-COV have not been evaluated for interference in this assay. Samples from individuals treated with relevant medicines such as antiviral drugs, antibacterial drugs, acetylsalicylic acid, paracetamol, ibuprofen, anti-hypertensive drugs, anti-diabetic drugs, and hydroxychloroquine have not been evaluated for interference in this assay.

### Materials Provided & Storage Conditions

The materials and storage conditions are the same as described in Table 12, supra, in Example 12 with the exception that (i) 4 RBD Antigen Microplates and 5 Spike Protein Plates are provided, and (ii) Conjugate buffer IgG ELISA is supplied as 1 bottle of 120 mL of a buffered protein base solution with preservatives.

### Sample Collection & Storage

Serum—Use a serum separator tube (SST) and allow samples to clot for 30 minutes at room temperature before centrifugation for 15 minutes at 1000×g. Remove serum and assay immediately or aliquot and store samples at 4° C. for up to 7 days.

Plasma—Collect plasma using K2-EDTA or Li-Heparin as an anticoagulant. Centrifuge for 15 minutes at 1000×g within 30 minutes of collection. Assay immediately or aliquot and store samples at 4° C. for up to 7 days.

Note: Citrate plasma has not been validated for use in this assay. It is recommended not to freeze samples more than three times.

Reagent Preparation: See the Reagent Preparation described in Example 12 for the Reagent Preparation.

Sample Preparation: See Sample Preparation in Example 12 for Sample Preparation—with the exception that aliquots of inactivated samples are stored at 4° C. for up to 7 days post collection.

RBD ELISA Procedure: See RBD ELISA Procedure in Examples 12 and 13 for RBD ELISA Procedure (including Chart 1).

RBD ELISA Calculation: See RBD ELISA Calculation in Example 13 for RBD ELISA Calculation.

### RBD Quality Control:

Each testing laboratory should establish a quality control program to monitor the performance of the COVID-SeroIndex Immunoassay. As part of this program, controls with known anti-SARS-CoV-2 IgG concentrations (provided) should be tested in each assay. Satisfactory performance is obtained when controls fall within the established ranges provided in the Certificate of Analysis or within a lab's interval, as determined by an appropriate internal laboratory quality control procedure. A laboratory's quality control procedures may be followed; if the results obtained do not fall within the acceptable limits, the assay results may be invalid.

The corrected OD of the blank should be <0.03 OD.

Spike ELISA Assay Procedure: See Spike ELISA Procedure in Example 13 for Spike ELISA Procedure (including Chart 3).

### Spike ELISA Calculation of Results

Read the absorbance of each well on a microplate reader using 450 nm as the primary wavelength and 540 nm or 570 nm as the reference wavelength. Average the duplicate readings for each calibrator and control.

Create a standard curve by reducing the calibrator values using computer software capable of generating a four-parameter logistic (4-PL) curve fit. See Table 14 and FIG. 24 for exemplary data and a standard curve, respectively.

Samples falling below the Limit of Quantification (LoQ) of 3.20 AU/mL are considered negative. Values above the analytical measuring range should be reported as >160 AU/mL.

### Spike Quality Control:

Each testing laboratory should establish a quality control program to monitor the performance of the COVID-SeroIndex Immunoassay. As part of this program, controls with known anti-SARS-CoV-2 IgG concentrations (provided) should be tested in each assay. Satisfactory performance is obtained when controls fall within the established ranges provided in the Certificate of Analysis or within a lab's interval, as determined by an appropriate internal laboratory quality control procedure. A laboratory's quality control procedures may be followed; if the results obtained do not fall within the acceptable limits, the assay results may be invalid.

### Interpretation of Results:

Assessment of the kit results should be performed after the positive and negative controls have been examined and determined to be valid and acceptable. If the controls are not valid, the patient results cannot be interpreted.

NEGATIVE RBD Screen Result: Indicates that a 100-fold dilution of the sample tested contained no detectable levels of specific antibodies to the RBD protein fragment of SARS-CoV-2 Spike protein and no evidence of a detectable level of immune response to SARS-CoV-2 virus. The patient from whom the sample was obtained is presumed not to have been infected with SARS-CoV-2 virus at the time the sample was obtained. A negative result does not preclude the possibility of a very early immune response, which is not yet producing detectable levels of IgG antibodies specific to the antigen.

PRESUMPTIVE POSITIVE RBD Screen Result: The 100-fold dilution of the sample produced a positive reaction to the RBD protein fragment of SARS-CoV-2 Spike protein. This must be confirmed by testing its reactivity against the full-length Spike protein of the virus to confirm an appropriate level of circulating antibody in the tested sample.

ANTIBODY Concentration: The Quantitative ELISA Assay can reproducibly measure levels of SARS-CoV-2 IgG antibodies and the results are expressed in arbitrary units per milliliter of test sample. These numerical values have been experimentally shown to correlate with viral neutralizing activity in vitro. The experimentally determined measurable range is 3.2-160 AU/mL.

The circulating levels of IgG antibodies specific to the Spike protein of SARS-CoV-2 virus in AU/mL can be separated into clinically relevant levels correlated with SARS-CoV-2 neutralizing activity in vitro.

The use of AU/mL is an accepted method of quantitation in the absence of a traceable standard for exact measurement of the substance of analytical interest. These units are related in a direct proportional manner used to show the ratio of amount of analyte to a predetermined reference material. See Table 32, infra.

TABLE 32

| Antibody | | |
|---|---|---|
| Concentration (AU/mL) | | Clinical Interpretation |
| <3.2 AU/mL (LoQ) | | Negative. No or very early immune response. Indeterminate |
| | | immune status against SARS-CoV-2. If clinically indicated, |
| | | repeat in 3 weeks. |
| 3.2-10 | AU/mL | Low positive. Likely very early immune response. If clinically |
| | | indicated, repeat in 3 weeks. |
| 10-25 | AU/mL | Moderate antibody level. It has been demonstrated that |
| | | greater than 90% of persons with this concentration of IgG |
| | | level specific for SARS-CoV-2 exhibit viral neutralizing |
| | | activity in vitro. |
| >25 | AU/mL | High antibody level. Indicates a significantly elevated serum |
| | | concentration, potentially a strong immune status. It has been |
| | | demonstrated that 90-100% of persons with this |
| | | concentration of IgG level specific for SARS-CoV-2 exhibit |
| | | viral neutralizing activity in vitro. |

### Analytical Measuring Range

The RBD ELISA is a qualitative ELISA and there is no defined analytical measuring range (AMR). The output of the device is given in CI values. CI values are calculated by dividing the corrected OD value for unknown samples by the corrected OD value for the mean of the RBD Positive Control. Unknown samples with a CI≥0.70 are considered positive on the RBD ELISA and unknown samples with a CI<0.70 are considered negative on the RBD ELISA. Unknown samples that test positive on the RBD ELISA are then tested on the Spike ELISA while unknown samples that test negative on the RBD ELISA are given a final determination of negative.

The AMR for the Spike protein ELISA was determined through the results of the analytical validation studies as described below. This range is based on the LoQ for the lower limit of the measuring interval, the determination of the linear range as described in the linearity study, and the high calibrator, which is set at 200 AU/mL. The studies described below demonstrate precision and linearity across the AMR. Results of the quantitative Spike ELISA are reported in AU/mL. The claimed AMR is 3.2-160 AU/mL.

### Within-Site Precision:

RBD ELISA—Within-site repeatability was determined by measuring four serum samples in two tests per day, three replicates per test for three days. Positive and negative controls were also measured in two replicates per test, two tests per day for three days. See Table 33, infra.

TABLE 33

| | | | | | Total Within- | |
|---|---|---|---|---|---|---|
| | | Mean | Repeatability | | Laboratory Precision | |
| Sample | n | (CI) | SD | % CV | SD | % CV |
| Negative Control | 12 | 0.040 | 0.004 | 11.0 | 0.005 | 12.3 |
| Positive Control | 12 | 1.00 | 0.035 | 3.5 | 0.035 | 3.5 |
| Sample 1 | 18 | 0.153 | 0.005 | 3.0 | 0.012 | 7.9 |
| Sample 2 | 18 | 0.756 | 0.031 | 4.1 | 0.077 | 10.2 |
| Sample 3 | 18 | 1.06 | 0.073 | 6.9 | 0.093 | 8.7 |
| Sample 4 | 18 | 1.88 | 0.062 | 3.3 | 0.116 | 6.2 |

Spike ELISA—Within-site repeatability was determined by measuring three serum samples in two tests per day, three replicates per test for three days. The Low, Mid, and High controls were also measured in two replicates per test, two tests per day for three days. See Table 34, infra.

TABLE 34

| | | | | | Total Within- | |
|---|---|---|---|---|---|---|
| | | Mean | Repeatability | | Laboratory Precision | |
| Sample | n | (AU/mL) | SD | % CV | SD | % CV |
| Low Control | 30 | 2.29 | 0.150 | 6.6 | 0.170 | 7.5 |
| Mid Control | 30 | 9.68 | 0.590 | 6.1 | 0.640 | 6.6 |
| High Control | 30 | 38.1 | 2.57 | 6.8 | 3.30 | 8.7 |
| Sample 1 | 18 | 3.47 | 0.090 | 2.5 | 0.100 | 2.9 |
| Sample 2 | 18 | 4.34 | 0.120 | 2.7 | 0.190 | 4.4 |
| Sample 3 | 18 | 41.8 | 2.30 | 5.5 | 3.49 | 8.4 |
| Sample 4 | 18 | 127 | 13.6 | 10.7 | 16.9 | 13.3 |

### Lot-to-Lot Precision:

RBD ELISA—Lot-to-lot imprecision was determined by measuring four serum samples in two tests per day, three replicates per test for three days using two different lots of reagents. Positive and negative controls were also measured in two replicates per test, two tests per day for three days with two lots of reagents.

TABLE 35

| | | Mean | Within-Run | | Between-Run | | Between-Day | | Between-Lot | | Total | |
|---|---|---|---|---|---|---|---|---|---|---|---|---|
| Sample | n | (CI) | SD | % CV | SD | % CV | SD | % CV | SD | % CV | SD | % CV |
| Negative | 24 | 0.041 | 0 | 9.4 | 0 | 8.1 | 0 | 0 | 0 | 0 | 0.010 | 12.4 |
| Control | | | | | | | | | | | | |
| Positive | 24 | 1.00 | 0.040 | 3.9 | 0 | 0 | 0 | 0 | 0 | 0 | 0.040 | 3.9 |
| Control | | | | | | | | | | | | |
| Sample 1 | 36 | 0.143 | 0.010 | 4.4 | 0.010 | 4.20 | 0.010 | 4.4 | 0.010 | 9.5 | 0.020 | 12.1 |
| Sample 2 | 36 | 0.686 | 0.030 | 4.7 | 0.040 | 6.50 | 0.030 | 4.4 | 0.100 | 13.9 | 0.110 | 16.7 |
| Sample 3 | 36 | 0.963 | 0.050 | 5.4 | 0.030 | 3.10 | 0.040 | 4.1 | 0.140 | 14.4 | 0.160 | 16.2 |
| Sample 4 | 36 | 1.70 | 0.060 | 3.5 | 0.100 | 6.00 | 0.030 | 1.7 | 0.240 | 14.1 | 0.270 | 15.8 |

Spike ELISA—Lot-to-lot imprecision was determined by measuring three serum samples in two tests per day, three replicates per test for three days with two different lots of reagents. The Low, Mid, and High controls were also measured in two replicates per test, two tests per day for three days with two lots of reagents. See Table 36, infra.

TABLE 36

| | | Mean | Within-Run | | Between-Run | | Between- Day | | Between-Lot | | Total | |
|---|---|---|---|---|---|---|---|---|---|---|---|---|
| Sample | n | (AU/mL) | SD | % CV | SD | % CV | SD | % CV | SD | % CV | SD | % CV |
| Low | 66 | 2.23 | 0.140 | 6.2 | 0.080 | 3.4 | 0 | 0 | 0.060 | 2.8 | 0.170 | 7.6 |
| Control | | | | | | | | | | | | |
| Mid | 66 | 9.80 | 0.510 | 5.2 | 0.300 | 3.0 | 0 | 0 | 0.100 | 1.0 | 0.600 | 6.1 |
| Control | | | | | | | | | | | | |
| High | 66 | 38.3 | 3.13 | 8.2 | 0.790 | 2.1 | 1.50 | 3.9 | 0 | 0 | 3.56 | 9.3 |
| Control | | | | | | | | | | | | |
| Sample 1 | 36 | 3.46 | 0.130 | 3.6 | 0.120 | 3.5 | 0 | 0 | 0 | 0 | 0.170 | 5.0 |
| Sample 2 | 36 | 4.29 | 0.130 | 3.0 | 0.130 | 3.0 | 0.020 | 0.5 | 0.050 | 1.1 | 0.190 | 4.4 |
| Sample 3 | 36 | 41.8 | 3.07 | 7.3 | 1.09 | 2.6 | 3.41 | 8.1 | 0 | 0 | 4.71 | 11.3 |
| Sample 4 | 36 | 122 | 12.6 | 10.4 | 8.64 | 7.1 | 7.58 | 6.2 | 2.77 | 2.3 | 17.3 | 14.2 |

### Site-to-Site Reproducibility

RBD ELISA—Site-to-site reproducibility was determined by measuring four serum samples in two tests per day, three replicates per test for three days using at two different sites. Positive and negative controls were also measured in two replicates per test, two tests per day for three days at the two sites. See Table 37, infra.

TABLE 37

| | | Mean | Within-Run | | Between-Run | | Between- Day | | Between-Site | | Total | |
|---|---|---|---|---|---|---|---|---|---|---|---|---|
| Sample | n | (CI) | SD | % CV | SD | % CV | SD | % CV | SD | % CV | SD | % CV |
| Negative | 24 | 0.092 | 0.010 | 5.7 | 0.010 | 7.2 | 0 | 0 | 0.070 | 79.0 | 0.070 | 79.5 |
| Control | | | | | | | | | | | | |
| Positive | 24 | 1.00 | 0.030 | 3.3 | 0 | 0 | 0 | 0 | 0 | 0 | 0.030 | 3.3 |
| Control | | | | | | | | | | | | |
| Sample 1 | 36 | 0.228 | 0.010 | 5.2 | 0.020 | 10.9 | 0 | 0 | 0.110 | 46.2 | 0.110 | 47.8 |
| Sample 2 | 36 | 0.718 | 0.030 | 4.2 | 0.070 | 10.2 | 0 | 0 | 0.050 | 6.4 | 0.090 | 12.8 |
| Sample 3 | 36 | 1.04 | 0.060 | 6.2 | 0.050 | 5.0 | 0.080 | 7.6 | 0 | 0 | 0.110 | 11.0 |
| Sample 4 | 36 | 1.81 | 0.100 | 5.6 | 0.060 | 3.2 | 0.060 | 3.5 | 0.080 | 4.4 | 0.160 | 8.6 |

Spike ELISA—Site-to-site reproducibility was determined by measuring three serum samples in two tests per day, three replicates per test for three days at two different sites. The Low, Mid, and High controls were also measured in two replicates per test, two tests per day for three days at the two sites. See Table 38, infra.

TABLE 38

| | | Mean | Within-Run | | Between-Run | | Between- Day | | Between-Site | | Total | |
|---|---|---|---|---|---|---|---|---|---|---|---|---|
| Sample | n | (AU/mL) | SD | % CV | SD | % CV | SD | % CV | SD | % CV | SD | % CV |
| Low | 42 | 2.23 | 0.150 | 6.9 | 0.080 | 3.8 | 0 | 0 | 0.15 | 6.7 | 0.230 | 10.4 |
| Control | | | | | | | | | | | | |
| Mid | 42 | 9.54 | 0.550 | 5.7 | 0.350 | 3.7 | 0 | 0 | 0.310 | 3.2 | 0.720 | 7.5 |
| Control | | | | | | | | | | | | |
| High | 42 | 38.8 | 3.86 | 10.0 | 0.720 | 1.8 | 1.23 | 3.2 | 1.36 | 3.5 | 4.33 | 11.2 |
| Control | | | | | | | | | | | | |
| Sample 1 | 36 | 3.54 | 0.160 | 4.6 | 0.270 | 7.6 | 0 | 0 | 0 | 0.0 | 0.310 | 8.9 |
| Sample 2 | 36 | 4.52 | 0.150 | 3.4 | 0.450 | 9.9 | 0 | 0 | 0.190 | 4.1 | 0.510 | 11.3 |
| Sample 3 | 33 | 42.5 | 2.54 | 6.0 | 1.28 | 3.0 | 2.04 | 4.8 | 0 | 0 | 3.50 | 8.2 |
| Sample 4 | 35 | 138 | 13.58 | 9.9 | 15.1 | 11.0 | 0 | 0 | 15.12 | 11.0 | 25.31 | 18.4 |

Analytical Sensitivity: See Analytical Sensitivity in Example 12 and Table 19 for Analytical Sensitivity data.

Linearity: Linearity was demonstrated according to recommendations in CLSI guideline EP06-A. Three individual samples were proportionally diluted with negative serum samples. The negative serum samples used to make the dilutions were preCOVID-19 samples collected prior to September 2019. The linear range is 3.1-160 AU/mL and the Analytical Measuring Range (AMR) is 3.2-160 AU/mL. See Table 39, infra.

TABLE 39

| | # Dilution Levels in | Linear Range |
|---|---|---|
| Sample | the Linear Range | (AU/mL) |
| 1 | 11 | 8.2-145 |
| 2 | 11 | 4.2-161 |
| 3 | 10 | 3.1-72.1 |

### Clinical Significance:

To evaluate the Positive Percent Agreement (PPA) and Negative Percent Agreement (NPA) of the Quantitative SARS-CoV-2 IgG Antibody Kit, 92 positive samples and 284 negative samples were tested. These samples were all tested according to the IFU of the device. If the samples were negative on the RBD ELISA, they were not tested on the Spike ELISA. If they tested positive on the RBD ELISA, they were subsequently tested on the Spike ELISA.

Positive Percent Agreement: For the positive samples confirmed with a known EUA-authorized molecular test, PPA was 97.8%. We note that two samples that tested negative with the COVID-SeroIndex Kantaro Quantitative SARS-CoV-2 IgG Antibody IVD Kit also tested negative on an existing EUA approved serology test, suggesting that these are true negative samples. See Table 40, infra.

TABLE 40

| Days between | | | | |
|---|---|---|---|---|
| Positive PCR | | | | |
| and Sample | | Number Non- | Number | |
| Collection | Total Samples | Reactive | Positive | PPA |
| ≤7 | 0 | 0 | 0 | N/A |
| 8-14 | 1 | 0 | 1 | 100% |
| ≥15 | 91 | 2 | 89 | 97.8% |
| Total | 92 | 2 | 90 | 97.8% |

Negative Percent Agreement: For the negative samples, the NPA was 99.6%. There were 14 samples that tested positive on the RBD ELISA, below. Of these samples, 13 subsequently tested negative on the Spike ELISA, therefore the number of negative samples is 281 out of 282. See Table 41, infra.

TABLE 41

| | | Number | Number | |
|---|---|---|---|---|
| | Total Samples | Negative | Positive | NPA |
| PreCovid-19 | 272 | 271 | 1 | 99.6% |
| HIV Positive | 10 | 10 | 0 | 100.0% |
| Total | 282 | 281 | 1 | 99.6% |

### Calibration: See Calibration in Example 13 for Calibration.

Class Specificity: See Class Specificity in Example 13 and FIG. 25 for Class Specificity.

Specificity: See Specificity in Example 13—with the exception that instead of cold coronaviruses, coronavirus HKU1, coronavirus NL63, coronavirus OC43, and coronavirus 229E are identified.

### Interference:

RBD ELISA: Interference testing was performed following recommendations in CLSI guideline EP07-A3. Four serum samples were used to evaluate potential endogenous interferents. Data was evaluated quantitatively by comparing the percent difference between the mean CI value of the unspiked sample and the mean CI value of the spiked samples. All samples demonstrated a difference for the quantitative analysis of ≤15% at the specified concentration.

Spike ELISA: Interference testing was performed following recommendations in CLSI guideline EP07-A3. Two serum samples were used to evaluate potential endogenous interferents for the Spike ELISA, one at approximately 5.0 AU/mL, and one at approximately 50 AU/mL. Data was evaluated quantitatively by comparing the percent difference between the mean AU/mL value of the unspiked sample and the mean AU/mL value of the spiked samples. All samples demonstrated a difference for the quantitative analysis of ≤15% at the specified concentration.

TABLE 42

| Interferent | Highest Concentration | |
|---|---|---|
| Conjugated Bilirubin | 104 | mg/dL |
| Unconjugated Bilirubin | 96.6 | mg/dL |
| Hemoglobin | 10.6 | g/dL |
| Total Protein | 8.6 | g/dL |
| Cholesterol | 315 | mg/dL |
| Triglycerides | 6710 | mg/dL |

Microneutralization: See Microneutralization in Example 13 and FIGS. 26A-26B for Microneutralization.

### 6.15 Example 15: Detection of Antibody in Vaccinated Individuals

Serological assays described herein detected IgG antibody to SARS-CoV-2 spike protein in human subjects who had received two doses of SARS-CoV-2 mRNA vaccines.

For example, such IgG antibody was detected using the assay described in Example 12, supra.

### 6.16 Example 16: Quantitative Determination of SARS-Cov-2 IGG in Saliva or Oral Fluid

This example describes a SARS-CoV-2 enzyme-linked immunosorbent assay (ELISA), which is intended for the quantitative measurement of IgG antibodies to SARS-CoV-2 spike protein in human saliva. The SARS-CoV-2 assay is read on an IVD microplate reader at 450 nm. The SARS COV-2 quantitative assay: (1) assesses to what extent levels of SARS-CoV-2 antibodies in saliva reflect concentrations in serum; (2) determines whether antibody levels in saliva can accurately identify protective concentrations in serum; and (3) assesses the immune response to vaccination and a surrogate marker for systemic antibody responses. The SARS-CoV-2 assay may aid in minimizing person-to-person transmission of SARS-CoV-2.

Since December 2019, the world has struggled with a pandemic of coronavirus disease 2019 (COVID-19), caused by SARS-CoV-2, with a spectrum of symptoms that include fever, fatigue, dry cough, myalgia, and dyspnea. It is vital to evaluate the prevalence of antibodies quantitatively in both asymptomatic and symptomatic of SARS-CoV-2 patients. SARS-CoV-2, the salivary gland could be a major source of the virus in saliva (Liu et al. 2011. Epithelial cells lining salivary gland ducts are early target cells of severe acute respiratory syndrome coronavirus infection in the upper respiratory tracts of rhesus macaques. J Virol. 85(8):4025-4030) and saliva samples can also cultivate the live virus (KK, Tsang et al. 2020. Consistent detection of 2019 novel coronavirus in saliva. Clin Infect Dis 71(15):841-843) suggesting that SARS-CoV-2 can be transmitted by asymptomatic carriers saliva. Saliva testing is also helpful in understanding about immunity dynamics during the disease, and how long it remains after the recovery from symptoms or treatment methods. Finally the impact of salivary particulates in Health Care Workers.

### Diagnostic Application

SARS-CoV-2 IgG is an enzyme-linked immunosorbent assay (ELISA) used for the quantitative detection of IgG antibodies to SARS-CoV-2 in human saliva (Salivette®-Sarstedt or FDA approved Oral antibody collection device from Orasure).

Rationale: ELISA is widely used for several Clinical Immunology/Serological assays. Calibrators and controls are prepared by spiking NIBSC reference material (20/136) into a pool of saliva of healthy individual donors. International units are assigned to calibrators and test samples are reported in the same units, a common practice in CLIA/CAP laboratories. The advantages of quantitative determination are three-fold: (1) to provide reliable values for antibody levels that can be correlated with serum antibody levels, (2) to determine vaccine response (quantitative ELISA that measures IgG levels is used in the evaluation of vaccines as a surrogate assay), and (3) to establish a gold standard assay that is traceable to WHO reference material.

Methodology: The reagents used are those described in Example 12. See also Kantaro Seroklir kit reagents are used in this indirect ELISA. The methodology described in Example 12 is essentially the same for saliva, however, RBD ELISA has been excluded for SARS-CoV-2 IgG measurement in saliva.

In brief, pre-coated 96-well plates with recombinant SARS-CoV-2 spike protein were added patient specimens (1:5 dilution), controls (neat) to the respective assigned wells. A 8-point calibration curve is generated by spiking a reference calibration standard in a normal healthy donors saliva pool. Following incubation, the plate is washed three times with wash buffer. Monoclonal antibody specific to human IgG conjugated to horseradish peroxidase, is added to all wells, then the plate is incubated and washed, followed by the addition of 3,3′,5,5′-Tetramethylbenzidine (TMB) substrate. Within 20 minutes the reaction is suspended by the addition of 3 M HCl or methylsulfonic acid stop solution. Optical density (OD) readings for each well are obtained at 450 nm.

### Data Analysis:

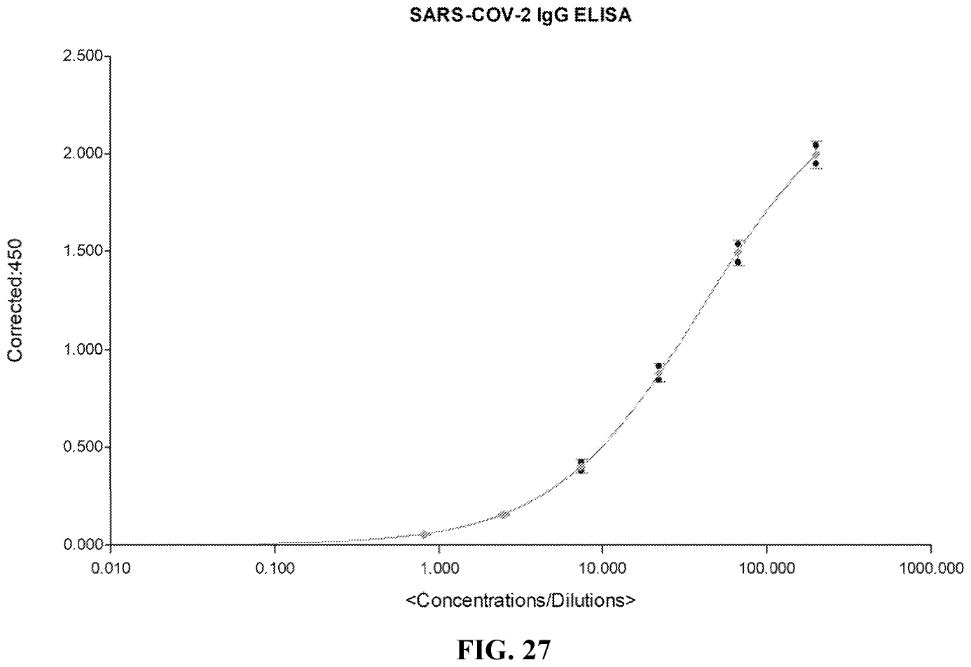

Calculation of International units for reference standard saliva: A 8 point calibration curve is generated and optical density (OD 450 nm) values are plotted against the assigned values using a 4-Parameter Logistic (4PL) curve (FIG. 27). The test samples OD values are read as unknown using eight point calibration curve by Biotek Gen 5 software (Winooski, VT). The results are reported in IU/mL for unknowns.

### Results:

Establishing a cut off:

a. Thirty pre-COVID saliva specimens (from 2018; Mount Sinai Clinical Laboratory obtained a Lab Developed Test approval from NY Department of Health for Salivary Cortisol measurement using LC-MS/MS) were run in duplicate by quantitative SARS-CoV-2 IgG ELISA and a cut-off has been established to differentiate a negative from a positive test results (Table 43).

b. Twelve de-identified RT-PCR negative saliva specimens were measured for SARS-CoV-2 IgG levels. Though RT-PCR was negative for these saliva specimens, detectable levels of SARS-CoV-2 IgG were measured. These data suggest saliva SARS-CoV-2 IgG measurement in conjunction with RT-PCR increases the sensitivity of the assay (Table 44).

c. One of ten RT-PCR positive specimens has undetectable measurement of SARS-CoV-2 IgG level (Table 44).

d. A paired (Serum from Gold-top tube and Saliva collected in Salivette) post vaccinated specimens data were compared using ELISA assay (Table 45). The vaccination immune response was evidently measured in saliva.

TABLE 43
Thirty pre-COVID specimens' quantitative SARS-CoV-2
IgG ELISA results were used to establish cut-off value.

| | | SARS-CoV-2 | | | | |
|---|---|---|---|---|---|---|
| | | IgG in | | | | |
| | OD450 | precovid saliva | | | | |
| | nm | (AU/mL) | | Average | SD | CV |
| Specimen 1 | 0.005 | 0.5 | | 0.5 | 0.0 | 2.3 |
| | 0.006 | 0.5 | | | | |
| Specimen 2 | 0.006 | 0.5 | | 0.5 | 0.0 | 4.2 |
| | 0.007 | 0.5 | | | | |
| Specimen 3 | 0.008 | 0.6 | | 0.6 | 0.0 | 4.8 |
| | 0.008 | 0.6 | | | | |
| Specimen 4 | 0.008 | 0.6 | | 0.5 | 0.1 | 15.6 |
| | 0.006 | 0.5 | | | | |
| Specimen 5 | 0.006 | 0.5 | | 0.5 | 0.0 | 2.3 |
| | 0.006 | 0.5 | | | | |
| Specimen 6 | 0.007 | 0.5 | | 0.5 | 0.0 | 2.0 |
| | 0.007 | 0.5 | | | | |
| Specimen 7 | 0.006 | 0.5 | | 0.5 | 0.0 | 0.8 |
| | 0.006 | 0.5 | | | | |
| Specimen 8 | 0.006 | 0.5 | | 0.5 | 0.0 | 3.4 |
| | 0.007 | 0.5 | | | | |
| Specimen 9 | 0.01 | 0.7 | | 0.6 | 0.1 | 11.0 |
| | 0.008 | 0.6 | | | | |
| Specimen 10 | 0.007 | 0.5 | | 0.5 | 0.0 | 5.8 |
| | 0.008 | 0.6 | | | | |
| Specimen 11 | 0.007 | 0.5 | | 0.5 | 0.1 | 17.5 |
| | 0.005 | 0.4 | | | | |
| Specimen 12 | 0.008 | 0.6 | | 0.6 | 0.1 | 8.3 |
| | 0.01 | 0.7 | | | | |
| Specimen 13 | 0.006 | 0.5 | | 0.5 | 0.1 | 10.4 |
| | 0.007 | 0.5 | | | | |
| Specimen 14 | 0.007 | 0.5 | | 0.5 | 0.0 | 2.1 |
| | 0.006 | 0.5 | | | | |
| Specimen 15 | 0.034 | 2.0 | | 2.0 | 0.0 | 1.2 |
| | 0.034 | 2.0 | | | | |
| Specimen 16 | 0.006 | 0.5 | | 0.5 | 0.0 | 1.4 |
| | 0.006 | 0.5 | | | | |
| Specimen 17 | 0.006 | 0.5 | | 0.5 | 0.0 | 3.7 |
| | 0.006 | 0.5 | | | | |
| Specimen 18 | 0.006 | 0.5 | | 0.5 | 0.0 | 5.8 |
| | 0.006 | 0.5 | | | | |
| Specimen 19 | 0.011 | 0.8 | | 0.8 | 0.0 | 5.8 |
| | 0.013 | 0.8 | | | | |
| Specimen 20 | 0.006 | 0.5 | | 0.5 | 0.0 | 5.1 |
| | 0.006 | 0.5 | | | | |
| Specimen 21 | 0.016 | 1.0 | | 1.0 | 0.0 | 1.1 |
| | 0.016 | 1.0 | | | | |
| Specimen 22 | 0.011 | 0.7 | | 0.8 | 0.1 | 10.6 |
| | 0.013 | 0.9 | | | | |
| Specimen 23 | 0.007 | 0.5 | | 0.5 | 0.0 | 4.2 |
| | 0.006 | 0.5 | | | | |
| Specimen 24 | 0.008 | 0.6 | | 0.5 | 0.1 | 12.0 |
| | 0.006 | 0.5 | | | | |
| Specimen 25 | 0.021 | 1.3 | | 1.4 | 0.1 | 8.0 |
| | 0.024 | 1.4 | | | | |
| Specimen 26 | 0.006 | 0.5 | | 0.5 | 0.0 | 0.7 |
| | 0.006 | 0.5 | | | | |
| Specimen 27 | 0.007 | 0.5 | | 0.5 | 0.0 | 4.0 |
| | 0.007 | 0.5 | | | | |
| Specimen 28 | 0.006 | 0.5 | | 0.5 | 0.0 | 1.5 |
| | 0.006 | 0.5 | | | | |
| Specimen 29 | 0.007 | 0.5 | | 0.5 | 0.0 | 6.1 |
| | 0.006 | 0.5 | | | | |
| Specimen 30 | 0.006 | 0.5 | | 0.5 | 0.0 | 1.5 |
| | 0.006 | 0.5 | | | | |
| | | | Cut-off | 0.715 | | |
| | | | (AU/mL) | | | |

TABLE 44
SARS-CoV-2 IgG level was measured in saliva
specimens that were also tested by RT-PCR.

| | SARS-CoV-2 IgG in Saliva (AU/mL) | RT PCR |
|---|---|---|
| Specimen 1 | 1 | POS |
| Specimen 2 | 4 | POS |
| Specimen 3 | 1 | POS |
| Specimen 4 | 50 | POS |
| Specimen 5 | 19 | POS |
| Specimen 6 | 13 | POS |
| Specimen 7 | 4 | POS |
| Specimen 8 | 0 | POS |
| Specimen 9 | 3 | POS |
| Specimen 10 | 1 | POS |
| Specimen 11 | 107 | NEG |
| Specimen 12 | 1 | NEG |
| Specimen 13 | 135 | NEG |
| Specimen 14 | 22 | NEG |
| Specimen 15 | 18 | NEG |
| Specimen 16 | 2 | NEG |
| Specimen 17 | 10 | NEG |
| Specimen 18 | 73 | NEG |
| Specimen 19 | 391 | NEG |
| Specimen 20 | 302 | NEG |
| Specimen 21 | 35 | NEG |
| Specimen 22 | 10 | NEG |

TABLE 45
Comparison of SARS-COV-2 IgG levels in paired specimens after vaccination.

| | | | Saliva | | | Time | | |
|---|---|---|---|---|---|---|---|---|
| | SST | EDTA | collected in | Vaccine | Vaccine | after | | |
| | Serum | Plasma | Salivette | Received | Name | dose | | Dose |
| Specimen 1 | 533 | 555 | 82 | Mar. 4, 2021 | Pfizer | 3 | Weeks | 2nd Dose |
| Specimen 2 | 2145 | 2437 | 361 | Mar. 4, 2021 | PFIZER | 4 | Weeks | 2nd Dose |
| Specimen 3 | 318 | 404 | 74 | Mar. 4, 2021 | PFIZER | >1 | month | 2nd Dose |
| Specimen 4 | 176 | 142 | 34 | Mar. 4, 2021 | PFIZER | 3 | Weeks | 2nd Dose |
| Specimen 5 | 103 | 84 | 13 | Mar. 4, 2021 | MODERNA | 4 | Weeks | 2nd Dose |
| Specimen 6 | 209 | 212 | 9 | Mar. 4, 2021 | MODERNA | ~1 | month | 2nd Dose |
| Specimen 7 | 643 | 541 | 157 | Mar. 4, 2021 | MODERNA | 4 | Weeks | 2nd Dose |
| Specimen 8 | 788 | 705 | 21 | Mar. 4, 2021 | MODERNA | 5 | Weeks | 2nd Dose |
| Specimen 9 | 336 | 348 | 44 | Mar. 4, 2021 | MODERNA | 5 | Weeks | 2nd Dose |
| Specimen 10 | 476 | 442 | 15 | Mar. 4, 2021 | | 3 | Weeks | 2nd Dose |
| Specimen 11 | 175 | | 33 | Mar. 4, 2021 | | | | 2nd Dose |

### 7. EMBODIMENTS

1. A recombinant soluble SARS-CoV-2 spike protein comprising amino acids 1-1213 of the spike protein found at GenBank Accession No. MN908947.3, a C-terminal thrombin cleavage site, T4 foldon trimerization domain and hexahistidine tag, wherein the protein does not contain the polybasic cleavage site (RRAR to A).

2. A recombinant soluble SARS-CoV-2 spike protein comprising amino acids 1-1213 of the spike protein found at GenBank Accession No. MN908947.3, a C-terminal thrombin cleavage site, T4 foldon trimerization domain and hexahistidine tag, wherein the protein does not contain the polybasic cleavage site (RRAR to A) and the protein contains two stabilizing mutations (K986P and V987P, wild type numbering).

3. A recombinant soluble SARS-CoV-2 spike protein comprising amino acid residues 319-541 of the spike protein found at GenBank Accession No. MN908947.3 and a tag.

4. The recombinant soluble SARS-CoV-2 spike protein of embodiment 3, wherein the tag is a hexahistidine tag.

5. A recombinant SARS-CoV2 spike protein comprising the amino acid sequence of SEQ ID NO: 2, 4, or 6.

6 An isolated nucleic acid sequence comprising a nucleotide sequence encoding the recombinant soluble SARS-CoV-2 spike protein of any one of embodiments 1 to 5.

7. An isolated nucleic acid sequence comprising the nucleotide sequence of SEQ ID NO: 1, 3 or 5.

8. An isolated cell engineered to express the recombinant soluble SARS-CoV-2 spike protein of any one of embodiments 1 to 5.

9 A method for detecting an antibody that specifically binds to SARS-CoV-2 spike protein, comprising contacting a recombinant soluble SARS-CoV-2 spike protein with a biological sample obtained from a subject and detecting the binding of antibody(ies) present in the biological sample to the recombinant soluble SARS-CoV-2 spike protein.

10. The method of embodiment 9, wherein the recombinant soluble SARS-CoV-2 spike protein comprises the amino acid sequence of SEQ ID NO: 2, 4, or 6.

11. The method of embodiment 9 or 10, wherein the biological sample is plasma or sera.

12. The method of any one of embodiments 9 to 11, wherein the subject is human.

13. The method of any one of embodiments 9 to 12, wherein the method comprises quantitating the amount of antibody.

14. A method for detecting an antibody that specifically binds to SARS-CoV-2 spike protein, comprising: (1) incubating a specimen in a well coated with a recombinant SARS-CoV-2 spike protein for a first period time; (2) washing the well; (3) incubating a labeled antibody that binds to an isotype or subtype of immunoglobulin in the well for a second period of time; (4) washing the well; and (5) detecting the binding of the labeled antibody to the recombinant SARS-CoV-2 spike protein in the well.

15. The method of embodiment 14, wherein the specimen is a biological sample or antibody sample.

16. The method of embodiment 15, wherein the biological sample is blood, sera or plasma from a subject.

17. The method of embodiment 16, wherein the subject is human.

18. The method of embodiment 15, 16 or 17, wherein the biological sample is inactivated.

19. The method of any one of embodiments 14 to 18, wherein the recombinant SARS-CoV-2 spike protein comprises the amino acid sequence of SEQ ID NO: 2, 4, or 6.

20. A recombinant soluble SARS-CoV-2 spike protein, wherein the recombinant soluble SARS-CoV-2 spike protein comprises a receptor binding domain of a SARS-CoV-2 protein corresponding to amino acid residues 319-541 of GenBank Accession No. MN908947.3 and a tag.

21. The recombinant soluble SARS-CoV-2 spike protein of embodiment 20, wherein the tag is a hexahistidine tag.

22. A recombinant soluble SARS-CoV-2 spike protein, wherein the recombinant soluble SARS-CoV-2 spike protein comprises the ectodomain of a SARS-CoV-2 spike protein, a C-terminal cleavage site, trimerization domain, and a tag, wherein the recombinant soluble SARS-CoV-2 spike protein does not contain a polybasic cleavage site.

23. The recombinant soluble SARS-CoV-2 spike protein of embodiment 22, wherein the tag is a hexahistidine tag.

24. The recombinant soluble SARS-CoV-2 spike protein of embodiment 22 or 23, wherein the polybasic cleavage site (RRAR) is replaced by a single A.

25. The recombinant soluble SARS-CoV-2 spike protein of embodiment 22, 23 or 24, wherein the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3.

26. The recombinant soluble SARS-CoV-2 spike protein of embodiment 22, 23, or 24, wherein the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3.

27. The recombinant soluble SARS-CoV-2 spike protein of any one of embodiments 22 to 26, wherein the C-terminal cleavage site is a C-terminal thrombin cleavage site.

28. The recombinant soluble SARS-CoV-2 spike protein of any one of embodiments 22 to 27, wherein the trimerization domain is a T4 foldon trimerization domain.

29. The recombinant soluble SARS-CoV-2 spike protein of embodiment 22, wherein the tag is a hexahistidine tag, the C-terminal cleavage site is a C-terminal thrombin cleavage site, and the trimerization domain is a T4 foldon trimerization domain.

30. A recombinant soluble SARS-CoV-2 spike protein, wherein the recombinant soluble SARS-CoV-2 spike protein comprises amino acid residues 319-541 of GenBank Accession No. MN908947.3 and a tag or

31. The recombinant soluble SARS-CoV-2 spike protein of embodiment 30, wherein the tag is a hexahistidine tag.

32. A recombinant soluble SARS-CoV-2 spike protein, wherein the recombinant soluble SARS-CoV-2 spike protein comprises amino acid residues 1-1213 of GenBank Accession No. MN908947.3, a C-terminal cleavage site, trimerization domain, and a tag, wherein the recombinant soluble SARS-CoV-2 spike protein does not contain a polybasic cleavage site.

33. The recombinant soluble SARS-CoV-2 spike protein of embodiment 32, wherein the tag is a hexahistidine tag.

34. The recombinant soluble SARS-CoV-2 spike protein of embodiment 32 or 33, wherein the polybasic cleavage site (RRAR) is replaced by a single A.

35. The recombinant soluble SARS-CoV-2 spike protein of embodiment 32, 33 or 34, wherein the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3.

36. The recombinant soluble SARS-CoV-2 spike protein of embodiment 32, 33, or 34, wherein the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3.

37. The recombinant soluble SARS-CoV-2 spike protein of any one of embodiments 32 to 36, wherein the C-terminal cleavage site is a C-terminal thrombin cleavage site.

38. The recombinant soluble SARS-CoV-2 spike protein of any one of embodiments 32 to 37, wherein the trimerization domain is a T4 foldon trimerization domain.

39. The recombinant soluble SARS-CoV-2 spike protein of embodiment 32, wherein the tag is a hexahistidine tag, the C-terminal cleavage site is a C-terminal thrombin cleavage site, and the trimerization domain is a T4 foldon trimerization domain.

40. The recombinant soluble SARS-CoV-2 spike protein of embodiment 30, wherein the recombinant soluble SARS-CoV-2 spike protein comprises the amino acid sequence of SEQ ID NO:2, 4 or 6.

41. The recombinant soluble SARS-CoV-2 spike protein of embodiment 32, wherein the recombinant soluble SARS-CoV-2 spike protein comprises the amino acid sequence of SEQ ID NO: 4 or 6.

42. A composition comprising the recombinant soluble SARS-CoV-2 spike protein of any one of embodiments 20 to 41.

43. An isolated nucleotide sequence comprising a nucleic acid sequence encoding the recombinant soluble SARS-CoV-2 spike protein of any one of embodiments 20 to 41.

44. An isolated nucleotide sequence comprising the nucleic acid sequence of SEQ ID NO: 1, 3, or 5.

45. A vector comprising the nucleotide sequence of embodiment 43 or 44.

46. Isolated cells stably transfected with the nucleotide sequence of embodiment 43 or 44.

47. Isolated cells engineered stably transfected with the vector of embodiment 45.

48. Isolated cells engineered to express a recombinant soluble SARS-CoV-2 spike protein of any one of embodiments 20 to 41.

49. The cells of any one of embodiments 46 to 48, wherein the cells are Vero, MDCK, 293 T, HeLa, CHO, Cos, 293, HEK293, or Expi293F cells.

50. A method for immunizing against SARS-CoV-2, comprising administering to a subject the recombinant soluble SARS-CoV-2 spike protein of any one of embodiments 20 to 41, or the nucleotide sequence of embodiment 43 or 44.

51. A method for preventing COVID-19, comprising administering to a subject the recombinant soluble SARS-CoV-2 spike protein of any one of embodiments 20 to 41, or the nucleotide sequence of embodiment 43 or 44.

52. A method for inducing an immune response in a SARS-CoV-2 spike protein, comprising administering to a subject the recombinant soluble SARS-CoV-2 spike protein of any one of embodiments 20 to 41, or the nucleotide sequence of embodiment 43 or 44.

53. The method of embodiments 50 to 52, wherein the subject is human.

54. A kit comprises in one or more containers:
(a) a multi-well microtiter ELISA plate coated with a first recombinant soluble SARS-CoV-2 spike protein, wherein the first recombinant soluble SARS-CoV-2 spike protein comprises amino acid residues 319-541 of GenBank Accession No. MN908947.3 and a tag; and
(b) a multi-well ELISA microtiter plate coated with a second recombinant soluble SARS-CoV-2 spike protein, wherein the second recombinant soluble SARS-CoV-2 spike protein comprises amino acid residues 1-1213 of GenBank Accession No. MN908947.3, a C-terminal cleavage site, trimerization domain, and a tag, and wherein the second recombinant soluble SARS-CoV-2 spike protein does not contain a polybasic cleavage site.

55. The kit of embodiment 54, wherein the tag is a hexahistidine tag.

56. The kit of embodiment 54 or 55, wherein the polybasic cleavage site (RRAR) is replaced by a single A.

57. The kit of embodiment 54, 55 or 56, wherein the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3.

58. The kit of embodiment 54, 55, or 56, wherein the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3.

59. The kit of any one of embodiments 54 to 58, wherein the C-terminal cleavage site is a C-terminal thrombin cleavage site.

60. The kit of any one of embodiments 54 to 59, wherein the trimerization domain is a T4 foldon trimerization domain.

61. The kit of embodiment 54, wherein the tag is a hexahistidine tag, the C-terminal cleavage site is a C-terminal thrombin cleavage site, and the trimerization domain is a T4 foldon trimerization domain.

62. The kit of embodiment 54, wherein the first recombinant soluble SARS-CoV-2 spike protein comprises the amino acid sequence of SEQ ID NO:2.

63. The kit of embodiment 54 or 55, wherein the second recombinant soluble SARS-CoV-2 spike protein comprises the amino acid sequence of SEQ ID NO:4 or 6.

64. The kit of any one of embodiments 54 to 63, wherein the kit further comprises a labeled secondary antibody.

65. The kit of any one of embodiments 54 to 63, wherein the labeled secondary antibody is anti-human IgG horseradish perioxidase.

66. The kit of embodiment 65, wherein the kit further comprises o-pheylenediamine dihydrochloride.

67. The kit of any one of embodiments 54 to 66, wherein the kit further comprises a positive control antibody that binds to the recombinant soluble SARS-CoV-2 spike protein.

68. The kit of embodiment 67, wherein the positive control antibody is monoclonal antibody CR3022 or antibodies from COVID-19 patients.

69. The kit of any one of embodiments 54 to 68, wherein the kit further comprises a negative control antibody.

70. A method for detecting the presence of antibodies in a subject that are specific for the SARS-CoV-2 spike protein comprising contacting in vitro a biological sample from said subject with the recombinant soluble SARS-CoV-2 spike protein of any one of embodiments 20 to 41 and detecting the binding of the antibody to the recombinant soluble SARS-CoV2 spike protein using a labeled antibody that binds to the constant region of the antibodies specific for the SARS-CoV-2 spike protein.

71. The method of embodiment 70, wherein the biological sample is blood plasma or sera.

72. The method of embodiment 70 or 71, wherein the labeled antibody is labled with a chemiluminescent agent or radioactive label.

73. A method for the detection of antibody that specifically binds to human SARS-CoV-2 spike protein, comprising:
(a) incubating a specimen in a well coated with the recombinant soluble SARS-CoV-2 spike protein of any one of embodiments 20 to 41 for a first period of time;
(b) washing the well;
(c) incubating a labeled antibody that binds to an isotype or subtype of an immunoglobulin for a second period of time;
(d) washing the well; and
(e) detecting the binding of the labeled antibody to the recombinant SARS-CoV-2 spike protein.

74. The method of embodiment 73, wherein the first time period, second time period or both is 30 minutes, 45 minutes, 1 hour, 2 hours, 2.5 hours or 3 hours.

75. The method of embodiment 73 or 74, wherein the specimen is a biological sample. 76. The method of embodiment 75, wherein the biological sample is blood, sera, or plasma.

77. The method of embodiment 75 or 76, wherein the biological sample is inactivated before being incubated with the well coated with recombinant SARS-CoV-2 spike protein.

78. The method of any one of embodiments 73 to 77, wherein the specimen is serially diluted.

79. The method of any one of embodiment 73 to 77, wherein the labeled antibody is labeled with a radioactive moiety, a chemiluminescent moiety, or a fluorescent moiety.

80. The method of any one of embodiments 73 to 77, wherein the labeled antibody is labeled with a horseradish perioxidase.

81. The method of any one of embodiments 73 to 80, wherein the labeled antibody is an anti-human IgG antibody.

82. A method for the detection of antibody that specifically binds to human SARS-CoV-2 spike protein, comprising:
(a) incubating a specimen in a well coated with a first recombinant soluble SARS-CoV-2 spike protein for a first period of time, wherein the first recombinant soluble SARS-CoV-2 spike protein comprises amino acid residues 319-541 of GenBank Accession No. MN908947.3 and a tag;
(b) washing the well;
(c) incubating a labeled antibody that binds to an isotype or subtype of an immunoglobulin for a second period of time in the well;
(d) washing the well;
(e) detecting the binding of the labeled antibody to the first recombinant SARS-CoV-2 spike protein;
(f) incubating the specimen in a well coated with a second recombinant soluble SARS-CoV-2 spike protein for a third period of time, wherein the second recombinant soluble SARS-CoV-2 spike protein comprises amino acid residues 1-1213 of GenBank Accession No. MN908947.3, a C-terminal cleavage site, trimerization domain, and a tag, and wherein the recombinant soluble SARS-CoV-2 spike protein does not contain a polybasic cleavage site;
(g) washing the well;
(h) incubating a second labeled antibody that binds to an isotype or subtype of an immunoglobulin for a fourth period of time in the well;
(i) washing the well; and
(j) detecting the binding of the labeled antibody to the second recombinant soluble SARS-CoV-2 spike protein.

83. The method of embodiment 82, wherein the tag is a hexahistidine tag.

84. The method of embodiment 82 or 83, wherein the polybasic cleavage site (RRAR) is replaced by a single A.

85. The method of embodiment 82, 83 or 84, wherein the recombinant soluble SARS-CoV-2 spike protein further comprises a stabilizing mutation of lysine to proline at the amino acid residue corresponding to amino acid residue 986 of the amino acid sequence found at GenBank Accession No. MN908947.3, or valine to proline at the amino acid residue corresponding to amino acid residue 987 of the amino acid sequence found at GenBank Accession No. MN908947.3.

86. The method of embodiment 82, 83, or 84, wherein the recombinant soluble SARS-CoV-2 spike protein further comprises two stabilizing mutations of lysine to proline at the amino acid residue corresponding to amino acid residue 986 of the amino acid sequence found at GenBank Accession No. MN908947.3, and valine to proline at the amino acid residue corresponding to amino acid residue 987 of the amino acid sequence found at GenBank Accession No. MN908947.3.

87. The method of any one of embodiments 82 to 86, wherein the C-terminal cleavage site is a C-terminal thrombin cleavage site.

88. The method of any one of embodiments 82 to 87, wherein the trimerization domain is a T4 foldon trimerization domain.

89. The method of embodiment 82, wherein the tag is a hexahistidine tag, the C-terminal cleavage site is a C-terminal thrombin cleavage site, and the trimerization domain is a T4 foldon trimerization domain.

90. The method of embodiment 82, wherein the first recombinant soluble SARS-CoV-2 spike protein comprises the amino acid sequence of SEQ ID NO:2.

91. The method of embodiment 82 or 90, wherein the first recombinant soluble SARS-CoV-2 spike protein comprises the amino acid sequence of SEQ ID NO: 4 or 6.

92. The method of any one of embodiments 82 to 91, wherein the first time period and second time period are 30 minutes, 45 minutes, 1 hour, 2 hours, 2.5 hours or 3 hours.

93. The method of any one of embodiments 82 to 92, wherein the specimen is a biological sample.

94. The method of embodiment 93, wherein the biological sample is blood, sera, plasma, or saliva.

95. The method of embodiment of 93 or 94, wherein the biological sample is inactivated before being incubated with the well coated with recombinant SARS-CoV-2 spike protein.

96. The method of any one of embodiments 82 to 95, wherein the specimen is serially diluted.

97. The method of any one of embodiments 82 to 96, wherein the third and fourth time periods are 30 minutes, 45 minutes, 1 hour, 2 hours, 2.5 hours or 3 hours.

98. A kit comprising:
(a) a first multi-well microtiter plate coated with a first recombinant soluble SARS-CoV-2 spike protein, wherein the first recombinant soluble SARS-CoV-2 spike protein comprises amino acid residues 319 to 541 of a SARS-CoV-2 spike protein and a histidine tag at the C-terminus;
(b) a second multi-well microtiter plate coated with a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof;
(c) a vial containing concentrated monoclonal antibody specific to human IgG conjugated to horseradish peroxidase;
(d) a vial containing 3,3′,5,5′-Tetramethylbenzidine (TMB) substrate;
(e) a vial containing a stop solution comprising an acid;
(f) a vial containing a sample buffer comprising phosphate buffered saline (PBS) with 0.5% to 5% milk; and

(g) a vial containing a wash buffer comprising phosphate buffered saline and 0.5% to 2% of a surfactant.

99. A kit comprising:
(a) a vial containing a first recombinant soluble SARS-CoV-2 spike protein, wherein the first recombinant soluble SARS-CoV-2 spike protein comprises amino acid residues 319 to 541 of a SARS-CoV-2 spike protein and a histidine tag at the C-terminus;
(b) a vial containing a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof;
(c) a vial containing concentrated monoclonal antibody specific to human IgG conjugated to horseradish peroxidase;
(d) a vial containing 3,3′,5,5′-Tetramethylbenzidine (TMB) substrate;
(e) a vial containing a stop solution comprising an acid;
(f) a vial containing a sample buffer comprising phosphate buffered saline (PBS) with 0.5% to 5% milk; and
(g) a vial containing a wash buffer comprising 0.5% to 2% of a surfactant.

100. The kit of embodiment 99, wherein the kit further comprises 2 or more multi-well-microtiter plates.

101. The kit of embodiment 98, wherein first and second multi-well microtiter plates are 96-well microtiter plates.

102. The kit of embodiment 98 or 101, wherein the kit comprises 4 of the first multi-well microtiter plate and 5 of the second multi-well microtiter plate.

103. The kit of embodiment 98 or 101, wherein the kit comprises 7 of the first multi-well microtiter plate and 3 of the second multi-well microtiter plate. 104. The kit of any one of embodiments 98 to 103, wherein the first recombinant SARS-CoV-2 spike protein comprises the amino acid sequence set forth in SEQ ID NO:10.

105. The kit of any one of embodiment 98 to 104, wherein the vial containing the concentrated monoclonal antibody specific to human IgG conjugated to horseradish peroxidase comprises 100 to 500 μl of the concentrated antibody.

106. The kit of any one of embodiments 98 to 105, wherein the concentrated monoclonal antibody specific to human IgG conjugated to horseradish peroxidase is 100× to 1000× concentrated monoclonal antibody.

107. The kit of any one of embodiments 98 to 106, wherein the kit further comprises a second vial containing concentrated monoclonal antibody specific to human IgG conjugated to horseradish peroxidase.

108. The kit of any one of embodiments 98 to 107, wherein the acid is an alkyl sulfonic acid, 1 N HCl or 2 N H₂SO₄.

109. The kit of any one of embodiments 98 to 108, wherein the alkyl sulfonic acid is 1-5% methanesulfonic acid.

110. The kit of any one of embodiments 109, wherein the vial comprises 100 to 500 mL of methanesulfonic acid.

111. The kit of any one of embodiments 98 to 110, wherein the vial containing 3,3′,5,5′-Tetramethylbenzidine (TMB) substrate comprises 100 to 500 mL of 3,3′,5,5′-Tetramethylbenzidine (TMB) substrate.

112. The kit of any one of embodiments 98 to 111, wherein the vial containing the sample buffer comprises 50 to 500 ml of the sample buffer.

113. The kit of any one of embodiments 98 to 112, wherein the vial containing the wash buffer comprises 100 to 500 ml of the wash buffer.

114. The kit of any one of embodiments 98 to 113, wherein the surfactant is a polysorbate or Triton X-100.

115. The kit of embodiment 114, wherein the polysorbate is polysorbate-20 or polysorbate 80.

116. The kit of any one of embodiments 98 to 114, wherein the kit further comprises a vial containing a positive control monoclonal antibody specific for SARS-CoV-2 spike protein.

117. The kit of embodiment 116, wherein the positive control monoclonal antibody specific for SARS-CoV-2 spike protein binds to the receptor binding domain.

118. The kit of embodiment 116 or 117, wherein the positive control monoclonal antibody specific for SARS-CoV-2 spike protein is an IgG antibody.

119. The kit of any one of embodiments 116 to 118, wherein the vial containing the positive control monoclonal antibody comprises 500 μl to 2 ml of the positive control monoclonal antibody.

120. The kit of any one of embodiments 116 to 119, wherein the positive control monoclonal antibody is diluted 1:5 in the sample buffer.

121. The kit of any one of embodiments 98 to 120, wherein the kit further comprises a vial containing a negative control.

122. The kit of embodiment 121, wherein the negative control is a control buffer.

123. The kit of embodiment 121, wherein the negative control is a monoclonal antibody that does not bind to the SARS-CoV-2 spike protein.

124. The kit of any one of embodiments 121 to 123, wherein the vial containing the negative control comprises 500 μl to 2 ml of the negative control.

125. The kit of any one of embodiments 98 to 124, wherein the kit further comprises a second vial comprising a monoclonal antibody specific for SARS-CoV-2 spike protein.

126. The kit of embodiment 125, wherein the second vial comprises 500 μl to 2 ml of the monoclonal antibody specific for SARS-CoV-2 spike protein.

127. The kit of any one of embodiments 98 to 126, wherein the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml.

128. The kit of any one of embodiments 98 to 126, wherein the kit further comprises 7 calibrators, wherein each calibrator is a monoclonal antibody specific for SARS-CoV-2 spike protein and each calibrator has 0 to 200 AU/ml, and wherein each calibrator has a different AU/ml.

129. The kit of any one of embodiments 98 to 128, which is stored at 2° to 8° C.

130. A method for detecting antibody specific for SARS-CoV-2 spike protein, comprising:
(a) incubating a diluted heat inactivated biological sample in a well of a multi-well microtiter plate coated with a first recombinant soluble SARS-CoV-2 spike protein for 1.5 to 2.5 hours at room temperature, wherein the first recombinant soluble SARS-CoV-2 spike protein comprises amino acid residues 319 to 541 of a SARS-CoV-2 spike protein and a histidine tag at the C-terminus, and wherein the biological sample is serum or plasma from a human subject;
(b) washing the well with wash buffer, wherein the wash buffer comprises phosphate buffered saline and 0.5% to 2% of a surfactant;
(c) incubating a monoclonal antibody specific to human IgG conjugated to horseradish peroxidase in the well for 30 minutes to 1.5 hours at room temperature;
(d) washing the well with the wash buffer;
(e) incubating a substrate solution in the well for 10 to 40 minutes at room temperature, wherein the substrate solution comprises 3,3′,5,5′-Tetramethylbenzidine (TMB) substrate;
(f) adding stop solution to the well, wherein the stop solution comprises an acid; and
(g) determining the optical density of the well within 30 minutes using a microtiter plate reader set to 450 nm to detect antibody specific for SARS-CoV-2 spike protein.

131. The method of embodiment 130, wherein if wavelength correction is available set at 540 nm or 570 nm, or if wavelength correction is not available, readings at 540 nm or 570 nm are taken and subtracted from readings at 450 nm.

132. The method of embodiment 130 or 131, wherein the acid is an alkylsuphonic acid, 1 N HCl or 2 N H₂SO₄.

133. The method of embodiment 132, wherein the alkylsuphonic acid is 1-5% methanesulfonic acid.

134. The method of any one of embodiments 130 to 133, wherein the 100 μl of diluted heat inactivated biological sample is incubated in the well of the multi-well microtiter plate in step (a).

135. The method of any one of embodiments 130 to 134, wherein the biological sample was heat inactivated at 56° C. for 30 minutes to 1 hour.

136. The method of any one of embodiments 130 to 135, wherein the heat inactivated biological sample is diluted to a final dilution of 1:100 in sample buffer, wherein the sample buffer comprises phosphate buffered saline (PBS) with 0.5% to 5% milk.

137. The method of any one of embodiments 130 to 136, wherein the well is washed in steps (b) and (d) two or three times.

138. The method of embodiment 137, wherein each time the well is washed 400 μl of the wash buffer is used.

139. The method of any one of embodiments 130 to 138, wherein 100 μl of the substrate solution is incubated in the well in step (e).

140. The method of any one of embodiments 130 to 139, wherein the incubation in step (e) is for about 20 minutes.

141. The method of any one of embodiments 130 to 140, wherein 100 μl of the stop solution is added to the well.

142. The method of any one of embodiments 130 to 141, wherein the microtiter plate comprises a second well and a third well, and wherein steps (a) through (g) are concurrently performed with respect to the second and third wells except that in step (a): (i) a positive control monoclonal antibody specific for SARS-CoV-2 spike protein is used in the second well instead of the diluted biological sample and (ii) a negative control is used in the third well instead of the diluted biological sample.

143. The method of embodiment 142, wherein the positive control monoclonal antibody is diluted in the sample buffer.

144. The method of embodiment 142, wherein the positive control monoclonal antibody specific is diluted 1:5 in the sample buffer.

145. The method of any one of embodiments 142 to 144, wherein the positive control monoclonal antibody binds to the receptor binding domain of the SARS-CoV-2 spike protein.

146. The method of any one of embodiments 142 to 145, wherein the negative control is a monoclonal antibody that does not bind to the SARS-CoV-2 spike protein.

147. The method of embodiment 146, wherein the negative control antibody is diluted 1:5 in the sample buffer.

148. The method of any one of embodiments 142 to 147, wherein the negative control is a control buffer.

149. The method of any one of embodiments 130 to 148, wherein corrected optical density values for the biological sample are divided by corrected optical density values for the second well to calculate the confidence interval (CI) for the biological sample.

150. The method of embodiment 149, wherein a CI value of greater than or equal to 0.5 indicates that the biological sample is positive for antibody specific for the SARS-CoV-2 spike protein and a CI value of less than 0.5 indicates that the biological sample is negative for antibody specific for the SARS-CoV-2 spike protein.

151. The method of embodiment 149, wherein a CI value of greater than or equal to 0.6 indicates that the biological sample is positive for antibody specific for the SARS-CoV-2 spike protein and a CI value of less than 0.6 indicates that the biological sample is negative for antibody specific for the SARS-CoV-2 spike protein.

152. The method of embodiment 149, wherein a CI value of greater than or equal to 0.7 indicates that the biological sample is positive for antibody specific for the SARS-CoV-2 spike protein and a CI value of less than 0.7 indicates that the biological sample is negative for antibody specific for the SARS-CoV-2 spike protein.

153. The method of embodiment 149, wherein a CI value of 0.8 indicates that the biological sample is positive for antibody specific for the SARS-CoV-2 spike protein and a CI value of less than 0.8 indicates that the biological sample is negative for antibody specific for the SARS-CoV-2 spike protein.

154. The method of embodiment 149, wherein a CI value of greater than or equal to 0.9 indicates that the biological sample is positive for antibody specific for the SARS-CoV-2 spike protein and a CI value of less than 0.9 indicates that the biological sample is negative for antibody specific for the SARS-CoV-2 spike protein.

155. The method of embodiment 149, wherein a CI value of greater than or equal to 1 indicates that the biological sample is positive for antibody specific for the SARS-CoV-2 spike protein and a CI value of less than 1 indicates that the biological sample is negative for antibody specific for the SARS-CoV-2 spike protein.

156. The method of any one of embodiments 150 to 155, wherein the method further comprises a quantitative assay to assess quantitative levels of IgG antibodies against SARS-CoV-2 spike protein for those biological samples identified as positive, wherein the quantitative assay comprises:

(I) incubating for 1.5 to 2.5 hours at room temperature of a multi-well microtiter plate coated with a second recombinant soluble SARS-CoV-2 spike protein, wherein different wells of the microtiter plate contain either a diluted heat inactivated biological sample(s), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the second recombinant soluble SARS-CoV-2 spike protein comprises the amino acid sequence of a SARS-CoV-2 spike protein ectodomain or a derivative thereof, and wherein the biological sample is serum or plasma from a human subject;
(II) washing each of the wells with the wash buffer;
(III) incubating monoclonal antibody specific to human IgG conjugated to horseradish peroxidase in each of the wells for 30 minutes to 1.5 hours at room temperature;
(IV) washing each of the wells with the wash buffer;
(V) incubating the substrate solution in each of the wells for 10 to 40 minutes at room temperature;
(VI) adding the stop solution to each of the wells; and
(VII) determining the optical density of each of the wells within 30 minutes using a microtiter plate reader set to 450 nm to detect antibody specific for SARS-CoV-2 spike protein.

157. A method assessing the quantitative levels of IgG antibodies against SARS-CoV-2 spike protein, wherein the method comprises a quantitative assay comprising:
(I) incubating for 1.5 to 2.5 hours at room temperature of a multi-well microtiter plate coated with a first recombinant soluble SARS-CoV-2 spike protein, wherein different wells of the microtiter plate contain either a diluted heat inactivated biological sample(s), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the first recombinant soluble SARS-CoV-2 spike protein comprises the amino acid sequence of a SARS-CoV-2 spike protein ectodomain or a derivative thereof, and wherein the biological sample is serum or plasma from a human subject;
(II) washing each of the wells with the wash buffer, wherein the wash buffer comprises phosphate buffered saline and 0.5% to 2% of a surfactant;
(III) incubating monoclonal antibody specific to human IgG conjugated to horseradish peroxidase in each of the wells for 30 minutes to 1.5 hours at room temperature;
(IV) washing each of the wells with the wash buffer;
(V) incubating a substrate solution in each of the wells for 10 to 40 minutes at room temperature, wherein the substrate solution comprises 3,3,5,5-Tetramethylbenzidine (TMB) substrate;
(VI) adding a stop solution to each of the wells, wherein the stop solution comprises an acid; and
(VII) determining the optical density of each of the wells within 30 minutes using a microtiter plate reader set to 450 nm to detect antibody specific for SARS-CoV-2 spike protein.

158. The method of embodiment 157, wherein the acid is an alkyl sulfonic acid, 1 N HCl or 2 N H₂SO₄.

159. The method of embodiment 158, wherein the alkyl sulfonic acid is 1-5% methanesulfonic acid.

160. The method of any one of embodiments 157 to 159, wherein the surfactant is a polysorbate or Triton X-100.

161. The method of embodiment 160, wherein the polysorbate is polysorbate-20 or polysorbate-80.

162. The method of any one of embodiments 156 to 161, wherein each well is washed in steps (II) or (IV) two or three times.

163. The method of embodiment 162, wherein each time the well is washed 400 μl of the wash buffer is used.

164. The method of any one of embodiments 156 to 163, wherein 100 μl of the monoclonal antibody specific to human IgG conjugated to horseradish peroxidase is incubated in each of the wells in step (III).

165. The method of any one of embodiments 156 to 164, wherein 100 μl of the substrate solution is incubated in each of the wells in step (V).

166. The method of any one of embodiments 156 to 165, wherein 100 μl of the stop solution is incubated in each of the wells in step (VI).

167. The method of any one of embodiments 156 to 166, wherein the biological sample is heat inactivated at 56° C. for 30 minutes to 1 hour.

168. The method of any one of embodiments 156 to 167, the heat inactivated biological sample is diluted to a final dilution of 1:200 in sample buffer.

169. The method of any one of embodiments 156 to 168, wherein the three controls are diluted 1:5 in the sample buffer.

170. The method of any one of embodiments 156 to 169, wherein if wavelength correction is available set at 540 nm or 570 nm, or if wavelength correction is not available, readings at 540 nm or 570 nm are taken and subtracted from readings at 450 nm.

171. The method of any one of embodiments 156 to 170, wherein the quantitative assay further comprises: (VIII) generating a calibration curve and comparing the signal from the diluted heat inactivated biological sample(s) to the calibration curve to generate a final result of IgG levels in arbitrary units per milliliter (AU/ml).

172. A kit comprising:
(a) a multi-well microtiter plate (e.g., a 96 well microtiter plate) coated with a recombinant soluble SARS-CoV-2 spike protein, wherein the recombinant soluble SARS-CoV-2 spike protein comprises a SARS-CoV-2 receptor binding domain (e.g., amino acid residues 319 to 541 of a SARS-CoV-2 spike protein) or a derivative thereof and optionally a tag (e.g., a histidine tag, at the C-terminus);
(b) a vial containing concentrated monoclonal antibody specific to human IgG or another immunoglobulin isotype or subtype conjugated to horseradish peroxidase, alkaline phosphatase or another detectable moiety;
(c) a vial containing a substrate (e.g., 3,3′,5,5′-Tetramethylbenzidine (TMB) substrate);
(d) a vial containing a stop solution (e.g., a stop solution comprising an acid);
(e) a vial containing a sample buffer (e.g., a sample buffer comprising phosphate buffered saline (PBS) with 0.5% to 5% milk); and
(f) a vial containing a wash buffer (e.g., a wash buffer comprising phosphate buffered saline and 0.5% to 2% of a surfactant).

173. A kit comprising:
(a) a vial containing a recombinant soluble SARS-CoV-2 spike protein, wherein the recombinant soluble SARS-CoV-2 spike protein comprises a SARS-CoV-2 receptor binding domain (e.g., amino acid residues 319 to 541 of a SARS-CoV-2 spike protein) or a derivative thereof and optionally a tag (e.g., a histidine tag, at the C-terminus);
(b) a vial containing concentrated monoclonal antibody specific to human IgG or another immunoglobulin isotype or subtype conjugated to horseradish peroxidase, alkaline phosphatase or another detectable moiety;
(c) a vial containing a substrate (e.g., 3,3′,5,5′-Tetramethylbenzidine (TMB) substrate);
(d) a vial containing a stop solution (e.g., a stop solution comprising an acid);
(e) a vial containing a sample buffer (e.g., a sample buffer comprising phosphate buffered saline (PBS) with 0.5% to 5% milk); and
(f) a vial containing a wash buffer (e.g., a wash buffer comprising phosphate buffered saline and 0.5% to 2% of a surfactant).

174. The kit of embodiment 172, wherein the kit further comprises a second multi-well microtiter plate (e.g., a 96 well microtiter plate) coated with a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof.

175. The kit of embodiment 173, wherein the kit further comprises a vial comprising a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof.

176. The kit of embodiment 174 or 175, wherein the second recombinant soluble SARS-CoV-2 spike protein comprises the amino acid sequence of SEQ ID NO: 4 or 6.

177. The kit of any one of embodiments 172 to 176, wherein the recombinant SARS-CoV-2 spike protein comprises the amino acid sequence set forth in SEQ ID NO:10.

178. A kit comprising:
(a) a multi-well microtiter plate coated (e.g., a 96 well microtiter plate) with a recombinant soluble SARS-CoV-2 spike protein, wherein the recombinant soluble SARS-CoV-2 spike protein comprises the amino acid sequence of a SARS-CoV-2 spike protein ectodomain or a derivative thereof;
(b) a vial containing concentrated monoclonal antibody specific to human IgG or another immunoglobulin isotype or subtype conjugated to horseradish peroxidase, alkaline phosphatase or another detectable moiety;
(c) a vial containing a substrate (e.g., 3,3′,5,5′-Tetramethylbenzidine (TMB) substrate);
(d) a vial containing a stop solution (e.g., a stop solution comprising an acid);
(e) a vial containing a sample buffer (e.g., a sample buffer comprising phosphate buffered saline (PBS) with 0.5% to 5% milk);
(f) a vial containing a wash buffer (e.g., a wash buffer comprising phosphate buffered saline and 0.5% to 2% of a surfactant).

179. A kit comprising:
(a) a vial comprising a recombinant soluble SARS-CoV-2 spike protein, wherein the recombinant soluble SARS-CoV-2 spike protein comprises the amino acid sequence of a SARS-CoV-2 spike protein ectodomain or a derivative thereof;
(b) a vial containing concentrated monoclonal antibody specific to human IgG or another immunoglobulin isotype or subtype conjugated to horseradish peroxidase, alkaline phosphatase or another detectable moiety;
(c) a vial containing a substrate (e.g., 3,3′,5,5′-Tetramethylbenzidine (TMB) substrate);
(d) a vial containing a stop solution (e.g., a stop solution comprising an acid);
(e) a vial containing a sample buffer (e.g., a sample buffer comprising phosphate buffered saline (PBS) with 0.5% to 5% milk);
(f) a vial containing a wash buffer (e.g., a wash buffer comprising phosphate buffered saline and 0.5% to 2% of a surfactant).

180. The kit of any one of embodiments 178 or 179, wherein the recombinant SARS-CoV-2 spike protein comprises the amino acid sequence set forth in SEQ ID NO:10.

181. The kit of any one of embodiment 172 to 180, wherein the vial containing the concentrated monoclonal antibody specific to human IgG conjugated to horseradish peroxidase comprises 100 to 500 μl of the concentrated antibody.

182. The kit of any one of embodiments 172 to 181, wherein the concentrated monoclonal antibody specific to human IgG conjugated to horseradish peroxidase is 100× to 1000× concentrated monoclonal antibody.

183. The kit of any one of embodiments 172 to 182, wherein the kit further comprises a vial containing a positive control monoclonal antibody specific for SARS-CoV-2 spike protein.

184. The kit of embodiment 183, wherein the positive control monoclonal specific for SARS-CoV-2 spike protein binds to the receptor binding domain.

185. The kit of 183 or 184, wherein the positive control monoclonal antibody specific for SARS-CoV-2 spike protein is an IgG antibody.

186. The kit of any one of embodiments 183 to 185, wherein the vial containing the positive control monoclonal antibody comprises 500 μl to 2 ml of the positive control monoclonal antibody.

187. The kit of any one of embodiments 183 to 186, wherein the positive control monoclonal antibody is diluted 1:5 in the sample buffer.

188. The kit of any one of embodiments 172 to 187, wherein the kit further comprises a vial containing a negative control.

189. The kit of embodiment 188, wherein the negative control is a control buffer.

190. The kit of embodiment 188, wherein the negative control is a monoclonal antibody that does not bind to the SARS-CoV-2 spike protein.

191. The kit of any one of embodiments 188 to 190, wherein the vial containing the negative control comprises 500 μl to 2 ml of the negative control.

192. The kit of any one of embodiments 172 to 191, wherein the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml.

193. The kit of any one of embodiments 172 to 191, wherein the kit further comprises 7 calibrators, wherein each calibrator is a monoclonal antibody specific for SARS-CoV-2 spike protein and each calibrator has 0 to 200 AU/ml, and wherein each calibrator has a different AU/ml.

194. The kit of any one of embodiments 172 to 193, which is stored at 2° to 8° C.

195. A kit comprising a multi-well microtiter plate (e.g., a 96 well microtiter plate) coated with a recombinant soluble SARS-CoV-2 spike protein, wherein the recombinant soluble SARS-CoV-2 spike protein comprises a SARS-CoV-2 receptor binding domain (e.g., amino acid residues 319 to 541 of a SARS-CoV-2 spike protein) or a derivative thereof and optionally a tag (e.g., a histidine tag, at the C-terminus).

196. A kit comprising a multi-well microtiter plate (e.g., a 96 well microtiter plate) coated with a recombinant soluble SARS-CoV-2 spike protein, wherein the recombinant soluble SARS-CoV-2 spike protein comprises a SARS-CoV-2 ectodomain or a derivative thereof and optionally a tag (e.g., a histidine tag, at the C-terminus).

197. The kit of embodiment 195 or 196, wherein the kit further comprises a vial containing a monocloncal antibody specific for a human immunoglobulin isotype or subtype (e.g., Ig) or a pan-specific for human immunoglobulin.

198. The kit of any one of embodiments 195 to 197, wherein the kit further comprises a vial containing a positive control antibody specific for SARS-CoV-2 spike protein.

199. The kit of any one of embodiments 195 to 198, wherein the kit further comprises one, two or all of the following: (i) a wash buffer, (ii) a sample buffer, (iii) substrate, and (iv) a stop solution.

200. The kit of embodiment 178, 179 or 196, wherein the recombinant soluble SARS-CoV-2 spike protein comprises the amino acid sequence of SEQ ID NO: 4 or SEQ ID NO:6 without the first 14 amino acid residues.

201. The kit of embodiment 178, 179 or 196, wherein the recombinant soluble SARS-CoV-2 spike protein comprises the amino acid sequence of SEQ ID NO: 4 or SEQ ID NO:6.

202. The kit of embodiment 178, 179 or 196, wherein the recombinant soluble SARS-CoV-2 spike protein comprises the ectodomain of a SARS-CoV-2 spike protein with the polybasic/furin cleavage site replaced by a single A.

203. The kit of embodiment 202, wherein the second recombinant soluble SARS-CoV-2 spike protein further comprises a C-terminal cleavage site and a trimerization domain.

204. The kit of any one of embodiments 98 to 129, wherein the second recombinant soluble SARS-CoV-2 spike protein comprises the amino acid sequence of SEQ ID NO: 4 or SEQ ID NO:6 without the first 14 amino acid residues.

205. The kit of any one of embodiments 98 to 129, wherein the second recombinant soluble SARS-CoV-2 spike protein comprises the amino acid sequence of SEQ ID NO: 4 or SEQ ID NO:6.

206. The kit of any one of embodiments 98 to 129, wherein the second recombinant soluble SARS-CoV-2 spike protein comprises the ectodomain of a SARS-CoV-2 spike protein with the polybasic/furin cleavage site replaced by a single A.

207. The kit of embodiment 206, wherein the second recombinant soluble SARS-CoV-2 spike protein further comprises a C-terminal cleavage site and a trimerization domain.

8. SEQUENCES
8.1 SARS-CoV-2 Spike Protein and DNA Sequences

| | |
|---|---|
| SARS-CoV-2 RBD | ATGTTCGTGTTTCTGGTGCTGCTGCCTCTGGTGTCCAGCCAGCGGGTGCAGCCCACCGAATC |
| (codon-optimized) | CATCGTGCGGTTCCCCAATATCACCAATCTGTGCCCCTTCGGCGAGGTGTTCAATGCCACC |
| DNA Sequence (SEQ | AGATTCGCCTCTGTGTACGCCTGGAACCGGAAGCGGATCAGCAATTGCGTGGCCGACTACT |
| ID NO: 1) | CCGTGCTGTACAACTCCGCCAGCTTCAGCACCTTCAAGTGCTACGGCGTGTCCCCTACCAA |
| | GCTGAACGACCTGTGCTTCACAAACGTGTACGCCGACAGCTTCGTGATCCGGGGAGATGAA |
| | GTGCGGCAGATTGCCCCTGGACAGACAGGCAAGATCGCCGACTACAACTACAAGCTGCCC |
| | GACGACTTCACCGGCTGTGTGATTGCCTGGAACAGCAACAACCTGGACTCCAAAGTCGGCG |
| | GCAACTACAATTACCTGTACCGGCTGTTCCGGAAGTCCAATCTGAAGCCCTTCGAGCGGGA |
| | CATCTCCACCGAGATCTATCAGGCCGGCAGCACCCCTTGTAACGGCGTGGAAGGCTTCAAC |
| | TGCTACTTCCCACTGCAGTCCTACGGCTTTCAGCCCACAAATGGCGTGGGCTATCAGCCCT |
| | ACAGAGTGGTGGTGCTGAGCTTCGAACTGCTGCATGCCCCTGCCACAGTGTGCGGCCCTAA |
| | GAAAAGCACCAATCTCGTGAAGAACAAATGCGTGAACTTCcaccatcaccatcaccatTGATAA |
| SARS-CoV-2 RBD | MFVFLVLLPLVSSQRVQPTESIVRFPNITNLCPFGEVFNATRFASVYAWNRKRISNCVADYSVL |
| Amino acid sequence | YNSASFSTFKCYGVSPTKLNDLCFTNVYADSFVIRGDEVRQIAPGQTGKIADYNYKLPDDFTGC |
| (SEQ ID NO: 2): | VIAWNSNNLDSKVGGNYNYLYRLFRKSNLKPFERDISTEIYQAGSTPCNGVEGENCYFPLQSYG |
| | FQPTNGVGYQPYRVVVLSFELLHAPATVCGPKKSTNLVKNKCVNFHHHHHH** |
| SARS-CoV-2 Spike | ATGTTCGTGTTTCTGGTGCTGCTGCCTCTGGTGTCCAGCCAGTGTGTGAACCTGACCACAAG |
| with CS and PP | AACCCAGCTGCCTCCAGCCTACACCAACAGCTTTACCAGAGGCGTGTACTACCCCGACAAG |
| (codon-optimized) | GTGTTCAGATCCAGCGTGCTGCACTCTACCCAGGACCTGTTCCTGCCTTTCTTCAGCAACGT |
| DNA Sequence (SEQ | GACCTGGTTCCACGCCATCCACGTGTCCGGCACCAATGGCACCAAGAGATTCGACAACCCC |
| ID NO: 3) | GTGCTGCCCTTCAACGACGGGGTGTACTTTGCCAGCACCGAGAAGTCCAACATCATCAGAG |
| | GCTGGATCTTCGGCACCACACTGGACAGCAAGACCCAGAGCCTGCTGATCGTGAACAACG |
| | CCACCAACGTGGTCATCAAAGTGTGCGAGTTCCAGTTCTGCAACGACCCCTTCCTGGGCGT |
| | CTACTATCACAAGAACAACAAGAGCTGGATGGAAAGCGAGTTCCGGGTGTACAGCAGCGC |
| | CAACAACTGCACCTTCGAGTACGTGTCCCAGCCTTTCCTGATGGACCTGGAAGGCAAGCAG |
| | GGCAACTTCAAGAACCTGCGCGAGTTCGTGTTCAAGAACATCGACGGCTACTTCAAGATCT |
| | ACAGCAAGCACACCCCTATCAACCTCGTGCGGGATCTGCCTCAGGGCTTCTCTGCTCTGGA |
| | ACCCCTGGTGGATCTGCCCATCGGCATCAACATCACCCGGTTTCAGACACTGCTGGCCCTG |
| | CACAGAAGCTACCTGACACCTGGCGATAGCAGCAGCGGATGGACAGCTGGTGCCGCCGCT |
| | TACTATGTGGGCTACCTGCAGCCTAGAACCTTTCTGCTGAAGTACAACGAGAACGGCACCA |
| | TCACCGACGCCGTGGATTGTGCTCTGGATCCTCTGAGCGAGACAAAGTGCACCCTGAAGTC |
| | CTTCACCGTGGAAAAGGGCATCTACCAGACCAGCAACTTCCGGGTGCAGCCCACCGAATCC |
| | ATCGTGCGGTTCCCCAATATCACCAATCTGTGCCCCTTCGGCGAGGTGTTCAATGCCACCA |
| | GATTCGCCTCTGTGTACGCCTGGAACCGGAAGCGGATCAGCAATTGCGTGGCCGACTACTC |
| | CGTGCTGTACAACTCCGCCAGCTTCAGCACCTTCAAGTGCTACGGCGTGTCCCCTACCAAG |
| | CTGAACGACCTGTGCTTCACAAACGTGTACGCCGACAGCTTCGTGATCCGGGGAGATGAAG |
| | TGCGGCAGATTGCCCCTGGACAGACAGGCAAGATCGCCGACTACAACTACAAGCTGCCCG |
| | ACGACTTCACCGGCTGTGTGATTGCCTGGAACAGCAACAACCTGGACTCCAAAGTCGGCGG |
| | CAACTACAATTACCTGTACCGGCTGTTCCGGAAGTCCAATCTGAAGCCCTTCGAGCGGGAC |
| | ATCTCCACCGAGATCTATCAGGCCGGCAGCACCCCTTGTAACGGCGTGGAAGGCTTCAACT |
| | GCTACTTCCCACTGCAGTCCTACGGCTTTCAGCCCACAAATGGCGTGGGCTATCAGCCCTA |
| | CAGAGTGGTGGTGCTGAGCTTCGAACTGCTGCATGCCCCTGCCACAGTGTGCGGCCCTAAG |
| | AAAAGCACCAATCTCGTGAAGAACAAATGCGTGAACTTCAACTTCAACGGCCTGACCGGC |
| | ACCGGCGTGCTGACAGAGAGCAACAAGAAGTTCCTGCCATTCCAGCAGTTTGGCCGGGAT |
| | ATCGCCGATACCACAGACGCCGTTAGAGATCCCCAGACACTGGAAATCCTGGACATCACCC |
| | CTTGCAGCTTCGGCGGAGTGTCTGTGATCACCCCTGGCACCAACACCAGCAATCAGGTGGC |
| | AGTGCTGTACCAGGACGTGAACTGTACCGAAGTGCCCGTGGCCATTCACGCCGATCAGCTG |
| | ACACCTACATGGCGGGTGTACTCCACCGGCAGCAATGTGTTTCAGACCAGAGCCGGCTGTC |
| | TGATCGGAGCCGAGCACGTGAACAATAGCTACGAGTGCGACATCCCCATCGGCGCTGGCA |
| | TCTGTGCCAGCTACCAGACACAGACAAACAGCCCCGCCTCTGTGGCCAGCCAGAGCATCAT |
| | TGCCTACACAATGTCTCTGGGCGCCGAGAACAGCGTGGCCTACTCCAACAACTCTATCGCT |
| | ATCCCCACCAACTTCACCATCAGCGTGACCACAGAGATCCTGCCTGTGTCCATGACCAAGA |
| | CCAGCGTGGACTGCACCATGTACATCTGCGGCGATTCCACCGAGTGCTCCAACCTGCTGCT |
| | GCAGTACGGCAGCTTCTGCACCCAGCTGAATAGAGCCCTGACAGGGATCGCCGTGGAACA |
| | GGACAAGAACACCCAAGAGGTGTTCGCCCAAGTGAAGCAGATCTACAAGACCCCTCCTAT |
| | CAAGGACTTCGGCGGCTTCAATTTCAGCCAGATTCTGCCCGATCCTAGCAAGCCCAGCAAG |
| | CGGAGCTTCATCGAGGACCTGCTGTTCAACAAAGTGACACTGGCCGACGCCGGCTTCATCA |
| | AGCAGTATGGCGATTGTCTGGGCGACATTGCCGCCAGGGATCTGATTTGCGCCCAGAAGTT |
| | TAACGGACTGACAGTGCTGCCTCCTCTGCTGACCGATGAGATGATCGCCCAGTACACATCT |
| | GCCCTGCTGGCCGGCACAATCACAAGCGGCTGGACATTTGGAGCTGGCGCCGCTCTGCAGA |
| | TCCCCTTTGCTATGCAGATGGCCTACCGGTTCAACGGCATCGGAGTGACCCAGAATGTGCT |
| | GTACGAGAACCAGAAGCTGATCGCCAACCAGTTCAACAGCGCCATCGGCAAGATCCAGGA |
| | CAGCCTGAGCAGCACAGCAAGCGCCCTGGGAAAGCTGCAGGACGTGGTCAACCAGAATGC |
| | CCAGGCACTGAACACCCTGGTCAAGCAGCTGTCCTCCAACTTCGGCGCCATCAGCTCTGTG |
| | CTGAACGATATCCTGAGCAGACTGGACcctcctGAAGCCGAGGTGCAGATCGACAGACTGATC |
| | ACCGGAAGGCTGCAGTCCCTGCAGACCTACGTTACCCAGCAGCTGATCAGAGCCGCCGAG |
| | ATTAGAGCCTCTGCCAATCTGGCCGCCACCAAGATGTCTGAGTGTGTGCTGGGCCAGAGCA |
| | AGAGAGTGGACTTTTGCGGCAAGGGCTACCACCTGATGAGCTTCCCTCAGTCTGCCCCTCA |
| | CGGCGTGGTGTTTCTGCACGTGACATACGTGCCCGCTCAAGAGAAGAATTTCACCACCGCT |
| | CCAGCCATCTGCCACGACGGCAAAGCCCACTTTCCTAGAGAAGGCGTGTTCGTGTCCAACG |
| | GCACCCATTGGTTCGTGACCCAGCGGAACTTCTACGAGCCCCAGATCATCACCACCGACAA |
| | CACCTTCGTGTCTGGCAACTGCGACGTCGTGATCGGCATTGTGAACAATACCGTGTACGAC |
| | CCTCTGCAGCCCGAGCTGGACAGCTTCAAAGAGGAACTGGATAAGTACTTTAAGAACCAC |
| | ACAAGCCCCGACGTGGACCTGGGCGATATCAGCGGAATCAATGCCAGCGTCGTGAACATC |
| | CAGAAAGAGATCGACCGGCTGAACGAGGTGGCCAAGAATCTGAACGAGAGCCTGATCGAC |
| | CTGCAAGAACTGGGGAAGTACGAGCAGTACATCAAGTGGCCCAGCGGCCGCTTGGTCCCA |
| | CGTGGCTCACCCGGATCTGGATACATCCCGGAGGCCCCTAGGGACGGTCAAGCTTACGTGA |
| | GAAAGGACGGCGAATGGGTTCTGCTGTCGACCTTCTTGGGACATCATCATCATCATCACTA |
| | ATGA |
| SARS-CoV-2 Spike | MFVFLVLLPLVSSQCVNLTTRTQLPPAYTNSFTRGVYYPDKVFRSSVLHSTQDLFLPFFSNVTW |
| with CS and PP | FHAIHVSGTNGTKRFDNPVLPFNDGVYFASTEKSNIIRGWIFGTTLDSKTQSLLIVNNATNVVIK |
| Amino acid sequence | VCEFQFCNDPFLGVYYHKNNKSWMESEFRVYSSANNCTFEYVSQPFLMDLEGKQGNFKNLRE |
| (SEQ ID NO: 4) | FVFKNIDGYFKIYSKHTPINLVRDLPQGFSALEPLVDLPIGINITRFQTLLALHRSYLTPGDSSSG |
| | WTAGAAAYYVGYLQPRTFLLKYNENGTITDAVDCALDPLSETKCTLKSFTVEKGIYQTSNFRV |
| | QPTESIVRFPNITNLCPFGEVFNATRFASVYAWNRKRISNCVADYSVLYNSASFSTFKCYGVSPT |
| | KLNDLCFTNVYADSFVIRGDEVRQIAPGQTGKIADYNYKLPDDFTGCVIAWNSNNLDSKVGGN |
| | YNYLYRLFRKSNLKPFERDISTEIYQAGSTPCNGVEGENCYFPLQSYGFQPTNGVGYQPYRVVV |
| | LSFELLHAPATVCGPKKSTNLVKNKCVNFNFNGLTGTGVLTESNKKFLPFQQFGRDIADTTDA |
| | VRDPQTLEILDITPCSFGGVSVITPGTNTSNQVAVLYQDVNCTEVPVAIHADQLTPTWRVYSTG |
| | SNVFQTRAGCLIGAEHVNNSYECDIPIGAGICASYQTQTNSPASVASQSIIAYTMSLGAENSVAY |
| | SNNSIAIPTNFTISVTTEILPVSMTKTSVDCTMYICGDSTECSNLLLQYGSFCTQLNRALTGIAVE |
| | QDKNTQEVFAQVKQIYKTPPIKDFGGFNFSQILPDPSKPSKRSFIEDLLFNKVTLADAGFIKQYG |
| | DCLGDIAARDLICAQKFNGLTVLPPLLTDEMIAQYTSALLAGTITSGWTFGAGAALQIPFAMQM |
| | AYRFNGIGVTQNVLYENQKLIANQFNSAIGKIQDSLSSTASALGKLQDVVNQNAQALNTLVKQ |
| | LSSNFGAISSVLNDILSRLDPPEAEVQIDRLITGRLQSLQTYVTQQLIRAAEIRASANLAATKMSE |
| | CVLGQSKRVDFCGKGYHLMSFPQSAPHGVVFLHVTYVPAQEKNFTTAPAICHDGKAHFPREG |
| | VFVSNGTHWFVTQRNFYEPQIITTDNTFVSGNCDVVIGIVNNTVYDPLQPELDSFKEELDKYFK |
| | NHTSPDVDLGDISGINASVVNIQKEIDRLNEVAKNLNESLIDLQELGKYEQYIKWPSGRLVPRGS |
| | PGSGYIPEAPRDGQAYVRKDGEWVLLSTFLGHHHHHH** |
| SARS-CoV-2 Spike | ATGTTCGTGTTTCTGGTGCTGCTGCCTCTGGTGTCCAGCCAGTGTGTGAACCTGACCACAAG |
| with CS (codon- | AACCCAGCTGCCTCCAGCCTACACCAACAGCTTTACCAGAGGCGTGTACTACCCCGACAAG |
| optimized) | GTGTTCAGATCCAGCGTGCTGCACTCTACCCAGGACCTGTTCCTGCCTTTCTTCAGCAACGT |
| DNA Sequence (SEQ | GACCTGGTTCCACGCCATCCACGTGTCCGGCACCAATGGCACCAAGAGATTCGACAACCCC |
| ID NO: 5) | GTGCTGCCCTTCAACGACGGGGTGTACTTTGCCAGCACCGAGAAGTCCAACATCATCAGAG |
| | GCTGGATCTTCGGCACCACACTGGACAGCAAGACCCAGAGCCTGCTGATCGTGAACAACG |
| | CCACCAACGTGGTCATCAAAGTGTGCGAGTTCCAGTTCTGCAACGACCCCTTCCTGGGCGT |
| | CTACTATCACAAGAACAACAAGAGCTGGATGGAAAGCGAGTTCCGGGTGTACAGCAGCGC |
| | CAACAACTGCACCTTCGAGTACGTGTCCCAGCCTTTCCTGATGGACCTGGAAGGCAAGCAG |
| | GGCAACTTCAAGAACCTGCGCGAGTTCGTGTTCAAGAACATCGACGGCTACTTCAAGATCT |
| | ACAGCAAGCACACCCCTATCAACCTCGTGCGGGATCTGCCTCAGGGCTTCTCTGCTCTGGA |
| | ACCCCTGGTGGATCTGCCCATCGGCATCAACATCACCCGGTTTCAGACACTGCTGGCCCTG |
| | CACAGAAGCTACCTGACACCTGGCGATAGCAGCAGCGGATGGACAGCTGGTGCCGCCGCT |
| | TACTATGTGGGCTACCTGCAGCCTAGAACCTTTCTGCTGAAGTACAACGAGAACGGCACCA |
| | TCACCGACGCCGTGGATTGTGCTCTGGATCCTCTGAGCGAGACAAAGTGCACCCTGAAGTC |
| | CTTCACCGTGGAAAAGGGCATCTACCAGACCAGCAACTTCCGGGTGCAGCCCACCGAATCC |
| | ATCGTGCGGTTCCCCAATATCACCAATCTGTGCCCCTTCGGCGAGGTGTTCAATGCCACCA |
| | GATTCGCCTCTGTGTACGCCTGGAACCGGAAGCGGATCAGCAATTGCGTGGCCGACTACTC |
| | CGTGCTGTACAACTCCGCCAGCTTCAGCACCTTCAAGTGCTACGGCGTGTCCCCTACCAAG |
| | CTGAACGACCTGTGCTTCACAAACGTGTACGCCGACAGCTTCGTGATCCGGGGAGATGAAG |
| | TGCGGCAGATTGCCCCTGGACAGACAGGCAAGATCGCCGACTACAACTACAAGCTGCCCG |
| | ACGACTTCACCGGCTGTGTGATTGCCTGGAACAGCAACAACCTGGACTCCAAAGTCGGCGG |
| | CAACTACAATTACCTGTACCGGCTGTTCCGGAAGTCCAATCTGAAGCCCTTCGAGCGGGAC |
| | ATCTCCACCGAGATCTATCAGGCCGGCAGCACCCCTTGTAACGGCGTGGAAGGCTTCAACT |
| | GCTACTTCCCACTGCAGTCCTACGGCTTTCAGCCCACAAATGGCGTGGGCTATCAGCCCTA |
| | CAGAGTGGTGGTGCTGAGCTTCGAACTGCTGCATGCCCCTGCCACAGTGTGCGGCCCTAAG |
| | AAAAGCACCAATCTCGTGAAGAACAAATGCGTGAACTTCAACTTCAACGGCCTGACCGGC |
| | ACCGGCGTGCTGACAGAGAGCAACAAGAAGTTCCTGCCATTCCAGCAGTTTGGCCGGGAT |
| | ATCGCCGATACCACAGACGCCGTTAGAGATCCCCAGACACTGGAAATCCTGGACATCACCC |
| | CTTGCAGCTTCGGCGGAGTGTCTGTGATCACCCCTGGCACCAACACCAGCAATCAGGTGGC |
| | AGTGCTGTACCAGGACGTGAACTGTACCGAAGTGCCCGTGGCCATTCACGCCGATCAGCTG |
| | ACACCTACATGGCGGGTGTACTCCACCGGCAGCAATGTGTTTCAGACCAGAGCCGGCTGTC |
| | TGATCGGAGCCGAGCACGTGAACAATAGCTACGAGTGCGACATCCCCATCGGCGCTGGCA |
| | TCTGTGCCAGCTACCAGACACAGACAAACAGCCCCGCCTCTGTGGCCAGCCAGAGCATCAT |
| | TGCCTACACAATGTCTCTGGGCGCCGAGAACAGCGTGGCCTACTCCAACAACTCTATCGCT |
| | ATCCCCACCAACTTCACCATCAGCGTGACCACAGAGATCCTGCCTGTGTCCATGACCAAGA |
| | CCAGCGTGGACTGCACCATGTACATCTGCGGCGATTCCACCGAGTGCTCCAACCTGCTGCT |
| | GCAGTACGGCAGCTTCTGCACCCAGCTGAATAGAGCCCTGACAGGGATCGCCGTGGAACA |
| | GGACAAGAACACCCAAGAGGTGTTCGCCCAAGTGAAGCAGATCTACAAGACCCCTCCTAT |
| | CAAGGACTTCGGCGGCTTCAATTTCAGCCAGATTCTGCCCGATCCTAGCAAGCCCAGCAAG |
| | CGGAGCTTCATCGAGGACCTGCTGTTCAACAAAGTGACACTGGCCGACGCCGGCTTCATCA |
| | AGCAGTATGGCGATTGTCTGGGCGACATTGCCGCCAGGGATCTGATTTGCGCCCAGAAGTT |
| | TAACGGACTGACAGTGCTGCCTCCTCTGCTGACCGATGAGATGATCGCCCAGTACACATCT |
| | GCCCTGCTGGCCGGCACAATCACAAGCGGCTGGACATTTGGAGCTGGCGCCGCTCTGCAGA |
| | TCCCCTTTGCTATGCAGATGGCCTACCGGTTCAACGGCATCGGAGTGACCCAGAATGTGCT |
| | GTACGAGAACCAGAAGCTGATCGCCAACCAGTTCAACAGCGCCATCGGCAAGATCCAGGA |
| | CAGCCTGAGCAGCACAGCAAGCGCCCTGGGAAAGCTGCAGGACGTGGTCAACCAGAATGC |
| | CCAGGCACTGAACACCCTGGTCAAGCAGCTGTCCTCCAACTTCGGCGCCATCAGCTCTGTG |
| | CTGAACGATATCCTGAGCAGACTGGACAAGGTGGAAGCCGAGGTGCAGATCGACAGACTG |
| | ATCACCGGAAGGCTGCAGTCCCTGCAGACCTACGTTACCCAGCAGCTGATCAGAGCCGCCG |
| | AGATTAGAGCCTCTGCCAATCTGGCCGCCACCAAGATGTCTGAGTGTGTGCTGGGCCAGAG |
| | CAAGAGAGTGGACTTTTGCGGCAAGGGCTACCACCTGATGAGCTTCCCTCAGTCTGCCCCT |
| | CACGGCGTGGTGTTTCTGCACGTGACATACGTGCCCGCTCAAGAGAAGAATTTCACCACCG |
| | CTCCAGCCATCTGCCACGACGGCAAAGCCCACTTTCCTAGAGAAGGCGTGTTCGTGTCCAA |
| | CGGCACCCATTGGTTCGTGACCCAGCGGAACTTCTACGAGCCCCAGATCATCACCACCGAC |
| | AACACCTTCGTGTCTGGCAACTGCGACGTCGTGATCGGCATTGTGAACAATACCGTGTACG |
| | ACCCTCTGCAGCCCGAGCTGGACAGCTTCAAAGAGGAACTGGATAAGTACTTTAAGAACC |
| | ACACAAGCCCCGACGTGGACCTGGGCGATATCAGCGGAATCAATGCCAGCGTCGTGAACA |
| | TCCAGAAAGAGATCGACCGGCTGAACGAGGTGGCCAAGAATCTGAACGAGAGCCTGATCG |
| | ACCTGCAAGAACTGGGGAAGTACGAGCAGTACATCAAGTGGCCCAGCGGCCGCTTGGTCC |
| | CACGTGGCTCACCCGGATCTGGATACATCCCGGAGGCCCCTAGGGACGGTCAAGCTTACGT |
| | GAGAAAGGACGGCGAATGGGTTCTGCTGTCGACCTTCTTGGGACATCATCATCATCATCAC |
| | TAATGA |
| SARS-CoV-2 Spike | MFVFLVLLPLVSSQCVNLTTRTQLPPAYTNSFTRGVYYPDKVFRSSVLHSTQDLFLPFFSNVTW |
| with CS | FHAIHVSGTNGTKRFDNPVLPFNDGVYFASTEKSNIIRGWIFGTTLDSKTQSLLIVNNATNVVIK |
| Amino acid sequence | VCEFQFCNDPFLGVYYHKNNKSWMESEFRVYSSANNCTFEYVSQPFLMDLEGKQGNFKNLRE |
| (SEQ ID NO: 6): | FVFKNIDGYFKIYSKHTPINLVRDLPQGFSALEPLVDLPIGINITRFQTLLALHRSYLTPGDSSSG |
| | WTAGAAAYYVGYLQPRTFLLKYNENGTITDAVDCALDPLSETKCTLKSFTVEKGIYQTSNFRV |
| | QPTESIVRFPNITNLCPFGEVFNATRFASVYAWNRKRISNCVADYSVLYNSASFSTFKCYGVSPT |
| | KLNDLCFTNVYADSFVIRGDEVRQIAPGQTGKIADYNYKLPDDFTGCVIAWNSNNLDSKVGGN |
| | YNYLYRLFRKSNLKPFERDISTEIYQAGSTPCNGVEGFNCYFPLQSYGFQPTNGVGYQPYRVVV |
| | LSFELLHAPATVCGPKKSTNLVKNKCVNFNFNGLTGTGVLTESNKKFLPFQQFGRDIADTTDA |
| | VRDPQTLEILDITPCSFGGVSVITPGTNTSNQVAVLYQDVNCTEVPVAIHADQLTPTWRVYSTG |
| | SNVFQTRAGCLIGAEHVNNSYECDIPIGAGICASYQTQTNSPASVASQSIIAYTMSLGAENSVAY |
| | SNNSIAIPTNFTISVTTEILPVSMTKTSVDCTMYICGDSTECSNLLLQYGSFCTQLNRALTGIAVE |
| | QDKNTQEVFAQVKQIYKTPPIKDFGGFNFSQILPDPSKPSKRSFIEDLLFNKVTLADAGFIKQYG |
| | DCLGDIAARDLICAQKFNGLTVLPPLLTDEMIAQYTSALLAGTITSGWTFGAGAALQIPFAMQM |
| | AYRFNGIGVTQNVLYENQKLIANQFNSAIGKIQDSLSSTASALGKLQDVVNQNAQALNTLVKQ |
| | LSSNFGAISSVLNDILSRLDKVEAEVQIDRLITGRLQSLQTYVTQQLIRAAEIRASANLAATKMSE |
| | CVLGQSKRVDFCGKGYHLMSFPQSAPHGVVFLHVTYVPAQEKNFTTAPAICHDGKAHFPREG |
| | VFVSNGTHWFVTQRNFYEPQIITTDNTFVSGNCDVVIGIVNNTVYDPLQPELDSFKEELDKYFK |
| | NHTSPDVDLGDISGINASVVNIQKEIDRLNEVAKNLNESLIDLQELGKYEQYIKWPSGRLVPRGS |
| | PGSGYIPEAPRDGQAYVRKDGEWVLLSTFLGHHHHHH** |
| SARS-CoV-2 Spike | RVQPTESIVRFPNITNLCPFGEVFNATRFASVYAWNRKRISNCVADYSVLYNSASFSTFKCYGVS |
| protein receptor | PTKLNDLCFTNVYADSFVIRGDEVRQIAPGQTGKIADYNYKLPDDFTGCVIAWNSNNLDSKVG |
| binding domain and | GNYNYLYRLFRKSNLKPFERDISTEIYQAGSTPCNGVEGFNCYFPLQSYGFQPTNGVGYQPYRV |
| hexahistidine tag | VVLSFELLHAPATVCGPKKSTNLVKNKCVNFHHHHHH |
| Amino acid sequence | |
| (SEQ ID NO: 10) | |

8.2 Plasmid Sequences
SEQ ID NO: 7

| | |
|---|---|
| LOCUS | pCAGGs_seq_with_8529 bp ds-DNA linear 15-MAR.-2020 |
| DEFINITION | . |
| ACCESSION | |
| VERSION | |
| SOURCE | . |
| ORGANISM | . |
| COMMENT | |
| COMMENT | ApEinfo: methylated: 1 |
| FEATURES | Location/Qualifiers |
| misc_feature | 802 . . . 1533 |
| | /locus_tag = “B-actin promoter” |
| | /label = “B-actin promoter” |
| | /ApEinfo_label = “B-actin promoter” |
| | /ApEinfo_fwdcolor = “#0446ff” |
| | /ApEinfo_revcolor = “#0446ff” |
| | /ApEinfo_graphicformat = “arrow_data {{0 1 2 0 0 −1} {} 0} |
| | width 5 offset 0” |
| misc_feature | 84 . . . 371 |
| | /locus_tag = “CAG enhancer” |
| | /label = “CAG enhancer” |
| | /ApEinfo_label = “CAG enhancer” |
| | /ApEinfo_fwdcolor = “#fb0eff” |
| | /ApEinfo_revcolor = “#fb0eff” |
| | /ApEinfo_graphicformat = “arrow_data {{0 1 2 0 0 −1} {} 0} |
| | width 5 offset 0” |
| misc_feature | 7538 . . . 8398 |
| | /locus_tag = “AMP” |
| | /label = “AMP” |
| | /ApEinfo_label = “AMP” |
| | /ApEinfo_fwdcolor = “#ffef00” |
| | /ApEinfo_revcolor = “#00ff00” |
| | /ApEinfo_graphicformat = “arrow_data {{0 1 2 0 0 -1} {} 0} |
| | width 5 offset 0” |
| misc_feature | 1719 . . . 1725 |
| | /locus_tag = “EcorI” |
| | /label = “EcorI” |
| | /ApEinfo_label = “EcorI” |
| | /ApEinfo_fwdcolor = “#ff051e” |
| | /ApEinfo_revcolor = “#ff051e″ |
| | /ApEinfo_graphicformat = “arrow_data {{0 1 2 0 0 −1} {} 0} |
| | width 5 offset 0” |
| misc_feature | 5537 . . . 5542 |
| | /locus_tag = “XhoI” |
| | /label = “XhoI” |
| | /ApEinfo_label = “XhoI” |
| | /ApEinfo_fwdcolor = “#00b301” |
| | /ApEinfo_revcolor = “#d92725” |
| | /ApEinfo_graphicformat = “arrow_data {{0 1 2 0 0 −1} {} 0} |
| | width 5 offset 0” |
| misc_feature | 5505 . . . 5536 |
| | /locus_tag = “Gene insertion site” |
| | /label = “Gene insertion site” |
| | /ApEinfo_label = “Gene insertion site” |
| | /ApEinfo_fwdcolor = “#00ffff” |
| | /ApEinfo_revcolor = “#00ff04” |
| | /ApEinfo_graphicformat = “arrow_data {{0 1 2 0 0 −1} {} 0} |
| | width 5 offset 0” |
| misc_feature | 5500 . . . 5504 |
| | /locus_tag = “EcorI (1) “ |
| | /label = “EcorI (1) “ |
| | /ApEinfo_label = “EcorI” |
| | /ApEinfo_fwdcolor = “#ff051e” |
| | /ApEinfo_revcolor = “#ff051e” |
| | /ApEinfo_graphicformat = “arrow_data {{0 1 2 0 0 −1} {} 0} |
| | width 5 offset 0” |

ORIGIN

| | |
|---|---|
| 1 | GTCGACATTG ATTATTGACT AGTTATTAAT AGTAATCAAT TACGGGGTCA TTAGTTCATA |
| 61 | GCCCATATAT GGAGTTCCGC GTTACATAAC TTACGGTAAA TGGCCCGCCT GGCTGACCGC |
| 121 | CCAACGACCC CCGCCCATTG ACGTCAATAA TGACGTATGT TCCCATAGTA ACGCCAATAG |
| 181 | GGACTTTCCA TTGACGTCAA TGGGTGGACT ATTTACGGTA AACTGCCCAC TTGGCAGTAC |
| 241 | ATCAAGTGTA TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG TACATCTACG |
| 361 | TATTAGTCAT CGCTATTACC ATGGGTCGAG GTGAGCCCCA CGTTCTGCTT CACTCTCCCC |
| 421 | ATCTCCCCCC CCTCCCCACC CCCAATTTTG TATTTATTTA TTTTTTAATT ATTTTGTGCA |
| 481 | GCGATGGGGG CGGGGGGGGG GGGGGCGCGC GCCAGGCGGG GCGGGGGGGG GCGAGGGGCG |
| 541 | GGGGGGGGCG AGGCGGAGAG GTGCGGCGGC AGCCAATCAG AGCGGCGCGC TCCGAAAGTT |
| 601 | TCCTTTTATG GCGAGGCGGC GGCGGCGGCG GCCCTATAAA AAGCGAAGCG CGCGGCGGGC |
| 661 | GGGAGTCGCT GCGTTGCCTT CGCCCCGTGC CCCGCTCCGC GCCGCCTCGC GCCGCCCGCC |
| 721 | CCGGCTCTGA CTGACCGCGT TACTCCCACA GGTGAGCGGG CGGGACGGCC CTTCTCCTCC |
| 781 | GGGCTGTAAT TAGCGCTTGG TTTAATGACG GCTCGTTTCT TTTCTGTGGC TGCGTGAAAG |
| 841 | CCTTAAAGGG CTCCGGGAGG GCCCTTTGTG CGGGGGGGAG CGGCTCGGGG GGTGCGTGCG |
| 901 | TGTGTGTGTG CGTGGGGAGC GCCGCGTGCG GCCCGCGCTG CCCGGCGGCT GTGAGCGCTG |
| 961 | CGGGCGCGGC GCGGGGCTTT GTGCGCTCCG CGTGTGCGCG AGGGGAGCGC GGCCGGGGGC |
| 1021 | GGTGCCCCGC GGTGCGGGGG GGCTGCGAGG GGAACAAAGG CTGCGTGCGG GGTGTGTGCG |
| 1081 | TGGGGGGGTG AGCAGGGGGT GTGGGCGCGG CGGTCGGGCT GTAACCCCCC CCTGCACCCC |
| 1141 | CCTCCCCGAG TTGCTGAGCA CGGCCCGGCT TCGGGTGCGG GGCTCCGTGC GGGGCGTGGC |
| 1201 | GCGGGGCTCG CCGTGCCGGG CGGGGGGTGG CGGCAGGTGG GGGTGCCGGG CGGGGGGGGG |
| 1261 | CCGCCTCGGG CCGGGGAGGG CTCGGGGGAG GGGCGCGGCG GCCCCGGAGC GCCGGCGGCT |
| 1321 | GTCGAGGCGC GGCGAGCCGC AGCCATTGCC TTTTATGGTA ATCGTGCGAG AGGGCGCAGG |
| 1381 | GACTTCCTTT GTCCCAAATC TGGCGGAGCC GAAATCTGGG AGGCGCCGCC GCACCCCCTC |
| 1441 | TAGCGGGCGC GGGCGAAGCG GTGCGGCGCC GGCAGGAAGG AAATGGGCGG GGAGGGCCTT |
| 1501 | CGTGCGTCGC CGCGCCGCCG TCCCCTTCTC CATCTCCAGC CTCGGGGCTG CCGCAGGGGG |
| 1561 | ACGGCTGCCT TCGGGGGGGA CGGGGCAGGG CGGGGTTCGG CTTCTGGCGT GTGACCGGCG |
| 1621 | GCTCTAGAGC CTCTGCTAAC CATGTTCATG CCTTCTTCTT TTTCCTACAG CTCCTGGGCA |
| 1681 | ACGTGCTGGT TGTTGTGCTG TCTCATCATT TTGGCAAAGG CCACCATGTT CGTGTTTCTG |
| 1741 | GTGCTGCTGC CTCTGGTGTC CAGCCAGTGT GTGAACCTGA CCACAAGAAC CCAGCTGCCT |
| 1801 | CCAGCCTACA CCAACAGCTT TACCAGAGGC GTGTACTACC CCGACAAGGT GTTCAGATCC |
| 1861 | AGCGTGCTGC ACTCTACCCA GGACCTGTTC CTGCCTTTCT TCAGCAACGT GACCTGGTTC |
| 1921 | CACGCCATCC ACGTGTCCGG CACCAATGGC ACCAAGAGAT TCGACAACCC CGTGCTGCCC |
| 1981 | TTCAACGACG GGGTGTACTT TGCCAGCACC GAGAAGTCCA ACATCATCAG AGGCTGGATC |
| 2041 | TTCGGCACCA CACTGGACAG CAAGACCCAG AGCCTGCTGA TCGTGAACAA CGCCACCAAC |
| 2101 | GTGGTCATCA AAGTGTGCGA GTTCCAGTTC TGCAACGACC CCTTCCTGGG CGTCTACTAT |
| 2161 | CACAAGAACA ACAAGAGCTG GATGGAAAGC GAGTTCCGGG TGTACAGCAG CGCCAACAAC |
| 2221 | TGCACCTTCG AGTACGTGTC CCAGCCTTTC CTGATGGACC TGGAAGGCAA GCAGGGCAAC |
| 2281 | TTCAAGAACC TGCGCGAGTT CGTGTTCAAG AACATCGACG GCTACTTCAA GATCTACAGC |
| 2341 | AAGCACACCC CTATCAACCT CGTGCGGGAT CTGCCTCAGG GCTTCTCTGC TCTGGAACCC |
| 2401 | CTGGTGGATC TGCCCATCGG CATCAACATC ACCCGGTTTC AGACACTGCT GGCCCTGCAC |
| 2461 | AGAAGCTACC TGACACCTGG CGATAGCAGC AGCGGATGGA CAGCTGGTGC CGCCGCTTAC |
| 2521 | TATGTGGGCT ACCTGCAGCC TAGAACCTTT CTGCTGAAGT ACAACGAGAA CGGCACCATC |
| 2581 | ACCGACGCCG TGGATTGTGC TCTGGATCCT CTGAGCGAGA CAAAGTGCAC CCTGAAGTCC |
| 2641 | TTCACCGTGG AAAAGGGCAT CTACCAGACC AGCAACTTCC GGGTGCAGCC CACCGAATCC |
| 2701 | ATCGTGCGGT TCCCCAATAT CACCAATCTG TGCCCCTTCG GCGAGGTGTT CAATGCCACC |
| 2761 | AGATTCGCCT CTGTGTACGC CTGGAACCGG AAGCGGATCA GCAATTGCGT GGCCGACTAC |
| 2821 | TCCGTGCTGT ACAACTCCGC CAGCTTCAGC ACCTTCAAGT GCTACGGCGT GTCCCCTACC |
| 2881 | AAGCTGAACG ACCTGTGCTT CACAAACGTG TACGCCGACA GCTTCGTGAT CCGGGGAGAT |
| 2941 | GAAGTGCGGC AGATTGCCCC TGGACAGACA GGCAAGATCG CCGACTACAA CTACAAGCTG |
| 3001 | CCCGACGACT TCACCGGCTG TGTGATTGCC TGGAACAGCA ACAACCTGGA CTCCAAAGTC |
| 3061 | GGCGGCAACT ACAATTACCT GTACCGGCTG TTCCGGAAGT CCAATCTGAA GCCCTTCGAG |
| 3121 | CGGGACATCT CCACCGAGAT CTATCAGGCC GGCAGCACCC CTTGTAACGG CGTGGAAGGC |
| 3181 | TTCAACTGCT ACTTCCCACT GCAGTCCTAC GGCTTTCAGC CCACAAATGG CGTGGGCTAT |
| 3241 | CAGCCCTACA GAGTGGTGGT GCTGAGCTTC GAACTGCTGC ATGCCCCTGC CACAGTGTGC |
| 3301 | GGCCCTAAGA AAAGCACCAA TCTCGTGAAG AACAAATGCG TGAACTTCAA CTTCAACGGC |
| 3361 | CTGACCGGCA CCGGCGTGCT GACAGAGAGC AACAAGAAGT TCCTGCCATT CCAGCAGTTT |
| 3421 | GGCCGGGATA TCGCCGATAC CACAGACGCC GTTAGAGATC CCCAGACACT GGAAATCCTG |
| 3481 | GACATCACCC CTTGCAGCTT CGGCGGAGTG TCTGTGATCA CCCCTGGCAC CAACACCAGC |
| 3541 | AATCAGGTGG CAGTGCTGTA CCAGGACGTG AACTGTACCG AAGTGCCCGT GGCCATTCAC |
| 3601 | GCCGATCAGC TGACACCTAC ATGGGGGGTG TACTCCACCG GCAGCAATGT GTTTCAGACC |
| 3661 | AGAGCCGGCT GTCTGATCGG AGCCGAGCAC GTGAACAATA GCTACGAGTG CGACATCCCC |
| 3721 | ATCGGCGCTG GCATCTGTGC CAGCTACCAG ACACAGACAA ACAGCCCCGC CTCTGTGGCC |
| 3781 | AGCCAGAGCA TCATTGCCTA CACAATGTCT CTGGGCGCCG AGAACAGCGT GGCCTACTCC |
| 3841 | AACAACTCTA TCGCTATCCC CACCAACTTC ACCATCAGCG TGACCACAGA GATCCTGCCT |
| 3901 | GTGTCCATGA CCAAGACCAG CGTGGACTGC ACCATGTACA TCTGCGGCGA TTCCACCGAG |
| 3961 | TGCTCCAACC TGCTGCTGCA GTACGGCAGC TTCTGCACCC AGCTGAATAG AGCCCTGACA |
| 4021 | GGGATCGCCG TGGAACAGGA CAAGAACACC CAAGAGGTGT TCGCCCAAGT GAAGCAGATC |
| 4081 | TACAAGACCC CTCCTATCAA GGACTTCGGC GGCTTCAATT TCAGCCAGAT TCTGCCCGAT |
| 4141 | CCTAGCAAGC CCAGCAAGCG GAGCTTCATC GAGGACCTGC TGTTCAACAA AGTGACACTG |
| 4201 | GCCGACGCCG GCTTCATCAA GCAGTATGGC GATTGTCTGG GCGACATTGC CGCCAGGGAT |
| 4261 | CTGATTTGCG CCCAGAAGTT TAACGGACTG ACAGTGCTGC CTCCTCTGCT GACCGATGAG |
| 4321 | ATGATCGCCC AGTACACATC TGCCCTGCTG GCCGGCACAA TCACAAGCGG CTGGACATTT |
| 4381 | GGAGCTGGCG CCGCTCTGCA GATCCCCTTT GCTATGCAGA TGGCCTACCG GTTCAACGGC |
| 4441 | ATCGGAGTGA CCCAGAATGT GCTGTACGAG AACCAGAAGC TGATCGCCAA CCAGTTCAAC |
| 4501 | AGCGCCATCG GCAAGATCCA GGACAGCCTG AGCAGCACAG CAAGCGCCCT GGGAAAGCTG |
| 4561 | CAGGACGTGG TCAACCAGAA TGCCCAGGCA CTGAACACCC TGGTCAAGCA GCTGTCCTCC |
| 4621 | AACTTCGGCG CCATCAGCTC TGTGCTGAAC GATATCCTGA GCAGACTGGA CcctcctGAA |
| 4681 | GCCGAGGTGC AGATCGACAG ACTGATCACC GGAAGGCTGC AGTCCCTGCA GACCTACGTT |
| 4741 | ACCCAGCAGC TGATCAGAGC CGCCGAGATT AGAGCCTCTG CCAATCTGGC CGCCACCAAG |
| 4801 | ATGTCTGAGT GTGTGCTGGG CCAGAGCAAG AGAGTGGACT TTTGCGGCAA GGGCTACCAC |
| 4861 | CTGATGAGCT TCCCTCAGTC TGCCCCTCAC GGCGTGGTGT TTCTGCACGT GACATACGTG |
| 4921 | CCCGCTCAAG AGAAGAATTT CACCACCGCT CCAGCCATCT GCCACGACGG CAAAGCCCAC |
| 4981 | TTTCCTAGAG AAGGCGTGTT CGTGTCCAAC GGCACCCATT GGTTCGTGAC CCAGCGGAAC |
| 5041 | TTCTACGAGC CCCAGATCAT CACCACCGAC AACACCTTCG TGTCTGGCAA CTGCGACGTC |
| 5101 | GTGATCGGCA TTGTGAACAA TACCGTGTAC GACCCTCTGC AGCCCGAGCT GGACAGCTTC |
| 5161 | AAAGAGGAAC TGGATAAGTA CTTTAAGAAC CACACAAGCC CCGACGTGGA CCTGGGCGAT |
| 5221 | ATCAGCGGAA TCAATGCCAG CGTCGTGAAC ATCCAGAAAG AGATCGACCG GCTGAACGAG |
| 5281 | GTGGCCAAGA ATCTGAACGA GAGCCTGATC GACCTGCAAG AACTGGGGAA GTACGAGCAG |
| 5341 | TACATCAAGT GGCCCAGCGG CCGCTTGGTC CCACGTGGCT CACCCGGATC TGGATACATC |
| 5401 | CCGGAGGCCC CTAGGGACGG TCAAGCTTAC GTGAGAAAGG ACGGCGAATG GGTTCTGCTG |
| 5461 | TCGACCTTCT TGGGACATCA TCATCATCAT CACTAATGAA ATTCGAGCTC GCGGCCGCAT |
| 5521 | CGATCTTAAG TCGCGACTCG AGCTAGCAGA TCTTTTTCCC TCTGCCAAAA ATTATGGGGA |
| 5581 | CATCATGAAG CCCCTTGAGC ATCTGACTTC TGGCTAATAA AGGAAATTTA TTTTCATTGC |
| 5641 | AATAGTGTGT TGGAATTTTT TGTGTCTCTC ACTCGGAAGG ACATATGGGA GGGCAAATCA |
| 5701 | TTTAAAACAT CAGAATGAGT ATTTGGTTTA GAGTTTGGCA ACATATGCCC ATATGCTGGC |
| 5761 | TGCCATGAAC AAAGGTTGGC TATAAAGAGG TCATCAGTAT ATGAAACAGC CCCCTGCTGT |
| 5821 | CCATTCCTTA TTCCATAGAA AAGCCTTGAC TTGAGGTTAG ATTTTTTTTA TATTTTGTTT |
| 5881 | TGTGTTATTT TTTTCTTTAA CATCCCTAAA ATTTTCCTTA CATGTTTTAC TAGCCAGATT |
| 5941 | TTTCCTCCTC TCCTGACTAC TCCCAGTCAT AGCTGTCCCT CTTCTCTTAT GAAGATCCCT |
| 6001 | CGACCTGCAG CCCAAGCTTG GCGTAATCAT GGTCATAGCT GTTTCCTGTG TGAAATTGTT |
| 6061 | ATCCGCTCAC AATTCCACAC AACATACGAG CCGGAAGCAT AAAGTGTAAA GCCTGGGGTG |
| 6121 | CCTAATGAGT GAGCTAACTC ACATTAATTG CGTTGCGCTC ACTGCCCGCT TTCCAGTCGG |
| 6181 | GAAACCTGTC GTGCCAGCGG ATCCGCATCT CAATTAGTCA GCAACCATAG TCCCGCCCCT |
| 6241 | AACTCCGCCC ATCCCGCCCC TAACTCCGCC CAGTTCCGCC CATTCTCCGC CCCATGGCTG |
| 6301 | ACTAATTTTT TTTATTTATG CAGAGGCCGA GGCCGCCTCG GCCTCTGAGC TATTCCAGAA |
| 6361 | GTAGTGAGGA GGCTTTTTTG GAGGCCTAGG CTTTTGCAAA AAGCTAACTT GTTTATTGCA |
| 6421 | GCTTATAATG GTTACAAATA AAGCAATAGC ATCACAAATT TCACAAATAA AGCATTTTTT |
| 6481 | TCACTGCATT CTAGTTGTGG TTTGTCCAAA CTCATCAATG TATCTTATCA TGTCTGGATC |
| 6541 | CGCTGCATTA ATGAATCGGC CAACGCGCGG GGAGAGGCGG TTTGCGTATT GGGCGCTCTT |
| 6601 | CCGCTTCCTC GCTCACTGAC TCGCTGCGCT CGGTCGTTCG GCTGCGGCGA GCGGTATCAG |
| 6661 | CTCACTCAAA GGCGGTAATA CGGTTATCCA CAGAATCAGG GGATAACGCA GGAAAGAACA |
| 6721 | TGTGAGCAAA AGGCCAGCAA AAGGCCAGGA ACCGTAAAAA GGCCGCGTTG CTGGCGTTTT |
| 6781 | TCCATAGGCT CCGCCCCCCT GACGAGCATC ACAAAAATCG ACGCTCAAGT CAGAGGTGGC |
| 6841 | GAAACCCGAC AGGACTATAA AGATACCAGG CGTTTCCCCC TGGAAGCTCC CTCGTGCGCT |
| 6901 | CTCCTGTTCC GACCCTGCCG CTTACCGGAT ACCTGTCCGC CTTTCTCCCT TCGGGAAGCG |
| 6961 | TGGCGCTTTC TCAATGCTCA CGCTGTAGGT ATCTCAGTTC GGTGTAGGTC GTTCGCTCCA |
| 7021 | AGCTGGGCTG TGTGCACGAA CCCCCCGTTC AGCCCGACCG CTGCGCCTTA TCCGGTAACT |
| 7081 | ATCGTCTTGA GTCCAACCCG GTAAGACACG ACTTATCGCC ACTGGCAGCA GCCACTGGTA |
| 7141 | ACAGGATTAG CAGAGCGAGG TATGTAGGCG GTGCTACAGA GTTCTTGAAG TGGTGGCCTA |
| 7201 | ACTACGGCTA CACTAGAAGG ACAGTATTTG GTATCTGCGC TCTGCTGAAG CCAGTTACCT |
| 7261 | TCGGAAAAAG AGTTGGTAGC TCTTGATCCG GCAAACAAAC CACCGCTGGT AGCGGTGGTT |
| 7321 | TTTTTGTTTG CAAGCAGCAG ATTACGCGCA GAAAAAAAGG ATCTCAAGAA GATCCTTTGA |
| 7381 | TCTTTTCTAC GGGGTCTGAC GCTCAGTGGA ACGAAAACTC ACGTTAAGGG ATTTTGGTCA |
| 7441 | TGAGATTATC AAAAAGGATC TTCACCTAGA TCCTTTTAAA TTAAAAATGA AGTTTTAAAT |
| 7501 | CAATCTAAAG TATATATGAG TAAACTTGGT CTGACAGTTA CCAATGCTTA ATCAGTGAGG |
| 7561 | CACCTATCTC AGCGATCTGT CTATTTCGTT CATCCATAGT TGCCTGACTC CCCGTCGTGT |
| 7621 | AGATAACTAC GATACGGGAG GGCTTACCAT CTGGCCCCAG TGCTGCAATG ATACCGCGAG |
| 7681 | ACCCACGCTC ACCGGCTCCA GATTTATCAG CAATAAACCA GCCAGCCGGA AGGGCCGAGC |
| 7741 | GCAGAAGTGG TCCTGCAACT TTATCCGCCT CCATCCAGTC TATTAATTGT TGCCGGGAAG |
| 7801 | CTAGAGTAAG TAGTTCGCCA GTTAATAGTT TGCGCAACGT TGTTGCCATT GCTACAGGCA |
| 7861 | TCGTGGTGTC ACGCTCGTCG TTTGGTATGG CTTCATTCAG CTCCGGTTCC CAACGATCAA |
| 7921 | GGCGAGTTAC ATGATCCCCC ATGTTGTGCA AAAAAGCGGT TAGCTCCTTC GGTCCTCCGA |
| 7981 | TCGTTGTCAG AAGTAAGTTG GCCGCAGTGT TATCACTCAT GGTTATGGCA GCACTGCATA |
| 8041 | ATTCTCTTAC TGTCATGCCA TCCGTAAGAT GCTTTTCTGT GACTGGTGAG TACTCAACCA |
| 8101 | AGTCATTCTG AGAATAGTGT ATGCGGCGAC CGAGTTGCTC TTGCCCGGCG TCAATACGGG |
| 8161 | ATAATACCGC GCCACATAGC AGAACTTTAA AAGTGCTCAT CATTGGAAAA CGTTCTTCGG |
| 8221 | GGCGAAAACT CTCAAGGATC TTACCGCTGT TGAGATCCAG TTCGATGTAA CCCACTCGTG |
| 8281 | CACCCAACTG ATCTTCAGCA TCTTTTACTT TCACCAGCGT TTCTGGGTGA GCAAAAACAG |
| 8341 | GAAGGCAAAA TGCCGCAAAA AAGGGAATAA GGGCGACACG GAAATGTTGA ATACTCATAC |
| 8401 | TCTTCCTTTT TCAATATTAT TGAAGCATTT ATCAGGGTTA TTGTCTCATG AGCGGATACA |
| 8461 | TATTTGAATG TATTTAGAAA AATAAACAAA TAGGGGTTCC GCGCACATTT CCCCGAAAAG |
| 8521 | TGCCACCTG |
| // | |

SEQ ID NO: 8

| | |
|---|---|
| LOCUS | pCAGGs_seq_with_ 5490 bp ds-DNA linear 16-FEB.-2020 |
| DEFINITION | . |
| ACCESSION | |
| VERSION | |
| SOURCE | . |
| ORGANISM | . |
| COMMENT | |
| COMMENT | ApEinfo: methylated: 1 |
| FEATURES | Location/Qualifiers |
| misc_feature | 802 . . . 1533 |
| | /locus_tag = “B-actin promoter” |
| | /label = “B-actin promoter” |
| | /ApEinfo_label = “B-actin promoter” |
| | /ApEinfo_fwdcolor = “#0446ff” |
| | /ApEinfo_revcolor = “#0446ff” |
| | /ApEinfo_graphicformat = “arrow_data {{0 1 2 0 0 −1} {} 0} |
| | width 5 offset 0” |
| misc_feature | 1768 . . . 2436 |
| | /locus_tag = “frame” |
| | /label = “frame” |
| | /ApEinfo_label = “frame” |
| | /ApEinfo_fwdcolor = “cyan” |
| | /ApEinfo_revcolor = “green” |
| | /ApEinfo_graphicformat = “arrow_data {{0 1 2 0 0 −1} {} 0} |
| | width 5 offset 0” |
| misc_feature | 84 . . . 371 |
| | /locus_tag = “CAG enhancer” |
| | /label = “CAG enhancer” |
| | /ApEinfo_label = “CAG enhancer” |
| | /ApEinfo_fwdcolor = “#fb0eff” |
| | /ApEinfo_revcolor = “#fb0eff” |
| | /ApEinfo_graphicformat = “arrow_data {{0 1 2 0 0 −1} {} 0} |
| | width 5 offset 0” |
| misc_feature | 4499 . . . 5359 |
| | /locus_tag = “AMP” |
| | /label = “AMP” |
| | /ApEinfo_label = “AMP” |
| | /ApEinfo_fwdcolor = “#ffef00″ |
| | /ApEinfo_revcolor = “#00ff00” |
| | /ApEinfo_graphicformat = “arrow_data {{0 1 2 0 0 −1} {} 0} |
| | width 5 offset 0” |
| misc_feature | 1768 . . . 1770 |
| | /locus_tag = “start RBD″ |
| | /label = “start RBD” |
| | /ApEinfo_label = “start RBD” |
| | /ApEinfo_fwdcolor = “#8080ff” |
| | /ApEinfo_revcolor = “green″ |
| | /ApEinfo_graphicformat = “arrow_data {{0 1 2 0 0 −1} {} 0} |
| | width 5 offset 0” |
| misc_feature | 1719 . . . 1725 |
| | /locus_tag = “EcorI″ |
| | /label = “EcorI” |
| | /ApEinfo_label = “EcorI” |
| | /ApEinfo_fwdcolor = “#ff051e” |
| | /ApEinfo_revcolor = “#ff051e” |
| | /ApEinfo_graphicformat = “arrow_data {{0 1 2 0 0 −1} {} 0} |
| | width 5 offset 0” |
| misc_feature | 2434 . . . 2436 |
| | /locus_tag = “RBD stop” |
| | /label = “RBD stop” |
| | /ApEinfo_label = “RBD stop” |
| | /ApEinfo_fwdcolor = “#8080ff″ |
| | /ApEinfo_revcolor = “green” |
| | /ApEinfo_graphicformat = “arrow_data {{0 1 2 0 0 −1} {} 0} |
| | width 5 offset 0” |
| misc_feature | 2498 . . . 2503 |
| | /locus_tag = “XhoI” |
| | /label = “XhoI” |
| | /ApEinfo_label = “XhoI” |
| | /ApEinfo_fwdcolor = “#00b301″ |
| | /ApEinfo_revcolor = “#d92725” |
| | /ApEinfo_graphicformat = “arrow_data {{0 1 2 0 0 −1} {} 0} |
| | width 5 offset 0” |
| misc_feature | 2466 . . . 2497 |
| | /locus_tag = “Gene insertion site” |
| | /label = “Gene insertion site” |
| | /ApEinfo_label = “Gene insertion site” |
| | /ApEinfo_fwdcolor = “#00ffff” |
| | /ApEinfo_revcolor = “#00ff04” |
| | /ApEinfo_graphicformat = “arrow_data {{0 1 2 0 0 −1} {} 0} |
| | width 5 offset 0” |
| misc_feature | 2461 . . . 2465 |
| | /locus_tag = “EcorI (1) “ |
| | /label = “EcorI (1) “ |
| | /ApEinfo_label = “EcorI” |
| | /ApEinfo_fwdcolor = “#ff051e” |
| | /ApEinfo_revcolor = “#ff051e” |
| | /ApEinfo_graphicformat = “arrow_data {{0 1 2 0 0 −1} {} 0} |
| | width 5 offset 0” |
| misc_feature | 1726 . . . 2460 |
| | /locus_tag = “RBD with his tag” |
| | /label = “RBD with his tag” |
| | /ApEinfo_label = “RBD with his tag” |
| | /ApEinfo_fwdcolor = “#ff8000” |
| | /ApEinfo_revcolor = “green” |
| | /ApEinfo_graphicformat = “arrow_data {{0 1 2 0 0 −1} {} 0} |
| | width 5 offset 0” |

ORIGIN

| | |
|---|---|
| 1 | GTCGACATTG ATTATTGACT AGTTATTAAT AGTAATCAAT TACGGGGTCA TTAGTTCATA |
| 61 | GCCCATATAT GGAGTTCCGC GTTACATAAC TTACGGTAAA TGGCCCGCCT GGCTGACCGC |
| 121 | CCAACGACCC CCGCCCATTG ACGTCAATAA TGACGTATGT TCCCATAGTA ACGCCAATAG |
| 181 | GGACTTTCCA TTGACGTCAA TGGGTGGACT ATTTACGGTA AACTGCCCAC TTGGCAGTAC |
| 241 | ATCAAGTGTA TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG TACATCTACG |
| 361 | TATTAGTCAT CGCTATTACC ATGGGTCGAG GTGAGCCCCA CGTTCTGCTT CACTCTCCCC |
| 421 | ATCTCCCCCC CCTCCCCACC CCCAATTTTG TATTTATTTA TTTTTTAATT ATTTTGTGCA |
| 481 | GCGATGGGGG CGGGGGGGGG GGGGGCGCGC GCCAGGCGGG GCGGGGGGGG GCGAGGGGCG |
| 541 | GGGGGGGGCG AGGCGGAGAG GTGCGGCGGC AGCCAATCAG AGCGGCGCGC TCCGAAAGTT |
| 601 | TCCTTTTATG GCGAGGCGGC GGCGGCGGCG GCCCTATAAA AAGCGAAGCG CGCGGCGGGC |
| 661 | GGGAGTCGCT GCGTTGCCTT CGCCCCGTGC CCCGCTCCGC GCCGCCTCGC GCCGCCCGCC |
| 721 | CCGGCTCTGA CTGACCGCGT TACTCCCACA GGTGAGCGGG CGGGACGGCC CTTCTCCTCC |
| 781 | GGGCTGTAAT TAGCGCTTGG TTTAATGACG GCTCGTTTCT TTTCTGTGGC TGCGTGAAAG |
| 841 | CCTTAAAGGG CTCCGGGAGG GCCCTTTGTG CGGGGGGGAG CGGCTCGGGG GGTGCGTGCG |
| 901 | TGTGTGTGTG CGTGGGGAGC GCCGCGTGCG GCCCGCGCTG CCCGGCGGCT GTGAGCGCTG |
| 961 | CGGGCGCGGC GCGGGGCTTT GTGCGCTCCG CGTGTGCGCG AGGGGAGCGC GGCCGGGGGC |
| 1021 | GGTGCCCCGC GGTGCGGGGG GGCTGCGAGG GGAACAAAGG CTGCGTGCGG GGTGTGTGCG |
| 1081 | TGGGGGGGTG AGCAGGGGGT GTGGGCGCGG CGGTCGGGCT GTAACCCCCC CCTGCACCCC |
| 1141 | CCTCCCCGAG TTGCTGAGCA CGGCCCGGCT TCGGGTGCGG GGCTCCGTGC GGGGCGTGGC |
| 1201 | GCGGGGCTCG CCGTGCCGGG CGGGGGGTGG CGGCAGGTGG GGGTGCCGGG CGGGGCGGGG |
| 1261 | CCGCCTCGGG CCGGGGAGGG CTCGGGGGAG GGGCGCGGCG GCCCCGGAGC GCCGGCGGCT |
| 1321 | GTCGAGGCGC GGCGAGCCGC AGCCATTGCC TTTTATGGTA ATCGTGCGAG AGGGCGCAGG |
| 1381 | GACTTCCTTT GTCCCAAATC TGGCGGAGCC GAAATCTGGG AGGCGCCGCC GCACCCCCTC |
| 1441 | TAGCGGGCGC GGGCGAAGCG GTGCGGCGCC GGCAGGAAGG AAATGGGCGG GGAGGGCCTT |
| 1501 | CGTGCGTCGC CGCGCCGCCG TCCCCTTCTC CATCTCCAGC CTCGGGGCTG CCGCAGGGGG |
| 1561 | ACGGCTGCCT TCGGGGGGGA CGGGGCAGGG CGGGGTTCGG CTTCTGGCGT GTGACCGGCG |
| 1621 | GCTCTAGAGC CTCTGCTAAC CATGTTCATG CCTTCTTCTT TTTCCTACAG CTCCTGGGCA |
| 1681 | ACGTGCTGGT TGTTGTGCTG TCTCATCATT TTGGCAAAGG CCACCATGTT CGTGTTTCTG |
| 1741 | GTGCTGCTGC CTCTGGTGTC CAGCCAGCGG GTGCAGCCCA CCGAATCCAT CGTGCGGTTC |
| 1801 | CCCAATATCA CCAATCTGTG CCCCTTCGGC GAGGTGTTCA ATGCCACCAG ATTCGCCTCT |
| 1861 | GTGTACGCCT GGAACCGGAA GCGGATCAGC AATTGCGTGG CCGACTACTC CGTGCTGTAC |
| 1921 | AACTCCGCCA GCTTCAGCAC CTTCAAGTGC TACGGCGTGT CCCCTACCAA GCTGAACGAC |
| 1981 | CTGTGCTTCA CAAACGTGTA CGCCGACAGC TTCGTGATCC GGGGAGATGA AGTGCGGCAG |
| 2041 | ATTGCCCCTG GACAGACAGG CAAGATCGCC GACTACAACT ACAAGCTGCC CGACGACTTC |
| 2101 | ACCGGCTGTG TGATTGCCTG GAACAGCAAC AACCTGGACT CCAAAGTCGG CGGCAACTAC |
| 2161 | AATTACCTGT ACCGGCTGTT CCGGAAGTCC AATCTGAAGC CCTTCGAGCG GGACATCTCC |
| 2221 | ACCGAGATCT ATCAGGCCGG CAGCACCCCT TGTAACGGCG TGGAAGGCTT CAACTGCTAC |
| 2281 | TTCCCACTGC AGTCCTACGG CTTTCAGCCC ACAAATGGCG TGGGCTATCA GCCCTACAGA |
| 2341 | GTGGTGGTGC TGAGCTTCGA ACTGCTGCAT GCCCCTGCCA CAGTGTGCGG CCCTAAGAAA |
| 2401 | AGCACCAATC TCGTGAAGAA CAAATGCGTG AACTTCcacc atcaccatca ccatTGATAA |
| 2461 | AATTCGAGCT CGCGGCCGCA TCGATCTTAA GTCGCGACTC GAGCTAGCAG ATCTTTTTCC |
| 2521 | CTCTGCCAAA AATTATGGGG ACATCATGAA GCCCCTTGAG CATCTGACTT CTGGCTAATA |
| 2581 | AAGGAAATTT ATTTTCATTG CAATAGTGTG TTGGAATTTT TTGTGTCTCT CACTCGGAAG |
| 2641 | GACATATGGG AGGGCAAATC ATTTAAAACA TCAGAATGAG TATTTGGTTT AGAGTTTGGC |
| 2701 | AACATATGCC CATATGCTGG CTGCCATGAA CAAAGGTTGG CTATAAAGAG GTCATCAGTA |
| 2761 | TATGAAACAG CCCCCTGCTG TCCATTCCTT ATTCCATAGA AAAGCCTTGA CTTGAGGTTA |
| 2821 | GATTTTTTTT ATATTTTGTT TTGTGTTATT TTTTTCTTTA ACATCCCTAA AATTTTCCTT |
| 2881 | ACATGTTTTA CTAGCCAGAT TTTTCCTCCT CTCCTGACTA CTCCCAGTCA TAGCTGTCCC |
| 2941 | TCTTCTCTTA TGAAGATCCC TCGACCTGCA GCCCAAGCTT GGCGTAATCA TGGTCATAGC |
| 3001 | TGTTTCCTGT GTGAAATTGT TATCCGCTCA CAATTCCACA CAACATACGA GCCGGAAGCA |
| 3061 | TAAAGTGTAA AGCCTGGGGT GCCTAATGAG TGAGCTAACT CACATTAATT GCGTTGCGCT |
| 3121 | CACTGCCCGC TTTCCAGTCG GGAAACCTGT CGTGCCAGCG GATCCGCATC TCAATTAGTC |
| 3181 | AGCAACCATA GTCCCGCCCC TAACTCCGCC CATCCCGCCC CTAACTCCGC CCAGTTCCGC |
| 3241 | CCATTCTCCG CCCCATGGCT GACTAATTTT TTTTATTTAT GCAGAGGCCG AGGCCGCCTC |
| 3301 | GGCCTCTGAG CTATTCCAGA AGTAGTGAGG AGGCTTTTTT GGAGGCCTAG GCTTTTGCAA |
| 3361 | AAAGCTAACT TGTTTATTGC AGCTTATAAT GGTTACAAAT AAAGCAATAG CATCACAAAT |
| 3421 | TTCACAAATA AAGCATTTTT TTCACTGCAT TCTAGTTGTG GTTTGTCCAA ACTCATCAAT |
| 3481 | GTATCTTATC ATGTCTGGAT CCGCTGCATT AATGAATCGG CCAACGCGCG GGGAGAGGCG |
| 3541 | GTTTGCGTAT TGGGCGCTCT TCCGCTTCCT CGCTCACTGA CTCGCTGCGC TCGGTCGTTC |
| 3601 | GGCTGCGGCG AGCGGTATCA GCTCACTCAA AGGCGGTAAT ACGGTTATCC ACAGAATCAG |
| 3661 | GGGATAACGC AGGAAAGAAC ATGTGAGCAA AAGGCCAGCA AAAGGCCAGG AACCGTAAAA |
| 3721 | AGGCCGCGTT GCTGGCGTTT TTCCATAGGC TCCGCCCCCC TGACGAGCAT CACAAAAATC |
| 3781 | GACGCTCAAG TCAGAGGTGG CGAAACCCGA CAGGACTATA AAGATACCAG GCGTTTCCCC |
| 3841 | CTGGAAGCTC CCTCGTGCGC TCTCCTGTTC CGACCCTGCC GCTTACCGGA TACCTGTCCG |
| 3901 | CCTTTCTCCC TTCGGGAAGC GTGGCGCTTT CTCAATGCTC ACGCTGTAGG TATCTCAGTT |
| 3961 | CGGTGTAGGT CGTTCGCTCC AAGCTGGGCT GTGTGCACGA ACCCCCCGTT CAGCCCGACC |
| 4021 | GCTGCGCCTT ATCCGGTAAC TATCGTCTTG AGTCCAACCC GGTAAGACAC GACTTATCGC |
| 4081 | CACTGGCAGC AGCCACTGGT AACAGGATTA GCAGAGCGAG GTATGTAGGC GGTGCTACAG |
| 4141 | AGTTCTTGAA GTGGTGGCCT AACTACGGCT ACACTAGAAG GACAGTATTT GGTATCTGCG |
| 4201 | CTCTGCTGAA GCCAGTTACC TTCGGAAAAA GAGTTGGTAG CTCTTGATCC GGCAAACAAA |
| 4261 | CCACCGCTGG TAGCGGTGGT TTTTTTGTTT GCAAGCAGCA GATTACGCGC AGAAAAAAAG |
| 4321 | GATCTCAAGA AGATCCTTTG ATCTTTTCTA CGGGGTCTGA CGCTCAGTGG AACGAAAACT |
| 4381 | CACGTTAAGG GATTTTGGTC ATGAGATTAT CAAAAAGGAT CTTCACCTAG ATCCTTTTAA |
| 4441 | ATTAAAAATG AAGTTTTAAA TCAATCTAAA GTATATATGA GTAAACTTGG TCTGACAGTT |
| 4501 | ACCAATGCTT AATCAGTGAG GCACCTATCT CAGCGATCTG TCTATTTCGT TCATCCATAG |
| 4561 | TTGCCTGACT CCCCGTCGTG TAGATAACTA CGATACGGGA GGGCTTACCA TCTGGCCCCA |
| 4621 | GTGCTGCAAT GATACCGCGA GACCCACGCT CACCGGCTCC AGATTTATCA GCAATAAACC |
| 4681 | AGCCAGCCGG AAGGGCCGAG CGCAGAAGTG GTCCTGCAAC TTTATCCGCC TCCATCCAGT |
| 4741 | CTATTAATTG TTGCCGGGAA GCTAGAGTAA GTAGTTCGCC AGTTAATAGT TTGCGCAACG |
| 4801 | TTGTTGCCAT TGCTACAGGC ATCGTGGTGT CACGCTCGTC GTTTGGTATG GCTTCATTCA |
| 4861 | GCTCCGGTTC CCAACGATCA AGGCGAGTTA CATGATCCCC CATGTTGTGC AAAAAAGCGG |
| 4921 | TTAGCTCCTT CGGTCCTCCG ATCGTTGTCA GAAGTAAGTT GGCCGCAGTG TTATCACTCA |
| 4981 | TGGTTATGGC AGCACTGCAT AATTCTCTTA CTGTCATGCC ATCCGTAAGA TGCTTTTCTG |
| 5041 | TGACTGGTGA GTACTCAACC AAGTCATTCT GAGAATAGTG TATGCGGCGA CCGAGTTGCT |
| 5101 | CTTGCCCGGC GTCAATACGG GATAATACCG CGCCACATAG CAGAACTTTA AAAGTGCTCA |
| 5161 | TCATTGGAAA ACGTTCTTCG GGGCGAAAAC TCTCAAGGAT CTTACCGCTG TTGAGATCCA |
| 5221 | GTTCGATGTA ACCCACTCGT GCACCCAACT GATCTTCAGC ATCTTTTACT TTCACCAGCG |
| 5281 | TTTCTGGGTG AGCAAAAACA GGAAGGCAAA ATGCCGCAAA AAAGGGAATA AGGGCGACAC |
| 5341 | GGAAATGTTG AATACTCATA CTCTTCCTTT TTCAATATTA TTGAAGCATT TATCAGGGTT |
| 5401 | ATTGTCTCAT GAGCGGATAC ATATTTGAAT GTATTTAGAA AAATAAACAA ATAGGGGTTC |
| 5461 | CGCGCACATT TCCCCGAAAA GTGCCACCTG |
| // | |

SEQ ID NO: 9

| | |
|---|---|
| LOCUS | pCAGGs_seq_with_ 8529 bp ds-DNA linear 23-MAR.-2020 |
| DEFINITION | . |
| ACCESSION | |
| VERSION | |
| SOURCE | . |
| ORGANISM | . |
| COMMENT | |
| COMMENT | ApEinfo: methylated: 1 |
| FEATURES | Location/Qualifiers |
| misc_feature | 802 . . . 1533 |
| | /locus_tag = “B-actin promoter” |
| | /label = “B-actin promoter” |
| | /ApEinfo_label = “B-actin promoter” |
| | /ApEinfo_fwdcolor = “#0446ff” |
| | /ApEinfo_revcolor = “#0446ff” |
| | /ApEinfo_graphicformat = “arrow_data {{0 1 2 0 0 −1} {} 0} |
| | width 5 offset 0” |
| misc_feature | 84 . . . 371 |
| | /locus_tag = “CAG enhancer” |
| | /label = “CAG enhancer” |
| | /ApEinfo_label = “CAG enhancer” |
| | /ApEinfo_fwdcolor = “#fb0eff” |
| | /ApEinfo_revcolor = “#fb0eff” |
| | /ApEinfo_graphicformat = “arrow_data {{0 1 2 0 0 −1} {} 0} |
| | width 5 offset 0” |
| misc_feature | 7538 . . . 8398 |
| | /locus_tag = “AMP” |
| | /label = “AMP” |
| | /ApEinfo_label = “AMP” |
| | /ApEinfo_fwdcolor = “#ffef00” |
| | /ApEinfo_revcolor = “#00ff00” |
| | /ApEinfo_graphicformat = “arrow_data {{0 1 2 0 0 −1} {} 0} |
| | width 5 offset 0” |
| misc_feature | 1719. . 1725 |
| | /locus_tag = “EcorI” |
| | /label = “EcorI” |
| | /ApEinfo_label = “EcorI” |
| | /ApEinfo_fwdcolor = “#ff051e” |
| | /ApEinfo_revcolor = “#ff051e” |
| | /ApEinfo_graphicformat = “arrow_data {{0 1 2 0 0 −1} {} 0} |
| | width 5 offset 0” |
| misc_feature | 5537 . . . 5542 |
| | /locus_tag = “XhoI” |
| | /label = “XhoI” |
| | /ApEinfo_label = “XhoI” |
| | /ApEinfo_fwdcolor = “#00b301” |
| | /ApEinfo_revcolor = “#d92725” |
| | /ApEinfo_graphicformat = “arrow_data {{0 1 2 0 0 −1} {} 0} |
| | width 5 offset 0” |
| misc_feature | 5505 . . . 5536 |
| | /locus_tag = “Gene insertion site” |
| | /label = “Gene insertion site” |
| | /ApEinfo_label = “Gene insertion site” |
| | /ApEinfo_fwdcolor = “#00ffff” |
| | /ApEinfo_revcolor = “#00ff04” |
| | /ApEinfo_graphicformat = “arrow_data {{0 1 2 0 0 −1} {} 0} |
| | width 5 offset 0” |
| misc_feature | 5500 . . . 5504 |
| | /locus_tag = “EcorI (1) “ |
| | /label = “EcorI (1) “ |
| | /ApEinfo_label = “EcorI” |
| | /ApEinfo_fwdcolor = “#ff051e” |
| | /ApEinfo_revcolor = “#ff051e” |
| | /ApEinfo_graphicformat = “arrow_data {{0 1 2 0 0 −1} {} 0} |
| | width 5 offset 0” |
| misc_feature | 1726 . . . 5355 |
| | /locus_tag = “Spike” |
| | /label = “Spike” |
| | /ApEinfo_label = “Spike” |
| | /ApEinfo_fwdcolor = “#ff8000” |
| | /ApEinfo_revcolor = “green” |
| | /ApEinfo_graphicformat = “arrow_data {{ 0 1 2 0 0 −1} {} o} |
| | width 5 offset 0” |

ORIGIN

| | |
|---|---|
| 1 | GTCGACATTG ATTATTGACT AGTTATTAAT AGTAATCAAT TACGGGGTCA TTAGTTCATA |
| 61 | GCCCATATAT GGAGTTCCGC GTTACATAAC TTACGGTAAA TGGCCCGCCT GGCTGACCGC |
| 121 | CCAACGACCC CCGCCCATTG ACGTCAATAA TGACGTATGT TCCCATAGTA ACGCCAATAG |
| 181 | GGACTTTCCA TTGACGTCAA TGGGTGGACT ATTTACGGTA AACTGCCCAC TTGGCAGTAC |
| 241 | ATCAAGTGTA TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG TACATCTACG |
| 361 | TATTAGTCAT CGCTATTACC ATGGGTCGAG GTGAGCCCCA CGTTCTGCTT CACTCTCCCC |
| 421 | ATCTCCCCCC CCTCCCCACC CCCAATTTTG TATTTATTTA TTTTTTAATT ATTTTGTGCA |
| 481 | GCGATGGGGG CGGGGGGGGG GGGGGCGCGC GCCAGGCGGG GCGGGGGGGG GCGAGGGGCG |
| 541 | GGGGGGGGCG AGGCGGAGAG GTGCGGCGGC AGCCAATCAG AGCGGCGCGC TCCGAAAGTT |
| 601 | TCCTTTTATG GCGAGGCGGC GGCGGCGGCG GCCCTATAAA AAGCGAAGCG CGCGGCGGGC |
| 661 | GGGAGTCGCT GCGTTGCCTT CGCCCCGTGC CCCGCTCCGC GCCGCCTCGC GCCGCCCGCC |
| 721 | CCGGCTCTGA CTGACCGCGT TACTCCCACA GGTGAGCGGG CGGGACGGCC CTTCTCCTCC |
| 781 | GGGCTGTAAT TAGCGCTTGG TTTAATGACG GCTCGTTTCT TTTCTGTGGC TGCGTGAAAG |
| 841 | CCTTAAAGGG CTCCGGGAGG GCCCTTTGTG CGGGGGGGAG CGGCTCGGGG GGTGCGTGCG |
| 901 | TGTGTGTGTG CGTGGGGAGC GCCGCGTGCG GCCCGCGCTG CCCGGCGGCT GTGAGCGCTG |
| 961 | CGGGCGCGGC GCGGGGCTTT GTGCGCTCCG CGTGTGCGCG AGGGGAGCGC GGCCGGGGGC |
| 1021 | GGTGCCCCGC GGTGCGGGGG GGCTGCGAGG GGAACAAAGG CTGCGTGCGG GGTGTGTGCG |
| 1081 | TGGGGGGGTG AGCAGGGGGT GTGGGCGCGG CGGTCGGGCT GTAACCCCCC CCTGCACCCC |
| 1141 | CCTCCCCGAG TTGCTGAGCA CGGCCCGGCT TCGGGTGCGG GGCTCCGTGC GGGGCGTGGC |
| 1201 | GCGGGGCTCG CCGTGCCGGG CGGGGGGTGG CGGCAGGTGG GGGTGCCGGG CGGGGGGGGG |
| 1261 | CCGCCTCGGG CCGGGGAGGG CTCGGGGGAG GGGCGCGGCG GCCCCGGAGC GCCGGCGGCT |
| 1321 | GTCGAGGCGC GGCGAGCCGC AGCCATTGCC TTTTATGGTA ATCGTGCGAG AGGGCGCAGG |
| 1381 | GACTTCCTTT GTCCCAAATC TGGCGGAGCC GAAATCTGGG AGGCGCCGCC GCACCCCCTC |
| 1441 | TAGCGGGCGC GGGCGAAGCG GTGCGGCGCC GGCAGGAAGG AAATGGGCGG GGAGGGCCTT |
| 1501 | CGTGCGTCGC CGCGCCGCCG TCCCCTTCTC CATCTCCAGC CTCGGGGCTG CCGCAGGGGG |
| 1561 | ACGGCTGCCT TCGGGGGGGA CGGGGCAGGG CGGGGTTCGG CTTCTGGCGT GTGACCGGCG |
| 1621 | GCTCTAGAGC CTCTGCTAAC CATGTTCATG CCTTCTTCTT TTTCCTACAG CTCCTGGGCA |
| 1681 | ACGTGCTGGT TGTTGTGCTG TCTCATCATT TTGGCAAAGG CCACCATGTT CGTGTTTCTG |
| 1741 | GTGCTGCTGC CTCTGGTGTC CAGCCAGTGT GTGAACCTGA CCACAAGAAC CCAGCTGCCT |
| 1801 | CCAGCCTACA CCAACAGCTT TACCAGAGGC GTGTACTACC CCGACAAGGT GTTCAGATCC |
| 1861 | AGCGTGCTGC ACTCTACCCA GGACCTGTTC CTGCCTTTCT TCAGCAACGT GACCTGGTTC |
| 1921 | CACGCCATCC ACGTGTCCGG CACCAATGGC ACCAAGAGAT TCGACAACCC CGTGCTGCCC |
| 1981 | TTCAACGACG GGGTGTACTT TGCCAGCACC GAGAAGTCCA ACATCATCAG AGGCTGGATC |
| 2041 | TTCGGCACCA CACTGGACAG CAAGACCCAG AGCCTGCTGA TCGTGAACAA CGCCACCAAC |
| 2101 | GTGGTCATCA AAGTGTGCGA GTTCCAGTTC TGCAACGACC CCTTCCTGGG CGTCTACTAT |
| 2161 | CACAAGAACA ACAAGAGCTG GATGGAAAGC GAGTTCCGGG TGTACAGCAG CGCCAACAAC |
| 2221 | TGCACCTTCG AGTACGTGTC CCAGCCTTTC CTGATGGACC TGGAAGGCAA GCAGGGCAAC |
| 2281 | TTCAAGAACC TGCGCGAGTT CGTGTTCAAG AACATCGACG GCTACTTCAA GATCTACAGC |
| 2341 | AAGCACACCC CTATCAACCT CGTGCGGGAT CTGCCTCAGG GCTTCTCTGC TCTGGAACCC |
| 2401 | CTGGTGGATC TGCCCATCGG CATCAACATC ACCCGGTTTC AGACACTGCT GGCCCTGCAC |
| 2461 | AGAAGCTACC TGACACCTGG CGATAGCAGC AGCGGATGGA CAGCTGGTGC CGCCGCTTAC |
| 2521 | TATGTGGGCT ACCTGCAGCC TAGAACCTTT CTGCTGAAGT ACAACGAGAA CGGCACCATC |
| 2581 | ACCGACGCCG TGGATTGTGC TCTGGATCCT CTGAGCGAGA CAAAGTGCAC CCTGAAGTCC |
| 2641 | TTCACCGTGG AAAAGGGCAT CTACCAGACC AGCAACTTCC GGGTGCAGCC CACCGAATCC |
| 2701 | ATCGTGCGGT TCCCCAATAT CACCAATCTG TGCCCCTTCG GCGAGGTGTT CAATGCCACC |
| 2761 | AGATTCGCCT CTGTGTACGC CTGGAACCGG AAGCGGATCA GCAATTGCGT GGCCGACTAC |
| 2821 | TCCGTGCTGT ACAACTCCGC CAGCTTCAGC ACCTTCAAGT GCTACGGCGT GTCCCCTACC |
| 2881 | AAGCTGAACG ACCTGTGCTT CACAAACGTG TACGCCGACA GCTTCGTGAT CCGGGGAGAT |
| 2941 | GAAGTGCGGC AGATTGCCCC TGGACAGACA GGCAAGATCG CCGACTACAA CTACAAGCTG |
| 3001 | CCCGACGACT TCACCGGCTG TGTGATTGCC TGGAACAGCA ACAACCTGGA CTCCAAAGTC |
| 3061 | GGCGGCAACT ACAATTACCT GTACCGGCTG TTCCGGAAGT CCAATCTGAA GCCCTTCGAG |
| 3121 | CGGGACATCT CCACCGAGAT CTATCAGGCC GGCAGCACCC CTTGTAACGG CGTGGAAGGC |
| 3181 | TTCAACTGCT ACTTCCCACT GCAGTCCTAC GGCTTTCAGC CCACAAATGG CGTGGGCTAT |
| 3241 | CAGCCCTACA GAGTGGTGGT GCTGAGCTTC GAACTGCTGC ATGCCCCTGC CACAGTGTGC |
| 3301 | GGCCCTAAGA AAAGCACCAA TCTCGTGAAG AACAAATGCG TGAACTTCAA CTTCAACGGC |
| 3361 | CTGACCGGCA CCGGCGTGCT GACAGAGAGC AACAAGAAGT TCCTGCCATT CCAGCAGTTT |
| 3421 | GGCCGGGATA TCGCCGATAC CACAGACGCC GTTAGAGATC CCCAGACACT GGAAATCCTG |
| 3481 | GACATCACCC CTTGCAGCTT CGGCGGAGTG TCTGTGATCA CCCCTGGCAC CAACACCAGC |
| 3541 | AATCAGGTGG CAGTGCTGTA CCAGGACGTG AACTGTACCG AAGTGCCCGT GGCCATTCAC |
| 3601 | GCCGATCAGC TGACACCTAC ATGGGGGGTG TACTCCACCG GCAGCAATGT GTTTCAGACC |
| 3661 | AGAGCCGGCT GTCTGATCGG AGCCGAGCAC GTGAACAATA GCTACGAGTG CGACATCCCC |
| 3721 | ATCGGCGCTG GCATCTGTGC CAGCTACCAG ACACAGACAA ACAGCCCCGC CTCTGTGGCC |
| 3781 | AGCCAGAGCA TCATTGCCTA CACAATGTCT CTGGGCGCCG AGAACAGCGT GGCCTACTCC |
| 3841 | AACAACTCTA TCGCTATCCC CACCAACTTC ACCATCAGCG TGACCACAGA GATCCTGCCT |
| 3901 | GTGTCCATGA CCAAGACCAG CGTGGACTGC ACCATGTACA TCTGCGGCGA TTCCACCGAG |
| 3961 | TGCTCCAACC TGCTGCTGCA GTACGGCAGC TTCTGCACCC AGCTGAATAG AGCCCTGACA |
| 4021 | GGGATCGCCG TGGAACAGGA CAAGAACACC CAAGAGGTGT TCGCCCAAGT GAAGCAGATC |
| 4081 | TACAAGACCC CTCCTATCAA GGACTTCGGC GGCTTCAATT TCAGCCAGAT TCTGCCCGAT |
| 4141 | CCTAGCAAGC CCAGCAAGCG GAGCTTCATC GAGGACCTGC TGTTCAACAA AGTGACACTG |
| 4201 | GCCGACGCCG GCTTCATCAA GCAGTATGGC GATTGTCTGG GCGACATTGC CGCCAGGGAT |
| 4261 | CTGATTTGCG CCCAGAAGTT TAACGGACTG ACAGTGCTGC CTCCTCTGCT GACCGATGAG |
| 4321 | ATGATCGCCC AGTACACATC TGCCCTGCTG GCCGGCACAA TCACAAGCGG CTGGACATTT |
| 4381 | GGAGCTGGCG CCGCTCTGCA GATCCCCTTT GCTATGCAGA TGGCCTACCG GTTCAACGGC |
| 4441 | ATCGGAGTGA CCCAGAATGT GCTGTACGAG AACCAGAAGC TGATCGCCAA CCAGTTCAAC |
| 4501 | AGCGCCATCG GCAAGATCCA GGACAGCCTG AGCAGCACAG CAAGCGCCCT GGGAAAGCTG |
| 4561 | CAGGACGTGG TCAACCAGAA TGCCCAGGCA CTGAACACCC TGGTCAAGCA GCTGTCCTCC |
| 4621 | AACTTCGGCG CCATCAGCTC TGTGCTGAAC GATATCCTGA GCAGACTGGA CAAGGTGGAA |
| 4681 | GCCGAGGTGC AGATCGACAG ACTGATCACC GGAAGGCTGC AGTCCCTGCA GACCTACGTT |
| 4741 | ACCCAGCAGC TGATCAGAGC CGCCGAGATT AGAGCCTCTG CCAATCTGGC CGCCACCAAG |
| 4801 | ATGTCTGAGT GTGTGCTGGG CCAGAGCAAG AGAGTGGACT TTTGCGGCAA GGGCTACCAC |
| 4861 | CTGATGAGCT TCCCTCAGTC TGCCCCTCAC GGCGTGGTGT TTCTGCACGT GACATACGTG |
| 4921 | CCCGCTCAAG AGAAGAATTT CACCACCGCT CCAGCCATCT GCCACGACGG CAAAGCCCAC |
| 4981 | TTTCCTAGAG AAGGCGTGTT CGTGTCCAAC GGCACCCATT GGTTCGTGAC CCAGCGGAAC |
| 5041 | TTCTACGAGC CCCAGATCAT CACCACCGAC AACACCTTCG TGTCTGGCAA CTGCGACGTC |
| 5101 | GTGATCGGCA TTGTGAACAA TACCGTGTAC GACCCTCTGC AGCCCGAGCT GGACAGCTTC |
| 5161 | AAAGAGGAAC TGGATAAGTA CTTTAAGAAC CACACAAGCC CCGACGTGGA CCTGGGCGAT |
| 5221 | ATCAGCGGAA TCAATGCCAG CGTCGTGAAC ATCCAGAAAG AGATCGACCG GCTGAACGAG |
| 5281 | GTGGCCAAGA ATCTGAACGA GAGCCTGATC GACCTGCAAG AACTGGGGAA GTACGAGCAG |
| 5341 | TACATCAAGT GGCCCAGCGG CCGCTTGGTC CCACGTGGCT CACCCGGATC TGGATACATC |
| 5401 | CCGGAGGCCC CTAGGGACGG TCAAGCTTAC GTGAGAAAGG ACGGCGAATG GGTTCTGCTG |
| 5461 | TCGACCTTCT TGGGACATCA TCATCATCAT CACTAATGAA ATTCGAGCTC GCGGCCGCAT |
| 5521 | CGATCTTAAG TCGCGACTCG AGCTAGCAGA TCTTTTTCCC TCTGCCAAAA ATTATGGGGA |
| 5581 | CATCATGAAG CCCCTTGAGC ATCTGACTTC TGGCTAATAA AGGAAATTTA TTTTCATTGC |
| 5641 | AATAGTGTGT TGGAATTTTT TGTGTCTCTC ACTCGGAAGG ACATATGGGA GGGCAAATCA |
| 5701 | TTTAAAACAT CAGAATGAGT ATTTGGTTTA GAGTTTGGCA ACATATGCCC ATATGCTGGC |
| 5761 | TGCCATGAAC AAAGGTTGGC TATAAAGAGG TCATCAGTAT ATGAAACAGC CCCCTGCTGT |
| 5821 | CCATTCCTTA TTCCATAGAA AAGCCTTGAC TTGAGGTTAG ATTTTTTTTA TATTTTGTTT |
| 5881 | TGTGTTATTT TTTTCTTTAA CATCCCTAAA ATTTTCCTTA CATGTTTTAC TAGCCAGATT |
| 5941 | TTTCCTCCTC TCCTGACTAC TCCCAGTCAT AGCTGTCCCT CTTCTCTTAT GAAGATCCCT |
| 6001 | CGACCTGCAG CCCAAGCTTG GCGTAATCAT GGTCATAGCT GTTTCCTGTG TGAAATTGTT |
| 6061 | ATCCGCTCAC AATTCCACAC AACATACGAG CCGGAAGCAT AAAGTGTAAA GCCTGGGGTG |
| 6121 | CCTAATGAGT GAGCTAACTC ACATTAATTG CGTTGCGCTC ACTGCCCGCT TTCCAGTCGG |
| 6181 | GAAACCTGTC GTGCCAGCGG ATCCGCATCT CAATTAGTCA GCAACCATAG TCCCGCCCCT |
| 6241 | AACTCCGCCC ATCCCGCCCC TAACTCCGCC CAGTTCCGCC CATTCTCCGC CCCATGGCTG |
| 6301 | ACTAATTTTT TTTATTTATG CAGAGGCCGA GGCCGCCTCG GCCTCTGAGC TATTCCAGAA |
| 6361 | GTAGTGAGGA GGCTTTTTTG GAGGCCTAGG CTTTTGCAAA AAGCTAACTT GTTTATTGCA |
| 6421 | GCTTATAATG GTTACAAATA AAGCAATAGC ATCACAAATT TCACAAATAA AGCATTTTTT |
| 6481 | TCACTGCATT CTAGTTGTGG TTTGTCCAAA CTCATCAATG TATCTTATCA TGTCTGGATC |
| 6541 | CGCTGCATTA ATGAATCGGC CAACGCGCGG GGAGAGGCGG TTTGCGTATT GGGCGCTCTT |
| 6601 | CCGCTTCCTC GCTCACTGAC TCGCTGCGCT CGGTCGTTCG GCTGCGGCGA GCGGTATCAG |
| 6661 | CTCACTCAAA GGCGGTAATA CGGTTATCCA CAGAATCAGG GGATAACGCA GGAAAGAACA |
| 6721 | TGTGAGCAAA AGGCCAGCAA AAGGCCAGGA ACCGTAAAAA GGCCGCGTTG CTGGCGTTTT |
| 6781 | TCCATAGGCT CCGCCCCCCT GACGAGCATC ACAAAAATCG ACGCTCAAGT CAGAGGTGGC |
| 6841 | GAAACCCGAC AGGACTATAA AGATACCAGG CGTTTCCCCC TGGAAGCTCC CTCGTGCGCT |
| 6901 | CTCCTGTTCC GACCCTGCCG CTTACCGGAT ACCTGTCCGC CTTTCTCCCT TCGGGAAGCG |
| 6961 | TGGCGCTTTC TCAATGCTCA CGCTGTAGGT ATCTCAGTTC GGTGTAGGTC GTTCGCTCCA |
| 7021 | AGCTGGGCTG TGTGCACGAA CCCCCCGTTC AGCCCGACCG CTGCGCCTTA TCCGGTAACT |
| 7081 | ATCGTCTTGA GTCCAACCCG GTAAGACACG ACTTATCGCC ACTGGCAGCA GCCACTGGTA |
| 7141 | ACAGGATTAG CAGAGCGAGG TATGTAGGCG GTGCTACAGA GTTCTTGAAG TGGTGGCCTA |
| 7201 | ACTACGGCTA CACTAGAAGG ACAGTATTTG GTATCTGCGC TCTGCTGAAG CCAGTTACCT |
| 7261 | TCGGAAAAAG AGTTGGTAGC TCTTGATCCG GCAAACAAAC CACCGCTGGT AGCGGTGGTT |
| 7321 | TTTTTGTTTG CAAGCAGCAG ATTACGCGCA GAAAAAAAGG ATCTCAAGAA GATCCTTTGA |
| 7381 | TCTTTTCTAC GGGGTCTGAC GCTCAGTGGA ACGAAAACTC ACGTTAAGGG ATTTTGGTCA |
| 7441 | TGAGATTATC AAAAAGGATC TTCACCTAGA TCCTTTTAAA TTAAAAATGA AGTTTTAAAT |
| 7501 | CAATCTAAAG TATATATGAG TAAACTTGGT CTGACAGTTA CCAATGCTTA ATCAGTGAGG |
| 7561 | CACCTATCTC AGCGATCTGT CTATTTCGTT CATCCATAGT TGCCTGACTC CCCGTCGTGT |
| 7621 | AGATAACTAC GATACGGGAG GGCTTACCAT CTGGCCCCAG TGCTGCAATG ATACCGCGAG |
| 7681 | ACCCACGCTC ACCGGCTCCA GATTTATCAG CAATAAACCA GCCAGCCGGA AGGGCCGAGC |
| 7741 | GCAGAAGTGG TCCTGCAACT TTATCCGCCT CCATCCAGTC TATTAATTGT TGCCGGGAAG |
| 7801 | CTAGAGTAAG TAGTTCGCCA GTTAATAGTT TGCGCAACGT TGTTGCCATT GCTACAGGCA |
| 7861 | TCGTGGTGTC ACGCTCGTCG TTTGGTATGG CTTCATTCAG CTCCGGTTCC CAACGATCAA |
| 7921 | GGCGAGTTAC ATGATCCCCC ATGTTGTGCA AAAAAGCGGT TAGCTCCTTC GGTCCTCCGA |
| 7981 | TCGTTGTCAG AAGTAAGTTG GCCGCAGTGT TATCACTCAT GGTTATGGCA GCACTGCATA |
| 8041 | ATTCTCTTAC TGTCATGCCA TCCGTAAGAT GCTTTTCTGT GACTGGTGAG TACTCAACCA |
| 8101 | AGTCATTCTG AGAATAGTGT ATGCGGCGAC CGAGTTGCTC TTGCCCGGCG TCAATACGGG |
| 8161 | ATAATACCGC GCCACATAGC AGAACTTTAA AAGTGCTCAT CATTGGAAAA CGTTCTTCGG |
| 8221 | GGCGAAAACT CTCAAGGATC TTACCGCTGT TGAGATCCAG TTCGATGTAA CCCACTCGTG |
| 8281 | CACCCAACTG ATCTTCAGCA TCTTTTACTT TCACCAGCGT TTCTGGGTGA GCAAAAACAG |
| 8341 | GAAGGCAAAA TGCCGCAAAA AAGGGAATAA GGGCGACACG GAAATGTTGA ATACTCATAC |
| 8401 | TCTTCCTTTT TCAATATTAT TGAAGCATTT ATCAGGGTTA TTGTCTCATG AGCGGATACA |
| 8461 | TATTTGAATG TATTTAGAAA AATAAACAAA TAGGGGTTCC GCGCACATTT CCCCGAAAAG |
| 8521 | TGCCACCTG |

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended embodiments.

All references cited herein are incorporated herein by reference in their entirety and for all purposes to the same extent as if each individual publication or patent or patent application was specifically.

What is claimed is:

1. A kit comprising:
(a) a multi-well microtiter ELISA plate coated with a first recombinant soluble SARS-CoV-2 spike protein, wherein the first recombinant soluble SARS-CoV-2 spike protein comprises amino acid residues 319-541 of GenBank Accession No. MN908947.3 and a tag; and
(b) a multi-well ELISA microtiter plate coated with a second recombinant soluble SARS-CoV-2 spike protein, wherein the second recombinant soluble SARS-CoV-2 spike protein comprises amino acid residues 15-1213 of GenBank Accession No. MN908947.3, a C-terminal cleavage site, trimerization domain, and a tag, and wherein the second recombinant soluble SARS-CoV-2 spike protein does not contain a polybasic cleavage site.

2. The kit of claim 1, wherein the SARS-CoV-2 spike protein comprises amino acid residues 1-1213 of GenBank Accession No. MN908947.3.

3. The kit of claim 1 or 2, wherein the tag is a hexahistidine tag.

4. The kit of claim 1 or 2, wherein the C-terminal cleavage site is a C-terminal thrombin cleavage site.

5. The kit of claim 1 or 2, wherein the trimerization domain is a T4 foldon trimerization domain.

6. The kit of claim 1 or 2, wherein the tag is a hexahistidine tag, the C-terminal cleavage site is a C-terminal thrombin cleavage site, and the trimerization domain is a T4 foldon trimerization domain.

7. The kit of claim 1 or 2, wherein the first recombinant soluble SARS-CoV-2 spike protein comprises the amino acid sequence of SEQ ID NO:2.

8. The kit of claim 1 or 2, wherein the second recombinant soluble SARS-CoV-2 spike protein comprises the amino acid sequence of SEQ ID NO:4 or 6.

9. The kit of claim 8, wherein the second recombinant soluble SARS-CoV-2 spike protein comprises the amino acid sequence of SEQ ID NO:4 or 6.

10. The kit of claim 9, wherein the kit further comprises a labeled secondary antibody.

11. The kit of claim 10, wherein the kit further comprises a positive control antibody that binds to the recombinant soluble SARS-CoV-2 spike protein.

12. The kit of claim 11, wherein the positive control antibody is monoclonal antibody CR3022 or antibodies from COVID-19 patients.

13. The kit of claim 10, wherein the kit further comprises a negative control antibody.

14. A kit comprising:
(a) a first multi-well microtiter plate coated with a first recombinant soluble SARS-CoV-2 spike protein, wherein the first recombinant soluble SARS-CoV-2 spike protein comprises amino acid residues 319 to 541 of a SARS-CoV-2 spike protein and a histidine tag at the C-terminus;
(b) a second multi-well microtiter plate coated with a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof;
(c) a vial containing concentrated monoclonal antibody specific to human IgG conjugated to horseradish peroxidase;
(d) a vial containing 3,3′,5,5′-Tetramethylbenzidine (TMB) substrate;
(e) a vial containing a stop solution comprising an acid;
(f) a vial containing a sample buffer comprising phosphate buffered saline (PBS) with 0.5% to 5% milk;
(g) a vial containing a wash buffer comprising phosphate buffered saline and 0.5% to 2% of a surfactant.

15. A kit comprising:
(a) a vial containing a first recombinant soluble SARS-CoV-2 spike protein, wherein the first recombinant soluble SARS-CoV-2 spike protein comprises amino acid residues 319 to 541 of a SARS-CoV-2 spike protein and a histidine tag at the C-terminus;
(b) a vial containing a second recombinant soluble SARS-CoV-2 spike protein, wherein the second recombinant soluble SARS-CoV-2 spike protein comprises the amino acid sequence of a SARS-CoV-2 spike protein ectodomain or a derivative thereof;
(c) a vial containing concentrated monoclonal antibody specific to human IgG conjugated to horseradish peroxidase;
(d) a vial containing 3,3′,5,5′-Tetramethylbenzidine (TMB) substrate;
(e) a vial containing a stop solution comprising an acid;
(f) a vial containing a sample buffer comprising phosphate buffered saline (PBS) with 0.5% to 5% milk; and
(g) a vial containing a wash buffer comprising 0.5% to 2% of a surfactant.

16. The kit of claim 15, wherein the kit further comprises 2 or more multi-well-microtiter plates.

17. The kit of claim 14, wherein first and second multi-well microtiter plates are 96-well microtiter plates.

18. The kit of claim 14 or 17, wherein the kit comprises 4 of the first multi-well microtiter plate and 5 of the second multi-well microtiter plate.

19. The kit of claim 14 or 17, wherein the kit comprises 7 of the first multi-well microtiter plate and 3 of the second multi-well microtiter plate.

20. The kit of any one of claims 14 to 19, wherein the first recombinant soluble SARS-CoV-2 spike protein comprises the amino acid sequence set forth in SEQ ID NO:10.

21. The kit of any one of claims 14 to 20, wherein the vial containing the concentrated monoclonal antibody specific to human IgG conjugated to horseradish peroxidase comprises 100 to 500 μl of the concentrated antibody.

22. The kit of any one of claims 14 to 21, wherein the concentrated monoclonal antibody specific to human IgG conjugated to horseradish peroxidase is 100× to 1000× concentrated monoclonal antibody.

23. The kit of any one of claims 14 to 22, wherein the kit comprises a second vial containing concentrated monoclonal antibody specific to human IgG conjugated to horseradish peroxidase.

24. The kit of any one of claims 14 to 23, wherein the acid is an alkyl sulfonic acid, 1 N HCl or 2 N H₂SO₄.

25. The kit of any one of claims 14 to 24, wherein the alkyl sulfonic acid is 1-5% methanesulfonic acid.

26. The kit of any one of claim 25, wherein the vial comprises 100 to 500 mL of methanesulfonic acid.

27. The kit of any one of claims 14 to 26, wherein the vial containing 3,3′,5,5′-Tetramethylbenzidine (TMB) substrate comprises 100 to 500 mL of 3,3′,5,5′-Tetramethylbenzidine (TMB) substrate.

28. The kit of any one of claims 14 to 27, wherein the vial containing the sample buffer comprises 50 to 500 ml of the sample buffer.

29. The kit of any one of claims 14 to 28, wherein the vial containing the wash buffer comprises 100 to 500 ml of the wash buffer.

30. The kit of any one of claims 14 to 29, wherein the surfactant is a polysorbate or Triton X-100.

31. The kit of claim 30, wherein the polysorbate is polysorbate-20 or polysorbate 80.

32. The kit of any one of claims 14 to 31, wherein the kit further comprises a vial containing a positive control monoclonal antibody specific for SARS-CoV-2 spike protein.

33. The kit of claim 32, wherein the positive control monoclonal antibody specific for SARS-CoV-2 spike protein binds to the receptor binding domain.

34. The kit of claim 32 or 33, wherein the positive control monoclonal antibody specific for SARS-CoV-2 spike protein is an IgG antibody.

35. The kit of any one of claims 32 to 34, wherein the vial containing the positive control monoclonal antibody comprises 500 μl to 2 ml of the positive control monoclonal antibody.

36. The kit of any one of claims 32 to 35, wherein the positive control monoclonal antibody is diluted 1:5 in the sample buffer.

37. The kit of any one of claims 14 to 36, wherein the kit further comprises a vial containing a negative control.

38. The kit of claim 37, wherein the negative control is a control buffer.

39. The kit of claim 38, wherein the negative control is a monoclonal antibody that does not bind to the SARS-CoV-2 spike protein.

40. The kit of any one of claims 37 to 39, wherein the vial containing the negative control comprises 500 μl to 2 ml of the negative control.

41. The kit of any one of claims 14 to 40, wherein the kit further comprises a second vial comprising a monoclonal antibody specific for SARS-CoV-2 spike protein.

42. The kit of claim 41, wherein the second vial comprises 500 μl to 2 ml of the monoclonal antibody specific for SARS-CoV-2 spike protein.

43. The kit of any one of claims 14 to 42, wherein the kit further comprises at least 3 calibrators, wherein each calibrator is a monoclonal antibody specific for SARS-CoV-2 spike protein and each calibrator has 0 to 200 arbitrary units (AU)/ml, and wherein each calibrator has a different AU/ml.

44. The kit of any one of claims 14 to 43, wherein the kit further comprises 7 calibrators, wherein each calibrator is a monoclonal antibody specific for SARS-CoV-2 spike protein and each calibrator has 0 to 200 AU/ml, and wherein each calibrator has a different AU/ml.

45. The kit of any one of claims 14 to 44, which is stored at 2° to 8° C.

46. The kit of any one of claims 14 to 45, wherein the second recombinant soluble SARS-CoV-2 spike protein comprises the amino acid sequence of SEQ ID NO: 4 or SEQ ID NO:6 without the first 14 amino acid residues.

47. The kit of any one of claims 14 to 45, wherein the second recombinant soluble SARS-CoV-2 spike protein comprises the amino acid sequence of SEQ ID NO: 4 or SEQ ID NO:6.

48. A method for detecting the presence of antibodies in a subject that are specific for the SARS-CoV-2 spike protein comprising contacting in vitro a biological sample from said subject with a recombinant soluble SARS-CoV-2 spike protein and detecting the binding of the antibody to the recombinant soluble SARS-CoV2 spike protein using a labeled antibody that binds to the constant region of the antibodies specific for the SARS-CoV-2 spike protein, wherein the recombinant soluble SARS-CoV-2 spike protein comprises (i) amino acid residues 319-541 of GenBank Accession No. MN908947.3 and a tag or (ii) amino acid residues 15-1213 of GenBank Accession No. MN908947.3, a C-terminal cleavage site, trimerization domain, and a tag, wherein the recombinant soluble SARS-CoV-2 spike protein does not contain a polybasic cleavage site.

49. The method of claim 48, wherein the recombinant soluble SARS-CoV-2 spike protein comprises amino acid residues 1-1213 of GenBank Accession No. MN908947.3.

50. The method of claim 48 or 49, wherein the biological sample is blood plasma, sera or saliva.

51. The method of claim 48 or 49, wherein the tag is a hexahistidine tag.

52. The method of claim 48 or 49, wherein the C-terminal cleavage site is a C-terminal thrombin cleavage site.

53. The method of claim 48 or 49, wherein the trimerization domain is a T4 foldon trimerization domain.

54. The method of claim 48 or 49, wherein the tag is a hexahistidine tag, the C-terminal cleavage site is a C-terminal thrombin cleavage site, and the trimerization domain is a T4 foldon trimerization domain.

55. The method of claim 48 or 49, wherein the recombinant soluble SARS-CoV-2 spike protein comprises the amino acid sequence of SEQ ID NO:2, 4, 6 or 10.

56. The method of claim 48 or 49, wherein the recombinant soluble SARS-CoV-2 spike protein comprises the amino acid sequence of SEQ ID NO:2, 4 or 6 without the first 14 amino acid residues.

57. A method for the detection of antibody that specifically binds to human SARS-CoV-2 spike protein, comprising:
(a) incubating a specimen in a well coated with a first recombinant soluble SARS-CoV-2 spike protein for a first period of time, wherein the first recombinant soluble SARS-CoV-2 spike protein comprises amino acid residues 319-541 of GenBank Accession No. MN908947.3 and a tag;
(b) washing the well;
(c) incubating a labeled antibody that binds to an isotype or subtype of an immunoglobulin for a second period of time in the well;
(d) washing the well;
(e) detecting the binding of the labeled antibody to the first recombinant SARS-CoV-2 spike protein;
(f) incubating the specimen in a well coated with a second recombinant soluble SARS-CoV-2 spike protein for a third period of time, wherein the second recombinant soluble SARS-CoV-2 spike protein comprises amino acid residues 15-1213 of GenBank Accession No. MN908947.3, a C-terminal cleavage site, trimerization domain, and a tag, and wherein the second recombinant soluble SARS-CoV-2 spike protein does not contain a polybasic cleavage site;
(g) washing the well;
(h) incubating a second labeled antibody that binds to an isotype or subtype of an immunoglobulin for a fourth period of time in the well;
(i) washing the well; and
(j) detecting the binding of the second labeled antibody to the second recombinant SARS-CoV-2 spike protein.

58. The method of claim 57, wherein the second recombinant SARS-CoV-2 spike protein comprises amino acid residues 1-1213 of GenBank Accession No. MN908947.3.

59. The method of claim 57 or 58, wherein the first time period, second time period or both is 30 minutes, 45 minutes, 1 hour, 2 hours, 2.5 hours or 3 hours.

60. The method of any one of claims 57 to 59, wherein the specimen is a biological sample.

61. The method of claim 60, wherein the biological sample is blood, sera, or plasma.

62. The method of claim 61, wherein the biological sample is inactivated before being incubated with the well coated with recombinant soluble SARS-CoV-2 spike protein.

63. The method of any one of claims 57 to 62, wherein the specimen is serially diluted.

64. The method of any one of claims 57 to 63, wherein the labeled antibody is labeled with a radioactive moiety, a chemiluminescent moiety, or a fluorescent moiety.

65. The method of any one of claims 57 to 64, wherein the tag is a hexahistidine tag.

66. The method of any one of claims 57 to 64, wherein the C-terminal cleavage site is a C-terminal thrombin cleavage site.

67. The method of any one of claims 57 to 64, wherein the trimerization domain is a T4 foldon trimerization domain.

68. The method of any one of claims 57 to 64, wherein the tag is a hexahistidine tag, the C-terminal cleavage site is a C-terminal thrombin cleavage site, and the trimerization domain is a T4 foldon trimerization domain.

69. The method of any one of claims 57 to 64, wherein the second recombinant soluble SARS-CoV-2 spike protein comprises the amino acid sequence of SEQ ID NO: 4 or 6.

70. The method of any one of claims 57 to 64, wherein the second recombinant soluble SARS-CoV-2 spike protein comprises the amino acid sequence of SEQ ID NO:4 or 6 without the first 14 amino acid residues.

71. The method of any one of claims 57 to 64, wherein the first recombinant soluble SARS-CoV-2 spike protein comprises the amino acid sequence of SEQ ID NO:2 or 10.

72. The method of any one of claims 57 to 71, wherein the third and fourth time periods are 30 minutes, 45 minutes, 1 hour, 2 hours, 2.5 hours or 3 hours.

73. A method for detecting antibody specific for SARS-CoV-2 spike protein, comprising:
(a) incubating a diluted heat inactivated biological sample in a well of a multi-well microtiter plate coated with a first recombinant soluble SARS-CoV-2 spike protein for 1.5 to 2.5 hours at room temperature, wherein the first recombinant soluble SARS-CoV-2 spike protein comprises the receptor binding domain of a SARS-CoV-2 spike protein and optionally a tag at the C-terminus, and wherein the biological sample is serum or plasma from a human subject;
(b) washing the well with wash buffer, wherein the wash buffer comprises phosphate buffered saline and 0.5% to 2% of a surfactant;
(c) incubating a monoclonal antibody specific to human IgG conjugated to horseradish peroxidase in the well for 30 minutes to 1.5 hours at room temperature;
(d) washing the well with the wash buffer;
(e) incubating a substrate solution in the well for 10 to 40 minutes at room temperature, wherein the substrate solution comprises 3,3′,5,5′-Tetramethylbenzidine (TMB) substrate;
(f) adding stop solution to the well, wherein the stop solution comprises an acid;
(g) determining the optical density of the well within 30 minutes using a microtiter plate reader set to 450 nm to detect antibody specific for SARS-CoV-2 spike protein, wherein if wavelength correction is available set at 540 nm or 570 nm, or if wavelength correction is not available, readings at 540 nm or 570 nm are taken and subtracted from readings at 450 nm;
and wherein the microtiter plate comprises a second well and a third well, and wherein steps (a) through (g) are concurrently performed with respect to the second and third wells except that in step (a): (i) a positive control monoclonal antibody specific for SARS-CoV-2 spike protein is used in the second well instead of the diluted biological sample and (ii) a negative control is used in the third well instead of the diluted biological sample;
(h) corrected optical density values for the biological sample are divided by corrected optical density values for the second well to calculate the cutoff interval for the biological sample, wherein a cutoff interval value of greater than or equal to 0.7 indicates that the biological sample is positive for antibody specific for the SARS-CoV-2 spike protein and a cutoff interval value of less than 0.7 indicates that the biological sample is negative for antibody specific for the SARS-CoV-2 spike protein, and wherein a quantitative assay to assess quantitative levels of IgG antibodies against SARS-CoV-2 spike protein for those biological samples identified as positive, and wherein the quantitative assay comprises:
(i) incubating for 1.5 to 2.5 hours at room temperature of a multi-well microtiter plate coated with a second recombinant soluble SARS-CoV-2 spike protein, wherein different wells of the microtiter plate contain either a diluted heat inactivated biological sample(s), one of seven calibrators, or one of three diluted positive controls, wherein each of the seven calibrators is a monoclonal antibody specific for SARS-CoV-2 spike protein and each calibrator has 0 to 200 AU/ml, wherein each calibrator has a different AU/ml, wherein each of the three positive controls is a monoclonal antibody specific for SARS-CoV-2 spike protein, wherein each of the three positive controls have a different AU/ml across a range of 0 to 200 AU/ml, wherein the second recombinant soluble SARS-CoV-2 spike protein comprises the amino acid sequence of a SARS-CoV-2 spike protein ectodomain or a derivative thereof, and wherein the biological sample is serum or plasma from a human subject;
(j) washing each of the wells with the wash buffer;
(k) incubating monoclonal antibody specific to human IgG conjugated to horseradish peroxidase in each of the wells for 30 minutes to 1.5 hours at room temperature;
(l) washing each of the wells with the wash buffer;
(m) incubating the substrate solution in each of the wells for 10 to 40 minutes at room temperature;
(n) adding the stop solution to each of the wells; and
(o) determining the optical density of each of the wells within 30 minutes using a microtiter plate reader set to 450 nm to detect antibody specific for SARS-CoV-2 spike protein.

74. The method of claim 73, wherein the first recombinant soluble SARS-CoV-2 spike protein comprises the amino acid sequence of SEQ ID NO: 2 or 10.

75. The method of claim 73 or 74, wherein the second recombinant soluble SARS-CoV-2 spike protein comprises a C-terminal cleavage site and trimerization domain, and optionally a tag.

76. The method of claim 73 or 74, wherein the second recombinant soluble SARS-CoV-2 spike protein comprises the amino acid residues of SEQ ID NO: 4 or 6 without the first 14 amino acid residues.

77. The method of any one of claims 73 to 76, wherein the acid is an alkylsuphonic acid, 1 N HCl or 2 N H₂SO₄.

78. The method of claim 77, wherein the alkylsuphonic acid is 1-5% methanesulfonic acid

79. The method of any one of claims 73 to 78, wherein the 100 μl of diluted heat inactivated biological sample is incubated in the well of the multi-well microtiter plate in step (a).

80. The method of any one of claims 73 to 79, wherein the biological sample is heat inactivated at 56° C. for 30 minutes to 1 hour.

81. The method of any one of claims 73 to 80, wherein the heat inactivated biological sample is diluted to a final dilution of 1:100 in sample buffer, wherein the sample buffer comprises phosphate buffered saline (PBS) with 0.5% to 5% milk.

82. The method of any one of claims 73 to 81, wherein the well is washed in steps (b), (d), (j) and (1) two or three times.

83. The method of claim 82, wherein each time the well is washed 400 μl of the wash buffer is used.

84. The method of any one of claims 73 to 83, wherein 100 μl of the substrate solution is incubated in the well in steps (e) and (m).

85. The method of any one of claims 73 to 84, wherein the incubation in step (e) is for about 20 minutes.

86. The method of any one of claims 73 to 85, wherein 100 μl of the stop solution is added to the well in steps (f) and (n).

87. The method of any one of claims 73 to 86, wherein the positive control monoclonal antibody in step (g) is diluted in the sample buffer.

88. The method of claim 87, wherein the positive control monoclonal antibody specific is diluted 1:5 in the sample buffer.

89. The method of any one of claims 73 to 87, wherein the positive control monoclonal antibody in step (g) binds to the receptor binding domain of the SARS-CoV-2 spike protein.

90. The method of any one of claims 73 to 89, wherein the negative control is a monoclonal antibody that does not bind to the SARS-CoV-2 spike protein.

91. The method of claim 90, wherein the negative control antibody is diluted 1:5 in the sample buffer.

92. The method of any one of claims 73 to 91, wherein the negative control is a control buffer.

93. The method of any one of claims 73 to 92, wherein 100 μl of the monoclonal antibody specific to human IgG conjugated to horseradish peroxidase is incubated in each of the wells in steps (c) and (k).

94. The method of any one of claims 73 to 93, wherein the three controls are diluted 1:5 in the sample buffer.

95. The method of any one of claims 73 to 94, wherein in step (o) if wavelength correction is available set at 540 nm or 570 nm, or if wavelength correction is not available, readings at 540 nm or 570 nm are taken and subtracted from readings at 450 nm.

96. The method of any one of claims 73 to 95, wherein the quantitative assay further comprises: (p) generating a calibration curve and comparing the signal from the diluted heat inactivated biological sample(s) to the calibration curve to generate a final result of IgG levels in arbitrary units per milliliter (AU/ml).

97. The method of claim 96, wherein less than 3.2 AU/ml indicates that the human subject is negative for SARS-CoV-2 antibody or that there was no detectable immune response, 3.2 AU/ml to 10 AU/Ml indicates that the human subject is likely positive, 10 AU/ml to 25 AU/ml indicates that the human subject has moderate antibody levels, and greater than 25 AU/mL indicates that the human subject has high antibody levels.

98. The method of claim 97, wherein 10 AU/ml to 25 AU ml and greater than 25 AU/ml indicates that the IgG levels specific for SARS-CoV-2 exhibit viral neutralizing activity in vitro.
